(19) United States
(12) Patent Application Publication
(10) Pub. No.: US 2007/0191272 A1
(43) Pub. Date: Aug. 16, 2007

(54) Proteinaceous pharmaceuticals and uses thereof

(76) Inventors: Willem P.C. Stemmer, Los Gatos, CA (US); Volker Schellenberger, Palo Alto, CA (US); Martin Bader, Mountain View, CA (US); Michael Scholle, Mountain View, CA (US)

Correspondence Address: WILSON SONSINI GOODRICH & ROSATI, 650 PAGE MILL ROAD, PALO ALTO, CA, 94304-1050, US

(21) Appl. No.: 11/528,927

(22) Filed: Sep. 27, 2006

Related U.S. Application Data
(60) Provisional application No. 60/721,270, filed on Sep. 27, 2005.
(60) Provisional application No. 60/721,188, filed on Sep. 27, 2005.
(60) Provisional application No. 60/743,622, filed on Mar. 21, 2006.

(51) Int. Cl. A61K 38/16; C40B 40/08; C40B 40/10; C07K 14/47

(52) U.S. Cl. 514/12; 435/7.1; 435/6; 530/324

(57) ABSTRACT

The present invention provides cysteine-containing scaffolds and/or proteins, expression vectors, host cell and display systems harboring and/or expressing such cysteine-containing products. The present invention also provides methods of designing libraries of such products, methods of screening such libraries to yield entities exhibiting binding specificities towards a target molecule. Further provided by the invention are pharmaceutical compositions comprising the cysteine-containing products of the present invention.

### CROSS-REFERENCE

This application claims priority to U.S. Provisional Application Nos. 60/721,270 and 60/721,188, both filed on Sep. 27, 2005, and U.S. Provisional Application No. 60/743,622 filed on Mar. 21, 2006, all which are incorporated herein by reference in their entirety.

### BACKGROUND OF THE INVENTION

One of the fundamental concepts of molecular biology is that each natural protein adopts a single ‘native’ structure or fold. Adoption of any fold other than the native fold is regarded as ‘misfolding’. Few or no examples exist of natural proteins adopting multiple native, functional folds. Misfolding is a serious problem, exemplified by the infectious nature of prions, whose ‘wrong’ fold causes other prion proteins to misfold in a catalytic manner and leads to brain disease and certain death. Almost any protein, when denatured, can misfold to form fibrillar polymers, which appear to be involved in a number of degenerative diseases. An example are the beta-amyloid fibrils involved in Alzheimer's disease. Misfolding of proteins generally results in the irreversible formation of insoluble aggregates, but denatured proteins can also occur as molten globules. From a molten globule state, which explores a huge diversity of unstable structures, the protein is thought to follow a funnel-shaped pathway, gradually reducing the diversity of folding intermediates until a single, stably folded native structure is achieved. The native protein can be altered structurally by allosteric regulation, lid/flap-type movements of one domain relative to other domains, induced fit upon binding to a ligand, or by crystallization forces, but these alterations generally involve movement in hinge-like structures rather than fundamental change in the basic fold. All of the available examples support the notion that natural proteins have evolved to adopt a single stable fold to effect their biological function, and that deviation from this native structure is deleterious.

There have been a few examples of the same protein sequence (excluding variants created by alternative splicing, glycosylation or proteolytic processing) existing naturally in more than one form, but the second form is usually simply an inactive by-product which has lost a disulfide bond (Schulz et al., 2005; Petersen et al., 2003; Lauber et al., 2003). In the microprotein family, which include small proteins with high disulfide density (mostly toxins and receptor-domains), examples have been found of closely related sequences adopting a different structure due to fully formed (not simply defective) but alternative disulfide bonding pattern. Examples are Somatomedin (Kamikubo et al., 2004) and Maurotoxin (Fajloun et al., 2000).

Protein display libraries have traditionally used a single fixed protein fold, like immunoglobulin domains of various species, Interferons, Protein A, Ankyrins, A-domains, T-cell receptors, Fibronectin III, gamma-Crystallin, Ubiquitin and many others, as reviewed in Binz, A. et al. (2005) Nature Biotechnology 23:1257. In some cases, like immunoglobulin libraries derived from the human immune repertoire, a single library uses many different V-region sequences as scaffolds, but they all share the basic immunoglobulin fold. A different type of library is the random peptide or cyclic peptide library, but these are not considered proteins since they do not have any defined fold and do not adopt a single stable structure.

There remains a considerable need for the design of novel protein structures that are amenable to rational selection via, e.g., directed evolution to create therapeutics that exhibit one or more desirable properties. Such desired properties include but are not limited to reduced immunogenicity, enhanced stability or half life, multispecificity, multivalency, and high target binding affinity.

### SUMMARY OF THE INVENTION

One aspect of the present invention is the design of novel protein structures exhibiting high disulfide density. The protein structures are particularly amenable to rational design and selection via, e.g., directed evolution to create therapeutics that exhibit one or more desirable properties. Such desired properties include but are not limited to high target binding affinity and/or avidity, reduced molecular weight and improved tissue penetration, enhanced thermal and protease stability, enhanced shelflife, enhanced hydrophilicity, enhanced formulation (esp. high concentration), and reduced immunogenicity.

In one embodiment, the present invention provides various protein structures in form of, e.g. scaffolds, and libraries of such protein structures. In one aspect, the scaffolds exhibit a diversity of folds or other non-primary structures. In another aspect, the scaffolds have defined topologies to effect the biological functions. In another embodiment, the present invention provides methods of constructing libraries of such protein structures, methods of displaying such libraries on genetic vehicles or packages (e.g., viral packages such as phages or the like, and non-viral packages (such as yeast display, E. coli surface display, ribosome display, or CIS (DNA-linked) display), as well as methods of screening such libraries to yield therapeutics or candidate therapeutics. The present invention further provides vectors, host cells and other in vitro systems expressing or utilizing the subject protein structures.

In another embodiment, the present invention provides a non-naturally occurring cysteine (C)-containing scaffold exhibiting a binding specificity towards a target molecule, wherein the non-naturally occurring cysteine (C)-containing scaffold comprise intra-scaffold cysteines according to a pattern selected from the group of permutations represented by the formula

$${\prod }_{i = 1}^{n} 2 i - 1 ,$$

wherein n equals to the predicted number of disulfide bonds formed by the cysteine residues, and wherein Π represents the product of (2i−1), where i is a positive integer ranging from 1 up to n.

In another embodiment, the present invention provides a non-naturally occurring cysteine (C)-containing protein comprising a polypeptide having no more than 35 amino acids, in which at least 10% of the amino acids in the polypeptide are cysteines, at least two disulfide bonds are formed by pairing intra-scaffold cysteines, and wherein said pairing yields a complexity index greater than 3.

In one aspect, the non-naturally occurring cysteine (C)-containing protein may comprise a polypeptide having no more than about 60 amino acids, in which at least 10% of the amino acids in the polypeptide are cysteines, at least four disulfide bonds are formed by pairing cysteines contained in the polypeptide, and wherein said pairing yields a complexity index greater than 4, 6, or 10.

In another aspect, the non-naturally occurring cysteine (C)-containing protein of the present invention exhibits the target binding capability after being heated to a temperature higher than about 50° C., preferably higher than about 80° C. or even higher than 100° C. for a given period of time, which may range from 0.001 second to 10 minutes.

In some aspects, the non-naturally occurring cysteine (C)-containing protein described herein is conjugated to a moiety selected from the group consisting of labels (i.e., GFP, HA-tag, Flag, Cy3, Cy5, FITC), effectors (ie enzymes, cytotoxic drugs, chelates), antibodies (ie whole antibodies, Fc region, dAbs, scFvs, diabodies), targeting modules (peptides or domains, such as the VEGF heparin binding exons) that concentrate the molecule in a desired tissue or compartment such as a tumor, barrier-transport conjugates that enhance transport across tissue barriers (transdermal, oral, intestinal, buccal, vaginal, rectal, nasal, pulmonary, blood-brain-barrier, transscleral) such as arginine rich peptides, alkyl saccharides, (ionic or non-ionic) amphipathic or amphiphilic peptides that mimic detergents and form micelles containing or displaying the protein, and half-life extending moieties including small molecules (for example those that bind to albumin or insert into the cell membrane), chemical polymers such as polethyleneglycol (PEG) or a variety of peptide and protein sequences (including hydrophobic peptides that may insert into the membrane or bind nonspecifically), (human) serum albumin, transferrin, polymeric glycine-rich sequences such as poly(GGGS) linkers. The linkages forming these conjugates may be formed genetically or chemically. The cysteine-containing proteins can also be homo- or hetero-multimerized to form 2-mers, 3-mers, 4-mers, 5-mers, 6-mers, 7-mers, 8-mers, 9-mers, 10-mers, 11-mers, 12-mers, 14-mers, 16-mers, 18-mers, 20-mers or even higher order multimers, which will extend the halflife of the protein, increase the concentration of binding sites and thus improve the apparent association constant and, depending on the target, may increase the binding avidity as well. The higher order multimers can be created via fusion into a single large gene, or by adding genetically encoded peptide-binding-peptides (‘association peptides’) onto the protein such that separately expressed proteins bind to each other via the association peptides at the N- and/or C-terminus, forming protein multimers, or via a variety of chemical linkages. Suitable half-life extending moieties include but are not limited to moieties that bind to serum albumin, IgG, erythrocytes, and proteins accessible to the serum. Each target and each therapeutic use favors a different combination of multiple of these elements.

The present invention also provides a non-natural protein containing a single domain of 20-60 amino acids which has 3 or more disulfides and binds to a human serum-exposed protein and has less than 5% aliphatic amino acids.

The present invention further provides a non-naturally occurring protein containing a single domain of 20-60 amino acids which has 3 or more disulfides and binds to a human serum-exposed protein and has a score in the T-Epitope program that is lower than 90% of the average for proteins in the database, preferably lower than 99% of the average for proteins in the database, and more preferably lower than 99% of average human proteins in the database. Also included in the present invention are libraries of the subject non-naturally occurring proteins, expression vectors including genetic packages encoding the proteins, as well as other host cells expressing or displaying the proteins.

Further included in the present invention are methods of producing the cysteine-containing microproteins disclosed herein.

Also encompassed in the present invention is a method of detecting the presence of a specific interaction between a target and an exogenous polypeptide that is displayed on a genetic package. The method involves the steps of (a) providing a genetic package displaying of the present invention; (b) contacting the genetic package with the target under conditions suitable to produce a stable polypeptide-target complex; and (c) detecting the formation of the stable polypeptide-target complex on the genetic package, thereby detecting the presence of a specific interaction. The method may further comprise the step of isolating the genetic package that displays a polypeptide having the desired property, or sequencing the portion of the sequence carried by the genetic package that encodes the desired polypeptide. Exemplary genetic packages include but are not limited viruses (e.g. phages), cells and spores.

### BRIEF DESCRIPTION OF THE DRAWINGS

FIGS. 1-12, 14-16, 20-35, 37-73, 75-83, 85-93, 95-97, 99, 101-102, 104-107, 111, 113-115, 123 depict various scaffolds and motifs contained therein.

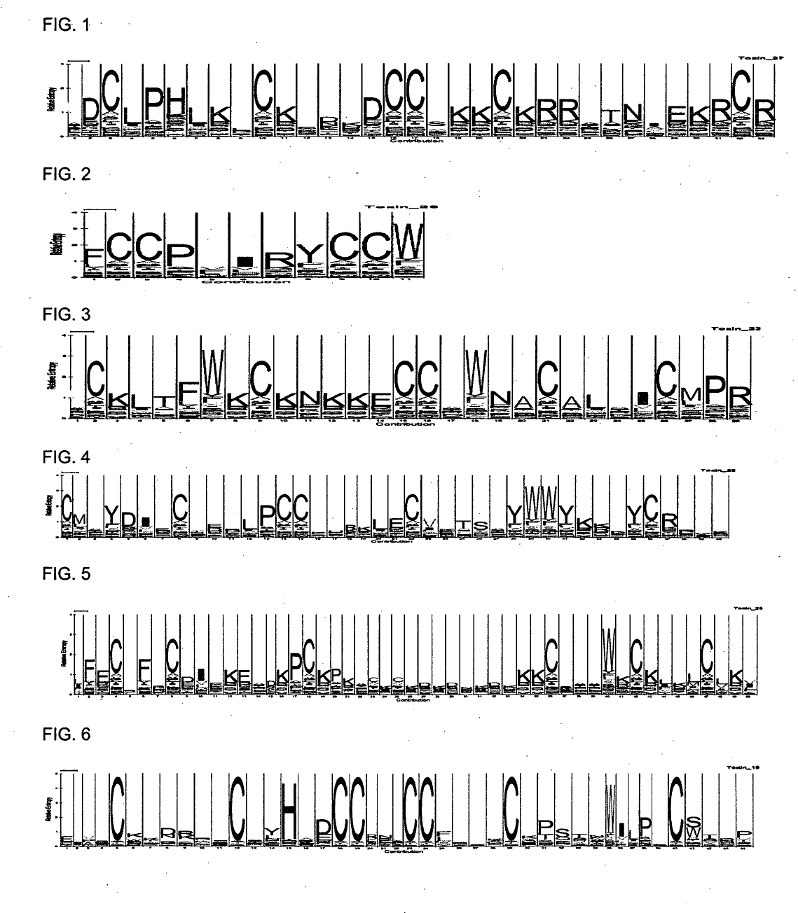

Motif for FIG. 1:

1) CxPhxxxCxxxxdCCxxxCxrrGxxxxxrC

2) CxPxxxxCxxxxxCCxxxCxxxxGxxxxxC

3) CxxxxxxCxxxxxCCxxxCxxxxxxxxxxC

CDP: C6C5C0C3C10C

Motif for FIG. 2:

1) fCCPxxryCCw

2) CCPxxxxCCW

3) CCxxxxxCC

CDP: C0C5C0C

Motif for FIG. 3:

1) CxxxfWxCxxxxxCCgwxxCxxgxC

2) CxxxxWxCxxxxxCCxWxxCxxxxC

3) CxxxxxxCxxxxxCCxxxxCxxxxC

CDP: C6C5C0C4C4

Motif for FIG. 4:

1) CxgydxxCxxxxpCCxxxxxxxCxxxxgyWWyxxxyC

2) CxxxxxxCxxxxxCCxxxxxxxCxxxxxxWWxxxxxC

3) CxxxxxxCxxxxxCCxxxxxxxCxxxxxxxxxxxxxC

CDP: C6C5C0C7C13C

Motif for FIG. 5:

1) CxfxCxxxxxgxxpCxxxxxxxxxxxxxxxxxCxggWxCxxxxC

2) CxxxCxxxxxxxxxCxxxxxxxxxxxxxxxxxCxxxWxCxxxxC

3) CxxxCxxxxxxxxxCxxxxxxxxxxxxxxxxxCxxxxxCxxxxC

CDP: C3C9C17C5C4C

Motif for FIG. 6:

1) CxxxxxxCxxHxxCCxxxCxxgxCxxxxxwxxxgC

2) CxxxxxxCxxHxxCCxxxCxxxxCxxxxxxxxxxC

3) CxxxxxxCxxxxxCCxxxCxxxxCxxxxxxxxxxC

CDP: C6C5C0C3C4C10C

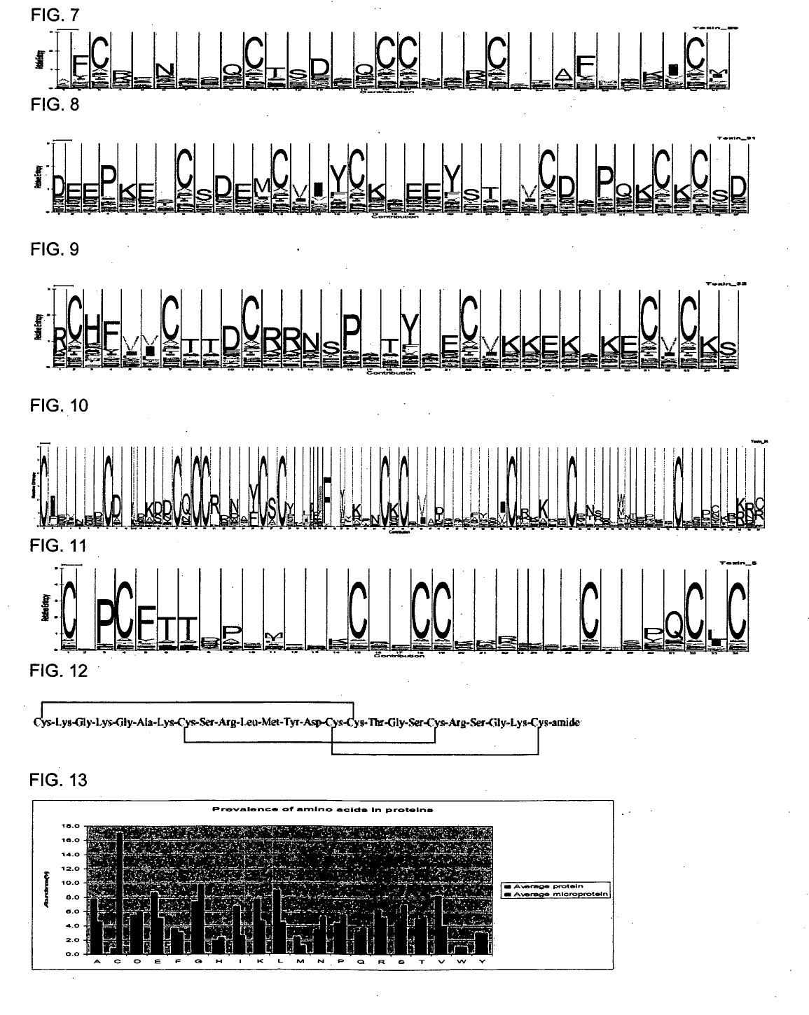

Motif for FIG. 7:

1) CxxxgxxCxxdgxCCxgxCxxxfxgxxC

2) CxxxxxxCxxxxxCCxxxCxxxxxxxxC

CDP: C6C5C0C3C8C

Motif for FIG. 8:

1) CxdxxCxxyCxgxxyxxgxCdgpxxCxC

2) CxxxxCxxxCxxxxxxxxxCxxxxxCxC

CDP: C4C3C9C5C1C

Motif for FIG. 9:

1) ChfxxCxxdCrrxxPGxyGxCxxxxxGxxCxC

2) CxxxxCxxxCxxxxPGxxGxCxxxxxGxxCxC

3) CxxxxCxxxCxxxxxxxxxxCxxxxxxxxCxC

CDP: C4C3C10C8C1C

Motif for FIG. 10:

1) CixxgxxCxG(xx)xxxxCxCCxxxxyCxCxxx(xxx)FG(x)xxxxCxC(x)xxxxxCxxxxxx(x)xxxxxC

2) CxxxxxxCxG(xx)xxxxCxCCxxxxxCxCxxx(xxx)FG(x)xxxxCxC(x)xxxxxCxxxxxx(x)xxxxxC

3) CxxxxxxCxx(xx)xxxxCxCCxxxxxCxCxxx(xxx)xx(x)xxxxCxC(x)xxxxxCxxxxxx(x)xxxxxC

Motif for FIG. 11:

1) CxPCfttxxxxxxxCxxCCxxx(x)xgxCxxxqCxC

2) CxPCxxxxxxxxxxCxxCCxxx(x)xxxCxxxxCxC

3) CxxCxxxxxxxxxxCxxCCxxx(x)xxxCxxxxCxC

CDP: C2C10C2C0C6(7)C4C1C

Motif for FIG. 12:

CxxxxxxCxxxxxxCCxxxCxxxxC

CDP: C6C6C0C3C4C

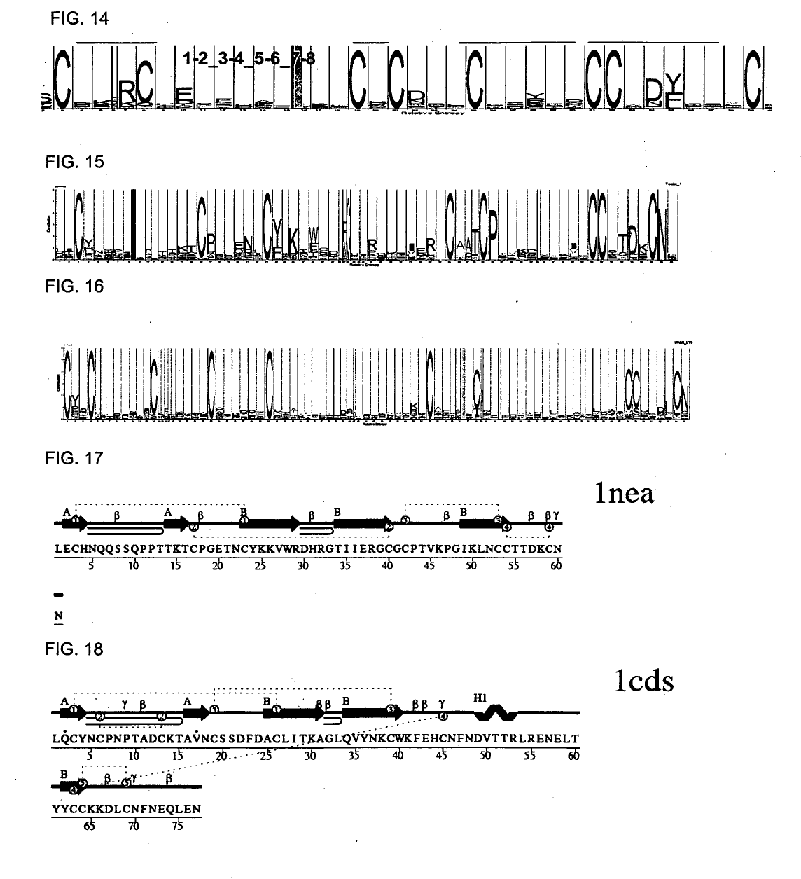

Motifs for FIG. 14:

1) Cxx(x)xCxxxxxxxxxxCxCxxxCxxxxxCCxxxxxxC

2) Cxx(x)RCxExxxxxxxxCxCxxxCxxxxxCCxD[yf]xxxC

CDP: C3-4C10C1C3C5C6C

Motifs for FIG. 15:

1) Cxxxxx(x)x(x)xxxxxCpxgxxxC[yf]xkxxxx(xx)CxxrxxxxxrGCxxtCPxkxx(x)xxxxxCCxtdxCN

2) Cxxxxx(x)x(x)xxxxxCxxxxxxCxxxxxxx(xx)CxxxxxxxxxGCxxxCPxxxx(x)xxxxxCCxxxxCN

3) Cxxxxx(x)x(x)xxxxxCxxxxxxCxxxxxxx(xx)CxxxxxxxxxxCxxxCxxxxx(x)xxxxxCCxxxxC

CDP: C6-8C6C7-9C10C3C10-11C0C4C

Motifs for FIG. 16:

1) CxxCxxxxxxxxC(xxx)xxxxxxCxxxxxxCxxxxxxxxxxxxxxxxxxxxCxx(xx)xC(p)xx(x)xxxxxxxxxxx(x)xxxxxCCxxxxC

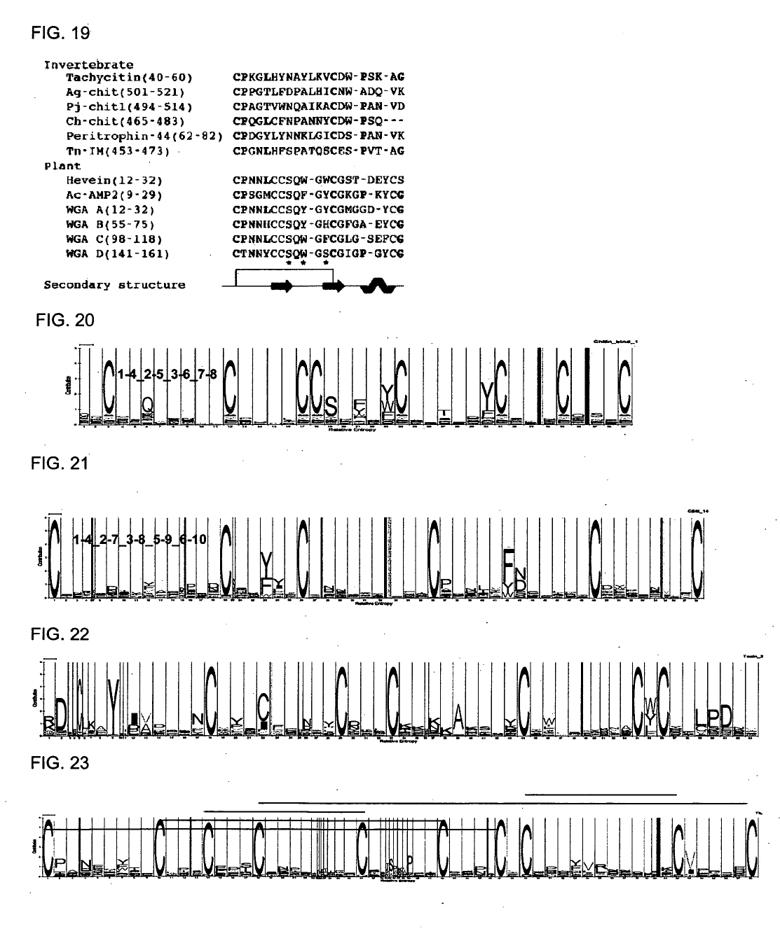

Motifs for FIG. 20:

1) CgxqxxxxxCxxxxCCsxxGxCGxxxxyCxx(x)xCx(x)xxC

2) CxxxxxxxxCxxxxCCxxxxxCxxxxxxCxx(x)xCx(x)xxC

CDP: C8C4C0C5C6C3-4C3-4C

Motifs for FIG. 21:

1) Cxxx(x)xxxxxxx(xx)xxxC(x)xxxxxCxxxxxx(x)xxxCxxxxxxxxxxxxCxxxxx(xx)xxC

2) Cxxx(x)xxxxxxx(xx)xxxC(x)xx[yf]xxCxxxxxx(x)xxxCxxxxx[yf]xxxxxxCxxxxx(xx)xxC

CDP: C13-16C5-6C9-10C12C7-9C

Motifs for FIG. 22:

1) C(xx)xY(gg)xxxxxxCxxxCxx(x)xxxCxxxCxx(x)xgaxxgxCxxxx(x)xxxxxC[wylf]C

2) C(xx)xx(xx)xxxxxxCxxxCxx(x)xxxCxxxCxx(x)xxxxxxxCxxxx(x)xxxxxCxC

CDP: C8-12C3C5-6C3C9-10C9-10C1C

Motifs for FIG. 23:

1) CxxxxxxxxCxxxCxxxCxxxxx(xxxx)xxxCxxxx(xxxx)xxCxxxxCxCxxxxxxxxxx(x)xCxxxxxC

2) CpxxxxxxxCxxxCxxxCxxxxx(xxxx)xxxCxxxx(xxxx)xxCxxxxCxCxxgxxxxxxx(x)xCvxxxxC

CDP: C8C3C3C8-12C6-10C4C1C

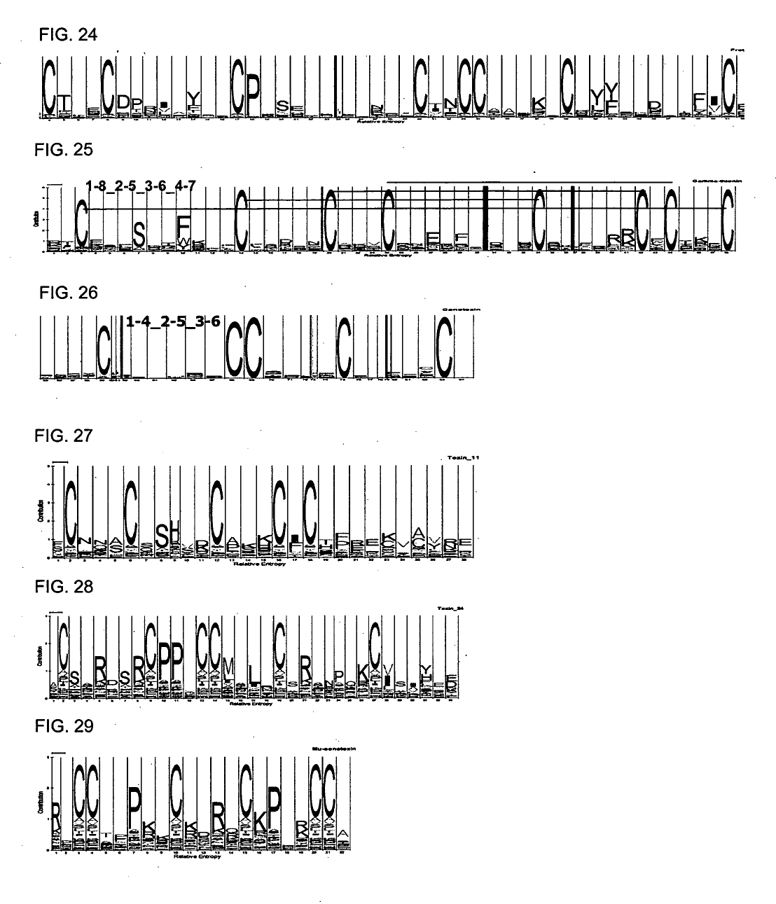

Motifs for FIG. 24:

1) CxxxCxxxxxxxxCPxxxxx(x)xxxxxCxxCCxxxxxCxxxxxxxxxxC

2) CtxxCdxxxxxxxCPxxxxx(xx)xxxxxCxxCCxxgxGCx[yfl][yfl]xxxxGxx[ivl]C

CDP: C3C8C11-12C2C0C5C10C

Motifs for FIG. 25:

1) CxxxxSxx[Fwy]xGxCxxxxxCxxxCxxexxx(xx)xGxCxx(xx)xxr[rk]CxCxxxC

2) CxxxxSxxFxGxCxxxxxCxxxCxxxxxx(xx)xGxCxx(xx)xxxxCxCxxxC

3) CxxxxxxxxxxxCxxxxxCxxxCxxxxxx(xx)xxxCxx(xx)xxxxCxCxxxC

CDP: C11C5C3C9-11C6-8C1C3C

Motifs for FIG. 26:

C(xxx)xxxxxxCCxxx(x)xCxx(xx)xxxC

CDP: C6-9C0C4-5C5-7C

Motifs for FIG. 27:

1) CxxxCxshxxCxxxCxCxxxx[xc]x[xc]

Motifs for FIG. 28:

1) CxgrxxrCppxCCxgxxCxrgxxxxC

2) CxxxxxxCxxxCCxxxxCxxxxxxxC

CDP: C6C3C0C4C7C

Motifs for FIG. 29:

1) CCxxpxxCxxrxCxpxxCC

2) CCxxxxxCxxxxCxxxxCC

CDP: C0C5C4C4C0C

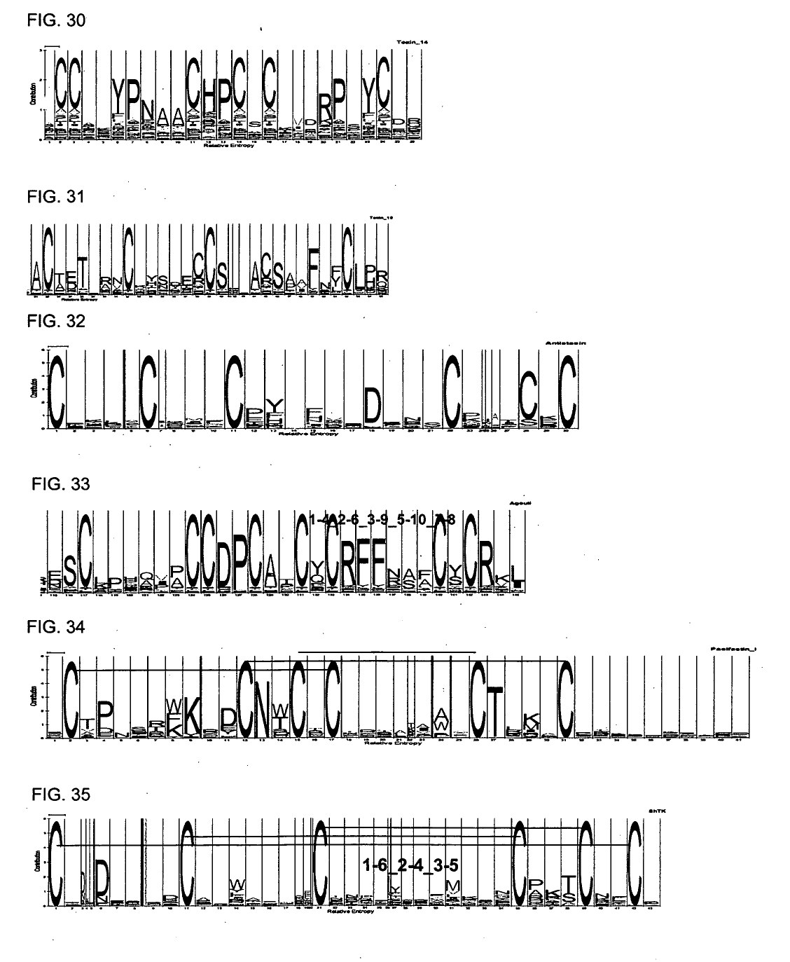

Motifs for FIG. 30:

1) CCgxypxxxChpCxCxxxrpxyC

2) CCxxxxxxxCxxCxCxxxxxxxC

CDP: C0C7C2C1C7C

Motifs for FIG. 31:

1) CxxtGxxCxxxxx[cx]Csx(x)Ga[cx]sxxFxxC

2) CxxxxxxCxxxxx[cx]Cxx(x)xx[cx]xxxxxxC

Motifs for FIG. 32:

1) CxxxxC(x)xxxCxxGxxxDxxgCxx(xx)xCxC

2) CxxxxC(x)xxxCxxxxxxxxxxCxx(xx)xCxC

CDP: C4C3-4C10C2-4C1C

Motifs for FIG. 33:

1) CxxxxxxCCDPCaxCxCRFFxxxCxCR

2) CxxxxxxCCxxCxxCxCxxxxxxCxC

CDP: C6C0C2C2C1C6C1C

Motifs for FIG. 34:

1) CxpgxxxkxxCNxCxCxxxx(x)xxxTxxxC

2) CxxxxxxxxxCNxCxCxxxx(x)xxxTxxxC

3) CxxxxxxxxxCxxCxCxxxx(x)xxxxxxxC

CDP: C9C2C1C11-12C

Motifs for FIG. 35:

1) Cxx(xx)xxxxxCxxxxxxx(x)CxxxxxxxxxxxxCxxxCxxC

2) Cxx(xx)DxxxxCxxxxxxx(x)CxxxxxxxxxxxxCxxxCxxC

3) Cxx(xx)DxxxxCxx[wylfim]xxxx(x)CxxxxxxxxxxxxCxxtCxxC

CDP: C7-9C7-8C12C3C2C

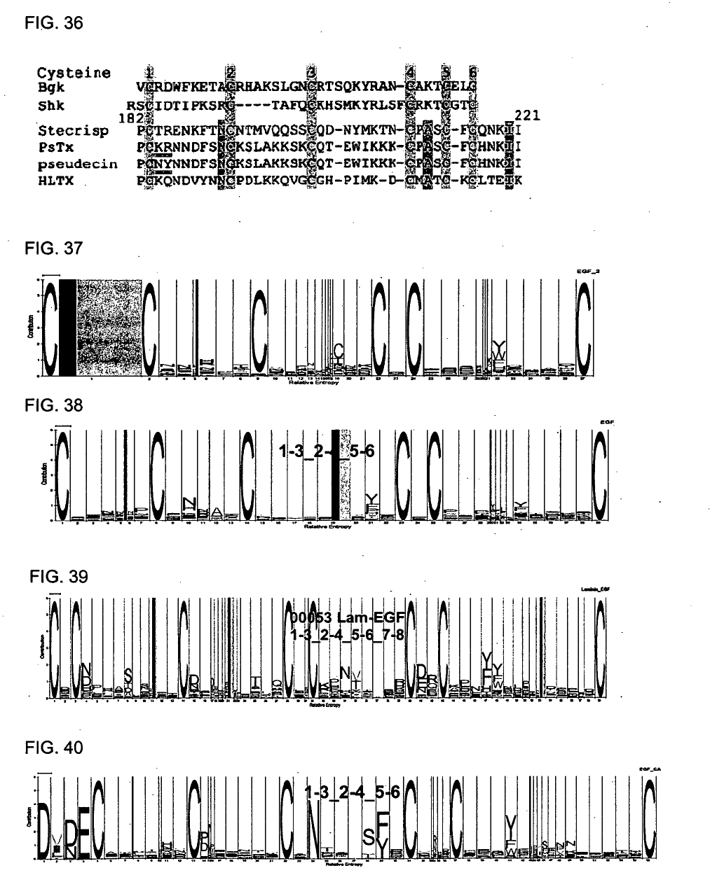

Motifs for FIG. 37:

1) C(xxxx)CxxxxxCxxx(xxxxxxx)xxxCxCxxxx(xx)xxxxxC

2) C(xxxx)CxxxGxCxxx(xxxxxxx)xxxCxCxxxx(xx)xxGxxC

3) C(xxxx)CxxxGxCxxx(xxxxxxx)xxxCxCxxxx(xx) [ywflh]xGxxC

CDP: C0-4C5C6-13C1C9-11C

Motifs for FIG. 38:

1) Cxxxx(x)xCxxxxxCxxxxx(xx)xxxCxCxxx(xxx)xxxxxxC

2) Cxxxx(x)xCxxxgxCxxxxx(xx)xxxCxCxxg(xxx)xxxgxxC

CDP: C5-6C5C8-10C1C9-12C

Motifs for FIG. 39:

1) CxCxxxxxxx(xx)xxCxxx(xxxxxxxx)xxxxxxCxCxxxxxxxxCxxCxxxxxxxxx(xx)xxxxxC

2) CxCxxxxxxx(xx)xxCxxx(xxxxxxxx)xxxxGxCxCxxxxxGxxCxxCxxxxxxxxx(xx)xxxxxC

CDP: C1C9-11C9-17C1C8C2C14-16C

Motifs for FIG. 40:

1) DxdECxxxxxxCx(xx)xxxxxCxNxxGx[fy]xCx(xxx)xCxxg[yf]x(xxxx)xxxxxxxC

2) DxxECxxxxxxCx(xx)xxxxxCxNxxGxxxCx(xxx)xCxxxxx(xxxx)xxxxxxxC

3) CxxxxxxCx(xx)xxxxxCxxxxxxxxCx(xxx)xCxxxxx(xxxx)xxxxxxxC

CDP: C6C6-8C8C2-5C12-16C

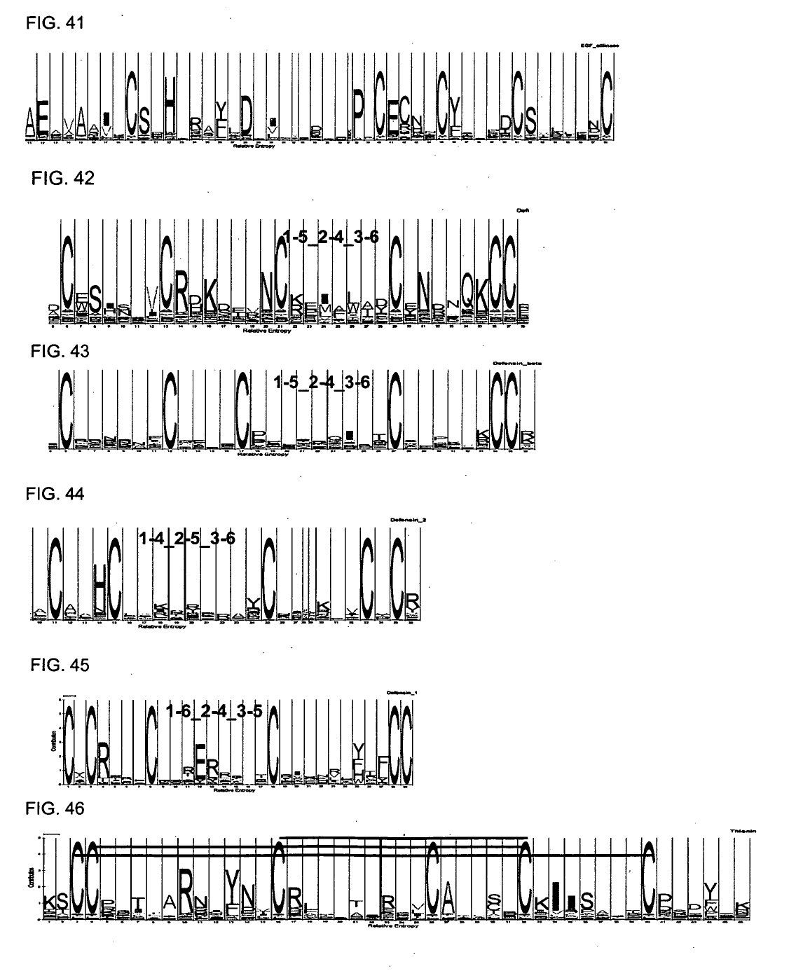

Motifs for FIG. 41:

1) CsxHGxxxxDGxx(x)xxGxxPxCeCxxCyxGxxCsxxxxxC

2) CxxHGxxxxDGxx(x)xxGxxPxCxCxxCxxGxxCxxxxxxC

3) Cxxxxxxxxxxxx(x)xxxxxxxCxCxxCxxxxxCxxxxxxC

CDP: C19-20C1C2C5C6C

Motifs for FIG. 42:

1) CxxxxGxCRxkxxxnCxxxxxxxCxnxxqkCC

2) CxxxxGxCRxxxxxxCxxxxxxxCxxxxxxCC

3) CxxxxxxCxxxxxxxCxxxxxxxCxxxxxxCC

CDP: C6C7C7C6C0C

Motifs for FIG. 43:

1) CxxxxxxCxxxxCxxxxxxxxxCxxxxxxCC

2) CxxxxgxCxxxxCxxxxxxxgxCxxxxxxCC

CDP: C6C4C9C6C0C

Motifs for FIG. 44:

1) CxxHCxxxgxxggxCxx(xxx)xxxCxC

2) CxxHCxxxxxxxxxCxx(xxx)xxxCxC

3) CxxxCxxxxxxxxCxx(xxx)xxxCxC

CDP: C3C8C5-8C1C

Motifs for FIG. 45:

1) CxCRxxxCxxxExxxGxCxxxxxx[yfh]x[yfl]CC

2) CxCRxxxCxxxExxxGxCxxxxxxxxxCC

3) CxCxxxCxxxxxxxxxCxxxxxxxxxCC

CDP: C1C3C9C9C0C

Motifs for FIG. 46:

1) CCxxxxxRxx[yf]nxCrxxGxxxxxCaxxxxCxiisgxxC

2) CCxxxxxRxxxxxCxxxGxxxxxCxxxxxCxxxxxxxC

3) CCxxxxxxxxxxxCxxxxxxxxxCxxxxxCxxxxxxxC

CDP: C0C11C9C5C7C

Motifs for FIG. 47:

1) CxxaxxxCxxxxCxxxCxx(x)xxxxxCxxx[vi]xx(x)xxC

2) CxxxxxxCxxxxCxxxCxx(x)xxxxxCxxxxxxx(x)xxC

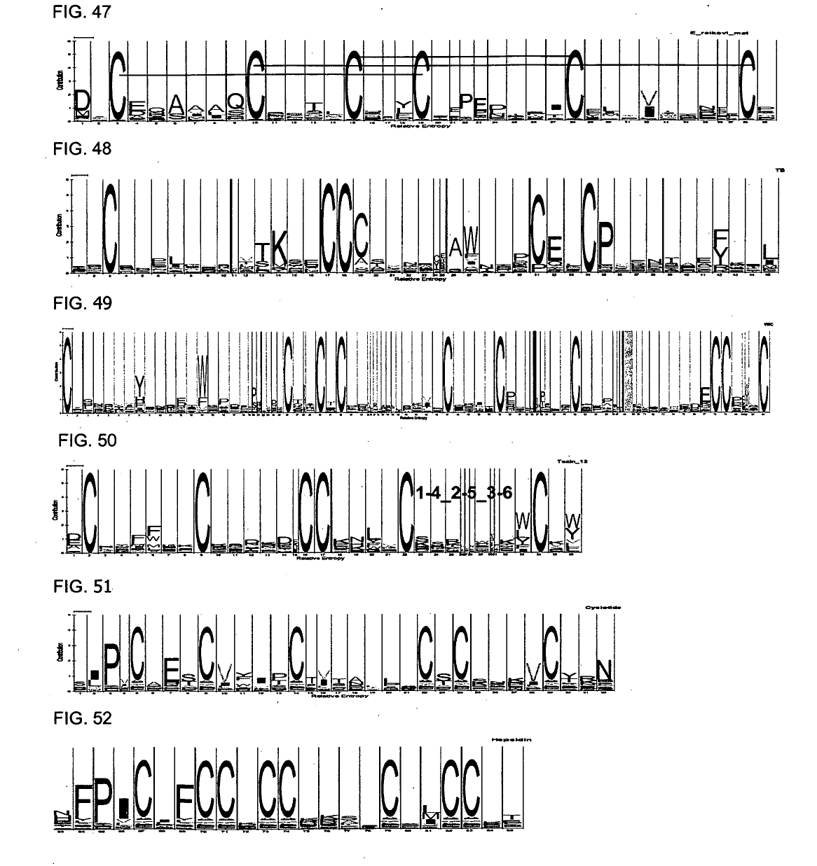

Motifs for FIG. 48:

1) Cxxxxxxx(x)xxxxxCCCxxxx(x)xxxxxxCxxC

2) Cxxxxxxx(x)xxkxxCCCxxxx(x)xx[wfiv]gxxCexC

CDP: C12-13C0C0C10-11C2C

Motifs for FIG. 49:

1) Cxxxxxx[yfb]xxxxxWxxxx(xxxx)xxxCx(x)xCxCxx(xxxxxxxx)xxxxCxxxxCxx(xxxxx)xxCxxx(xxx)xxxxxxxgeCCx(x X)xC

2) CxxxxxxxxxxxxWxxxx(xxxx)xxxCx(x)xCxCxx(xxxxxxxx)xxxxCxxxxCxx(xxxxx)xxCxx(xxx)xxxxxxxxCCx(xx)xC

3) Cxxxxxxxxxxxxxxxxx(xxxx)xxxCx(x)xCxCxx(xxxxxxxx)xxxxCxxxxCxx(xxxxx)xxCxxx(xxx)xxxxxxxxCCx(xx)xC

Motifs for FIG. 50:

1) CxxxxxxCxxxxxCCxxxxCxxx(xxx)x(xx)x[wylfi]C

2) CxxxxxxCxxxxxCCxxxxCxxx(xxx)x(xx)xxC

CDP: C6C5C0C4C6-11C

Motifs for FIG. 51:

1) CxexCvxxxCxxxxxxGCxCxxxvC

2) CxxxCxxxxCxxxxxxxCxCxxxxC

CDP: C3C4C7C1C4C

Motifs for FIG. 52:

1) CxfCCxCCxxxxCgxCC

2) CxxCCxCCxxxxCxxCC

CDP: C2C0C1C4C2C0C

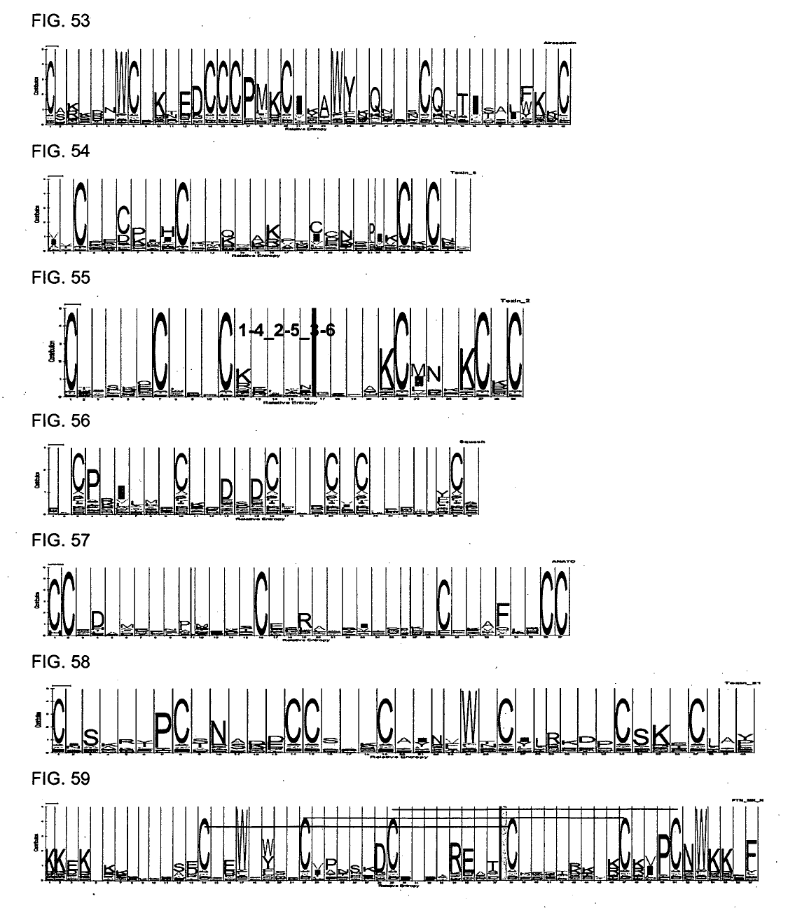

Motifs for FIG. 53:

1) CxxxxxWCgxxedCCCpmxCxxxWyxqxgxCqxxxxxxxxkxxC

2) CxxxxxWCxxxxxCCCxxxCxxxWxxxxxxCxxxxxxxxxxxxC

3) CxxxxxxCxxxxxCCCxxxCxxxxxxxxxxCxxxxxxxxxxxxC

CDP: C6C5C0C0C3C10C12C

Motifs for FIG. 54:

1) CxxCxxxCxxxxxxxxCxxx(xx)xCxC

Motifs for FIG. 55:

1) CxxxxxCxxxCxxxxx(x)xxxxxCxxxxCxC

2) CxxxxxCxxxCxxxxx(x)xxxgkCxxxkCxC

CDP: C5C3C10-11C4C1C

Motifs for FIG. 56:

1) CPxxxxxCxxdxdCxxxCxCxxxx(x)xC

2) CPxxxxxCxxxxxCxxxCxCxxxx(x)xC

2) CxxxxxxCxxxxxCxxxCxCxxxx(x)xC

CDP: C6C5C3C1C5-6C

Motifs for FIG. 57:

1) CCxdgxxxxx(x)xxxxCxxrxxxxxxxxxCxxxfxxCC

2) CCxxxxxxxx(x)xxxxCxxxxxxxxxxxxCxxxxxxCC

CDP: C0C12-13C12C6C0C

Motifs for FIG. 58:

1) CxsxxxPCxnxxxCCxgxCxxxxWxCxxxxxxCskxC

2) CxxxxxPCxxxxxCCxxxCxxxxWxCxxxxxxCxxxC

3) CxxxxxxCxxxxxCCxxxCxxxxxxCxxxxxxCxxxC

CDP: C6C5C0C3C6C6C3C

Motifs for FIG. 59:

1) CxxWx[wylf]xxCxxxxxdCgxgxrexx(xx)CxxxxxxxxCxxPC

2) CxxWxxxxCxxxxxxCxxxxxxxx(xx)CxxxxxxxxCxxPC

3) CxxxxxxxCxxxxxxCxxxxxxxx(xx)CxxxxxxxxCxxxC

CDP: C7C6C8-10C8C3C

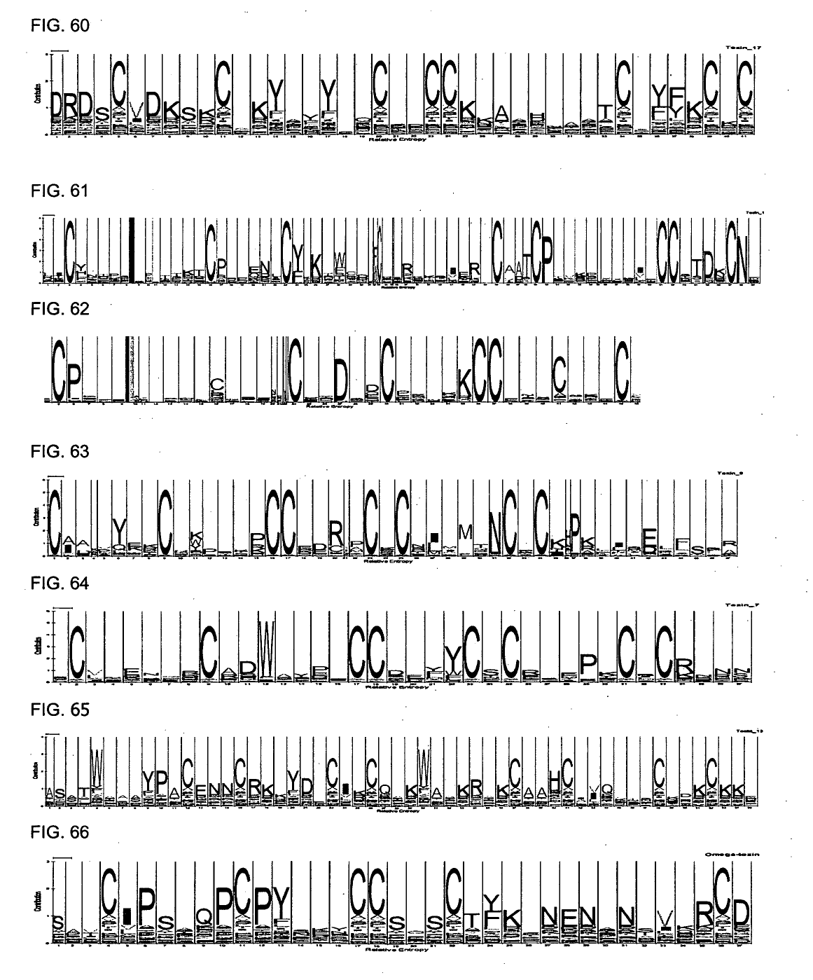

Motifs for FIG. 60:

1) CxdxxxCxxygxyxxCxxCCxxxgxxxgxCxxxxCxC

2) CxxxxxCxxxxxxxxCxxCCxxxxxxxxxCxxxxCxC

CDP: C5C8C2C0C9C4C1C

Motifs for FIG. 61:

1) Cxxxxx(x)x(x)xxxxxCpxgxxxC[yf]xkxxxx(xx)CxxxxxxxxxGCxxtCPxxxx(x)xxxxxCCxxdxC

2) Cxxxxx(x)x(x)xxxxxCxxxxxxCxxxxxxxx(xx)CxxxxxxxxxGCxxxCPxxxx(x)xxxxxCCxxxxC

3) Cxxxxx(x)x(x)xxxxxCxxxxxxCxxxxxxx(xx)CxxxxxxxxxxCxxxCxxxxx(x)xxxxxCCxxxxC

CDP: C11-13C6C7-9C10C3C10-11C0C4C

Motifs for FIG. 62:

1) CPxxx(xx)xxxxxCxxx(xxx)CxxDxxCxxxxkCCxxxCxxxC

2) CPxxx(xx)xxxxxCxxx(xxx)CxxDxxCxxxxCCxxxCxxxC

3) Cxxxx(xx)xxxxxCxxx(xxx)CxxxxxCxxxxxCCxxxCxxxC

CDP: C9-11C3-6C5C5C0C3C3C

Motifs for FIG. 63:

1) Cxx(x)xyxxCxxgxxxCCxxr(x)xCxCxxxxxNCxC

2) Cxx(x)xxxxCxxxxxxCCxxx(x)xCxCxxxxxNCxC

3) Cxx(x)xxxxCxxxxxxCCxxx(x)xCxCxxxxxxCxC

CDP: C6-7C6C0C4-5C1C6C1C

Motifs for FIG. 64:

1) CxxxxxxCxdWxxxxCCxgxyCxCxxxpxCxC

2) CxxxxxxCxxWxxxxCCxxxxCxCxxxxxCxC

3) CxxxxxxCxxxxxxxCCxxxxCxCxxxxxCxC

CDP: C6C7C0C4C1C5C1C

Motifs for FIG. 65:

1) CxxxCrxxydxCxxCxgxWxgxxgxCxxhCxxxxxxCxxxC

2) CxxxCxxxxxxCxxCxxxWxxxxxxCxxxCxxxxxxCxxxC

3) CxxxCxxxxxxCxxCxxxxxxxxxxCxxxCxxxxxxCxxxC

CDP: C3C6C2C10C3C6C3C

Motifs for FIG. 66:

1) CxPxGxPCPyxxxCCxxxCxxxxxxxgxxxxrC

2) CxxxxxxCxxxxxCCxxxCxxxxxxxxxxxxxC

3) CxPxGxPCPxxxxCCxxxCxxxxxxxxxxxxxC

CDP: C6C5C0C3C13C

Motifs for FIG. 67:

1) CxxxxxxxxxxxCPxgxxxxxCxCgxxCgsWxxxxxxxCxCxCxxxdWxxxrCC

2) CxxxxxxxxxxxCPxxxxxxxCxCxxxCxxWxxxxxxxCxCxCxxxxWxxxxCC

3) CxxxxxxxxxxxCxxxxxxxxCxCxxxCxxxxxxxxxxCxCxCxxxxxxxxxCC

CDP: C11C8C1C3C10C1C1C9C0C

Motifs for FIG. 68:

1) Cx(xx)xxxCxxxxx[nd]gxCx[wylf]DGxDC

2) Cx(xx)xxxCxxxxxxxxCxxDGxDC

3) Cx(xx)xxxCxxxxxxxxCxxxxxxC

CDP: C4-6C8C6C

Motifs for FIG. 69:

1) Cxxxx[yf]xx(xx)xxx(x)xxCxxCxxCxx(xx)gxxxxxxCxxxxxtxC

2) Cxxxxxxx(xx)xxx(x)xxCxxCxxCxx(xx)xxxxxxxCxxxxxxxC

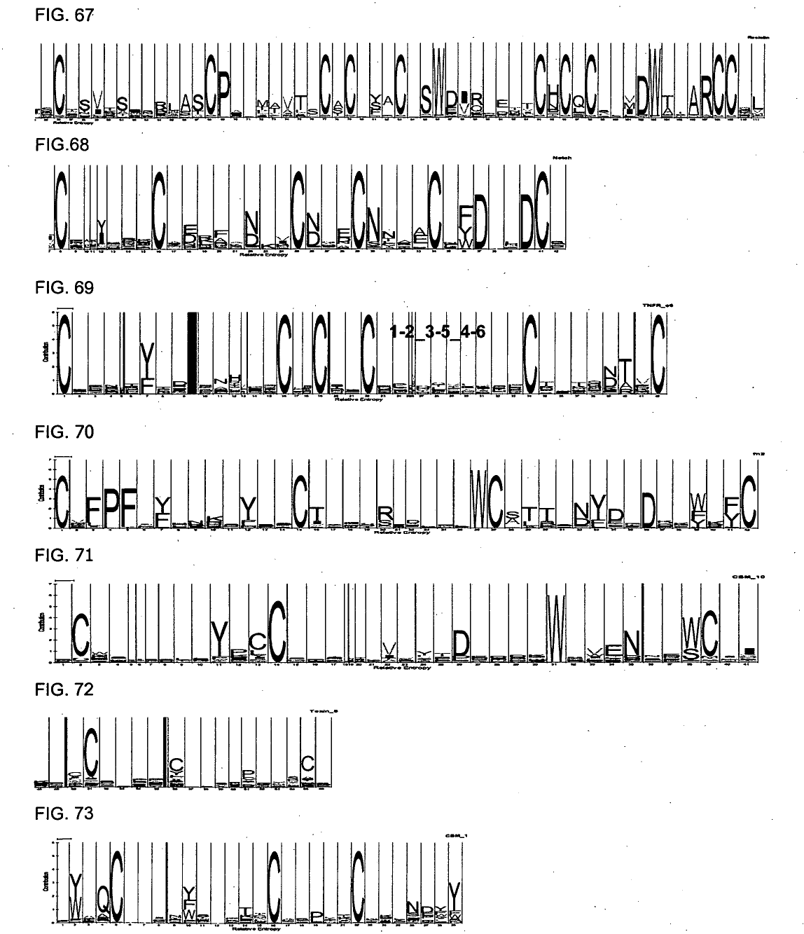

Motifs for FIG. 70:

1) CxfPFx[yf]xxxxxxxCtxxgxxxxxxWCxttxxxdxDxxxx[fy]C

2) CxxPFxxxxxxxxxCxxxxxxxxxxWCxxxxxxDxxxxxC

3) CxxxxxxxxxxxxxCxxxxxxxxxxxCxxxxxxxxxxxxxxC

CDP: C13C11C14C

Motifs for FIG. 71:

1) Cxx(xx)xxxxyxCCxxx(xx)xxxxxxdxxxxWgxxnxxwC

2) Cxx(xx)xxxxxxCCxxx(xx)xxxxxxxxxxxWxxxxxxxC

3) Cxx(xx)xxxxxxCCxxx(xx)xxxxxxxxxxxxxxxxxxxC

CDP: C8-10C0C22-24C

Motifs for FIG. 72:

1) CCxxxx(x)CxxxxpxxxCG

2) CCxxxx(x)CxxxxxxxxC

CDP: C0C4-5C8C

Motifs for FIG. 73:

1) CGGxxxxGxxxCxxgxxC

2) CGGxxxxGxxxCxxxxxC

CDP: C10C5C

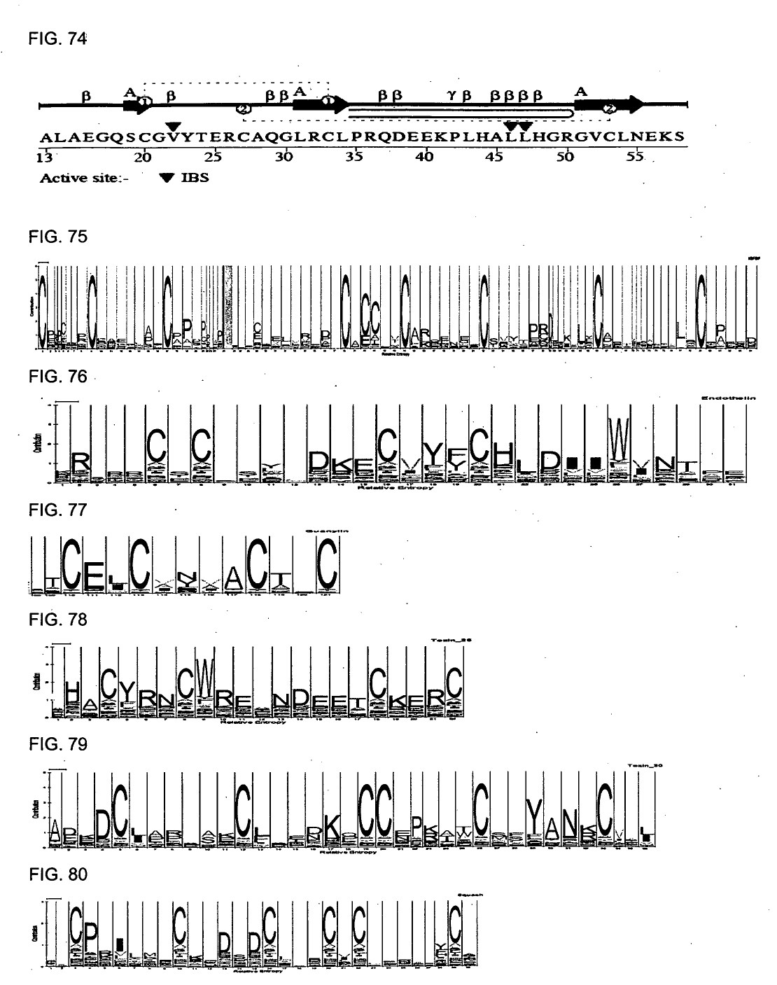

Motifs for FIG. 75:

1) Cx(xxc)xxxCxxxxxxxCxpxx(xxxx)xxxx(c)xxxxxxxGCgCCxxCxxxxgxxCxxxxxx(dx)xxglxCxxg(xx)xxxxxlxC

2) Cx(xxc)xxxCxxxxxxxCxxxx(xxxx)xxxx(c)xxxxxxxGCxCCxxCxxxxxxxCxxxxxx(xx)xxxxxCxxx(xx)xxxxxxxC

3) Cx(xxc)xxxCxxxxxxxCxxxx(xxxx)xxxx(c)xxxxxxxxCxCCxxCxxxxxxxCxxxxxx(xx)xxxxxCxxx(xx)xxxxxxxC

Motifs for FIG. 76:

1) CxCxxxxdkeCx[yfli]xChxd[ivl][ivl]W

2) CxCxxxxdkeCx[yfli]xC

3) CxCxxxxxxxCxxxC

CDP: C1C7C3C

Motifs for FIG. 77:

1) CExCxxxxaCtGC

2) CExCxxxxxCxGC

3) CxxCxxxxxCxxC

CDP: C2C5C2C

Motifs for FIG. 78:

1) CyrxCWregxdeetCkerC

2) CxxxCWxxxxxxxxCxxxC

CDP: C3C9C3C

Motifs for FIG. 79:

1) DCxxxGxxCxGxxkxCCxpxxxCxxYanxC

2) CxxxGxxCxGxxxxCCxxxxxCxxYxxxC

3) CxxxxxxCxxxxxCCxxxxxCxxxxxxC

CDP: C6C5C0C5C6C

Motifs for FIG. 80:

1) CPx[ivlf]xxxCxxdxdCxxxCxCxxxxxxCg

2) CPxxxxxCxxxxxCxxxCxCxxxxxxC

3) CxxxxxxCxxxxxCxxxCxCxxxxxxC

CDP: C6C5C3C1C6C

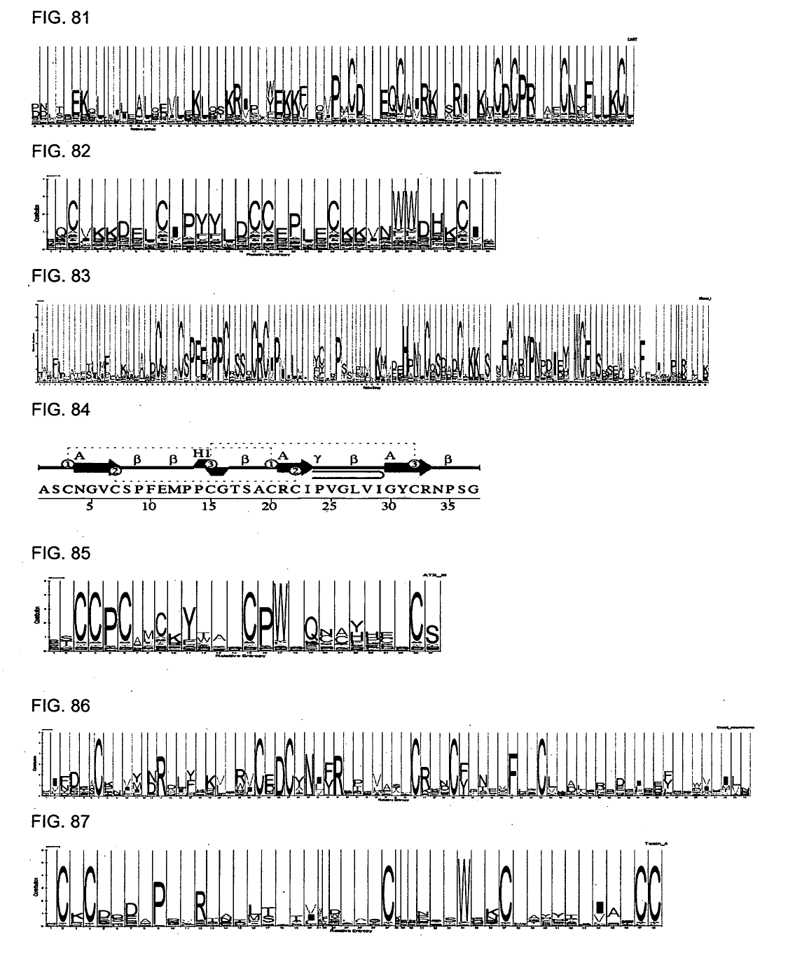

Motifs for FIG. 81:

1) CdxgeqCaxrkgxrxgkxCdCPrgxxCnxfllkC

2) CxxxxxCxxxxxxxxxxxCxCxxxxxCxxxxxxC

CDP: C5C11C1C5C6C

Motifs for FIG. 82:

1) CvkkdelCxpyyxdCCxpxxCxxxxWWdhkC

2) CxxxxxxCxxxxxxCCxxxxCxxxxWWxxxC

3) CxxxxxxCxxxxxxCCxxxxCxxxxxxxxxC

CDP: C6C6C0C4C9C

Motifs for FIG. 83:

1) CxGxCsPFExPPCxssxCrCxPxlxxGxcxxPxxxxxxxkxxxHxnlCxsxxxCxkkxsGcFCxxYPNxxixxGWC

2) CxGxCxPFExPPCxxxxCxCxPxxxxxGxcxxPxxxxxxxxxxxxHxxxCxxxxxCxxxxxGxFCxxYPNxxxxxGWC

3) CxxxCxxxxxxxCxxxxCxCxxxxxxxxxcxxxxxxxxxxxxxxxxxxxCxxxxxCxxxxxxxxCxxxxxxxxxxGxC

Motifs for FIG. [85]:

1) CCPCxxCxYxxGCPWGqxxxxxgC

2) CCPCxxCxYxxGCPWGxxxxxxxC

3) CCxCxxCxxxxxCxxxxxxxxxxC

CDP: C0C1C2C5C10C

Motifs for FIG. 86:

1) CxgxxgxRxxxxxxxxxCxDCxNxxRxxxxxxxCrxxCxxxxxFxxC

2) CxxxxxxRxxxxxxxxxCxDCxNxxRxxxxxxxCxxxCxxxxxFxxC

3) CxxxxxxxxxxxxxxxxCxxCxxxxxxxxxxxxCxxxCxxxxxxxxC

CDP: C16C2C12C3C8C

Motifs for FIG. 87:

1) CxCxxxxPxxrxxxxxGxx(x)xxxxxC(x)xxxxxWxxCxxxxxxxxxCC

2) CxCxxxxPxxxxxxxxGxx(x)xxxxxC(x)xxxxxWxxCxxxxxxxxxCC

3) CxCxxxxxxxxxxxxxxxx(x)xxxxxC(x)xxxxxxxxCxxxxxxxxxCC

CDP: C1C21-22C8-9C9C0C

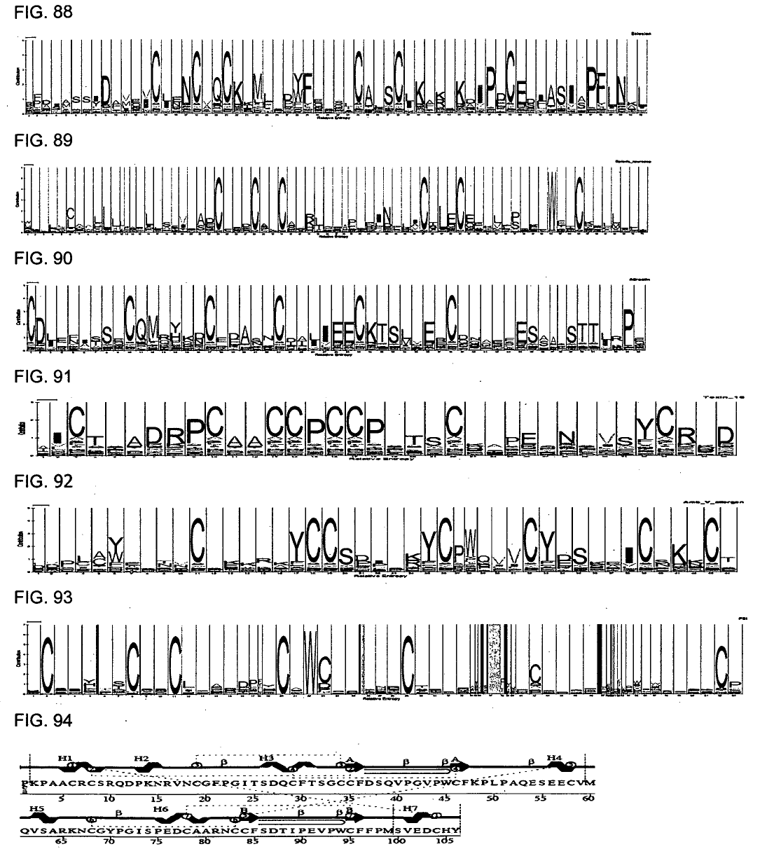

Motifs for FIG. 88:

1) CxxnCxqCkxmxgxxfxgxxCaxsCxkxxGkxxPxC

2) CxxxCxxCxxxxxxxxxxxxCxxxCxxxxGxxxPxC

3) CxxxCxxCxxxxxxxxxxxxCxxxCxxxxxxxxxxC

CDP: C3C2C12C3C10C

Motifs for FIG. 89:

1) CxxxCxxCxxxxxxxxxxxnxxxCxleCxxxxxxxxxWxxC

2) CxxxCxxCxxxxxxxxxxxxxxxCxxxCxxxxxxxxxWxxC

3) CxxxCxxCxxxxxxxxxxxxxxxCxxxCxxxxxxxxxxxxC

CDP: C3C2C15C3C12C

Motifs for FIG. 90:

1) CdxxxxxsxCqmxxxxCxxaxxCxxxieeCktsxxexC

2) CxxxxxxxxCxxxxxxCxxxxxCxxxxxxCxxxxxxxC

CDP: C8C6C5C6C7

Motifs for FIG. 91:

1) CxGxdrPCxxCCPCCPGxxCxxxexxgxxyC

2) CxGxxxPCxxCCPCCPGxxCxxxxxxxxxxC

3) CxxxxxxCxxCCxCCxxxxCxxxxxxxxxxC

CDP: C6C2C0C1C4C10C

Motifs for FIG. 92:

1) CxxxxxxCCxxxxxxCxxxxxCxxxxxxCxxxC

2) CgxxxxyCCsxxgxyCxwxxvCyxsxxxCxkxC

3) CxxxxxxCCxxxxxxCxxxxxCxxxxxxCxxxC

CDP: C6C0C6C5C6C3C

Motifs for FIG. 93:

1) CxxxxxCxxCxxxxxx(x)xCxWCxx(x)xxxCxxxx(xxxxxx)xCxxxx(xxxxxxxxx)xxxxxxC

2) CxxxxxCxxCxxxxxx(x)xCxxCxx(x)xxxCxxxx(xxxxxx)xCxxxx(xxxxxxxxx)xxxxxxC

CDP: C5C2C7-8C2C5-6C5-11C10-19C

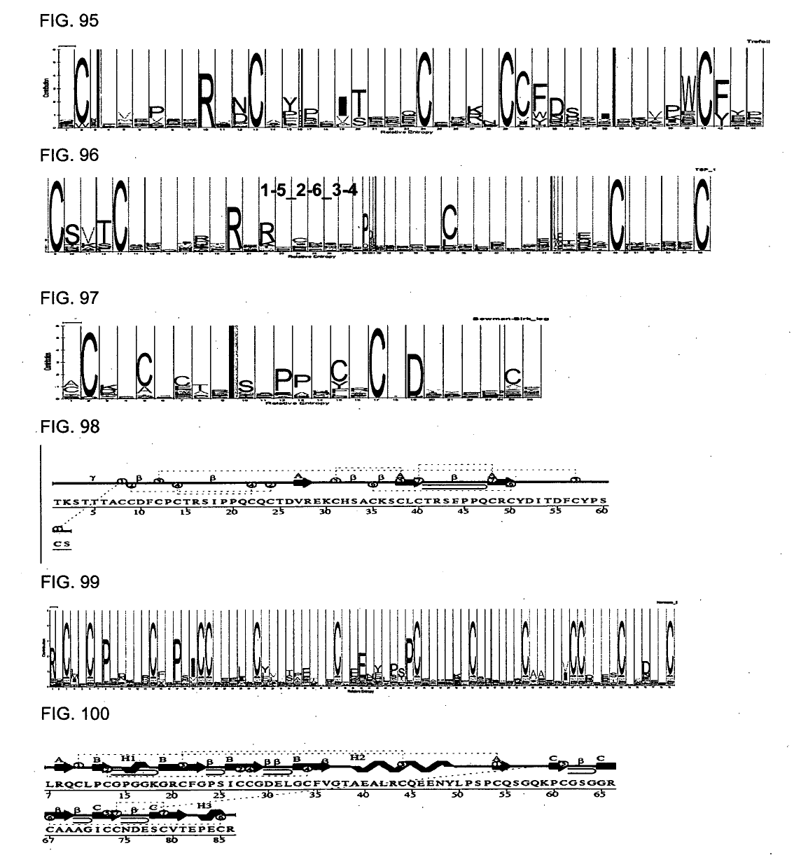

Motifs for FIG. 95:

1) CxxxxxxxRxxCgxxxitxxxCxxxgCCfdxxxxxxxwC

2) CxxxxxxxRxxCxxxxxxxxxCxxxxCCxxxxxxxxxxC

3) CxxxxxxxxxxCxxxxxxxxxCxxxxCCxxxxxxxxxxC

CDP: C10C9C4C0C10C

Motifs for FIG. 96:

1) CsvtCgxGxxxRxrxCxxxx(pxx)xxxxxCxxxxxx(xxx)xxxC(x)xxxxC

2) CxxxCxxGxxxRxxxCxxxx(xxx)xxxxxCxxxxxx(xxx)xxxC(x)xxxxC

3) CxxxCxxxxxxxxxxCxxxx(xxx)xxxxxCxxxxxx(xxx)xxxC(x)xxxxC

CDP: C3C10C9-12C9-12C4-5C

Motifs for FIG. 97:

1) CxxCxCxx(x)sxppxCxCxDxxxx(x)C

2) CxxCxCxx(x)xxxxxCxCxDxxxx(x)C

3) CxxCxCxx(x)xxxxxCxCxxxxxx(x)C

CDP: C2C1C7-8C1C6-7C

Motifs for FIG. 99:

1) CxxCGPxxxGxCxGPxiCCGxxxGCxxGxxxxxxCxxexxxxxPCxxxxxxCxxxxGxCxxxGxCCxxxxCxxdxxC

2) CxxCGPxxxGxCxGPxxCCGxxxGCxxGxxxxxxCxxxxxxxxPCxxxxxxCxxxxGxCxxxGxCCxxxxCxxxxxC

3) CxxCxxxxxxxCxxxxxCCxxxxxCxxxxxxxxxCxxxxxxxxxCxxxxxxCxxxxxxCxxxxxCCxxxxCxxxxxC

CDP: C2C7C5C0C5C9C9C6C6C5C0C4C5C

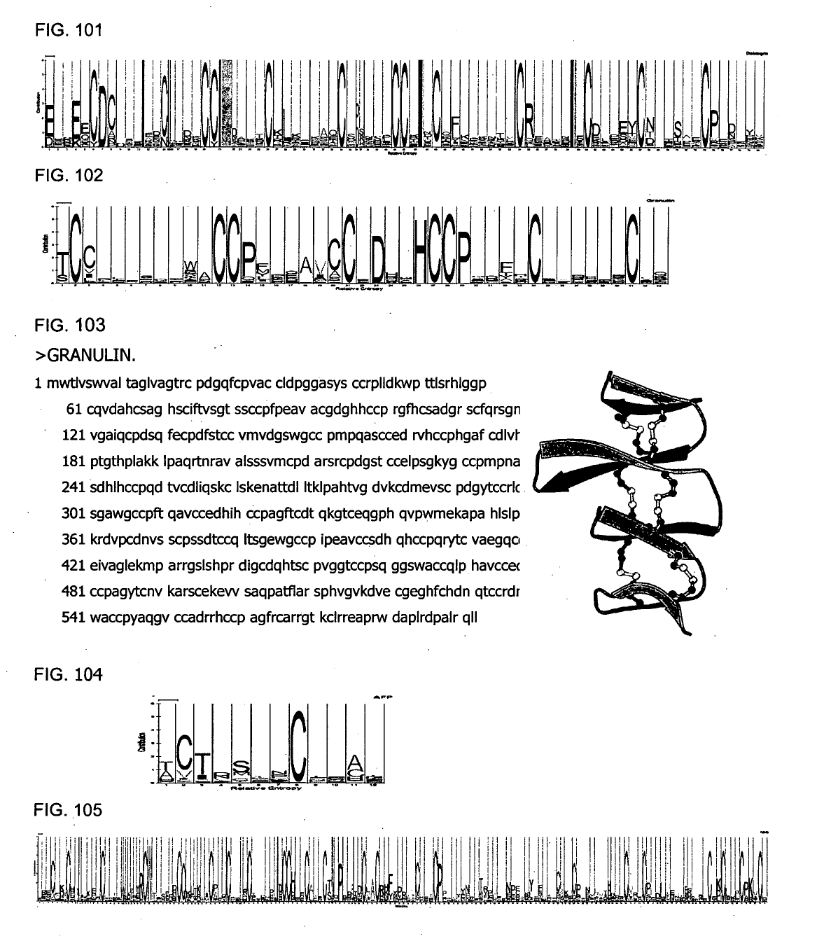

Motifs for FIG. 101:

1) CDCGxxxxC(xx)xxxCC(x)xxxxCxlxxxxxCx(xx)xgxCCx(x)xCxxxxxxxxCrxxxx(x)xCxxxxxCxGxxxxC

2) CDCGxxxxC(xx)xxxCC(x)xxxxCxxxxxxxCx(xx)xxxCCx(x)xCxxxxxxxxCxxxxx(x)xCxxxxxCxGxxxxC

3) CxCxxxxxC(xx)xxxCC(x)xxxxCxxxxxxxCx(xx)xxxCCx(x)xCxxxxxxxxCxxxxx(x)xCxxxxxCxxxxxxC

CDP: C1C5C3-5C0C4-5C7C4-6C0C1-3C8C6-7C5C6C

Motifs for FIG. 102:

1) CCxxxxgxxxCCPxxxxxCCxDxxHCCPxgxxCxxxxxxC

2) CCxxxxxxxxCCPxxxxxCCxDxxHCCPxxxxCxxxxxxC

3) CCxxxxxxxxCCxxxxxxCCxxxxxCCxxxxxCxxxxxxC

CDP: C0C8C0C6C0C5C0C5C6C

Motifs for FIG. 104: 1) Cap(tCtxxxxCxxax)ₙ 2) Cap(xCxxxxxCxxxx)ₙ

Motifs for FIG. 105

1) Cxx(x)Cxx(xxxx)xxxxCxxxx(xxxx)xxxRCWxxxxxxCQxxxxxxCxxxCxx(x)xxCxxxxxxxCChxxCxggCx(xx)xPxx(x)x xCxaCxxfxxxgxCxxxCP

2) Cxx(x)Cxx(xxxx)xxxxCxxxx(xxxx)xxxRCWxxxxxxCQxxxxxxCxxxCxx(x)xxCxxxxxxxCCxxxCxgxCx(xx)xPxx(x)x xCxxCxxxxxxxxCxxxCP

3) Cxx(x)Cxx(xxxx)xxxxCxxxx(xxxx)xxxxCxxxxxxxCxxxxxxxCxxxCxx(x)xxCxxxxxxxCCxxxCxxxCx(xx)xxxx(x)xxC xxCxxxxxxxxCxxxC

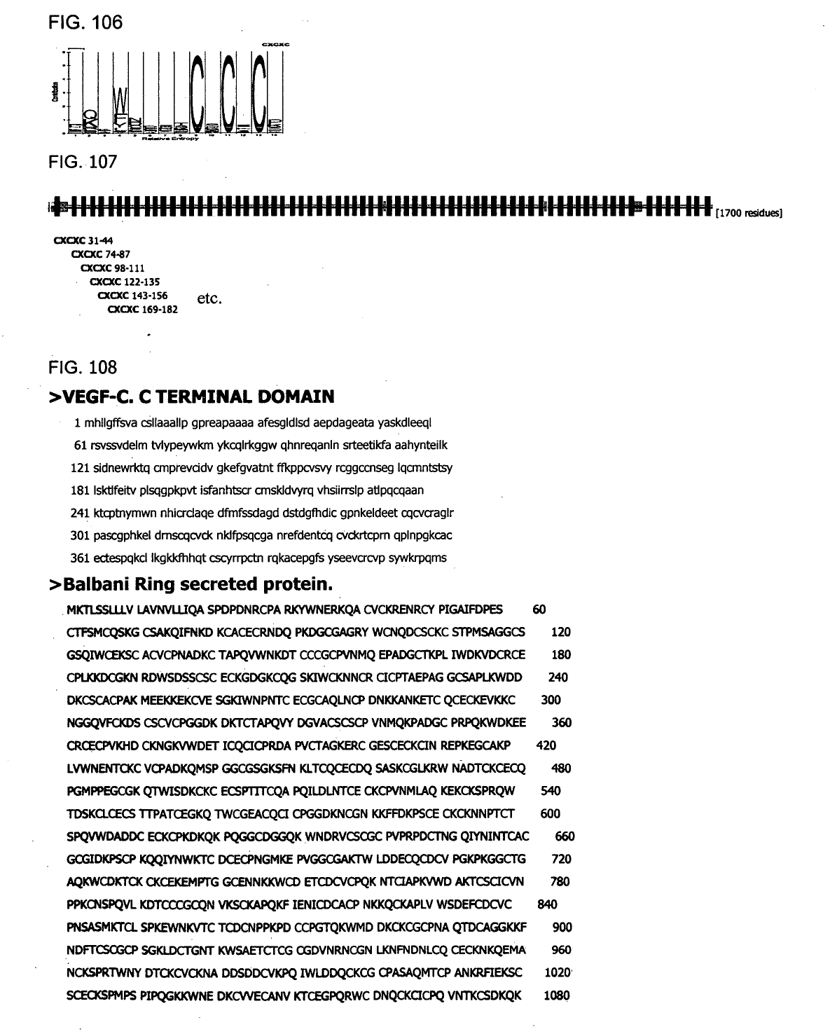

Motifs for FIG. 106:

1) xxx[wyfl]xxxxCxCxCx

2) xxxxxxxxCxCxCx

Motifs for FIG. 110:

1) CxsxxxxxCxxxxxxx(xx)xxxxxCxx(x)xxxxCxxxxxx(x)xxxxrGCxxxxxxxxxxxCx(x)xxxxCxxCxxx(x)xCNxxxxxpxxxx xCxqCxgxxxxx[cx]xxxxxxlxxxxCxxxx(x)xxxxCyxxxxx(xxx)xxxxRGCxxxxxxxxx[cx]xdxxCxxC

2) CxxxxxxxCxxxxxxx(xx)xxxxxCxx(x)xxxxCxxxxxx(x)xxxxxGCxxxxxxxxxxxCx(x)xxxxCxxCxxx(x)xCNxxxxxxxxx xxCxxCxxxxxxx[cx]xxxxxxxxxxxCxxxx(x)xxxxCxxxxxx(xxx)xxxxRGCxxxxxxxxx[cx]xxxxCxxC

3) CxxxxxxxCxxxxxxx(xx)xxxxxCxx(x)xxxxCxxxxxx(x)xxxxxxCxxxxxxxxxxxCx(x)xxxxCxxCxxx(x)xCxxxxxxxxxxx xCxxCxxxxxxx[cx]xxxxxxxxxxxCxxxx(x)xxxxCxxxxxx(xxx)xxxxxxCxxxxxxxxx[cx]xxxxCxxC

Motifs for FIG. 111: xxxxxxCxxxxxx(x)Ctxxx(xx)xg(x)xxCxxxxxxCxxyxxxxxCxxxx(xx)xxxxxCxWxxxx(x)xxCxxxx(xxxx)Cx

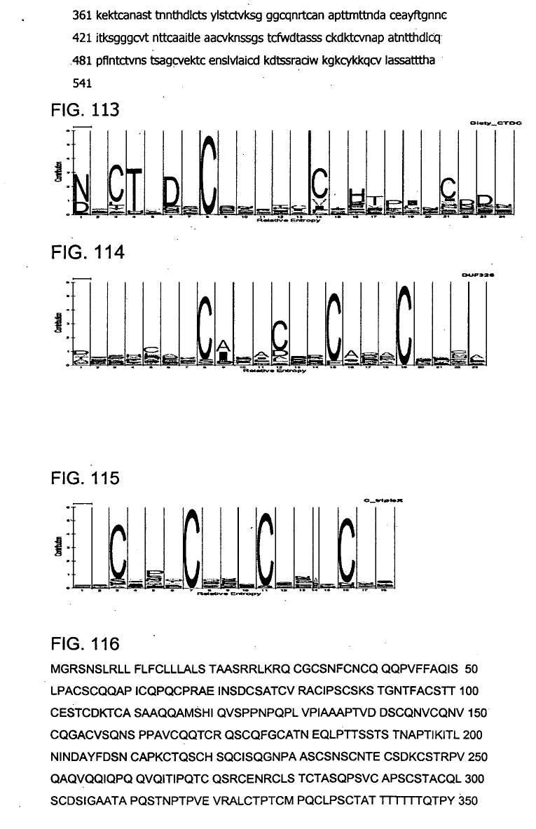

Motif for FIG. 113:

1) nxCtxdxCxxxxgCxxxxxxCxxx

2) CxxxxCxxxxxCxxxxxxCxxx

CDP: C4C5C6C3

Motif for FIG. 114: xxxx[cx]xxCxxx[cx]xxCxxxCxxxx

Motif for FIG. 210: xxCxxxCxxxCxx(x)xCxxCDP: 2C3C3C3-4C2

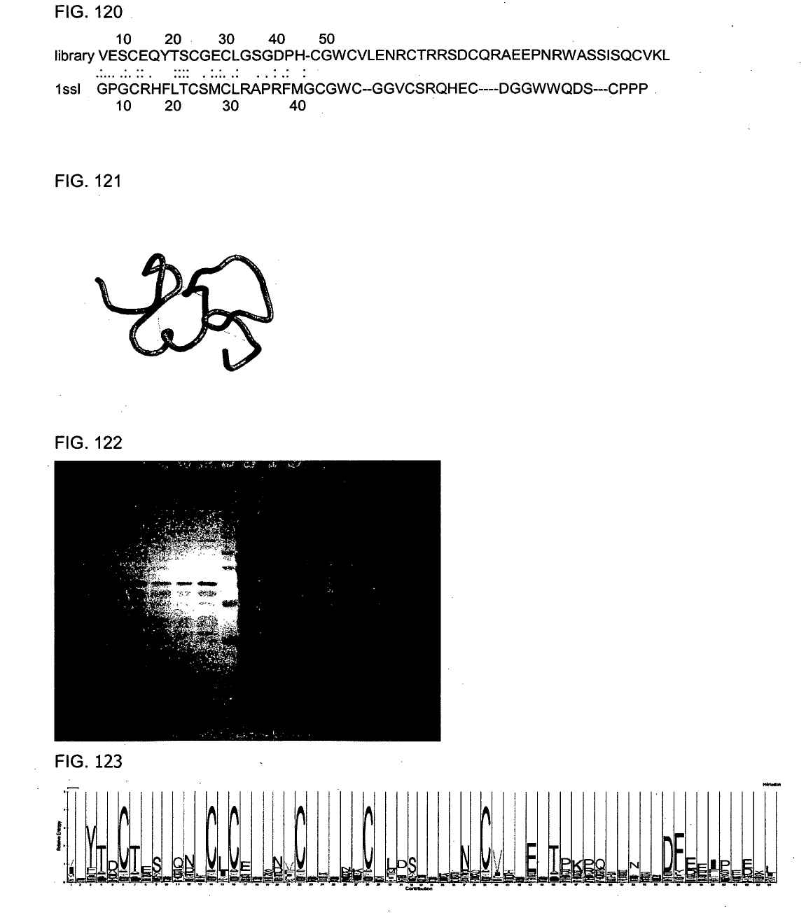

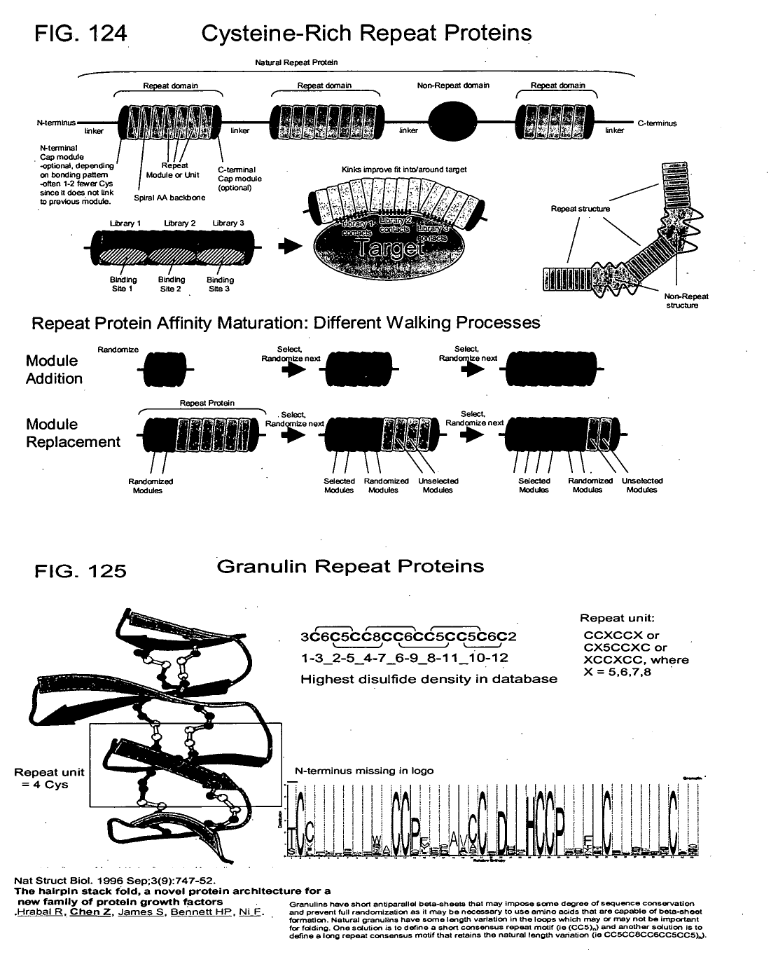

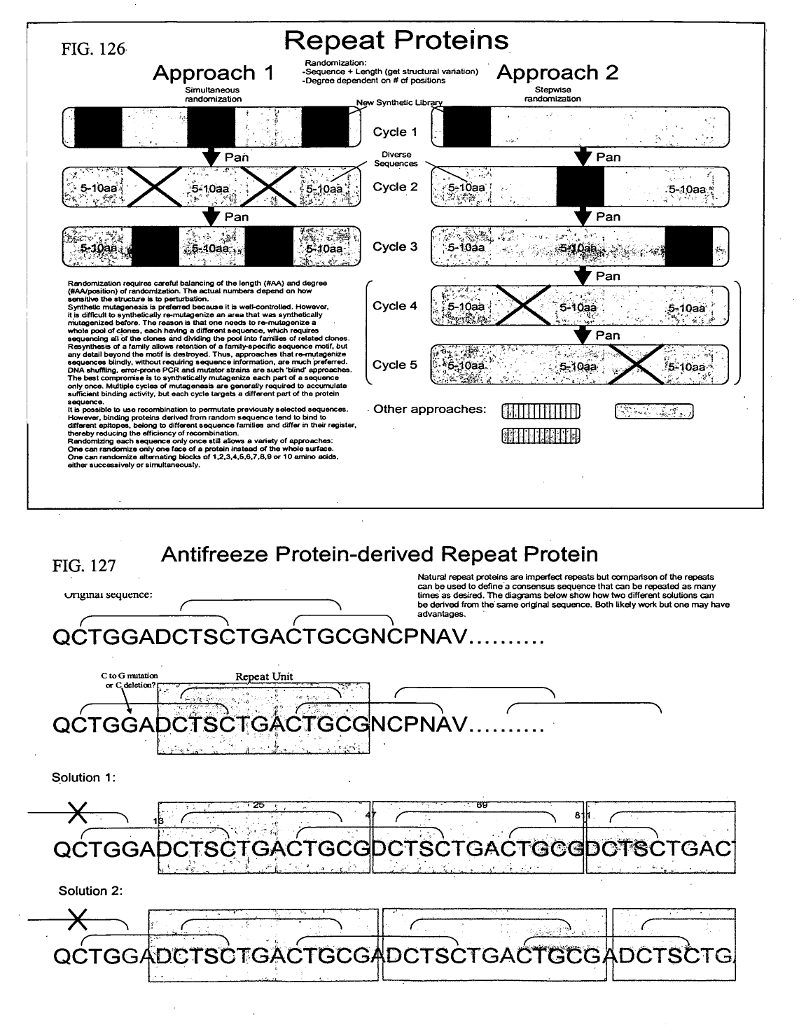

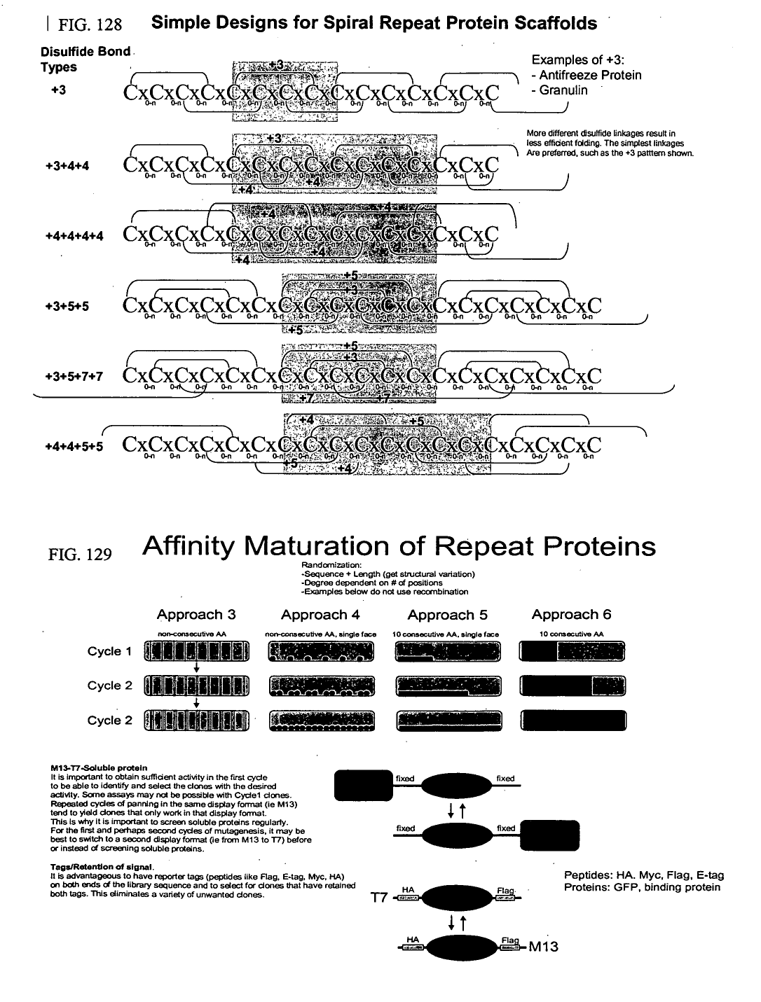

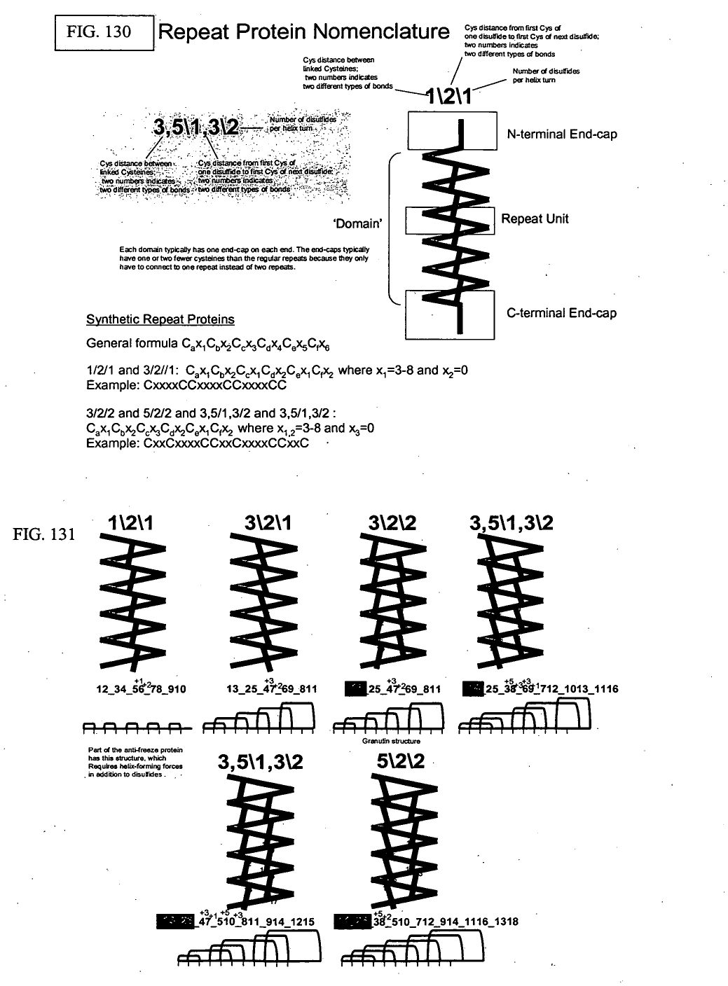

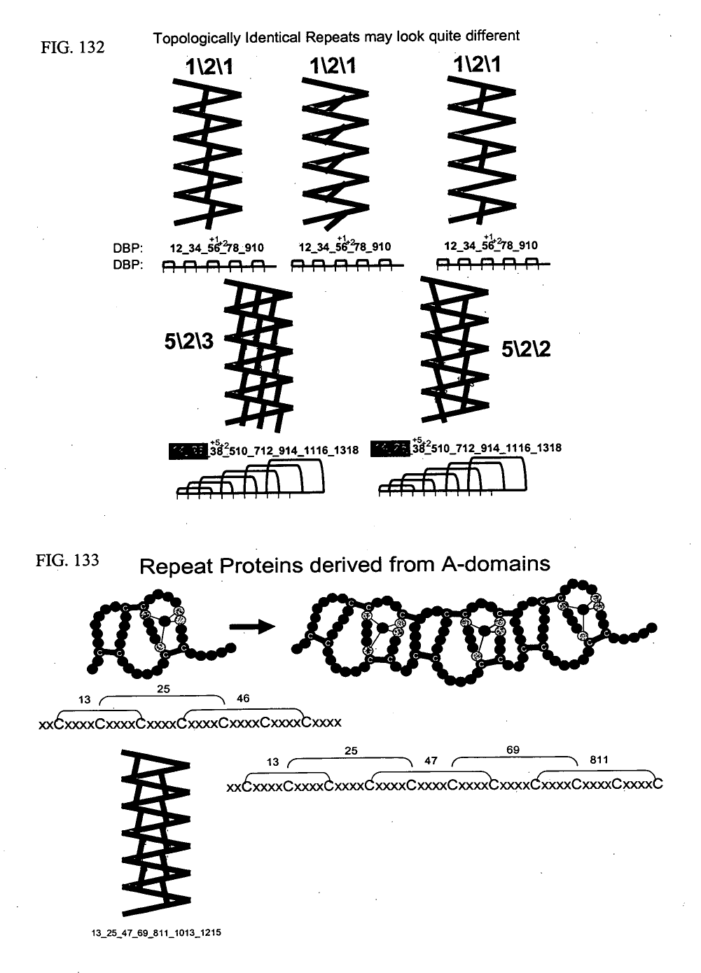

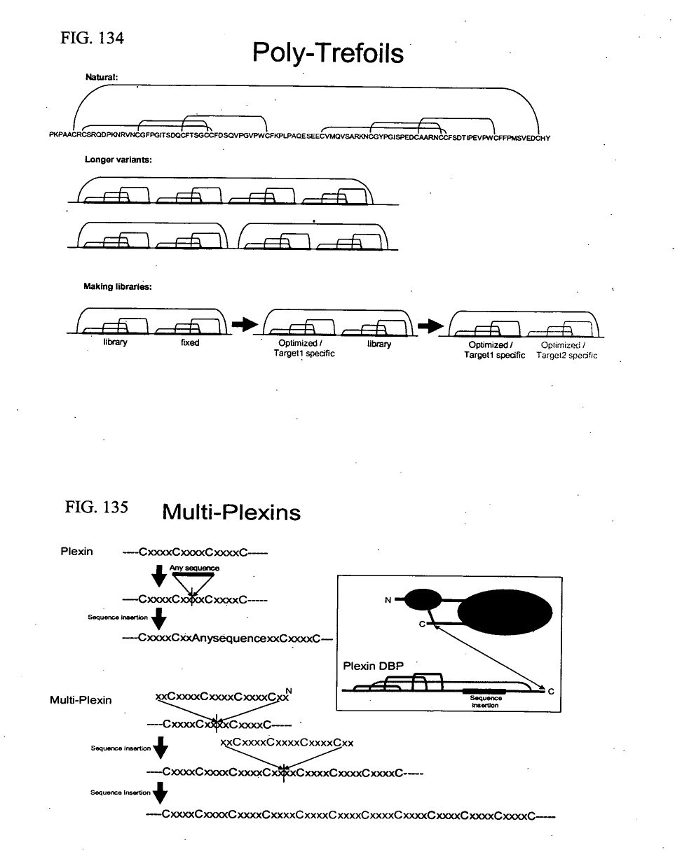

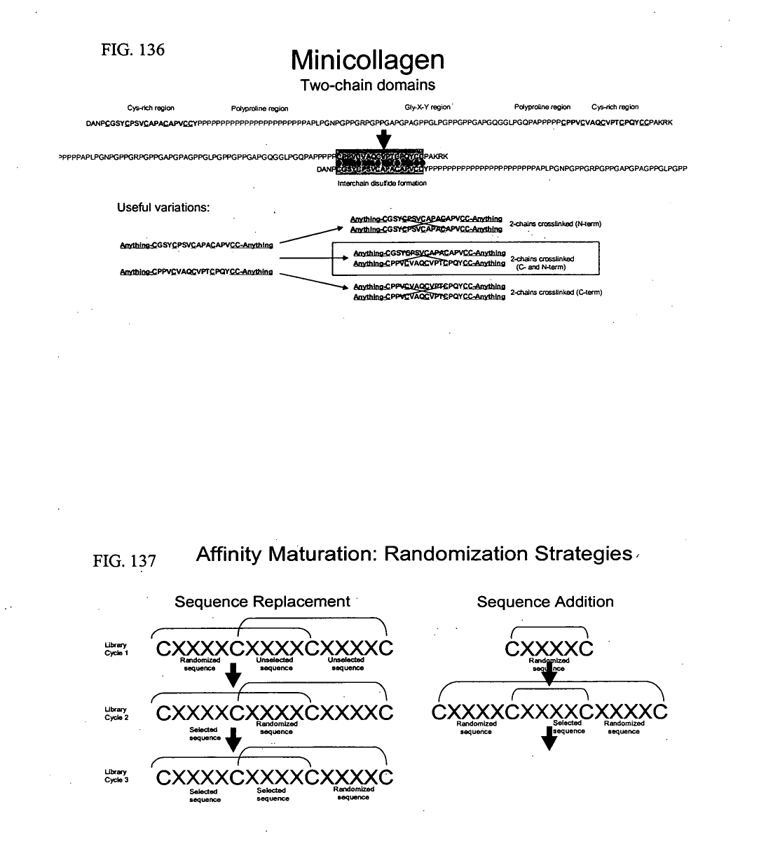

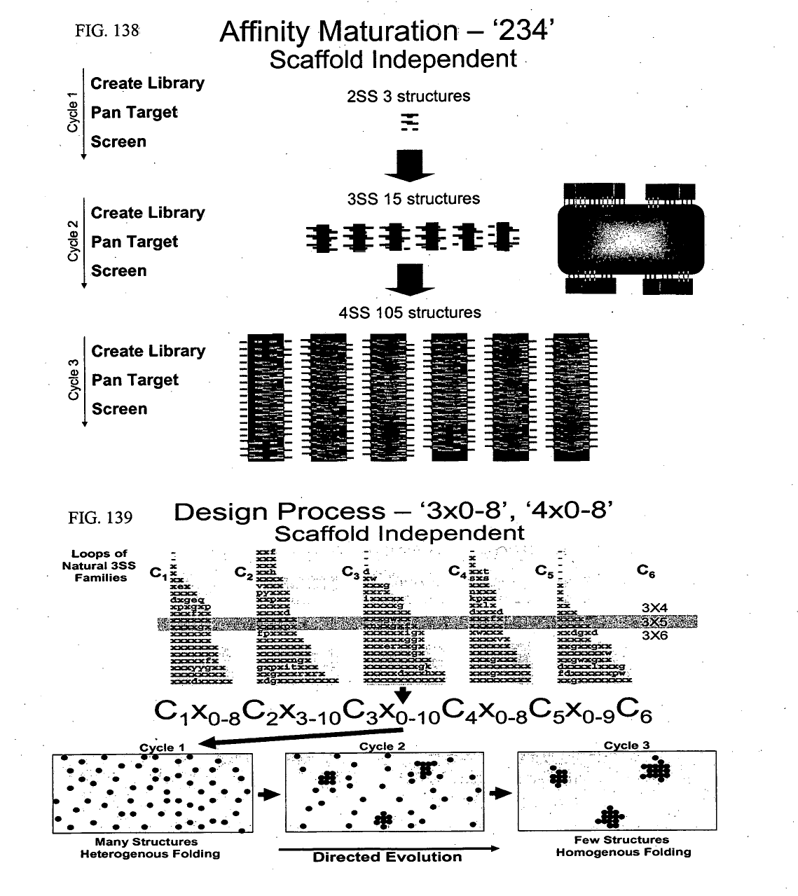

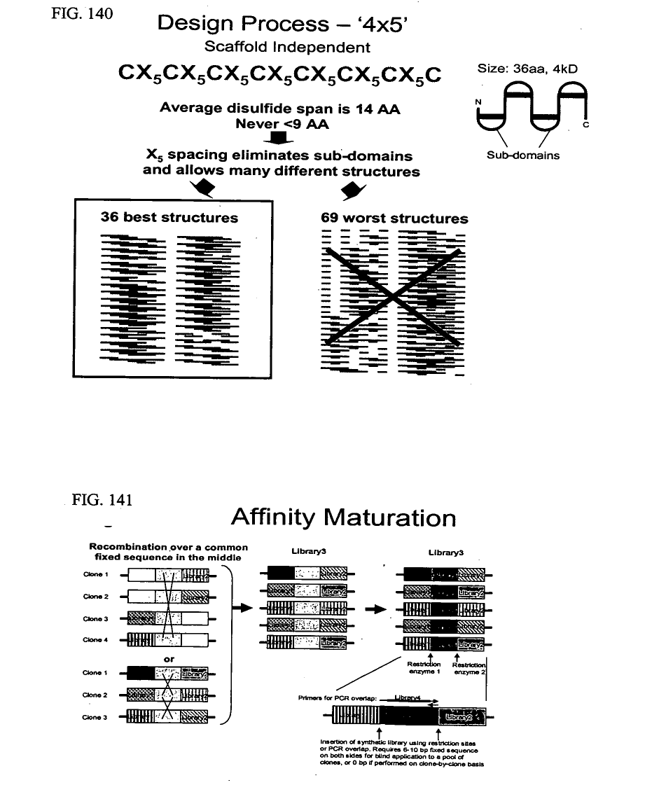

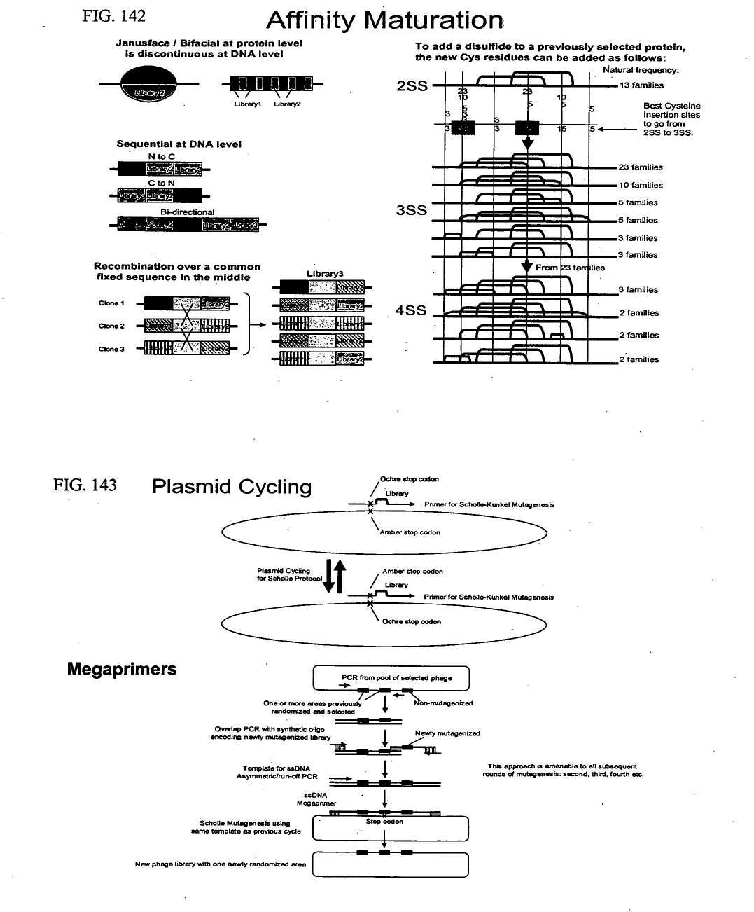

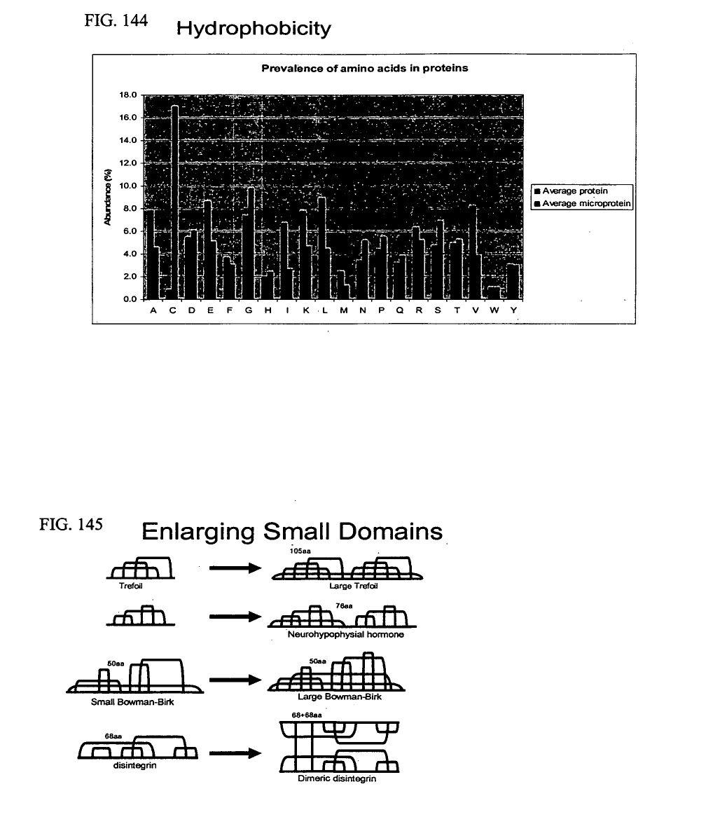

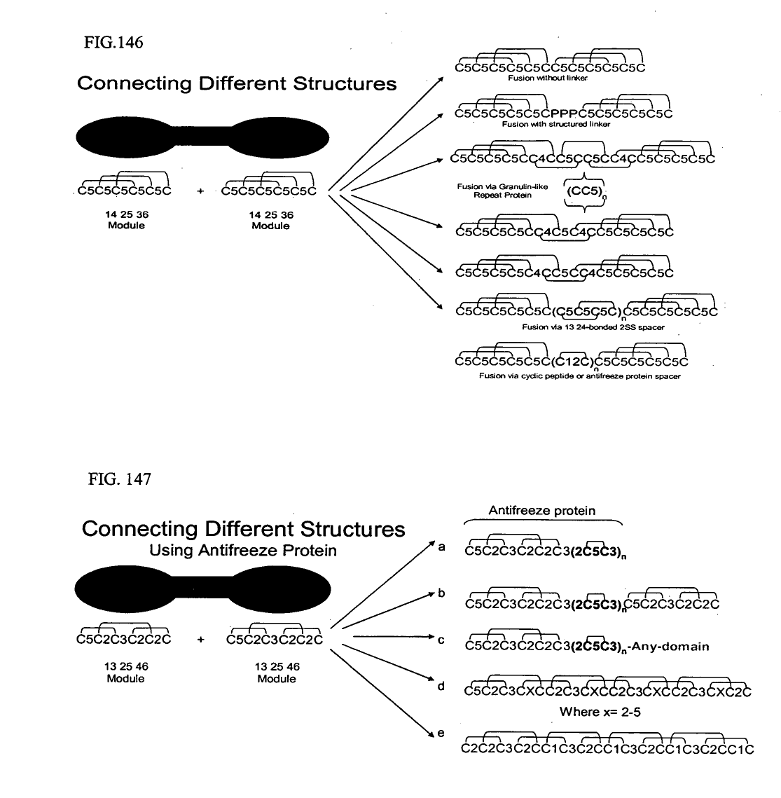

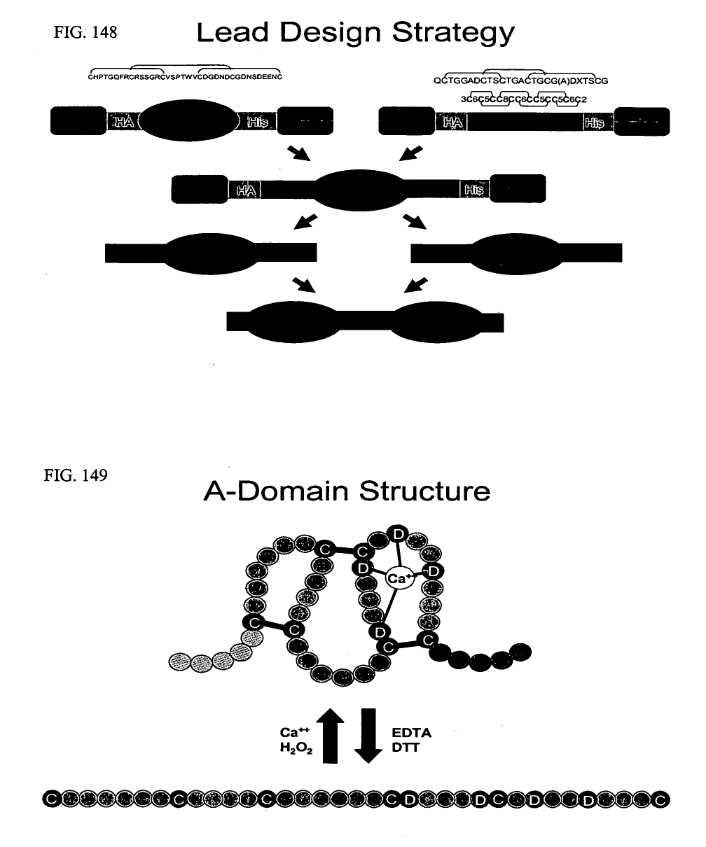

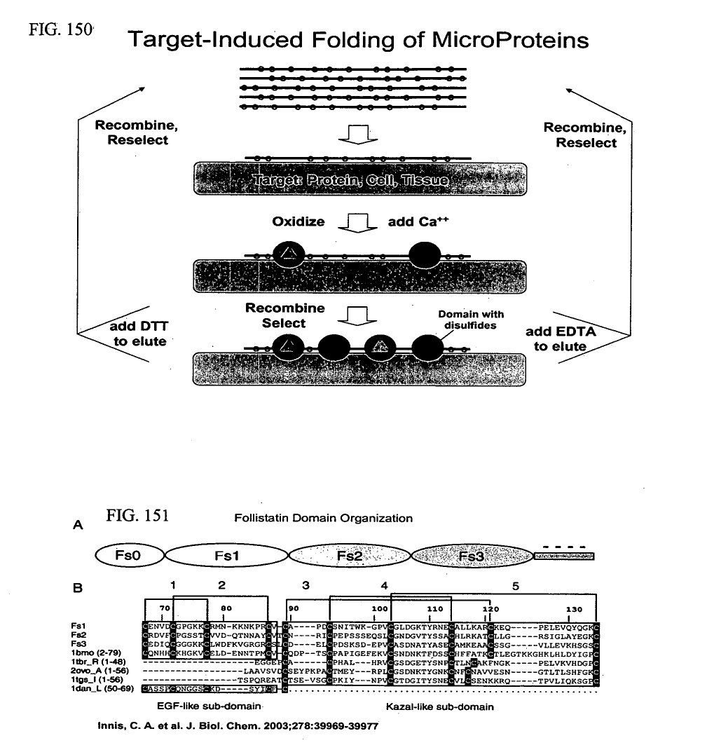

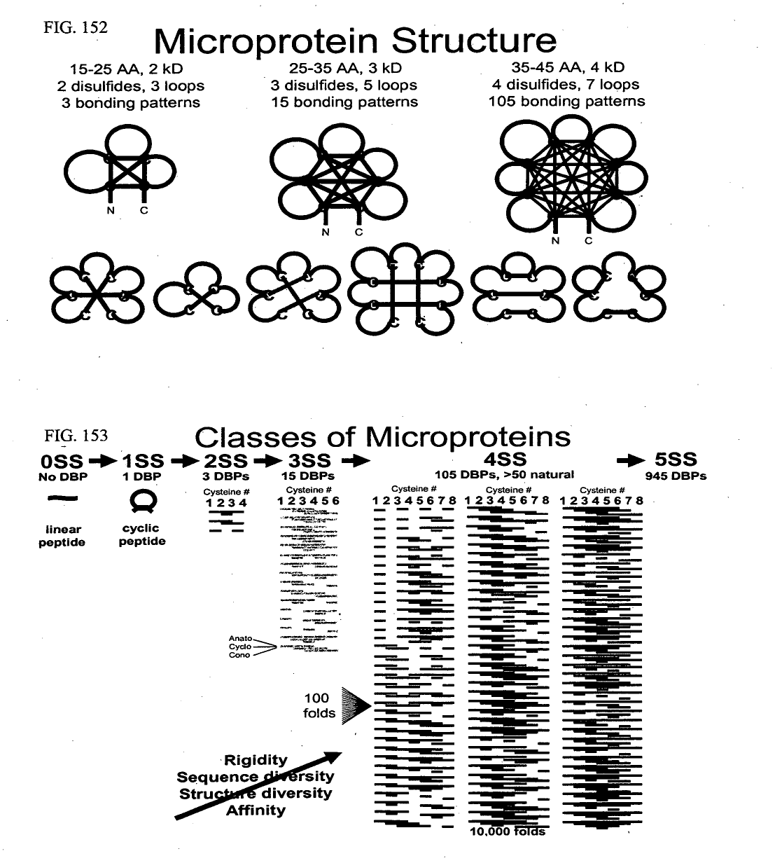

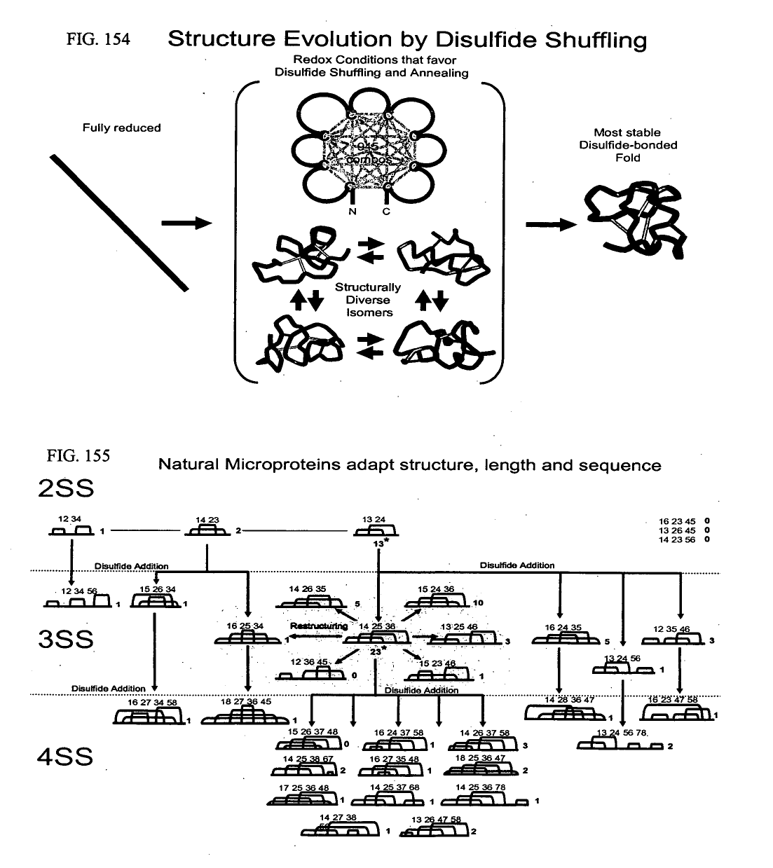

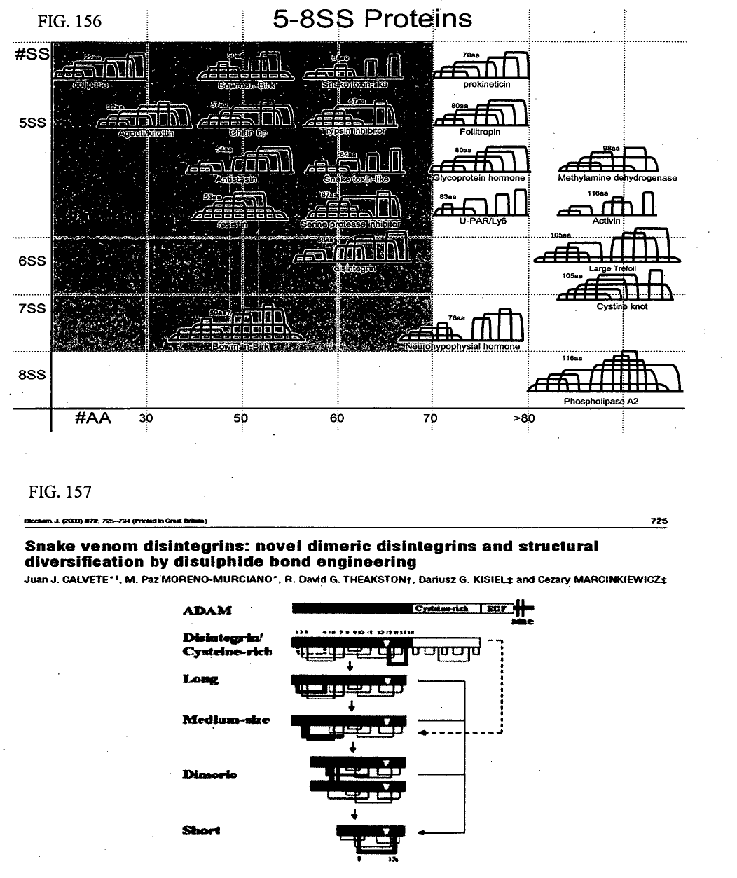

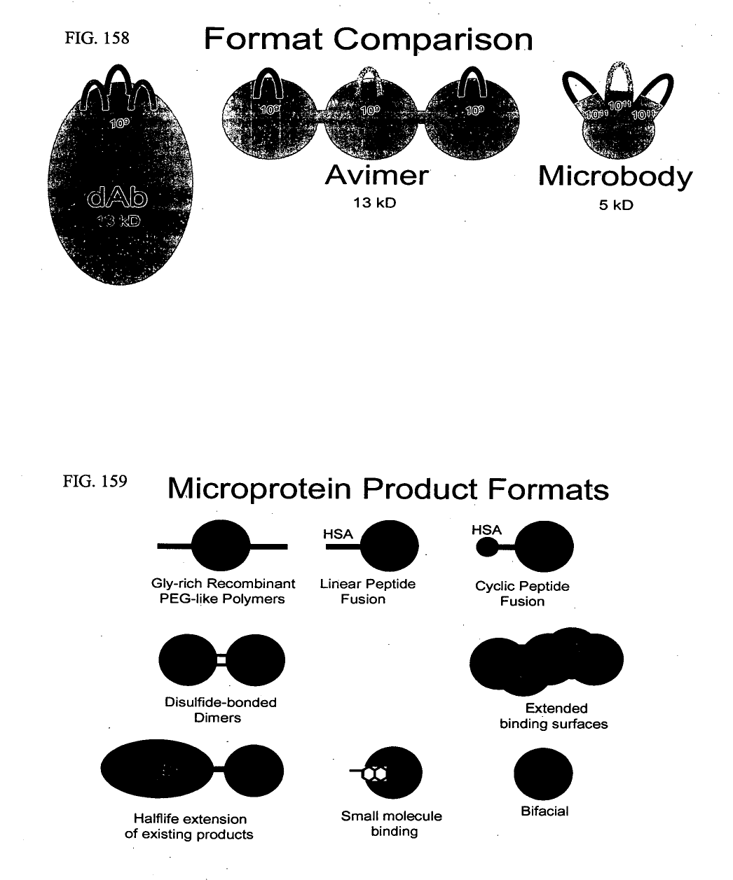

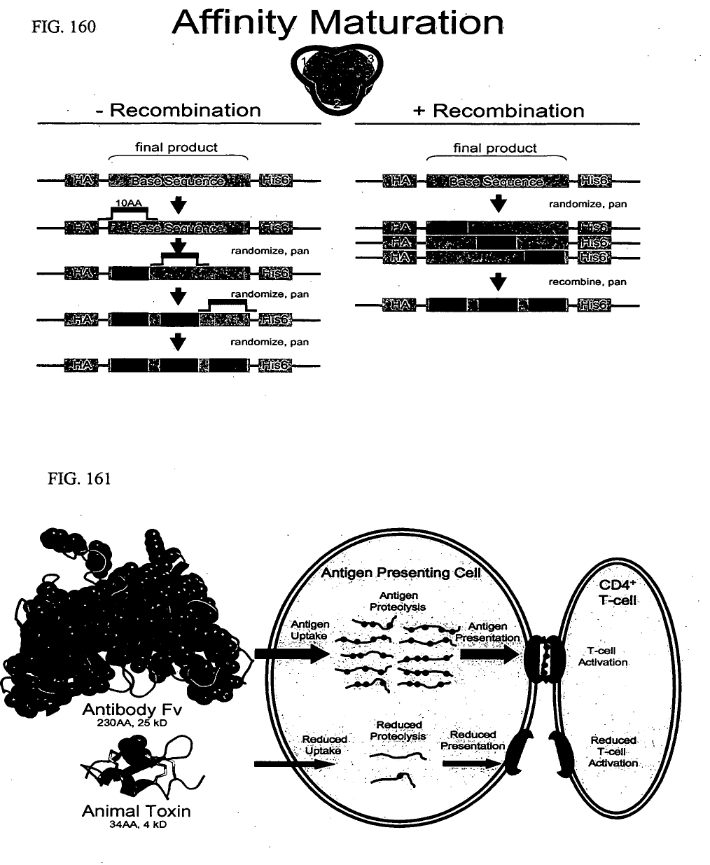

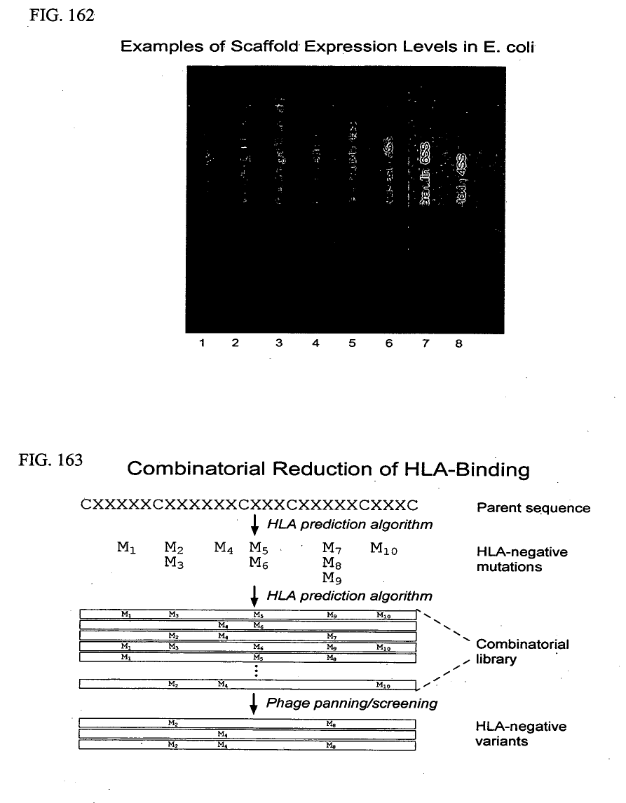

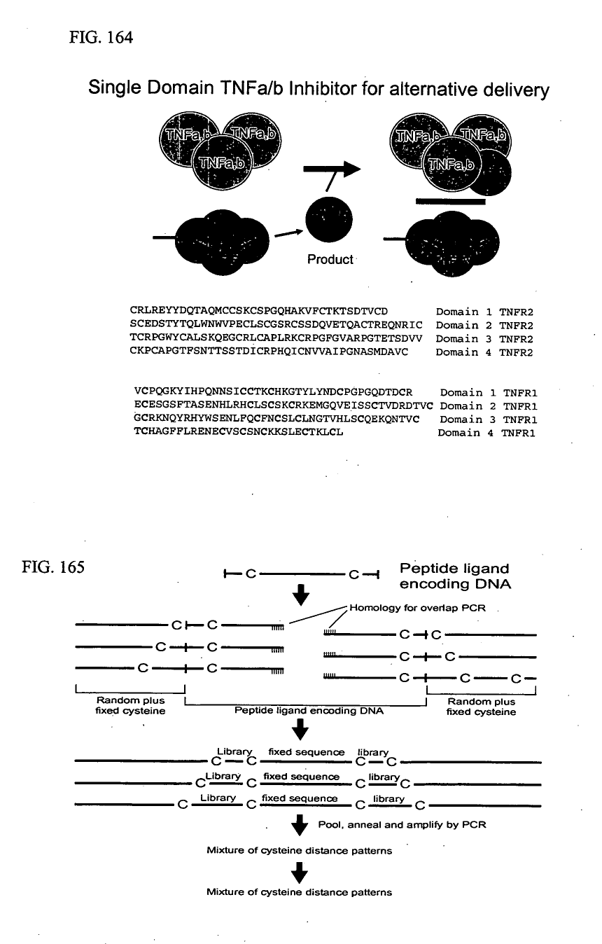

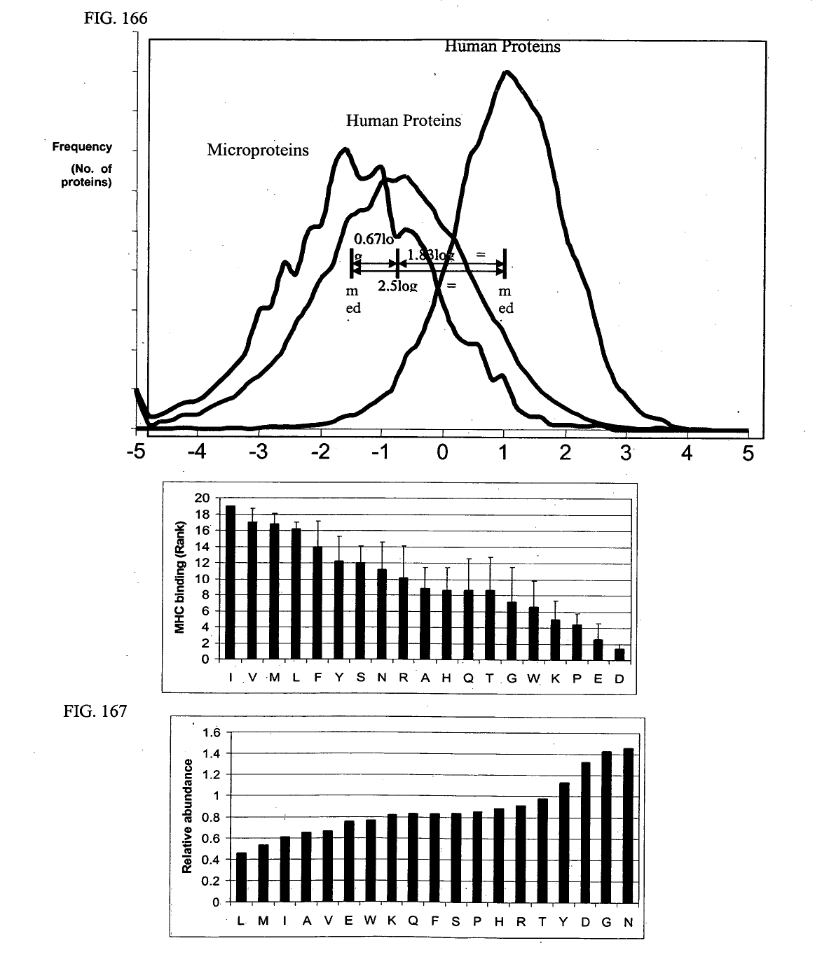

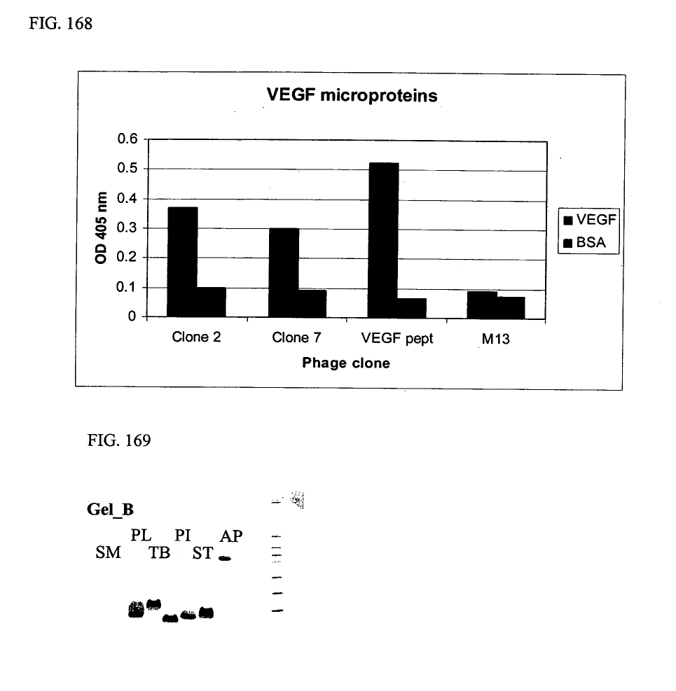

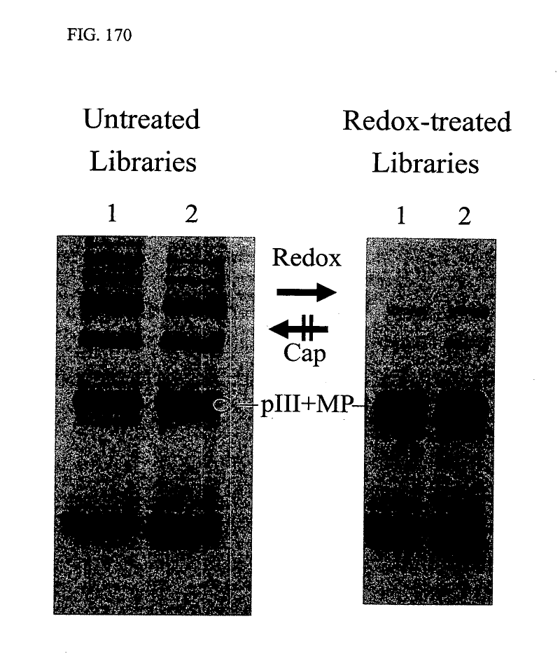

Motif for FIG. 123:

1) CtxxGxxxC(vilm)CxGxxxCGxGxxCxxxxxGxxnxC

2) CxxxGxxxCxCxGxxxCGxGxxCxxxxxGxxxxC

3) CxxxxxxxCxCxxxxxCxxxxxCxxxxxxxxxxC

CDP: C7C1C5C5C10C

Motif for FIG. 162:

1) CxxxxCxxxxxCxxx(x)xxxxxxCx(x)CxxxCxxxxxx(x)xxxCxxdxxtyxxxCxxxxaxCxxxxxxxxxxxgxC

2) CxxxxCxxxxxCxxx(x)xxxxxxCx(x)CxxxCxxxxxx(x)xxxCxxxxxxxxxxCxxxxxxCxxxxxxxxxxxxxC

CDP: C4C5C9-10C1-2C3C9-10C10C6C13C

FIG. 13 depicts the prevalence profile of amino acids in proteins.

FIGS. 17-18, 74, 84, 94, 98, 100 depict the primary and secondary structures of exemplary sequences.

FIGS. 19 and 36 depict sequence alignments amongst various invertebrate and plant proteins.

FIG. 103 depicts the sequence and tertiary structure of granulin.

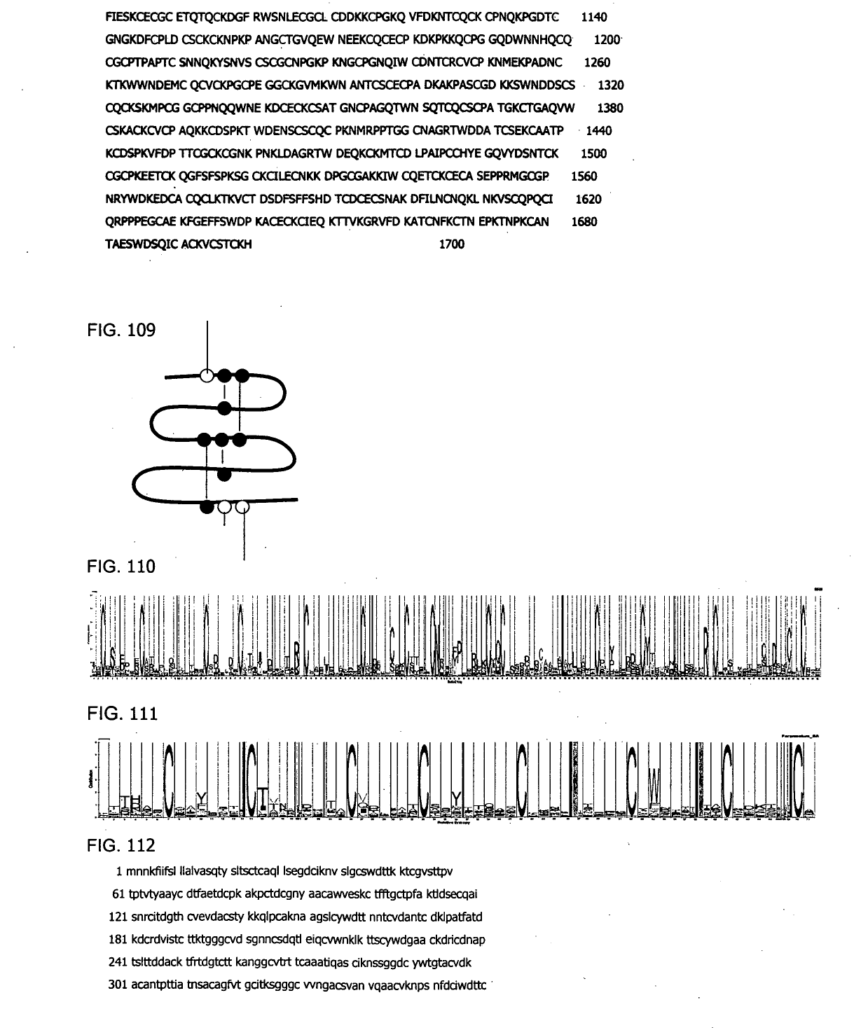

FIG. 107 depicts CXC motif repeats.

FIG. 108 depicts the sequence of VEGF C-terminal domain and balbani ring secreted protein.

FIG. 109 depicts the putative structure of a cysteine-containing repeat.

FIGS. 112 and 116 depict sequences of exemplary cysteine-containing repeat protein.

FIG. 117 depicts the structure of an exemplary anti-freeze protein.

FIG. 118 depicts the structure of erabutoxin.

FIG. 119 depicts the structure of plexin.

FIG. 120 depicts the sequence of plexin.

FIG. 121 depicts the structure of somatometin.

FIG. 122 depicts an SDS-PAGE gel separating expressed microproteins by molecular weight.

FIG. 124 depicts an affinity maturation scheme for cysteine-rich repeat proteins.

FIG. 125 depicts the structures of granulin repeat proteins.

FIG. 126 depicts a scheme for randomization.

FIG. 127 depicts the structures sand sequences of anti-freeze protein-derived repeat proteins.

FIG. 128 depicts a design of spiral repeat protein scaffolds.

FIG. 129 depicts a scheme for affinity maturation of repeat proteins.

FIGS. 130-132 depict cysteine-containing repeat protein nomenclatures.

FIG. 133 depicts repeat proteins derived from A-domains.

FIG. 134 depicts poly-trefoil scaffolds.

FIG. 135 depicts multi-plexin scaffolds.

FIG. 136 depicts minicollagen scaffolds.

FIGS. 137-142, 160 depict various schemes for affinity maturation.

FIG. 143 depicts plasmid cycling and megaprimers.

FIG. 144 is a hydrophobicity plot.

FIG. 145 depicts various was to enlarge small cysteine-containing domains.

FIGS. 146-147 depict various ways to connect different structures using anti-freeze proteins.

FIG. 148 depicts a strategy for designing libraries.

FIG. 149 depicts an A-domain structure.

FIG. 150 is a schematic representation of target-induced folding of microproteins.

FIG. 151 depicts the structural organization and sequence of the follistatin domain.

FIGS. 152-153 depicts structural diversity of cysteine-containing proteins.

FIGS. 154-155 depict structural evolution by disulfide shuffling and evolution of natural cysteine-containing proteins.

FIG. 156 depicts families of 508 disulfide containing proteins.

FIG. 157 depicts sequence relationship between different integrins.

FIG. 158 depicts a comparison of various product formats.

FIG. 159 depicts various microprotein product formats.

FIG. 161 depicts mechanisms for reducing immunogenicity.

FIG. 162 depicts a gel showing expression of various scaffolds from E. coli.

FIG. 163 depicts combinational reduction of HLA-binding.

FIG. 164 depicts sequences and structures of various TNFR family microproteins.

FIG. 165 depicts the 2-3-4 build-up approach.

FIG. 166 depicts predicted MHCII binding affinity of human and microproteins. The graph shows the distribution of scores for each protein calculated for five major HLA alleles. Red curve: 26,000 full length human proteins of median length 372AA. Blue curve: 10,525 microproteins of 25-90AA (medan 38AA) with at least 10% cysteine and an even number of cysteines, taken from a database of disulfide patterns (22). Green curve: 26,000 human protein fragments that match the size distribution of the microprotein data base. For each human protein sequence we randomly generated a fragment that matched the length of a randomly chosen protein from our microprotein data base. .MHCII binding was analyzed for 5 HLA alleles that occur with high frequency in the caucasian population, HLA*101, HLA*301, HLA*401, HLA*701, HLA*1501. MHCII binding matrices based on TEPITOPE were used. Binding matrices were downloaded from the program ProPred. TEPITOPE matrices do not contain scores for cysteine residues and alanine scores were used instead. For each protein and each HLA allele we identified the highest TEPITOPE score. Data for each allele were normalized by subtracting the average of the highest scores for all human proteins

FIG. 167 top panel shows affinity contribution of amino acids to MHCII binding. The P1 scores of all non-hydrophobic residues in the TEPITOPE matrices were changed from −999 to −2 to prevent the P1 score from dominating the average score. Amino acids were ranked according to their average score for each epitope. The figure shows the average ranks for the 5 most prevalent HLA alleles (*101, *301, *401, *701, *1501). The bottom panel shows relative abundance of amino acids in microproteins versus human proteins. Amino acid abundances were calculated for human proteins and microproteins using sequences as given in FIG. 166. The data show that the aliphatic hydrophobic residues I,V,M,L have the strongest contribution to immunogenicity and are the most underrepresented in microproteins compared to average human proteins. Reduction of the immunogenicity of proteins can thus be achieved by reducing the content of high-scoring amino acids, in the following rank order from high to low: IVMLFYSNRAHQTGWKPED.

FIG. 168 depicts the ELISA results of VEGF microproteins expressed from phage clones as a demonstration of the 2-3-4 build-up approach.

FIG. 169 depicts an SDS-PAGE gel of microproteins under reducing conditions. Lane 1: somatomedin, lane 2: plexin, lane 3: toxin B, lane 4: potato protease inhibitor, lane 5: spider toxin, lane 6: alkaline phosphatase control, lane 9: molecular weight marker.

FIG. 170 depicts a comparison of redox-treated libraries and untreated libraries

### INCORPORATION BY REFERENCE

All publications and patent applications mentioned in this specification are herein incorporated by reference for all purposes to the same extent as if each individual publication or patent application was specifically and individually indicated to be incorporated by reference.

### DETAILED DESCRIPTION OF THE INVENTION

All publications and patent applications mentioned in this specification are herein incorporated by reference for all purposes to the same extent as if each individual publication or patent application was specifically and individually indicated to be incorporated by reference for all purposes.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

General Techniques

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

Definitions

The term “protein” refers to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics. Proteins may comprise one or more domains.

The term ‘domain’ refers to as a single, stable three-dimensional structure, regardless of size. The tertiary structure of a typical domain is stable in solution and remains the same whether such a member is isolated or covalently fused to other domains. A domain as defined here has a particular tertiary structure formed by the spatial relationships of secondary structure elements, such as beta-sheets, alpha helices, and unstructured loops. In domains of the microprotein family, disulfide bridges are generally the primary elements that determine tertiary structure. In some instances, domains are modules that can confer a specific functional activity, such as avidity (multiple binding sites to the same target), multi-specificity (binding sites for different targets), halflife (using a domain, cyclic peptide or linear peptide) which binds to a serum protein like human serum albumin (HSA) or to IgG (hIgG1,2,3 or 4) or to red blood cells.

The ‘loops’ are the inter-cysteine sequences that contribute to the affinity and specificity of the interaction with the target, and their amino acid composition also affect the solubility of the protein which is important for high concentration formulations, such as those used in oral, intestinal, transdermal, nasal, pulmonary, blood-brain-barrier, home injection and other routes and formats of administration.

The term ‘microproteins’ refers to a classification in the SCOP database. Microproteins are usually the smallest proteins with a fixed structure and typically but not exclusively have as few as 15 amino acids with two disulfides or up to 200 amino acids with more than ten disulfides. A microprotein may contain one or more microprotein domains. Some microprotein domains or domain families can have multiple more-or-less stable and multiple more or less similar structures which are conferred by different disulfide bonding patterns, so the term stable is used in a relative way to differentiate microproteins from peptides and non-microprotein domains. Most microprotein toxins are composed of a single domain, but the cell-surface receptor microproteins often have multiple domains. Microproteins can be so small because their folding is stabilized either by disulfide bonds and/or by ions such as Calcium, Magnesium, Manganese, Copper, Zinc, Iron or a variety of other multivalent ions, instead of being stabilized by the typical hydrophobic core.

The term ‘scaffold’ refers to the minimal polypeptide ‘framework’ or ‘sequence motif’ that is used as the conserved, common sequence in the construction of protein libraries. In between the fixed or conserved residues/positions of the scaffold lie variable and hypervariable positions. A large diversity of amino acids is provided in the variable regions between the fixed scaffold residues to provide specific binding to a target molecule. A scaffold is typically defined by the conserved residues that are observed in an alignment of a family of sequence-related proteins. Fixed residues may be required for folding or structure, especially if the functions of the aligned proteins are different. A full description of a microprotein scaffold may include the number, position or spacing and bonding pattern of the cysteines, as well as position and identity of any fixed residues in the loops, including binding sites for ions such as Calcium.

The ‘fold’ of a microprotein is largely defined by the linkage pattern of the disulfide bonds (i.e., 1-4, 2-6, 3-5). This pattern is a topological constant and is generally not amenable to conversion into another pattern without unlinking and relinking the disulfides such as by reduction and oxidation (redox agents). In general, natural proteins with related sequences adopt the same disulfide bonding patterns. The major determinants are the cysteine distance pattern (CDP) and some fixed non-cys residues, as well as a metal-binding site, if present. In few cases the folding of proteins is also influenced by the surrounding sequences (ie pro-peptides) and in some cases by chemical derivatization (ie gamma-carboxylation) of residues that allow the protein to bind divalent metal ions (ie Ca++) which assists their folding. For the vast majority of microproteins such folding help is not required.

However, proteins with the same bonding pattern may still comprise multiple folds, based on differences in the length and composition of the loops that are large enough to give the protein a rather different structure. An example are the conotoxin, cyclotoxin and anato domain families, which have the same DBP but a very different CDP and are considered to be different folds. Determinants of a protein fold are any attributes that greatly alter structure relative to a different fold, such as the number and bonding pattern of the cysteines, the spacing of the cysteines, differences in the sequence motifs of the inter-cysteine loops (especially fixed loop residues which are likely to be needed for folding, or in the location or composition of the calcium (or other metal or co-factor) binding site.

The term ‘disulfide bonding pattern’ or ‘DBP’ refers to the linking pattern of the cysteines, which are numbered 1-n from the N-terminus to the C-terminus of the protein. Disulfide bonding patterns are topologically constant, meaning they can only be changed by unlinking one or more disulfides such as using redox conditions. The possible 2-, 3-, and 4-disulfide bonding patterns are listed below in paragraphs 0048-0075.

The term ‘cysteine distance pattern’ or ‘CDP’ refers to the number of non-cysteine amino acids that separate the cysteines on a linear protein chain. Several notations are used: C5C0C3C equals C5CC3C equals CxxxxxCCxxxC.

The term ‘Position n6’ or ‘n7=4’ refers to the intercysteine loops and ‘n6’ is defined as the loop between C6 and

C7; ‘n7=4’ means the loop between C7 and C8 is 4 amino acids long, not counting the cysteines.

The term ‘reductive unfolding’ involves the unfolding of a folded protein in the presence of a reducing agent (e.g. dithiothreitol). ‘Oxidative refolding’ involves the folding pathway from the fully unfolded and reduced state in the presence of oxidizing agent.

The term ‘complex’ refers to a cysteine bonding pattern in which the cysteines are disulfide bonded to cysteines that, on average, are separated by many amino acid positions on the linear alpha-chain backbone. ‘Complexity’ is quantified as the total (cumulative) linear backbone distance that the disulfides span. For example, the maximum for a 3-disulfide topology is 9 (1-4 2-5 3-6=3+3+3), and the minimum is 3 (i.e., 1-2 3-4 5-6). Complex patterns appear to offer more different folds due to length diversity but occur less frequently than less complex patterns. For example, the highest number of natural sequence families and the most rigid structure is observed for the patterns 1-4 2-5 3-6, 1-6 2-4 3-5, 1-5 2-4 3-6 and 1-4 2-6 3-5. All of these are the most complex pattern (complexity score of 9 on a 3-9 scale of 3SS proteins), showing that the more complex topologies appear to be able to yield more different cysteine spacings, ie more folds. Therefore, eliminating or reducing the frequency of simple disulfide bonding patterns (like 1-2 3-4 5-6) is expected to increase the average number of folds (i.e., very different cys-spacings, like conotoxin versus cyclotide versus anato) that is formed for each disulfide bonding pattern. A simple way to remove the majority of simple bonding patterns is to use loop lengths that are less than about 9 amino acids, since in natural proteins the minimum distance between cys residues that are disulfide-linked (called ‘span’) is generally about 9 amino acids. The complexity of 2SS proteins ranges from 2-4, and of 4SS proteins it is 4-16, and for 5SS proteins it ranges from 5-25.

The term ‘span’ of a disulfide bond refers to the amino acid distance between linked cysteines, excluding the cysteines themselves. The average span is 10-14AA, preferably about 12, as shown below in table 1. Spacing of cysteines such that multiples of 11-14aa are maximized can be used to encourage structural diversity by eliminating proximal disulfides (formed between neighboring cysteines) and by providing a large number of combinations of cysteine residues that have a span of about 12 amino acids (as well as 18, 24, etc). An example would be CX₆CX₆CX₆CX₆CX₆C (‘3×6’), CX₆CX₆CX₆CX₆CX₆CX₆CX₆C (‘4×6’), CX₅CX₅CX₅CX₅CX₅C (‘3×5’), CX₅CX₅CX₅CX₅CX₅CX₅CX₅C (‘4×5’), or similar motifs with a combination of loops ranging from 5-6, 4-7 or 3-8 amino acids. CX₆C and CX₅C are generally too short to allow the two adjacent cysteines to bond (minimum span is typically about 9 amino acids), preventing the formation of a cyclic peptide structure that is sometimes called a ‘sub-domain’ or ‘micro-domain’ but is generally not considered to be a full domain. Certain exemplary disulfide spans is show in the table below.

TABLE 1
Disulfide Span

| | C1-C6 distance | Disulfide Span (aa) | | |
|---|---|---|---|---|
| Family | (in aa) | 1 | 2 | 3 |
| A | 39 | 11 | 11 | 15 |
| EGF | 37 | 11 | 13 | 10 |
| TNFR | 42 | 12 | 12 | 17 |
| Kunitz | 52 | 50 | 23 | 20 |
| Notch | 34 | 23 | 12 | 15 |
| DSL | 43 | 24 | 15 | 28 |
| Trefoil | 40 | 19 | 14 | 16 |
| TSP1 | 45 | 33 | 36 | 10 |
| Anato | 37 | 25 | 31 | 19 |
| Thyroglobulin | 81 | 32 | 9 | 20 |
| Defensin 1 | 29 | 27 | 14 | 19 |
| Cyclotide | 24 | 16 | 14 | 14 |
| SHKT | 42 | 35 | 24 | 12 |
| Conotoxin | 29 | 15 | 13 | 10 |
| Toxin 2 | 29 | 20 | 21 | 15 |

The term “Cysteine-Rich Repeat Protein (‘CRRP’)” refers to a protein that typically but not exclusively has a single polypeptide chain and comprises ‘repeat units’ (also called ‘modules’, ‘repeats’ or ‘building blocks’) of a particular conserved amino acid sequence (‘repeat pattern’ or ‘repeat motif’) with a cysteine content of more than about 1%, preferably more than about 5% or even 10%. This family is unrelated in sequence from the Leucine-rich Repeat Proteins, which include the Ankyrin family. CRRP units interact with each other, resulting in one large domain that folds independently of other domains. CRRPs can be adjusted in size by adding or deleting repeat units. Preferred repeat proteins include but are not limited to head-to-tail repeats of the same motif, that are generally distinguishable from single repeats that are separated by unrelated sequences.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI., 15th Ed. (Mack Publ. Co., Easton (1975).

A “pharmaceutical composition” is intended to include the combination of an active agent with a carrier, inert or active, making the composition suitable for diagnostic or therapeutic use in vitro, in vivo or ex vivo.

The term “non-naturally occurring” as applied to a nucleic acid or a protein refers to a nucleic acid or a protein that is not found in nature. Examples of non-naturally occurring nucleic acids and proteins include but are not limited to those that have been modified recombinantly.

Design of Cysteine-Containing Proteins and Protein Libraries

As detailed below, one aspect of the present invention is to create protein libraries with vast structural diversity from which one can select and evolve binding proteins with desired properties for a wide variety of utilities, including but not limited to therapeutic, prophylactic, veterinary, diagnostic, reagent or material applications.

In one embodiment, the present invention provides cysteine-containing protein libraries with at least 2, 3, 4, 5, 10, 30, 100, 300, 1000, 3000, 10000 or more different structures that preferably are topologically distinct. In certain embodiments, the cysteine-containing protein libraries comprise high disulfide density (HDD) proteins. Proteins of the HDD family typically have 5-50% (5, 6, 7, 8, 9, 10, 12, 14, 16, 18, 20, 25, 30, 35, 40, 45 or 50%) cysteine residues and each domain typically contains at least two disulfides and optionally a co-factor such as calcium or another ion.

The presence of HDD scaffold allows these proteins to be small but still adopt a relatively rigid structure. Rigidity is important to obtain high binding affinities, resistance to proteases and heat, including the proteases (see below for classification of proteases) involved in antigen processing, and thus contributes to the low or non-immunogenicity of these proteins. The disulfide framework folds the protein without the need for a large number of hydrophobic side chain interactions in the interior of most proteins, called the hydrophobic core. All non-HDD scaffolds have a hydrophobic core which is a frequent source of specificity or folding problems. HDD proteins tend to be more hydrophilic than non-HDD proteins leading to improved binding specificity. The small size is also advantageous for fast tissue penetration and for alternative delivery such as oral, nasal, intestinal, pulmonary, blood-brain-barrier, etc. In addition, the small size also helps to reduce immunogenicity. A higher disulfide density is obtainable, either by increasing the number of disulfides or by using domains with the same number of disulfides but fewer amino acids. It is also desirable to decrease the number of non-cysteine fixed residues, so that a higher percentage of amino acids is available for target binding.

The disulfide framework allows extreme sequence diversity within each family in the intercysteine loops. Between families there exists vast variation in loop length and cysteine spacing. Due to the combinatorial nature of disulfide bond formation, the disulfide framework enables the formation of large numbers of different bonding patterns and different structures, and because folding can be heterogeneous, a gradual evolutionary path exists to optimize structures and sequences by directed evolution. The HDD proteins in particular are predicted to have the unique ability to allow a single sequence to adopt multiple different stable folds.

In order to generate a wide range of disulfide bonding patterns, the library can be subjected to a range of different conditions that may favor different isomers with different disulfide bonding patterns (DBPs). For example, one can exploit the redox potential of a solvent, which is determined by the relative concentration and strength of reducing and oxidizing agents, to effect formation of different DBPs. To create a reducing solvent, one can employ a variety of reducting agents including but not limited to 2-mercaptoethanol (beta-mercaptoethanol, BME), 2-mercaptoethylamine-HCl, TCEP (Tris(2-carboxyethyl) phosphine), Sodium borohydride, dithiothreitol (DTT, reduced form), reduced form of glutathione (GSH), and reduced form of cysteine. To create an oxidative solvent, one can employ a variety of oxidizing agents including without limitation dithiothreitol (DTT, oxidized form), hydrogen peroxide, glutathione (oxidized form, GSSG), copper phenanthroline (oxidized form), oxygen (air), trace metals and oxidized form of cysteine (cystine).

Particularly useful are mixtures and gradients of redox reagents that allow the protein to repeatedly form and break disulfides, sufficiently rapid to allow exploration of a vast diversity of disulfide bonding patterns and allowing stable forms to accumulate over time. If one wants maximum diversity of DBPs rather than stability, one can prevent a mixture from coming to equilibrium. Conditions that favor a large diversity of structures (fully reduced, high temperature) are suddenly changed into highly oxidizing, low temperature conditions such that the structures form with insufficient time to find the most stable DBP. An alternative approach to create structural diversity is to slowly form disulfides under a diversity of conditions, such as different chemicals (i.e., volume excluders like polyethyleneglycol, which accelerate formation of slow/difficult disulfide bonds with cysteines that are located far apart), different solvents (polar, non-polar, alcohols), different metal ions (Ca, Zn, Cu, Fe Mg, others) or different pHs (pH1,2,3,4,5,6,7,8,9,10,11,12). This variety of conditions alone or in any combination can be used to make the same protein sequence adopt a variety of alternative folds.

The formation of the disulfides and/or the presence of the co-factor can be easily controlled by providing reducing or oxidizing agents or by addition of a co-factor.

The ability of a protein to fold into multiple alternative stable structures will typically depend on the number and strength of the intra-protein bonding interactions as well as the properties of the available folding pathway(s). In the absence of disulfides, a large number of weak side chain contacts (salt bridges, van der Waals contacts, hydrophobic interactions, etc) are typically required to obtain a stably folded protein. Thus, many residues would need to be modified in order to direct the formation of a different, alternative stable fold or for binding to a target. In contrast, only a few (e.g., two or three) disulfide bonds are sufficient to give a protein a stable structure, leaving all of the other amino acid positions (typically around 65-80%) available to create binding surfaces for a desired target (conotoxins, at over 80%, are the most extreme example of this). Disulfides are thus a low information content approach (i.e., high frequency of occurrence in random sequences) to structure, leaving a maximum fraction of amino acids available for binding and various other functions.

The folding pathway and stability of a large, non-disulfide-containing protein require a large number of amino acid side chain interactions such that a large fraction of the residues must be more or less fixed, and therefore the ability of the protein to adapt its sequence is greatly reduced. This situation typically occurs in larger scaffold proteins, such as immunoglobulins, fibronectin and lipocalins, where usually only a few CDR-like loops can be randomized without causing misfolding, which for proteins such as these, containing a hydrophobic core, generally means irreversible protein aggregation. A single disulfide bridge, introduced by a couple of mutations, can take over the structural function of a large number of amino acid residues, freeing their sequence up to evolve towards a different purpose, such as binding to a desired protein target. Even in non-HDD proteins, the gradual addition of disulfides may play a key role in allowing the protein to continue to evolve towards increased complexity. Cysteine (C) appears to have been added late to the repertoire of 20 biological amino acids and the frequency of cysteines was shown to be rising gradually during protein evolution.

In addition, disulfide-mediated folding allows a protein to be more hydrophilic (because it replaces a hydrophobic core) and misfolding of such a protein generally does not lead to irreversible aggregation but allows the protein to stay soluble and renate eventually.

A unique feature of disulfides is that the same set of cysteines can, in principle, be linked in a variety of alternative disulfide bonding patterns, since disulfides are combinatorial. For example, two-disulfide proteins can have three different disulfide bonding patterns (DBPs), three-disulfide proteins can have 15 different DBPs and four-disulfide proteins have up to 105 different DBPs. Natural examples exist for all of the 2SS DBPs, the majority of the 3SS DBPs and less than half of the 4SS DBPs. In one aspect, the total number of disulfide bonding patterns can be calculated according to the formula:

$${\prod }_{i = 1}^{n} 2 i - 1 ,$$

wherein n=the predicted number of disulfide bonds formed by the cysteine residues, and wherein Π represents the product of (2i−1), where i is a positive integer ranging from 1 up to n.

Accordingly, in one embodiment, the present invention provides a non-naturally occurring cysteine (C)-containing scaffold exhibiting a binding specificity towards a target molecule, wherein the non-naturally occurring cysteine (C)-containing scaffold comprise intra-scaffold cysteines according to a pattern selected from the group of permutations represented by the formula

$${\prod }_{i = 1}^{n} 2 i - 1 ,$$

wherein n equals to the predicted number of disulfide bonds formed by the cysteine residues, and wherein Π represents the product of (2i−1), where i is a positive integer ranging from 1 up to n. In one aspect, the non-naturally occurring cysteine (C)-containing protein comprises a polypeptide having two disulfide bonds formed by pairing cysteines contained in the polypeptide according to a pattern selected from the group consisting of C^{1-2, 3-4}, C^{1-3, 2-4}, and C^{1-4, 2-3}, wherein the two numerical numbers linked by a hyphen indicate which two cysteines counting from N-terminus of the polypeptide are paired to form a disulfide bond. In another aspect, the non-naturally occurring cysteine (C)-containing scaffold comprises a polypeptide having three disulfide bonds formed by pairing intra-scaffold cysteines according to a pattern selected from the group consisting of C^{1-2, 3-4, 5-6}, C^{1-2, 3-5, 4-6}, C^{1-2, 3-6, 4-5}, C^{1-3, 2-4, 5-6}, C^{1-3, 2-5, 4-6}, C^{1-3, 2-6, 4-5}, C^{1-4, 2-3, 5-6}, C^{1-4, 2-6, 3-5}, C^{1-5, 2-3, 4-6}, C^{1-5, 2-4, 3-6}, C^{1-5, 2-6, 3-4}, C^{1-6, 2-3, 4-5}, and C^{1-6, 2-5, 3-4}, wherein the two numerical numbers linked by a hyphen indicate which two cysteines counting from N-terminus of the polypeptide are paired to form a disulfide bond. In another aspect, the non-naturally occurring cysteine (C)-containing protein comprises a polypeptide a non-naturally occurring cysteine (C)-containing protein exhibiting a binding specificity towards a target molecule, comprising a polypeptide having at least four disulfide bonds formed by pairing cysteines contained in the polypeptide according to a pattern selected from the group of permutations defined by the formula above. In yet another aspect, the non-naturally occurring cysteine (C)-containing protein comprises a polypeptide having at least five disulfide bonds formed by pairing intra-protein cysteines according to a pattern selected from the group consisting of C¹⁻⁹, C¹⁻¹⁰, C²⁻⁹, C²⁻¹⁰, C³⁻⁹, C³⁻¹⁰, C⁴⁻⁹, C⁴⁻¹⁰, C⁵⁻⁹, C⁵⁻¹⁰, C⁶⁻⁹, C⁶⁻¹⁰, C⁷⁻⁹, C⁷⁻¹⁰, C⁸⁻⁹, C⁸⁻¹⁰, and C⁹⁻¹⁰, wherein the two numerical numbers linked by a hyphen indicate which two cysteines counting from N-terminus of the polypeptide are paired to form a disulfide bond. In yet another aspect, the non-naturally occurring cysteine (C)-containing protein exhibiting a binding specificity towards a target molecule, comprising a polypeptide having at least six disulfide bonds formed by pairing intra-protein cysteines according to a pattern selected from the group consisting of C¹⁻¹¹, C¹⁻¹², C²⁻¹¹, C²⁻¹², C³⁻¹¹, C³⁻¹², C⁴⁻¹¹, C⁴⁻¹², C⁵⁻¹¹, C⁵⁻¹², C⁶⁻¹¹, C⁶⁻¹², C⁷⁻¹¹, C⁷⁻¹², C⁸⁻¹¹, C⁸⁻¹², and C⁹⁻¹¹, C⁹⁻¹², C¹⁰⁻¹¹, C¹⁰⁻¹², and C¹¹⁻¹², wherein the two numerical numbers linked by a hyphen indicate which two cysteines counting from N-terminus of the polypeptide are paired to form a disulfide bond.

Typically all of the cysteines are involved in disulfide bonding to other cysteines in the same domain. Microproteins with 2 disulfides (2SS) can adopt three different topologically distinct (ie not interconvertible by simple rotation) disulfide bonding patterns: 1-2 3-4, 1-3 2-4 or 1-4 2-3, each having a different alpha-chain backbone structure.

Similarly, microproteins with three disulfides can have up to 15 different disulfide bonding patterns, microproteins with 4 disulfides can have up to 105 disulfide bonding patterns, microproteins with 5 disulfides can have up to 945 disulfide bonding patterns, microproteins with 6 disulfides can have up to 10,395 disulfide bonding patterns and proteins with 7 disulfides can have up to 135,135 different bonding patterns, and so on for higher disulfide numbers (multipliers are 3,5,7,9,11,13-fold). The following lists the disulfide bonding patterns (DBP) for proteins with two, three or four disulfide bonds.

The 3 DBPs patterns for 2SS proteins are:
1-2 3-4, 1-3 2-4, 1-4 2-3

The 15 DBPs for 3SS proteins are:
1-6 2-5 3-4, 1-4 2-5 3-6, 1-6 2-4 3-5, 1-5 2-6 3-4, 1-5 2-4 3-6, 1-4 2-6 3-5, 1-2 3-4 5-6, 1-2 3-5 4-6, 1-2 3-6 4-5, 1-6 2-3 4-5, 1-4 2-3 5-6, 1-5 2-3 4-6, 1-3 2-6 4-5, 1-3 2-4 5-6, 1-3 2-5 4-6.

The 105 DBPs for 4SS proteins are:

| | | | | | |
|---|---|---|---|---|---|
| 1-2 3-4 5-6 7-8 | 1-2 3-4 5-7 6-8 | 1-2 3-4 5-8 6-7 | 1-2 3-5 4-6 7-8 | 1-2 3-5 4-7 6-8 | 1-2 3-5 4-8 6-7 |
| 1-2 3-6 4-5 7-8 | 1-2 3-6 4-7 5-8 | 1-2 3-6 4-8 5-7 | 1-2 3-7 4-5 6-8 | 1-2 3-7 4-6 5-8 | 1-2 3-7 4-8 5-6 |
| 1-2 3-8 4-5 6-7 | 1-2 3-8 4-6 5-7 | 1-2 3-8 4-7 5-6 | 1-3 2-4 5-6 7-8 | 1-3 2-4 5-7 6-8 | 1-3 2-4 5-8 6-7 |
| 1-3 2-5 4-6 7-8 | 1-3 2-5 4-7 6-8 | 1-3 2-5 4-8 6-7 | 1-3 2-6 4-5 7-8 | 1-3 2-6 4-7 5-8 | 1-3 2-6 4-8 5-7 |
| 1-3 2-7 4-5 6-8 | 1-3 2-7 4-6 5-8 | 1-3 2-7 4-8 5-6 | 1-3 2-8 4-5 6-7 | 1-3 2-8 4-6 5-7 | 1-3 2-8 4-7 5-6 |
| 1-4 2-3 5-6 7-8 | 1-4 2-3 5-7 6-8 | 1-4 2-3 5-8 6-7 | 1-4 2-5 3-6 7-8 | 1-4 2-5 3-7 6-8 | 1-4 2-5 3-8 6-7 |
| 1-4 2-6 3-5 7-8 | 1-4 2-6 3-7 5-8 | 1-4 2-6 3-8 5-7 | 1-4 2-7 3-5 6-8 | 1-4 2-7 3-6 5-8 | 1-4 2-7 3-8 5-6 |
| 1-4 2-8 3-5 6-7 | 1-4 2-8 3-6 5-8 | 1-4 2-8 3-7 5-6 | 1-5 2-3 4-6 7-8 | 1-5 2-3 4-7 6-8 | 1-5 2-3 4-8 6-7 |
| 1-5 2-4 3-6 7-8 | 1-5 2-4 3-7 6-8 | 1-5 2-4 3-8 6-7 | 1-5 2-6 3-4 7-8 | 1-5 2-6 3-7 4-8 | 1-5 2-6 3-8 4-7 |
| 1-5 2-7 3-4 6-8 | 1-5 2-7 3-6 4-8 | 1-5 2-7 3-8 4-6 | 1-5 2-8 3-4 4-7 | 1-5 2-8 3-6 4-7 | 1-5 2-8 3-7 4-6 |
| 1-6 2-3 4-5 7-8 | 1-6 2-3 4-7 5-8 | 1-6 2-3 4-8 5-7 | 1-6 2-4 3-5 7-8 | 1-6 2-4 3-7 5-8 | 1-6 2-4 3-8 5-7 |
| 1-6 2-5 3-4 7-8 | 1-6 2-5 3-7 4-8 | 1-6 2-5 3-8 4-7 | 1-6 2-7 3-4 5-8 | 1-6 2-7 3-5 4-8 | 1-6 2-7 3-8 4-5 |
| 1-6 2-8 3-4 5-7 | 1-6 2-8 3-5 4-7 | 1-6 2-8 3-7 4-5 | 1-7 2-3 4-5 6-8 | 1-7 2-3 4-6 5-8 | 1-7 2-3 4-8 5-6 |
| 1-7 2-4 3-5 6-8 | 1-7 2-4 3-6 5-8 | 1-7 2-4 3-8 5-6 | 1-7 2-5 3-4 6-8 | 1-7 2-5 3-6 4-8 | 1-7 2-5 3-8 4-6 |
| 1-7 2-6 3-4 5-8 | 1-7 2-6 3-5 4-8 | 1-7 2-6 3-8 4-5 | 1-7 2-8 3-4 5-6 | 1-7 2-8 3-5 4-6 | 1-7 2-8 3-6 4-5 |
| 1-8 2-3 4-5 6-7 | 1-8 2-3 4-6 5-7 | 1-8 2-3 4-7 5-6 | 1-8 2-4 3-5 6-7 | 1-8 2-4 3-6 5-7 | 1-8 2-4 3-7 5-6 |
| 1-8 2-5 3-4 6-7 | 1-8 2-5 3-6 4-7 | 1-8 2-5 3-7 4-6 | 1-8 2-6 3-4 5-7 | 1-8 2-6 3-5 4-7 | 1-8 2-6 3-7 4-5 |
| 1-8 2-7 3-4 5-6 | 1-8 2-7 3-5 4-6 | 1-8 2-7 3-6 4-5. | | | |

Large, low-cysteine proteins require extensive secondary, tertiary structure or even quaternary structure, which prevent the formation of alternative folds mediated by alternative disulfide bonding patterns. In microproteins there is little or no secondary or tertiary structure other than the disulfide induced structure and the intercysteine loop sequences (primary structure) are exceptionally variable in amino acid composition. Microproteins are therefore much more likely than other proteins to have enough sequence flexibility to allow them to adopt a variety of different bonding patterns.

A small number of cysteines are capable of providing a large diversity of completely different topological structures, meaning they cannot be interconverted without breaking the disulfides. These structures are typically obtained with no or minimal sequence requirements in the loops, leaving the loop sequences available for creating binding specificity and affinity for a specific target. A specific protein sequence is likely to show sharp preferences for some folds over others and may not be able to adopt some folds at all. From the sequence motifs of families of natural microproteins it appears that the spacing of the cysteines may contribute to the DBP, with a minor contribution from non-cys loop residues. The average length of inter-cysteine loops in high disulfide density proteins ranges from about 0 to about 10 for the most preferred scaffolds, to about 3 to about 15 amino acids for the majority of scaffolds, which provides a high density of cysteine ranging from about 50% for some scaffolds to 25%-20% (most preferred) to 15%-10% (less preferred) or even 5%, all of which are much higher than the density of Cysteine in average proteins, which is only 0.8%. Where desired, a close proximity of the cysteines is engineered to allow the disulfides to form efficiently and correctly. Efficient bond formation allows many cycles of breaking of the weakest bonds and reformation of new bonds, which gradually leads to the accumulation of the most stably bonded proteins. The low density of cysteines in large proteins appears to contribute to the inefficient and therefore likely incorrect formation of disulfides.

The different disulfide bonding patterns are expected to differ in their stability to temperature and to proteases. Accordingly, the present invention a non-naturally occurring cysteine (C)-containing scaffold (a) capable of binding to a target molecule, (b) having at least two disulfide bonds formed by pairing intra-scaffold cysteines, and (c) exhibiting the target binding capability after being heated to a temperature higher than about 50° C., preferably higher than about 80° C. or even higher than about 100° C. for a given period of time ranging from 0.01 second to 10 seconds. Where desired, the non-naturally occurring cysteine (C)-containing scaffold may be designed to contain at least three, four, five, six, seven, eight, nine, ten, eleven, twelve or more disulfide bonds, formed by pairing intra-scaffold cysteines.

Proteins that are more highly crosslinked (e.g., with high complexity number) are expected to be more stable than proteins that can form ‘sub-domains’, containing one or two disulfides but can freely rotate relative to the other part of the protein. Higher stability correlates with the (cumulative) length of the disulfides when drawn on a linear peptide (called ‘complexity’ of the fold) and with the number of times the disulfides intersect each other in a DBP diagram using a linear peptide sequence. However, the different disulfide bonding patterns are expected to form at different yields, with the most crosslinked versions being the least represented. To the extent that cysteine proximity drives disulfide formation, disulfides between adjacent cysteines are the most likely to occur but also the least desired from a stability perspective because they form micro- or sub-domains.

Accordingly, in some embodiments, the present invention provides protein libraries having non-naturally occurring cysteine (C)-containing proteins, each comprising no more than 35 amino acids, in which at least 10% of the amino acids in the polypeptide are cysteines, and at least two disulfide bonds are formed by pairing intra-scaffold cysteines, and wherein the pairing yields a complexity index greater than 3. In some other embodiments, the present invention provides protein libraries having non-naturally occurring cysteine (C)-containing proteins, each comprising no more than about 60 amino acids, in which at least 10% of the amino acids in the polypeptide are cysteines, at least four disulfide bonds are formed by pairing cysteines contained in the polypeptide, and wherein said pairing yields a complexity index greater than 4, 6, or 10.

In some aspects, the subject microproteins may exhibit picomolar activity toward a given target, and have high degree of resistance to heating (even boiling) and proteases. In other aspects, the subject micropteins tend to be highly hydrophilic, and tend to have two different binding faces per domain (bi-facial).

Although each disulfide bonding pattern is in theory compatible with a wide range of different spacings of the cysteines, some cysteine spacing patterns are more compatible with a specific bonding pattern than another cysteine spacing pattern. In natural sequences, there are multiple predominant cysteine spacing patterns associated with each disulfide bonding pattern. For example, the conotoxin, cyclotide and anato families (considered different folds) have very different cysteine spacing but the same disulfide bonding pattern. Thus, it is the spacing of the cysteines that primarily determines the frequency distribution of the disulfide bonding patterns, and design of the CDP is a practical way to control and evolve DBP and structure. The spacing of the cysteines determines the length of the intercysteine loops and to a large extent determines the ‘fold’ of the protein. Proteins belonging to the same family of sequences share the same scaffold sequence or scaffold motif, which is comprised of all of the highly conserved amino acid positions and their predominant spacings, and these are typically considered to have the same ‘fold’.

The subject microproteins can be monomers, dimers, trimers or higher multimers. Multi-domain microproteins can be homo-multimers or they can be hetero-multimers, in which the domains differ in disulfide number, disulfide bonding pattern, structure, fold, sequence, or scaffold. The subject microproteins can be fused to a variety of different structures including peptides (linear or cyclic) of a variety of different lengths, amino acid compositions and functions. Each domain can have one or more binding surfaces for different targets (i.e., bifacial), similar to or distinguished from many of the natural toxins.

The present invention also provides non-naturally occurring microproteins having a single protein chain that comprises one or more domains and optionally one or more (cyclic or linear) peptides. Generally each domain folds and functions separately. A microprotein domain has a high disulfide density ‘scaffold’ that largely determines the size of the domain, its stability to temperature and proteases and it's expression level in E. coli (and therefore the cost of goods). The scaffold also is expected to play a significant role in determining the immunogenicity of the protein. The scaffold comprises of 4,6,8,10,12,14,16,18 or more cysteines which form 2,3,4,5,6,7,8 or more disulfide bonds within the same domain.

Some of the preferred specific 3-disulfide scaffolds that offer improvements in multiple properties are the conotoxins (29aa total, 7aa fixed, no Ca-site, rigid structure due to 1-4 2-5 3-6 disulfide bonding pattern), the cyclotides (24aa total, 10aa fixed, No Ca-site, rigid 1-4 2-5 3-6 structure), the Anato scaffold (37aa total, 10aa fixed, No Ca-site, rigid 1-4 2-5 3-6 disulfide bonding pattern), the Defensin 1 scaffold (29aa total, 10aa fixed, No Ca-site, rigid 1-6 2-4 3-5 bonding pattern), the Toxin 2 scaffold (29aa total, 10 aa fixed, No Ca-site, rigid 1-4 2-6 3-5 disulfide bonded scaffold), but a wide variety of other existing and novel scaffolds also offer specific advantages. Other preferred scaffolds are Cellulose Binding domain (CB, CEB) which is Pfam family PF00734 with 173 members, 26AA long (from first to last Cys) with 4 cysteines linked 1-3 2-4 and a CDP of C10C5C9C; Alpha-conotoxin (AC), which is family PF07365 with 25 members, 15AA long and 4cysteines linked 1-3 2-4 and a CDP of C0C4C8C; Omega-toxin-like (OT) which is family PF00451 with 68 members and 28AA long with 6 cysteines linked 1-4 2-5 3-6 and a CDP of C5C3C10C4C1C; Pacifastin (PC) which is family PF05375 with 39 members and 29AA long and 6 cysteines linked 1-4 2-6 3-5 and a CDP of C9C2C1C8C4C; Serine Protease Inhibitor (SP) which is family PF00299 with 35 members and 26AA long and 6 cysteines linked 1-4 2-5 3-6 and a CDP of C6C5C3C1C6C; Notch (NO) which is family PF00066 with 175 members and 33AA long with 6 cysteines linked 1-5 2-4 3-6 and a CDP of C7C8C3C4C6C; Trefoil (TR) which is family PF00088 with 126 members and 39AA long with 6 cysteines linked 1-5 2-4 3-6 and a CDP of C10C10C4C0C10C; TNF-receptor-like (TN) which is family PF01821 with 123 members and 42AA long with 6 cysteines linked 1-2 3-5 4-6 and a CDP of C14C2C2C11C7C; Anaphylotoxin-like (AT) which is family PF01821 with 123 members and 37AA long with 6 cysteines linked 1-4 2-5 3-6 and a CDP of C5C2C8C2C5C1C; Plexin (PL) which is family PF01437 with 410 members and 61AA long with 8 cysteines linked 1-4 2-8 3-6 4-7 and a CDP of C5C2C8C2C5C12C19C; Other preferred scaffolds are Three Finger Toxin (TF) which is about 58AA long (first to last cys) and has 8 cysteines linked 1-3 2-4 5-6 7-8 and a CDP of C13C6C16C1C10C0C4C; Somatomedin which is 35AA long and has 8 cysteines linked 1-2 3-4 5-6 7-8 (note that alternate DBPs are known) and a CDP of C3C9C1C3C5C0C6C; Potato Protease Inhibitor (PI) which is 47AA long and has 8 cysteines and a CDP of C3C8C11C2C0C5C10C; Chitin Bindin Domian (CHB) which is 37AA long with 8 cysteines linked 1-4 2-5 3-6 7-8 and a CDP of C5C2C8C2C5C12C19C; Spider Toxin (ST) which is 34AA long with 6 cysteines and a CDP of C6C6C0C4C6C; Toxin B (TB) which is 34AA long and has 6 cysteines and a CDP of C6C5C0C3C8C; Cellulose Binding Domain (CEB) which is 26AA long with 4 cysteines linked 1-3 2-4 and a CDP of C10C5C9C; Alpha-Conotoxin (AC) which is 15AA long with 4 cysteines linked 1-3 2-4 and a CDP of C0C4C8C;

The subject non-naturally occurring microproteins may be designed based natural protein sequences. For example, numerous natural proteins or domains contained therein have attractive features for use as scaffold proteins. Non-limiting examples are listed in Table 2.

TABLE 2

| | Additional examplary members in the |
|---|---|
| Protein Family | family |
| Insulin-like | |
| Toxic hairpin | Heat stable enterotoxin, Neurotoxin B-IV |
| Knottins | Plant lectins, Antimicrobial peptides |
| | (Hevein-like agglutinin (lectin) domain), |
| | Antimicrobial peptide 2, AC-AMP2) |
| Plant inhibitors of proteinases and amylases | Trypsin inhibitor, Carboxypeptidase A |
| | inhibitor, Alpha-amylase inhibitor |
| Cyclotides | Kalata B1, Cycloviolacin O1, Circulin A, |
| | Palicourein |
| Gurmarin-like | |
| Agouti-related protein | |
| Omega-toxin-like | Conotoxin, Spider toxins, Insect toxins, |
| | Albumin 1 |
| Scorpion-toxin-like | Long chain scorpion toxins (Scorpion |
| | toxin, Alpha toxin, Tx10alpha-like toxin, |
| | LQH III alpha-like toxin) |
| | Short chain scorpion toxins, Defensin |
| | MGD-1, Insect defensins, Plant |
| | defensins |
| Cellulose binding domain | Cellobiohydrolase I |
| Growth factor receptor domain | Insulin-like growth factor-binding |
| | protein-5 IGFBP-5, Type 1 insulin-like |
| | growth factor receptor Cys-rich domain, |
| | Receptor protein-tyrosine kinase Erbb-3 |
| | Cys-rich domains, EGF receptor Cys- |
| | rich domains, Protooncoprotein Her2 |
| | extracellular domain |
| Colipase-like | (Pro)colipaseIntestinal toxin 1 |
| EGF/Laminin | EGF-type module (Factor IX, |
| | Coagulation factor VIIa, E-selectin, |
| | Factor X, N-terminal module, Activated |
| | protein C (autoprothrombin IIa), |
| | Prostaglandin H2 Synthase-1, EGF-like |
| | module, P-selectin, Epidermal Growth |
| | Factor (EGF), Transforming Growth |
| | Factor alpha, Epiregulin, EGF-domain, |
| | Betacellulin-2, Heparin-binding |
| | epidermal growth factor HBEGF, |
| | Plasminogen activator (urokinase type), |
| | Heregulin alpha, EGF domain, |
| | Thrombomodulin, Fibrillin-1, Mannose- |
| | binding protein associated serine |
| | protease 2, Complement C1S, |
| | Complement protease C1R, Plasminogen |
| | activator (tissue-type) (tPA), Low |
| | density lipoprotein (LDL) receptor) |
| | Integrin beta EGF-like domains, EGF- |
| | like domain of nidogen-1, Laminin-type |
| | module, Laminin gamma1 chain, |
| | Follistatin module N-terminal domain |
| | FS-N, Domain of BM- |
| | 40/SPARC/Osteonectin, Domain of |
| | Follistatin, Merozoite surface protein 1 |
| | (MSP-1) |
| Bromelain inhibitor VI (cysteine proteinase | |
| inhibitor) | |
| Bowman-Birk inhibitor | |
| Elafin-like | Elafin, elastase specific inhibitor, |
| | Nawaprin |
| Leech antihemostatic protein | Huristasin-like, Hirudin-like |
| Granulin repeat | N-terminal domain of granulin-1, |
| | Oryzain beta chain |
| Satiety factor CART (cocaine and amphetamine | |
| regulated transcript) | |
| DPY module | Dumpy |
| Bubble protein | |
| PMP inhibitors | |
| TSP-1 type 1 repeat | Thrombospondin-1 |
| AmbV | |
| Snake toxin like | Snake venom toxins (Erabutoxin B, |
| | gamma-Cardiotoxin, Faciculin, |
| | Muscarininc toxin, Erabutoxin A, |
| | Neurotoxin I, Cardiotoxin V4II (Toxin |
| | III), Cardiotoxin V, alpha-Cobratoxin, |
| | long Neurotoxin 1, FS2 toxin, |
| | Bungarotoxin, Bucandin, Cardiotoxin |
| | CTXI, Cardiotoxin CTX IIB, |
| | Cardiotoxin II, Cardiotoxin III, |
| | Cardiotoxin IV, Cobrotoxin 2, alpha- |
| | toxins, Neurotoxin II (cobrotoxin B), |
| | Toxin B (long neurotoxin), Candotoxin, |
| | Bucain) |
| | Dendroaspin |
| BPTI-like | |
| Extracellular domain of (human) cell surface | CD59, Type II activin receptor, BMP |
| receptors | receptor Ia ectodomain, TGF-beta type II |
| | receptor extracellular domain |
| Defensin-like | Defensin, Defensin 2, Myotoxin |
| Hairpin loop containing domain-like | APPLE domain |
| Neurotoxin III (ATXIII) | |
| LDL-receptor-like module | |
| Crambin-like | |
| Kringle-like | Kringle modules, Fibronectin type II |
| Kazal-type serine protease inhibitor | |
| Plant proteinase inhibitors | |
| Trefoil/Plexin domain-like | Trefoil, Plexin |
| Necrosis-inducing protein 1, NIP1 | |
| Cystine-knot cytokines | PDGF-like, TGF-beta-like, Noggin, |
| | Neurotrophin, Gonadotropin/Follitropin, |
| | Interleukin 17F, Coagulogen |
| Complement control module, SCR domain | CD46, beta2-glycoprotein, Complement |
| | receptor 1, 2 (cr1, cr2), Complement |
| | C1R and C1S protease domains, MASP-2 |
| Sea anemone toxin k | |
| Blood coagulation inhibitor (disintegrin) | Echistatin, Flavoridin, Kistrin, |
| | Obtustatin, Salmosin, Schistatin |
| Methylamine dehydrogenase, L chain | |
| Serine proterease inhibitors | ATI-like, BSTI-like |
| TB-module/8-cys domain | Fibrillin, TGFb-binding protein-1 |
| TNF receptor-like | TGF-R, NGF-R, BAFF-receptor |
| Heparin-binding domain from vascular endothelial | |
| growth factor | |
| Anti-fungal protein (AGAFP) | |
| Fibronectin type I module | Fibronectin, Tissue plasminogen |
| | activator, t-PA |
| Thyroglobulin type I domain | |
| Type X cellulose binding domain, CBDX | |
| Cellulose docking domain, dockering | |
| Carboxypeptidase inhibitor | |
| Invertebrate chitin binding proteins | |
| Pheromone ER-23 | |
| Mollusk pheromone | |
| Apical membrane antigen | |
| Somatomedin B domain | |
| Notch domain | |
| Mini-cllagen I, C-terminal domain | |
| Hormone receptor domain (HRM) | |
| Resistin | |
| YAP1 redox domain | |
| GLA domain | |
| Cholecystokinin A receptor N-domain | |
| HIV-1 VPU cytoplasmic domain | |
| HIPIP (high potential iron protein) | |
| Ferredoxin thioredoxin reductase (FTR), catalytic | |
| beta chain | |
| C2H2 and C2HC zinc fingers | |
| Zn2/Cys6 DNA-binding domain | |
| Glucocorticoid receptor-like | |
| SBT domain | |
| Retrovirus Zinc-finger-like domains | |
| Rubredoxin-like | |
| Ribosomal protein L36 | |
| Zinc-binding domain of translation initiation factor | |
| 2 beta | |
| B-box Zinc binding domain | |
| RING/U-box | |
| Pyk2-associated protein beta ARF-GAP domain | |
| Metallothionein | |
| Zinc domain conserved in yeast copper regulated | |
| transcription factors | |
| Ada DNA repair domain | |
| Cysteine rich domain | |
| FYVE/PHD zinc finger | |
| Zn-binding domains of ADDBP | |
| Inhibitor of apoptosis (IAP) repeat | |
| CCCH Zinc finger | |
| Zinc finger domain of DNA polymerase alpha | |
| TAZ domain | |
| Cysteine-rich DNA binding domain (DM) | |
| DnaJ/Hsp40 cysteine rich domain | |
| CCHHC domain | |
| SecC motif | |
| TSP type 3 repeat | |

The design of protease-resistant microproteins is important in terms of minimizing immunogenicity. Many natural microproteins are protease inhibitors. See, Rao, M. B. et al. (1998) Molecular and Biotechnological Aspects of Microbial Proteases. Microbiol Mol Biol Rev. 62(3): 597-635. According to the Nomenclature Committee of the International Union of Biochemistry and Molecular Biology, proteases are classified in subgroup 4 of group 3 (hydrolases). However, proteases do not comply easily with the general system of enzyme nomenclature due to their huge diversity of action and structure. Currently, proteases are classified on the basis of three major criteria: (i) type of reaction catalyzed, (ii) chemical nature of the catalytic site, and (iii) evolutionary relationship with reference to structure.

Proteases are grossly subdivided into two major groups, i.e., exopeptidases and endopeptidases, depending on their site of action. Exopeptidases cleave the peptide bond proximal to the amino or carboxy termini of the substrate, whereas endopeptidases cleave peptide bonds distant from the termini of the substrate. Based on the functional group present at the active site, proteases are further classified into four prominent groups, i.e., serine proteases, aspartic proteases, cysteine proteases, and metalloproteases. There are a few miscellaneous proteases which do not precisely fit into the standard classification, e.g., ATP-dependent proteases which require ATP for activity. Based on their amino acid sequences, proteases are classified into different families and further subdivided into “clans” to accommodate sets of peptidases that have diverged from a common ancestor. Each family of peptidases has been assigned a code letter denoting the type of catalysis, i.e., S, C, A, M, or U for serine, cysteine, aspartic, metallo-, or unknown type, respectively.

Exopeptidases: The exopeptidases act only near the ends of polypeptide chains. Based on their site of action at the N or C terminus, they are classified as amino- and carboxypeptidases, respectively.

Aminopeptidases: Aminopeptidases act at a free N terminus of the polypeptide chain and liberate a single amino acid residue, a dipeptide, or a tripeptide.

Carboxypeptidases: The carboxypeptidases act at C terminals of the polypeptide chain and liberate a single amino acid or a dipeptide. Carboxypeptidases can be divided into three major groups, serine carboxypeptidases, metallocarboxypeptidases, and cysteine carboxypeptidases, based on the nature of the amino acid residues at the active site of the enzymes.

Endopeptidases: Endopeptidases are characterized by their preferential action at the peptide bonds in the inner regions of the polypeptide chain away from the N and C termini. The presence of the free amino or carboxyl group has a negative influence on enzyme activity. The endopeptidases are divided into four subgroups based on their catalytic mechanism, (i) serine proteases, (ii) aspartic proteases, (iii) cysteine proteases, and (iv) metalloproteases.

Human proteases: Cathepsins B, C, H, L, S, V, X/Z/P and 1 are cysteine proteases of the papain family. Cathepsin L and Cathepsin S are known to be involved in antigen processing in antigen presenting cells. Cathepsin C is also known as DPPI (dipeptidyl-peptidase I). Cathepsin A is a serine carboxypeptidase and Cathepsin D and E are aspartic proteases. As lysosomal proteases, cathepsins play an important role in protein degradation. Because of their redistribution or increased levels in human and animal tumors, cathepsins may have a role in invasion and metastasis. Cathepsins are synthesized as inactive proenzymes and processed to become mature and active enzymes. Endogenous protein inhibitors, such as cystatins and some serpins, inhibit active enzymes. Other Cathepsins are Cathepsin G, D, and E.

Other human proteases one could engineer protein drugs to be resistant against are Tryptase, Chymase, Trypsin, Carboxypeptidase A, Carboxypeptidase B, Adipsin/Factor D, Kallikrein, Human Proteinase 3 (Sigma), Thrombin.

In addition, naturally-occurring HDD proteins can be used in designing the subject microproteins. Natural HDD proteins include many families of animal cell-surface receptor proteins, as well as defensive (ie ingested) and offensive (injectable) animal toxins, such as the venomous proteins of snakes, spiders, scorpions, snails and anemones. What these protein classes have in common is that they are at the host-environment/pathogen interface. These and any other natural proteins described herein serve as the exemplary scaffolds applicable for generating non-naturally occurring cysteine scaffolds of the present invention.

Of particular interest are proteins at this interface (in both host and pathogen) that tend to have specialized molecular support systems that allow them to rapidly adapt their sequence. Examples are the pilins in Neisseria and other bacteria, the antibody system in vertebrates, the trypanosome Variable Surface Glycoproteins, the Plasmodium surface proteins (which are in fact microproteins) and many other examples. Rapid adaptation of the AA sequence is clearly observed for microproteins, whose sequences tend to be much less similar than one would expect from the similarity of the genome sequences. The ability to rapidly adapt sequence while retaining a rigid structure (not necessarily the same structure, however) that prevents attack by proteases is likely the reason that this class of proteins has been recruited multiple (seven) times independently in the evolution of animals to serve as the origin of toxins. The repeated recruitment suggests that this class of proteins offers features that are especially useful for building toxins. Other constant features are the small size (these are the smallest folded proteins) and their extreme stability to proteases and temperature.

Receptor proteins and toxins show rapid rates of sequence variation, causing the toxins of closely related snails to appear completely unrelated. Rapid evolution is thought to be an essential feature of toxins because the venom needs to keep up with changes in a wide variety of receptor proteins (which show increased evolutionary rates for resistance to the toxins) in a wide and changing variety of prey species. One very useful feature of this group is the low degree of immunogenicity imparted by the protease stability of the high disulfide density scaffold, as described in multiple publications. This may be important to avoid creating resistance to toxins in prey that were bitten but got away. Since both the receptor and the toxin need to adapt sequence rapidly, it is not surprising that in some cases both are comprised of HDD microprotein domains. For example, the structure-based class of snake-toxin-like proteins (as defined by the Structural Classification of Proteins (SCOP) database) contains both snake venom toxins as well as the extracellular domains of human cell surface receptors, some of which interact with ligands of the same structure (i.e., TGFbeta-TGFbeta-receptor). Examplary proteins include snake-toxin-like proteins such as snake venom toxins and extracellular domain of human cell surface receptors. Non-limiting examples of snake venom toxins are Erabutoxin B, gamma-Cardiotoxin, Faciculin, Muscarininc toxin, Erabutoxin A, Neurotoxin I, Cardiotoxin V4II (Toxin III), Cardiotoxin V, alpha-Cobratoxin, long Neurotoxin 1, FS2 toxin, Bungarotoxin, Bucandin, Cardiotoxin CTXI, Cardiotoxin CTX IIB, Cardiotoxin II, Cardiotoxin III, Cardiotoxin IV, Cobrotoxin 2, alpha-toxins, Neurotoxin II (cobrotoxin B), Toxin B (long neurotoxin), Candotoxin, Bucain. Non-limting examples of extracellular domain of (human) cell surface receptors include CD59, Type II activin receptor, BMP receptor Ia ectodomain, TGF-beta type II receptor extracellular domain.

In most natural HDD protein families the disulfide scaffold alone is able to provide a high level of rigidity, which favors high affinity by avoiding an induced fit and the associated entropy penalty. In many microprotein families just 4, 6, 8 or 10 cysteine residues appear to be able to fully determine major properties such as the structure, thermo-resistance and protease resistance of the protein, while leaving all (as in conotoxins) or nearly all of the other residues in the loops free to adopt any sequence that is desired for binding specificity. The cysteines provide a critical function with a minimum of sequence definition (‘low information content’), which statistically favors independent recruitment of this scaffold over alternative scaffolds with more fixed amino acids and a higher information content. For example, 2 extra fixed amino acids increase the information content and reduce the predicted frequency of recruitment from or occurrence in a random pool of sequences by 20×20=400-fold. Similar levels of protein stability based on non-cys amino acids would take many more residues, resulting in a larger and/or evolutionarily less adaptable protein.

One source of structural diversity of natural toxins is caused by the length variation that HDD (high disulfide density) proteins have been demonstrated to exhibit on an evolutionary timescale. This is described in detail for snake disintegrins (Calvete, J. J., Moreno-Murciano, M. P., Theakston, R. D. G., Kisiel, D. G. and Marcinkiewicz, C. (2003) Snake venom disintegrins: Novel dimeric disintegrins and structural diversification by disulphide bond engineering. Biochem J. 372:725-734. Calvete, J. J., Marcinkiewcz, C., Monleon, D., Esteve, V., Celda, B., Juarez, P. and Sanz, L. (2005) Snake venom disintegrins: Evolution of structure and function. Toxicon 45:1063-1074).

Deletions (or insertions/additions) of parts of a gene encoding a large HDD protein can give rise to a large number of smaller (or larger) variants that, although homologous to the original sequence, would be regarded as different structures. In the published examples, most of the disulfides are conserved, but a minority of cysteines forms new bonding patterns. The natural mechanisms for this may involve modification at the DNA level, mRNA alternative splicing, degradation, protein (trans-)splicing or other forms of truncation or addition at either end, alternative translation, as well as degradation or other forms of truncation. Whatever the natural mechanism, this principle can be implemented using molecular biology and (phage) display libraries to evolve proteins with optimal potency and stability and minimal size.

One can also generate novel and modified scaffolds from natural protein sequences including the following preferred families: A-domains, EGF, Ca-EGF, TNF-R, Notch, DSL, Trefoil, PD, TSP1, TSP2, TSP3, Anato, Integrin Beta, Thyroglobulin, Defensin 1 as well as additional families disclosed herein. Existing protein domain families with 2 or more disulfides that function as animal toxins, include the preferred families: Toxin 1, 2, 3, 4, 5, 6, 7, 9, 11, 12, Defensin 1, Defensin 2, Cyclotide, SHKT, Disintegrins, Myotoxins, Gamma-Thioneins, Conotoxin, Mu-Conotoxin, Omega-Atracotoxins, Delta-Atracotoxins as well as additional families listed herein. The modified scaffold may differ from the natural ones in cysteine numbers, disulfide bonding pattern, spacing, size/length from first to last cysteine, loop structure (having different fixed residues or size), ion binding site (with different location, amino acid composition, and ion specificity), performance-related features (including safety, non-immunogenicity, more similar to human, less similar to human, temperature stability, protease stability, hydrophobicity Index, percentage of hydrophilic amino acids, formulation properties like eutectic point, high concentration, absence of specific residues, rigidity, disulfide density, percentage library residues, complexity of the disulfide bonding pattern, and etc.).

In some cases it is useful to reflect the sub-families that occur in natural diversity, which can be done by including in the same scaffold library multiple length variations of a specific loop design (typically using separate oligonucleotides), each for a different sub-family and reflecting length and sequence differences between sub-families.

In some applications it may be useful to generate improved variants of existing scaffolds. For example, novel variants of the LDL receptor type A-domains (‘A-domains’) or EGF domains can be generated by a variety of relatively conservative approaches that are likely to result in improved scaffolds compared to the original. There exists a variety of ways to modify the variants, including inverting the cysteine motif (incl. spacing) alone or the motif of conserved residues (incl. non-cys) of the A-domain, by switching the N-terminus to the C-terminus. Inversion has been shown to be feasible with some small peptides and in this case only a small number of amino acids is inverted. Other modifications may involve changing the length of the proteins (shorter or longer) to fall outside the length range of protein domains in the published libraries or in the natural sequences, moving the calcium binding site to a different set of loops, and changing one or more of the fixed non-cys residues in the loops. If the fixed residue is a D, the goal would be to get a non-D residue at this position. A good way to implement this and to test a large number of compositions that are novel for a specific amino acid position is to use a codon that provides a mix of amino acids that is the opposite (ie complementary) of the naturally occurring amino acids or of the mix used in the published libraries. If the published library contains I, L, V in a position, then a novel motif could be obtained by providing all 20 AA except I,L, V in that position. Each position will differ in it's amino acid requirements for structure, and even more so for function.

Libraries of scaffolds can also be used to find better variants of existing scaffold sequence motifs. One can look for scaffolds that are better than the known scaffold in one or more of the following aspects: different disulfide bonding pattern, and/or different spacing of the disulfides and/or different sequence motifs of the loops, and/or difference in the fixed loop residues and/or different location, absence or AA composition or ion specificity of the calcium binding site.

Those skilled in the art know how to apply these principles to scaffolds other than A-domains, including the domain families EGF, Ca-EGF, TNF-R, Kunitz, Notch/LNRIDSL, Trefoil/PD/P-type, TSP1, TSP2, TSP3, Anato, Integrin Beta, Thyroglobulin, Toxin 1, 2, 3, 4, 5, 6, 7, 9, 11, 12, Defensin 1, Defensin 2, Cyclotide, SHKT, Disintegrins, Myotoxins, Gamma-Thioneins, Conotoxin, Mu-Conotoxin, Omega-Atracotoxins, Delta-Atracotoxins as well as the additional families listed in table.

Exemplary modified and novel scaffolds derived from A-domains include protein domain with non-natural sequence (and less than 50aa) which contains the sequence C₁(xx)xxEDsxDxC₂DxxGDC₃xWxx[ps]xC₄(xx)xxxC₅xFxxx(xx)C₆ plus one additional disulfide. There are a number of 4-disulfide domains that are similar to, for example, the 3-disulfide A-domain but are more rigid because they have an extra cysteine in a location that stabilizes the relatively flexible A-domain structure. An example is the 1-8 2-4 3-6 5-7 bonding pattern that comprises the A-domain's 3SS fold (1-3 2-5 4-6), but stabilizes it with 1 disulfide on either side of the A-domain sequence and thereby fixes a key structural weakness. Other high-quality 4-disulfide versions of the A-domain (called ‘A+domains’) are: 1-5 2-4 3-7 6-8, 1-3 2-6 4-8 5-7, 1-4 2-7 3-6 5-8, 1-4 2-7 3-6 5-8, as well as many others. Size should be the similar to the A-domain, just a few AA longer (2-12, preferably less than 8AA). This same analysis and solution can be used for all other 3-disulfide families and also to 2- and 4-disulfide families having the general structures as follows:

Protein domain (with non-natural sequence and less than 50aa) containing the sequence C₁x(xxx)xFxC₂xxx(xxx)C₃xx(xx)xxxC₄DGxxDCsxDxSDE(xxxx)xC₆ and more than 36 aa between C₁ and C₆.

Protein domain (with non-natural sequence and less than 50aa) with the sequence C₁x(xxx)xFxC₂xxx(xxx)C₃xx(xx)xxxC₄DGxxDC₅xDxSDE(xxxx)xC₆ and less than 32 aa between C₁ and C₆.

Protein domain with non-natural sequence and less than 50aa, with three disulfides linked 1-3 2-5 4-6 and more than 36 aa between C1 and C6.

Protein domain with (non-natural sequence and less than 50aa) with the sequence C₁x(xxx)xFxC₂xxx(xxx)C₃xx(xx)xxxC₄DGxxDC₅xDxSDE(xxxx)xC₆ and less than 32 aa between C₁ and C₆.

Protein domain with non-natural sequence (and less than 50aa) which contains the sequence C₁(xx)xxxxxxxxC₂xxxxxC₃xxxxxxC₄(xx)xxxC₅xxxxx(xx)C₆ (inverted A-domain)

Protein domain (with non-natural sequence and less than 50aa) in which one of the underlined amino acids is not present:

C₁x[aps](x)[ekq]FxC₂xxxx(x)C₃[ilv][ps]xx[lw][lrv]C₄DG[dev][pnd]DC₅xD[dgns]SDE(aps)(lps)xxC₆.

A different presentation of the same approach is (3 different motif levels shown; desired changes underlined):

C₁x(xx)xxxnonFxC₂xxxx(xx)C₃xxxxxxC₄xxxxnonDC₅x(x)xxxnonDnonE(x)xxxC₆ or

C₁x(xx)xxxnonFxC₂XXXX(XX)C₃[nonILV][nonPS]xxxxC₄nonDnonGxxnonDC₅x(x)nonDxnonSnonDnonE(x) xxxC₆

Protein domain with (with non-natural sequence and) the Huwentoxin II fold, a spider toxin that has the same bonding pattern as the A-domain fold but a very different spacing of the cysteines and completely unrelated protein sequence.

Families of domains not containing duplicated sequences: This class contains mostly animal toxins scaffolds and scaffolds derived from cell-surface-receptors. The protein toxins in the venoms of snakes, spiders, scorpions, snails and anemones can be considered naturally occurring injectable biopharmaceuticals. These venoms typically contain over 100 different toxins, related and unrelated, with a range of receptor- and species-specificities. The majority of these toxins are small proteins with a high density of disulfides. Typical sizes are 15-25aa with 2 disulfides, 25-45 aa with 3 disulfides, 35-50 aa with 4 disulfides as well as many examples with 5,6,7,8 or more disulfides. Examples are delta-Atracotoxin (1-4 2-6 3-7 5-8), Scorpion toxin (1-8 2-5 3-6 4-7), omega-Agatoxin (1-4 2-5 3-4 7-8), Maurotoxin (1-5 2-6 3-4 7-8) and J-Atracotoxin (1-4 2-7 3-4 5-8).

Phylogenetic analysis has shown that these proteins are an example of convergent evolution, with unrelated animal groups independently generating similar toxin structures from unrelated starting points. Given that the same design principle has won out in at least seven independent occasions (each in an unrelated taxonomic group), this design is expected to have important advantages over other scaffolds that are being used to build other types of toxins (ie microbial protein toxins).

The only feature that appears to be shared by these proteins is the high density of disulfide bonds. The amino acid sequences of these proteins (other than cys) are highly variable (see conotoxin alignment) and a wide range of different structures (protein folds) has been created.

One of the desirable properties of these proteins is their exceptionally small size; microproteins are the smallest rigid proteins), which is needed for rapid tissue penetration. A second common feature is their rigidity, which is higher than other proteins of similar size and allows these proteins to avoid induced fit upon binding to a target, which enables higher binding affinities. A third property is the exceptional stability of these proteins, both thermal stability (most microproteins can be boiled without denaturing) as well as resistance to a wide range of proteases. Many of the natural proteins function as protease inhibitors. Stability is important for biopharmaceuticals that are injected intravenously (IV) or sub-cutaneously (SC), and even more important to proteins that are delivered transdermally, nasally, orally, intestinally, or via the blood brain barrier. Stability is also important for long shelflife and convenient shipping and storage. Another property that is of great interest is the non-immunogenicity of these proteins which has been reported to be mediated by their resistance to proteolysis in antigen presenting cells (APC), which was published to be conferred by the high disulfide density structure. Other factors that keep immunogenicity low are the small size of the proteins and their hydrophilicity.

Families of domains containing duplicated sequences can also be employed in generating the subject microproteins and libraries thereof. Numerous examples are described in the examples below.

Families of domains containing repetitive sequences: Cysteine-rich Repeat Proteins (CRRPs): The high cysteine content of cysteine-rich repeat proteins allows formation of multiple disulfide bonds either within the repeating unit and/or between two repeating units. This results in a repeating pattern of disulfide bonds. This pattern provides a fixed topology, although in rare cases the same sequence may adopt (or can be evolved to adopt) an alternative disulfide bonding pattern. Disulfide bonds in repeat proteins are characterized by the CRRP motif (X_{A1},X_{A2})/(X_{B1},X_{B2})/(X_{C}) where X_{A} is the cysteine distance between linked cysteines, which is the number of cysteines between the first cysteine to the second cysteine in the same disulfide bond. This cysteine distance can be 1,2,3,4,5,6,7,8,9 or 10. Two (or more) numbers in the CRRP motif indicate two different (or more) types of bonds with X_{A1} describing the first such bond and X_{A2} describing the second disulfide bond. For example, CxCxCxCxCxCxCxC with a 1-4 2-3 topology has a cysteine distance of +3 for the first disulfide bond type and +1 for the second disulfide bond type (‘3,1’).

X_{B} describes the cysteine distance (number of cysteines) from the first cysteine of one disulfide bond to the first cysteine of the next disulfide bond (e.g. for CxCxCxCxCxC with 1-4 2-3 topology, X_{B} is +1. In the case of two different types of disulfide bonds X_{B1} describes the cysteine distance from the first cysteine of one type of disulfide bond to the first cysteine of the adjacent disulfide bond, while X_{B2} describes the cysteine distance from the first cysteine of the second type of disulfide bond to the first cysteine of the next disulfide bond which in this case is located in the next repeat. In this example X_{B2} is +3 (from C2 to C5), but it can be 1,2,3,4,5,6,7,8,9,10. X_{C} describes the number of disulfide bonds per helix turn in helical repeat proteins, which can be a fraction of 1, or an integer such as 1,2,3,4,5,6,7,8,9,10.

Each domain typically (but not necessarily) has one end cap on the N- and/or C-terminus. The end caps typically have one or two fewer cysteines than the regular repeats because they only have to connect to one repeat instead of two repeats.

A more detailed description of repeat proteins would include the ‘span’ (number of non-cys amino acids between two linked cysteines) of each type of disulfide bond in the protein. Another way to describe repeat proteins is to describe the sequence of the repeat unit, for example (CxxxCxCxxxxCxxCCxx)ₙ. The Cₐ and C_{b} notation can be used to indicate which cysteines are linked, such as in (CₐxxxCₐxC_{b}xxxxC_{c}xxC_{b}C_{c}xx)ₙ.

An important feature of cysteine-rich repeat proteins is that they can be extended on either end, at the N- or the C-terminus. Two approaches for library design are 1) randomization of naturally occurring repeat proteins and 2) synthetic repeats, which are typically obtained by abstraction from natural repeat proteins and may have a somewhat different spacing from the natural repeat sequences (more idealized). Naturally occurring CRRPs include granulins (PF00396), insect antifreeze proteins (PF02420), a furin-like domain (PF00757), the CxCxCx repeat (PF03128), the Paramecium surface antigen (PF01508) and a Drosophila domain of unknown function (PF05444).

Where desired, the subject cysteine-containing proteins and/or scaffolds can be fused with a bioresponse modifier. Examples of bioresponse modifiers include, but are not limited to, fluorescent proteins such as green fluorescent protein (GFP), cytokines or lymphokines such as interleukin-2 (IL-2), interleukin 4 (IL-4), GM-CSF, and γ-interferon. Another useful fusion sequence is one that facilitates purification. Examples of such sequences are known in the art and include those encoding epitopes such as Myc, HA (derived from influenza virus hemagglutinin), His-6, or FLAG. Other fusion sequences that facilitate purification are derived from proteins such as glutathione S-transferase (GST), maltose-binding protein (MBP), or the Fc portion of immunoglobulin. Library Construction: The present invention provides libraries of the subject cysteine-containing scaffolds. Whereas proteins subject to natural selection need to fold homogenously, a protein with a novel, non-evolved sequence may in principle be able to fold into multiple stable structures, or at least be induced to do so by varying conditions. The folding of different copies of the same protein sequence into different stable structures expands the structural diversity of the library beyond the number of independent clones in the library. The number of independent clones in a library generally equals the number of different sequences and is referred to as ‘library size’, which is about 10¹⁰ for phage display libraries. However the actual number of phage particles used when panning a phage library is typically 10-10,000-fold larger than the library size. The fold excess is called the ‘number of library equivalents’ and there are ways to exploit this difference to obtain greater library performance. If each of the 10-10,000 copies of a clone (ie all having the same amino acid sequence) adopts a different, stable DBP and structure, then the structural diversity can greatly exceed the sequence diversity (10¹¹-10¹⁴). It is possible to further increase structural diversity by using unstable structures that temporarily adopt different structures. However, the diversity can be increased even further if each phage particle displays an unstable protein, which can adopt a wide variety of structures, similar to random peptides and with similar advantages and disadvantages. Proteins that are able to adopt a large number of unstable structures can expand the diversity beyond the number of phage particles (10¹²-10¹⁵). While the recovery of low-affinity clones may require a large number of library equivalents (ie about 100 library equivalents to recover a clone with 1% recovery efficiency), high affinity clone recovery tends to be 100% efficient (as demonstrated by affinity chromatography) and increasing the structural diversity is expected to greatly increase the fraction of high affinity clones. There is a trade-off to increasing the structural diversity with unstable structures since the need to induce a structure in the displayed protein (induced fit of the binding protein, likely not of the target) upon target binding is expected to reduce the binding affinity of these clones.

One approach is to construct libraries with 4 cysteines (up to 2 disulfides and up to 3 bonding patterns), 6 cysteines (up to 3 disulfides and up to 15 different disulfide bonding patterns), 8 cysteines (up to 4 disulfides and up to 105 bonding patterns) or 10 cysteines (up to 5 disulfides and up to 945 bonding patterns), or 12, 14, 16, 18, 20 or even more cysteines.

In one aspect, the total number of disulfide bonding pattern can be generalized according to the following formula:

$${\prod }_{i = 1}^{n} 2 i - 1 ,$$

wherein n=the predicted number of disulfide bonds formed by the cysteine residues, and wherein

Π represents the product of (2i−1), where i is a positive integer ranging from 1 up to n.

Where desired, a much larger construct encoding a large but variable number (ie 10-30) cysteines can be generated. The resulting cysteine-containing products can fold in a wide diversity of different ways, creating different combinations of structured elements, each containing 2, 3, 4 or 5 disulfides and with potential crosslinking between them. During the directed evolution process of these larger constructs one could break the previously selected constructs up into smaller pieces, for example by random fragmentation, PCR (eg with random primers) or (eg 4 bp) restriction digestion. Once the library diversity of long proteins has been reduced, one can increase diversity again by creating a variety of fragments from each large construct and later on by recombination or other directed evolution methods.

One potential concern with such libraries of HDD proteins is the presence of unpaired cysteines after most of the disulfides have formed. The free thiols can interact with each other, creating aggregates which tend to score overly high in blocking assays, due to their multivalent binding to the target. However, these free thiols can be blocked, for example, with iodoacetamide or other well-known blocking agents for sulfhydryls to prevent them from forming aggregates or attacking correctly formed disulfides.

Alignment of the consensus sequences of multiple families of microproteins with the same number of disulfides (ie three disulfides giving 15 possible linkage patterns) shows that the spacing between the cysteines forms an approximately equal distribution ranging from 0 to about 12 amino acids; for simplicity and to keep the average loop length small we prefer families with 0-10 amino acids per intercysteine loop.

Using synthetic oligonucleotides, one can construct a library such that the DNA encodes the six cysteines and 0-10 NNK (or similar ambiguous codons) residues in the inter-cysteine loops. NNK codons encode all 20 aa but only 1/64 codons will be a stop codon (3 fold less than using NNN codons), which results in a reduced fraction of proteins containing a premature stop codon. Given 5 intercysteine loops, these proteins would contain an average of 25 NNK codons (assuming 0 to 10aa/loop; average 5), leading to a low fraction of clones with a premature stopcodon. The fraction of complete proteins could be increased by using a lower number than 10 or an ambiguous (mixed base composition) codon that excludes stop codons. As shown in the drawing, each oligonucleotide starts and ends with a cysteine codon (sense at one end, antisense on the other end), with 0-10 NNK codons (or the opposite sense) in between the cysteine codons. In this approach to making the synthetic library, all of the loop sequences can be used in any loop location, so all of the cysteines are typically encoded by same codon. All of the oligos are mixed together and a pool of synthetic genes is created by overlap PCR as described previously (Stemmer et al. 1995. Gene).

A different and powerful approach to creating phage libraries is the Scholle variation of Kunkel mutagenesis (Scholle, M. et al. (2005) Comb. Chem. & HTP Screening 8:545-551) in which the library-encoding oligonucleotide causes a stopcodon in the plasmid to be converted into a non-stop codon. A new version of this involves cycling back and forth between any two stopcodons (typically an amber codon and an ochre codon). This allows application of the Scholle method recursively to an evolving pool of clones without having to reinsert a stopcodon after each cycle of mutagenesis.

The 3SS (3-disulfide; 15 potential structures) and 4SS (105 potential structures) mixed scaffold libraries are especially useful. The primary control we have over disulfide bonding pattern is the spacing of the cysteines. Which structure (disulfide bonding pattern, ‘DBP’) the protein adopts can be controlled to a certain extent by offering, for example, a range of environments for re-folding. The DBP can be analyzed by trypsin digest and/or MS/MS analysis.

The problem of structural diversity is similar for both multi-scaffold libraries and for single scaffold libraries, with the difference in magnitude being continuously adjustable. In practice, there is a continuity of library designs based on the spacing of the cysteines, which can be more or less varied (on average between 0 and 15 amino acids per loop) and more or less similar to an existing natural family. The single scaffold libraries typically also contain significant length variation (mimicking the natural variation). Note that the families are created by sequence similarity and that typically for only a few members the structure (bonding pattern) was experimentally determined, so it is possible that a significant number of the natural sequences have a different structure than is assumed from the sequence. It is expected that natural highly evolved, highly fine-tuned (ie high information content) sequences generally fold reliably one way, but that low information content, less highly fine-tuned proteins (such as the ones in early-stage phage display libraries and/or derived from a structurally diverse libraries after one cycle of panning and before directed evolution) would often show several different folds.

Libraries based on a conserved scaffold of a specific natural family of proteins, like Ig domains or Fibronectin III, typically contain about 5-10% clones that have various problems (ie heterogeneously folded, unfolded, aggregated or poorly expressed). Increasing the length diversity or allowing greater sequence and structural diversity may yield more poorly behaved clones. It is common to screen out the undesired monomers before applying additional cycles of mutagenesis, including making dimers and higher order multimers. However, directed evolution tends to be very effective in making non-optimal clones behave better and one can gradually improve the average quality of the pool of clones by directed evolution, by eliminating clones and/or by sequence alteration and/or by structural alteration). Directed evolution screens for improved activity and since improved folding can be an easy way to improve activity, directed evolution of activity is a proven and efficient approach to obtain increased protein folding efficiency (Leong, S. R., et al. (2003) Proc. Natl. Acad. Sci. USA 100:1163-1168; Crameri, A. et al. (1996) Nature Biotechnology 14:315-319) and increased temperature stability (many published examples). The reason is that clones that adopt the active structure more efficiently appear to be more active and are thus favored in the selection process. The process we aim for is one where the initial rounds of panning will yield many clones that have a variety of folds and while there are likely to have a high level of various problems (incomplete folding, heterogeneous folding, low expression, aggregation, etc), the application of directed evolution (many possible formats including error-prone PCR, homologous recombination, cassette-based recombination, or even simply multiple rounds of screening) in combination with a strong functional selection by (phage) panning is expected to strongly favor clones with homogeneous folding. It is also possible to reduce, refold and repan the same library multiple times (with or without phage amplification) in order to increase the frequency of clones that fold homogenously. Free-thiol affinity columns can be used at each cycle to remove incompletely folded proteins, or the free thiols can be reacted with various capping agents (FITC-maleimide, iodoacetamide, iodoacetic acid, DTNB, etc). It is also possible to refold the whole library or to reduce partially and reoxidize in order to reduce the frequency of free thiols. Phage display and soluble protein binding assays often favor multivalent solutions. Proteins with inter-protein disulfides are a common source of multivalency and need to be removed since they cannot be manufactured. Multiple cycles of phage display (without assaying the soluble proteins intermittently) tends to evolve solutions that only work when on the phage. Screening of soluble proteins is thus generally desired to prevent those clones from taking over. Diversity of protein structures is useful early on, but it is desirable to increasingly remove clones that form inter-protein disulfide bonds. Diversity of structure correlates with indecisive folding and the presence of interprotein disulfides, and structure evolution may be inseparable from inhomogenous folding, so methods need to be developed that tolerate some degree of inhomogeneity.

In order to evaluate different library designs' for the desired balance of structural diversity and folding homogeneity, one can make small libraries and screen a limited number of clones (30-1000) in order to rapidly evaluate a diversity of library designs.

Different disulfides in the same protein can react differently, allowing some control. One of the approaches for removing clones with interprotein disulfides from phage libraries may be to subject the phage library to a low level of reducing agents which only reduces the weakest disulfides, such as interprotein disulfides and intraprotein disulfides that are so weak that we prefer to eliminate those clones, and then pass this partially-reduced library over a free-thiol column to remove these clones.

Structural Evolution of HDD Proteins

As noted above, HDD proteins are amenable to evolution the structure of the protein at every level, including primary (sequence), secondary (alpha-helix, beta-sheet, etc), tertiary (fold, disulfide bonding pattern) and quaternary (association with other proteins). The ability to completely change tertiary structure renders HDD proteins most amenable for rationale design of therapeutics or pharmaceutical compositions. While limited secondary structure evolution (alpha-helix, beta-sheet) may occur with existing directed evolution approaches, creating high-quality modifications in tertiary structure has in practice been difficult with directed as well as rational design.

Evolution from 2SS to 3SS to 4SS by disulfide addition, and the reverse by deletion, appears to occur frequently and has also been documented for snake disintegrins (Calvete, J. J et al. (2003) Biochem. J. 372:725-734). The relatedness of the DBPs of the natural families is suggestive that re-structuring of the DBP may also occur in nature, which is supported by publications of specific families, such as the Somatomedins.

The 15 different 3SS structures, 105 4SS or 945 4SS structures are topologically different, meaning they cannot be interconverted without breaking and reforming a disulfide bond. Each 3SS protein has 6 (fully) disulfide-bonded isomers that are ‘nearest neighbor’ variants (2 disulfides with altered bonding pattern, 1 disulfide with retained bonding pattern) and each 4SS protein has 12 isomeric nearest neighbor variants, each with 2 retained disulfides 2 altered disulfides), thus creating a gradual path for structure evolution.

The process of directed evolution of structure involves initially encouraging a large diversity of structures (not all will be possible and frequencies will differ), followed by gradually tightening the structure as well as partially modifying the structures (ie via gradual DBP alterations) while selecting for better and better binders. The large initial diversity of structures serves to expand the effective library size beyond the number of different AA sequences. However, the more diverse the structures are, the more heterogenous their folding will be, so these proteins generally will require significant evolution for homogenous folding in order to become useful. Structures with optimized loop length will fold more homogenously and will be more protease resistant and less immunogenic. The sequence of the loops, except for an occasional specific position, does not appear to affect tertiary structure and the loops tend to have no secondary structure.

A preferred approach to optimizing the loop length is to start with relatively long loops (ie 6,7,8 amino acids) and then gradually reduce their length, replacing each loop with a range of other loops of different sizes (with lower average size). This process resembles tightening of a knot. The position of the loops is typically kept constant (ie C2-C3) but their position could be varied, especially if multiple small binding sites in a protein are a useful solution.

One preferred approach is to replace a loop (ie loop C1-C2, C2-C3, C3-C4, C4-C5, C5-C6, C6-C7 or C7-C8, C8-C9, C9-C10) in a pool of selected clones with a new set of loops of mostly random sequence that have never been selected before. Using different codons for the different cysteines and if necessary a few fixed bases flanking the cysteines, one can create PCR sites to perform the loop exchange in a PCR overlap reaction (preferred), or one could use a restriction site approach.

Different clones in a pool that are selected to bind to a protein target are likely to bind to different sites on the protein. Even if they use similar sequences to bind to the same site, the clones are likely to differ in their register, some clones having the active sequence in loop 1, other clones in loop 5, for example. It is possible that having more fixed amino acids will result in more clones with the same register, which would be advantageous for directed evolution by homologous recombination.

There are a large number of ways to perform recombination on the pool of selected clones. In most formats, the loops will be kept intact and permutated relative to each other, but there are also formats in which homology between loops can be used to drive homologous recombination. In general each loop will stay in the same location (ie C4-C5), but even this can be varied. In some formats all of the loops in the pool of selected clones are unlinked and then relinked, but a more conservative approach is to unlink only one specific loop (ie C4-C5) while keeping the other loops linked, creating a library of clones with only 1-2 crossovers instead of many crossovers. The goal is to create many different gradual paths, which requires permutation of many conservative alterations.

Rather than making a library with many folds or a library with only one fold, we could make a library with limited variability in spacing which is designed to allow a smaller number of structures (ie lower limit of 2, 5, 10, 30, 100, 300 and a higher limit of 10, 30, 100, 300, 1000, 3000) structures that are selected because their bonding patterns result in rigid structures or occur in natural families, providing detailed information for the best cysteine spacing. An example is cxxx(x)cxxcxxxx(xx)cxxxcxxx(x)xxcxxxx(x)cxxxc.

The effective diversity and quality of a library are both very important but tend to have opposite design requirements. Quality is largely determined by the fraction of clones that fold correctly. Opening up the theoretical diversity (more randomized AA positions) of the library tends to increase the fraction of non-folding clones. Steps to increase folding include the use of native AA in each AA position and conservation of naturally conserved residues. This is easily accomplished for a single-scaffold library, but not for multi-scaffold libraries, which therefore must have a higher fraction of non-folding clones. Randomizing just 2 AA that need to be fixed for folding, the fraction of folded clones is reduced 400-fold, reducing the effective library size.

It will be useful to create various libraries and measure the fraction of folded clones by measuring the fraction of remaining free thiols using FITC-maleimide (react, wash, measure bound FITC). In addition, it may be useful to remove unfolded clones using solid supports with free-thiols and/or to refold the entire library or the unfolded clones. One approach is to expose the library to e a level of reducing agent that is expected to reduce partially or poorly folded proteins but not reduced stably-folded proteins.

However, a poor library design will still have a much reduced level of folded clones. One approach is to construct many single scaffold libraries separately and mix the libraries before panning. This should result in a high quality, diverse library.

Heterogenous folding should be a benefit if it is properly handled. Since routine libraries are 10-e8-10e9 in size and one creates about 10e13 phage particles, each sequence is represented by 10e4-10e5 particles. If panning is performed such that is 100% efficient (ie every 1 nM-or-better clone is captured), then having each sequence present as 10e3 different structures should be a huge benefit to effective diversity and hit-rate and quality. Efficient panning requires high concentration of phage, high concentration of target, increased temperature (faster equilibrium), volume excluders such as 10-15% polyethyleneglycol (PEG), soluble targets versus immobilized targets, etc.

To facilitate proper folding of proteins, one approach may be to fold (initially) in the presence of a volume excluding agent like PEG, which dramatically increase oligonucleotide hybridization rates and also the efficiency of a shuffling reaction (complex fragment overlap PCR). PEG simply increases the effective concentration of the thiols, leading to more intra- as well as inter-chain disulfides.

In general, unfolded clones are undesired but heterogenous folding is desired. Unfolding and heterogenous folding clearly go hand-in-hand. Target-induced folding of otherwise unfolded clones is especially useful, but likely a rare occurrence. Because of the expected reduction in effective library size of mixed-scaffold libraries, effective mutagenesis strategies are generally preferred. One may either choose recombination or both length variation and point mutation. Recombination of sequences derived from random libraries can be difficult. Error-prone PCR has an error-rate that is rather low (0.7%) for such short genes and requires recloning. Resynthesis requires sequencing of the selected clones and resynthesis of the library and recloning. Alternatively, one can subject mutator strains of E. coli to many cycles of panning and amplification in order to favor properly folded clones. In addition, one can apply Evogenix' approach.

The attraction of the 2-3-4 approach is that it adds random sequences at each step by PCR and does not require other forms of mutagenesis. Microproteins can be built from novel or existing peptide ligands or protein fragments. This approach utilizes a short amino acid sequence with or without pre-existing binding properties. The binding amino acid sequence can be flanked on one or both ends by random or fixed amino acid sequences that encode a single cysteine. Oligonucleotides are designed to encode the binding sequence and the flanking cysteine-encoding DNA. The newly introduced cysteines can optionally be flanked with random or non-random sequences. All variations of cysteine-containing flanking sequence are mixed, assembled and converted to double-stranded DNA. These assembled sequences can optionally be flanked with DNA that encodes restriction enzyme recognition sites or annealing to a pre-existing DNA sequence. This approach can generate novel or existing cysteine distance patterns.

Cysteine-Rich Repeat Proteins (CRRP)

It has been shown that the cysteine-rich repeat antifreeze protein from the beetle Tenebrio molitor can be extended on the C-terminus (C. B. Marshall, et al. (2004) Biochemistry, 43: 11637-46). The extension contains the CRRP motif 1/2/1. The extreme regularity of the helical but beta-sheet-containing (‘beta-helix’) antifreeze protein (FIG. 104) was explored systematically to test the relationship between antifreeze activity and the area of the ice-binding site. Each of the 12-amino acid, disulfide-bonded central coils of the beta-helix contains a Thr-Xaa-Thr ice-binding motif. By adding coils to, and deleting coils from, the seven-coil parent antifreeze protein, a series of constructs with 6-11 coils have been made. Misfolded forms of these antifreezes were removed by ice affinity purification to accurately compare the specific activity of each construct. There was a 10-100-fold gain in anti-freeze activity upon going from six to nine coils, depending on the concentration that was compared.

Our interest is to make an antifreeze-derived protein with multiple repeats that has been randomized in the least conserved amino acid positions and used to select binders (agonists or antagonists) against selected human therapeutics targets.

Granulins (FIGS. 102 and 103) are naturally occurring CRRPs with a CRRP motif of 3/2/2 (helix, see FIGS. 130-132). Evidence was presented that individual repeat units possess highly modular nature and are therefore useful for extending the core unit by adding multiple repeats to the C-terminus. (D. Tolkatchev, et al. (2000) Biochemistry, 39: 2878-86; W. F. Vranken, et al. (1999) J Pept Res, 53: 590-7). Upon air oxidation, a peptide corresponding to the 30-residue N-terminal subdomain of carp granulin-1 spontaneously formed the disulfide pairing observed in the native protein. Structural characterization using NMR showed the presence of a defined secondary structure within this peptide. A structure calculation of the peptide indicates that the peptide fragment adopts the same conformation as formed within the native protein. The 30-residue N-terminal peptide of carp granulin-1 is the first example of an independently folded stack of two beta-hairpins reinforced by two interhairpin disulfide bonds.

Our interest is to make a granulin-derived protein with multiple repeats that has been randomized in the least conserved amino acid positions and used to select binders (agonists or antagonists) against selected human therapeutics targets (FIG. 102).

Repeat Protein Structure and Affinity maturation: The advantage of CRRPs is that they can be made as long or as short as needed for the specific application, in contrast to most other domains. Thus, they can be given 1,2,3,4,5,6,7,8,9,10 or more binding sites for the same or different targets.

The advantage of CRRPs over Leucine-rich and other non-cysteine containing repeat proteins is that more amino acids can be randomized in a library, because the folding of CRRPs depends on the presence of disulfide bonds rather than on the presence of a hydrophobic core, which requires many more fixed residues. Libraries of CRRPs thus contain clones with more variable positions (>50, 60, 70 or 80%) which increases the potential surface contact area and the potential for high affinity for the target. Leucine-rich Repeat proteins, such as Ankyrins, are typically varied in only 6AA out of each 33AA repeat, or 24AA per 6-repeat domain, because the endcaps are not randomized.

Various affinity maturation approaches are shown in FIGS. 140, 14, 142, and 160. These affinity maturation principles are best explained with repeat proteins but are similarly applicable to all other scaffolds described in this application.

Affinity maturation of CRRPs can be achieved by two different strategies: module addition and module replacement.

The ‘module addition approach’ starts with a relatively small number of repeat units (e.g. 1-3) and randomized repeat units are added at each step of affinity maturation, followed by selection for binders. At each cycle of evolution one or a few new, randomized modules are added, followed by selection for the most active clones. This process increases the size of the protein at each cycle, while selecting for the desired binding activity after each round of extension. This approach converts randomized sequences into selected sequences.

The ‘module replacement approach’ starts with a larger number of repeats (e.g. 4-10; the ‘final number’) and at each round of library generation a new group of repeats (typically 1-3) is randomized followed by selection for target binding. In this approach the size of the protein remains constant. Unselected sequences (typically fixed) are gradually converted into randomized sequences which are in turn converted into selected sequences.

Both approaches yield repeat proteins with a single large binding site or multiple separate binding sites that have been selected for improved binding affinity to 1,2,3,4,5,6 or more targets. The addition of repeats allows the binding site(s) to be extended leading to increased binding affinity compared to a domain that binds it's target at a single site. Repeat protein domains can be linked to other repeat protein domains through short linker sequences that do not contain repeat sequences. This is a similar repeat protein organization as found in natural repeat proteins which often occur in tandem linked by short amino acid sequences and interspersed with non-repeat proteins (H. K. Bins et al. (2005) Nature Biotechnology).

However, repeat proteins can also be used to form a stiff connection between two binding sites to allow the sites to bind the target simultaneously. In contrast to the flexible peptide linker that is typically present between separate domains, a stiff connector based on repeat proteins is expected to yield a higher binding affinity. Another way to create a stiff connector between binding sites is to use proline-rich sequence, which coils up on itself, or a collagen-like sequence.

Affinity maturation is carried out by (partial) randomization at the DNA level, targeting either a single continuous sequence or multiple discontinuous sequences. Sequential steps of DNA randomization can also be either discontinuous or continuous (ie sequential) at the DNA level. At the protein level, the mutagenesis may also be discontinuous or continuous, depending on the application. For example, for a helical repeat protein it would be typical to use discontinuous maturation at the DNA and protein chain level to obtain a continuous binding surface on the same side of the protein. It is called discontinuous because the randomized amino acids are discontinuous on the alpha-chain backbone and at the DNA level, even though on the surface of the protein the randomized area is continuous. On the other hand, sequential maturation involves randomization of a set of amino acids that is continuous at the DNA level and protein backbone level, so that all sides of the helix are randomized and can become binding sites for the target, thereby allowing more complex three-dimensional interactions between the repeat protein and the target protein. In the case of discontinuous (DNA-level) affinity maturation, a common fixed sequence in between the randomized sequences can be utilized to perform recombination by restriction enzymes or overlap PCR, either within a library or between multiple libraries, providing an additional step which increases the number of clones that can be screened for improved binding affinity.

A preferred approach to affinity maturation is sequential randomization, which involves first (partially) randomizing one area of the scaffold protein, selecting a pool of the best clones, then randomizing a second area in the clones of this selected pool, re-selecting a (second) pool of the best clones, and randomizing a third area of the clones in this second pool, and selecting a (third) pool of improved clones. This is shown in e.g., FIG. 136. A preferred approach is to have the three mutagenesis areas (n-term, middle and c-term) be non-overlapping. Any order of mutagenesis can be used, but n-term/middle/c-term and n-term/c-term/middle are preferred choices. It is useful to leave 1 5-20 bp of scaffold sequence unmutagenized between the mutagenesis areas, to serve as an annealing area for oligonucleotides for Kunkel-type mutagenesis. This approach avoids synthetic re-mutagenesis of previously mutagenized sequences, a time-consuming process which typically requires sequencing of the clones, alignment of the sequences, deduction of family motifs and resynthesis of oligos encoding these motifs and creation of new synthetic libraries. A preferred format is to use codon choice such that the randomization yields mostly the amino acids that occur naturally in each position.

Synthetic CRRPs

Synthetic CRRPs consist of the motif Cₐx₀₋ₙC_{b}x₀₋ₙC_{c}x₀₋ₙC_{d}X₀₋ₙCₑX₀₋ₙC_{f}x₀₋ₙC_{g}x₀₋ₙCᵢx₀₋ₙCᵢx_{0-n n}Cᵢx₀₋ⱼ where C is a cysteine residue at a defined position and x can be any number of amino acids between 0 and 12 between each individual cysteine. These designs are defined by the CRRP motif, e.g. the cysteine distance between individual disulfide bonds and the cysteine distance between the first cysteine of a disulfide bond to the first cysteine of the next disulfide bond. The following motifs are useful for library design: 3/4/1, Cₐx₀₋ₙC_{b}x₀₋ₙC_{c}X₀₋ₙC_{d}X₀₋ₙ CₑX₀₋ₙC_{f}x₀₋ₙC_{g}x₀₋ₙ, where Cₐ forms a disulfide bond with C_{d}; (3,4)/(1,4)/2, Cₐx₀₋ₙC_{b}x₀₋ₙC_{c}X₀₋ₙC_{d}X₀₋ₙCₑX₀₋ₙC_{f}x₀₋ₙC_{g}x₀₋ₙ, where Cₐ forms a disulfide bond with C_{d} and C_{c} forms a disulfide bond with C_{g}; (4/2),(3/1), Cₐx₀₋ₙC_{b}x₀₋ₙC_{c}X₀₋ₙC_{d}X₀₋ₙCₑX₀₋ₙC_{f}x₀₋ₙC_{g}x₀₋ₙ, where Cₐ forms a disulfide bond with Cₑ, (3,5)/(1,2)/2, Cₐx₀₋ₙC_{b}x₀₋ₙC_{c}X₀₋ₙC_{d}X₀₋ₙCₑX₀₋ₙC_{f}x₀₋ₙC_{g}x₀₋ₙ, where Cₐ forms a disulfide bond with C_{f}, C_{b} forms a disulfide bond with Cₑ, C_{d} forms a disulfide bond with Cᵢ; (3,5,7)/(1,2,3)/3, where Cₐ forms a disulfide bond with C_{f}, C_{b} forms a disulfide with Cₑ, C_{c} forms a disulfide with Cⱼ; (4,5)/(1,4)/2, where Cd forms a disulfide with Ci, Cf forms a disulfide with Cj (see FIGS. 125-133).

Novel CRRP can be designed by starting with a single domain family containing disulfide bonds of a known topology and extending this motif at the N- or C-terminus. In order to achieve disulfide connectivity between the two repeat units, an additional two cysteine residues may need to be introduced by site-directed mutagenesis. The topology 1-4 2-5 3-6 is the most commonly observed disulfide topology among small cysteine-rich microproteins. Domains with this topology can be extended by adding repeats with a related topology. Cysteine residues are introduced at positions between cysteine 1 and cysteine 2, and after cysteine 6. Even in the presence of two additional cysteines there will be a strong tendency to form the 1-4 2-5 3-6 topology as the structural scaffold will only allow this topology.

Connection Different Structures: See FIGS. 146, 147, 148. Microprotein modules can be linked in a variety of different ways. For example, the C5C5C5C5C5C module with topology 1-4 2-5 3-6 can be linked to another such module without a linker yielding a C5C5C5C5C5CC5C5C5C5C5C module. Modules may be linked with a structured PPPP linker. In addition, cysteine-rich repeat modules can be used to link two modules. Granulin-like repeating units serve as linkers with the general repeating motif (CC5)ₙ. Fusion can also be achieved by a two disulfide containing linker with 13 24 topology and the motif (Cx₀₋ₙCx₀₋ₙCx₀₋ₙC)ₙ, where x is any number of amino acids from 0 to n=12. The antifreeze protein repeat (2C_{A}5C_{B}3)ₙ with a disulfide bond formed between C_{A} and C_{B} is used as a connector between different modules or to connect microproteins to other proteins.

Design of Typical Synthetic Repeat Protein: The natural design of repeat proteins is a repetition of single building blocks which are added to the core motif. This process can be mimicked during in vitro evolution. Antifreeze protein contains a typical 3-disulfide microprotein as a cap at the N-terminus (CₐxxxxxC_{b}xxC_{c}xxxC_{d}xxCₑxxC_{f}xxxx). A part of this structure can be added to the C-terminus of this sequence using molecular biology. There are two possibilities to chose the repeating unit: either xC_{b}xxC_{c}xxxC_{d}xxCₑx or xxC_{b}xxC_{c}xxxC_{d}xxCₑxxC_{f}x can be added to the C-terminus continuously to design a novel repeat protein. See FIG. 104.

Design of a synthetic scaffold based on the CXCXCCXCXC motif: Many microprotein families contain a motif consisting of the logo Cxxxxxx(xxxxxxx)Cxxxxxx(xxxxxxx)CCxxxxxx(xxxxxxx)Cxxxxxx(xxxxxxx)C, with a disulfide bond topology 1-4 2-5 3-6. This general consensus is used for library design. Spacings may include additional cysteines and disulfide bonds. Spacing between each disulfide bond averages 13-15. Extra cysteine pairs in addition to the basic motif are indicated in blue or green italics, with linked cysteines sharing the same color.

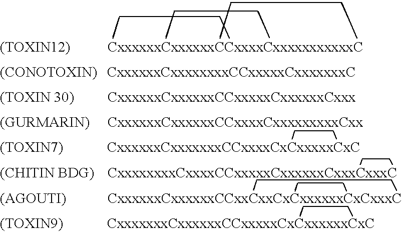

| | 1-4 | 2-5 | 3-6 | Additional SS |
|---|---|---|---|---|
| TOXIN12 | 13 | 12 | 17 | |
| CONOTOXIN | 15 | 15 | 14 | |
| TOXIN 30 | 14 | 13 | 13 | |
| GURMARIN | 14 | 12 | 15 | |
| TOXIN7 | 15 | 13 | 15 | 6-7 |
| CHITIN BDG | 14 | 11 | 13 | 7-8 |
| AGOUTI | 14 | 13 | 16 | 5-10, 7-8 |
| TOXIN9 | 15 | 15 | 15 | |
| AVERAGE | 14 | 13 | 15 | |

The Swissprot database contains 44 members with the spacing 6,5,0,3 and 57 members with the spacing 6,5,0,4 and 34 members with the spacing 6,6,03 and 27 members with the spacing 6,6,0,4. The last spacing (between Cys5 and Cys6) can be varied from 4 to 6 amino acids).

Cysteine Distance Patterns (CDP): The most commonly used approaches to group natural proteins into families are based on protein sequence homology. The goal of these algorithms is to group protein sequences based on their relatedness, which in most cases reflects evolutionary distance. These algorithms align sequences to maximize the number of matching identical or chemically related amino acids for each position. Frequently, gaps are introduced to improve the alignment. Such homology-based sequence families have been commonly used to identify protein scaffolds that can allow significant sequence variation and thus can serve as base for novel binding proteins. However, homology-based families have limited utility for the design of microprotein-based libraries due to the low degree of sequence conservation between related microproteins. The sequences of closely related microproteins frequently share little sequence homology other than conservation of their cysteine residues. The introduction of gaps by homology-based search algorithms complicates the alignment of microprotein sequences, which is critical to identify residues that can be mutated and residues that are important for protein structure and/or stability. Microproteins differ from most other proteins in their extremely high density of cysteine residues and this group requires an alignment approach that ranks Cysteine spacing as a key parameter, allowing one to group microproteins into clusters that share identical Cysteine Distance Patterns (CDP). Thus a cysteine distance cluster is a group of protein sequences that have several cysteine residues that are separated by identical numbers of amino acids. The sequences of all members of a cysteine distance cluster are aligned because all cluster members have identical total length. In addition, one can easily calculate the average amino acid composition for each position in the sequence. This greatly simplifies the identification of residues that can be varied as well as the degree of variation when constructing microprotein libraries. Large clusters of microproteins with identical CDPs are particularly useful to design microprotein libraries as they provide detailed information about the natural variability in each position.

CDP clusters are typically subsets of related microprotein sequences. In many cases, all members of a CDP cluster come from the same family of homologous proteins. However, there are CDP clusters that contain members from multiple protein families. An example is the CDP cluster 3_{—}5_{—}4_{—}1_{—}8 (sometimes shown as C3C5C4C1C8 or CxxxCxxxxxCxxxxCxCxxxxxxxxC) that contains 51 members, some from family PF00008 and others from family PF07974. A sequence with that CDP may (in principle) be able to adopt both structures. These structurally diverse CDPs are preferred to obtain structural evolution.

Since the DBP is difficult to control directly but CDP is easily controlled by gene synthesis, CDP becomes the most preferred way to control DBP and structure.

Identification of useful CDPs: Useful CDPs can be found by analyzing protein sequence data bases like Swiss-Prot or Translated EMBL (Trembl). A data base that combines information from Swiss-Prot and Pfam and annotates cystine bonding patterns was described by Gupta (Gupta, A., et al. (2004) Protein Sci, 13: 2045-58). Such data bases can be searched for protein sequences that contain a high percentage of cysteine residues, which are typical for microproteins. One can calculate the distance between consecutive or neighboring cysteine residues to get the CDP and then search for CDPs that occur many times. CDPs are of particular interest if many natural sequences share the same CDP, because this suggests that this CDP allows a wide diversity of sequences. Useful CDPs avoid long distances between neighboring cysteine residues (‘long loops’), because these are more likely to be attacked by proteases and more likely to yield peptides that are long enough to bind in the cleft of MHC molecules. Of particular interest are CDPs where none of the distances exceed 15, 14, 13, 12 or 11 amino acids. More preferred are CDPs where none of the distances between neighboring cysteine residues exceed 10, 9 or 8 residues. Of particular interest are CDPs from families that have a low abundance of hydrophobic amino acids like tryptophan, phenylalanine, tyrosine, leucine, valine, methionine, isoleucine. These hydrophobic residues occur with frequencies of ca 34% in typical proteins and are associated with non-specific, hydrophobic binding. CDPs of particular interest contain many members with less than 30, 28, 26, 24 or 22% hydrophobic residues. Preferred CDPs and individual members contain less then 20, 18, 16, 14, 12, or even as low as 8 or 6% hydrophobic residues. Of particular interest are CDPs were individual members show great sequence diversity. Table 2 gives examples of CDPs that can serve as very useful scaffolds for microprotein libraries. [Table 3] gives most preferred CDPs.

TABLE 2
List of exemplary CDPs.

| | | Domain | | | | | | | |
|---|---|---|---|---|---|---|---|---|---|
| | # | Length | Loop length | | | | | | |
| # members | disulfides | C—C; in AA | n1 | n2 | n3 | n4 | n5 | n6 | n7 |
| 124 | 3 | 37 | 6 | 4 | 8 | 1 | 12 | | |
| 107 | 3 | 43 | 3 | 10 | 11 | 9 | 4 | | |
| 103 | 3 | 51 | 8 | 15 | 7 | 12 | 3 | | |
| 93 | 3 | 58 | 12 | 12 | 3 | 13 | 12 | | |
| 92 | 3 | 49 | 7 | 7 | 10 | 2 | 17 | | |
| 90 | 3 | 36 | 6 | 3 | 8 | 1 | 12 | | |
| 77 | 4 | 46 | 1 | 9 | 6 | 1 | 8 | 2 | 11 |
| 74 | 4 | 37 | 8 | 4 | 0 | 5 | 6 | 3 | 3 |
| 70 | 7 | 65 | 1 | 5 | 3 | 0 | 4 | 7 | 4 |
| 69 | 4 | 57 | 10 | 6 | 16 | 3 | 10 | 0 | 4 |
| 65 | 3 | 46 | 15 | 2 | 12 | 3 | 8 | | |
| 60 | 2 | 22 | 4 | 13 | 1 | | | | |
| 59 | 2 | 40 | 3 | 29 | 4 | | | | |
| 54 | 3 | 38 | 6 | 5 | 6 | 5 | 10 | | |
| 54 | 6 | 61 | 1 | 6 | 0 | 4 | 7 | 4 | 0 |
| 49 | 3 | 31 | 6 | 4 | 9 | 6 | 0 | | |
| 49 | 4 | 61 | 1 | 6 | 17 | 2 | 8 | 2 | 17 |
| 47 | 3 | 56 | 11 | 28 | 0 | 3 | 8 | | |
| 45 | 2 | 21 | 4 | 12 | 1 | | | | |
| 45 | 4 | 38 | 8 | 4 | 0 | 5 | 6 | 4 | 3 |
| 44 | 3 | 45 | 3 | 7 | 10 | 6 | 13 | | |
| 44 | 4 | 48 | 1 | 6 | 6 | 2 | 8 | 2 | 15 |
| 42 | 4 | 58 | 13 | 6 | 16 | 1 | 10 | 0 | 4 |
| 41 | 4 | 47 | 3 | 8 | 11 | 2 | 0 | 5 | 10 |
| 40 | 4 | 52 | 3 | 5 | 3 | 9 | 9 | 1 | 14 |
| 40 | 5 | 59 | 8 | 3 | 3 | 6 | 10 | 3 | 1 |
| 39 | 2 | 15 | 1 | 7 | 3 | | | | |
| 39 | 3 | 35 | 5 | 3 | 8 | 1 | 12 | | |
| 38 | 4 | 31 | 1 | 4 | 0 | 5 | 6 | 3 | 4 |
| 37 | 3 | 30 | 12 | 0 | 0 | 10 | 2 | | |
| 36 | 4 | 38 | 8 | 4 | 0 | 5 | 6 | 3 | 4 |
| 36 | 7 | 65 | 1 | 5 | 4 | 0 | 3 | 7 | 4 |
| 35 | 3 | 36 | 0 | 12 | 12 | 6 | 0 | | |
| 34 | 3 | 38 | 9 | 9 | 4 | 0 | 10 | | |
| 33 | 3 | 29 | 12 | 0 | 0 | 9 | 2 | | |
| 31 | 3 | 45 | 2 | 5 | 16 | 2 | 14 | | |
| 31 | 7 | 76 | 2 | 7 | 5 | 0 | 5 | 9 | 9 |
| 30 | 3 | 36 | 7 | 4 | 10 | 1 | 8 | | |
| 29 | 3 | 34 | 6 | 5 | 8 | 1 | 8 | | |
| 29 | 2 | 40 | 13 | 9 | 14 | | | | |
| 29 | 3 | 47 | 16 | 2 | 12 | 3 | 8 | | |
| 28 | 2 | 9 | 0 | 3 | 2 | | | | |
| 28 | 3 | 26 | 6 | 5 | 3 | 1 | 5 | | |
| 28 | 3 | 46 | 3 | 10 | 12 | 11 | 4 | | |
| 27 | 3 | 39 | 9 | 7 | 12 | 3 | 2 | | |
| 26 | 2 | 23 | 5 | 11 | 3 | | | | |
| 26 | 4 | 48 | 1 | 9 | 6 | 1 | 8 | 2 | 13 |
| 25 | 3 | 26 | 8 | 2 | 1 | 8 | 1 | | |
| 25 | 3 | 36 | 6 | 5 | 8 | 1 | 10 | | |
| 24 | 2 | 25 | 3 | 7 | 11 | | | | |
| 24 | 3 | 47 | 3 | 9 | 10 | 6 | 13 | | |
| 23 | 3 | 41 | 12 | 6 | 12 | 3 | 2 | | |
| 23 | 3 | 42 | 10 | 8 | 13 | 3 | 2 | | |
| 23 | 4 | 45 | 1 | 9 | 5 | 1 | 8 | 2 | 11 |
| 23 | 3 | 46 | 3 | 8 | 10 | 6 | 13 | | |
| 23 | 5 | 61 | 2 | 4 | 5 | 6 | 17 | 3 | 10 |
| 22 | 2 | 14 | 3 | 1 | 6 | | | | |
| 22 | 3 | 24 | 0 | 4 | 7 | 1 | 6 | | |
| 22 | 3 | 29 | 4 | 5 | 5 | 1 | 8 | | |
| 22 | 3 | 29 | 5 | 3 | 10 | 4 | 1 | | |
| 22 | 3 | 31 | 12 | 0 | 0 | 9 | 4 | | |
| 22 | 3 | 38 | 0 | 11 | 9 | 5 | 7 | | |
| 22 | 4 | 51 | 1 | 11 | 6 | 1 | 8 | 2 | 14 |
| 22 | 7 | 77 | 2 | 7 | 5 | 0 | 5 | 9 | 9 |
| 21 | 3 | 37 | 7 | 5 | 6 | 5 | 8 | | |
| 21 | 3 | 48 | 6 | 7 | 10 | 2 | 17 | | |
| 20 | 3 | 30 | 13 | 0 | 0 | 9 | 2 | | |
| 20 | 2 | 33 | 9 | 10 | 10 | | | | |
| 20 | 4 | 50 | 1 | 11 | 6 | 2 | 8 | 2 | 12 |

The column labeled ‘members’ shows the number of natural sequences with the particular CDP that were identified in the data base described by Gupta (Gupta, A., et al. (2004) Protein Sci, 13: 2045-58). ‘n’ is the number of disulfides in the cluster. ‘Domain Length’ is the number of amino acid residues for the CDP (first cys to last cys). The column n1 through n7 list the number of non-cysteine residues that separate the cysteine residues of a cluster. n2=6 means the loop between C2 and C3 is 6AA long, excluding the cysteines.

TABLE 3
List of exemplary CDPs

| | | Domain | | | | | | | |
|---|---|---|---|---|---|---|---|---|---|
| | | Length | Loop Length | | | | | | |
| #members | # disulf. | AA | n1 | n2 | n3 | n4 | n5 | n6 | n7 |
| 575 | 3 | 35 | 6 | 4 | 6 | 5 | 8 | | |
| 518 | 3 | 32 | 4 | 5 | 8 | 1 | 8 | | |
| 190 | 3 | 37 | 6 | 4 | 6 | 5 | 10 | | |
| 155 | 3 | 36 | 6 | 5 | 6 | 5 | 8 | | |
| 93 | 3 | 36 | 6 | 4 | 6 | 5 | 9 | | |
| 72 | 3 | 38 | 7 | 4 | 6 | 5 | 10 | | |
| 71 | 3 | 23 | 2 | 1 | 7 | 1 | 6 | | |
| 67 | 3 | 37 | 6 | 6 | 6 | 5 | 8 | | |
| 64 | 3 | 36 | 5 | 4 | 8 | 1 | 12 | | |
| 62 | 3 | 36 | 7 | 4 | 6 | 5 | 8 | | |
| 59 | 3 | 34 | 4 | 5 | 10 | 1 | 8 | | |
| 57 | 3 | 28 | 3 | 5 | 5 | 1 | 8 | | |
| 57 | 3 | 33 | 4 | 5 | 9 | 1 | 8 | | |
| 56 | 3 | 35 | 6 | 6 | 12 | 3 | 2 | | |
| 54 | 4 | 44 | 1 | 9 | 6 | 1 | 8 | 2 | 9 |
| 51 | 3 | 27 | 3 | 5 | 4 | 1 | 8 | | |
| 49 | 3 | 29 | 1 | 4 | 9 | 9 | 0 | | |
| 45 | 3 | 37 | 6 | 5 | 6 | 5 | 9 | | |
| 43 | 3 | 31 | 4 | 4 | 8 | 1 | 8 | | |
| 43 | 4 | 45 | 10 | 5 | 3 | 9 | 6 | 1 | 3 |
| 38 | 4 | 45 | 1 | 9 | 6 | 1 | 8 | 2 | 10 |
| 34 | 5 | 54 | 8 | 3 | 3 | 8 | 3 | 3 | 1 |
| 33 | 3 | 41 | 3 | 10 | 9 | 9 | 4 | | |
| 29 | 2 | 23 | 6 | 5 | 8 | | | | |
| 27 | 3 | 37 | 6 | 3 | 9 | 1 | 12 | | |
| 26 | 4 | 35 | 3 | 9 | 1 | 3 | 5 | 0 | 6 |
| 25 | 3 | 26 | 4 | 3 | 10 | 2 | 1 | | |
| 25 | 3 | 35 | 4 | 5 | 11 | 1 | 8 | | |
| 24 | 3 | 34 | 5 | 4 | 6 | 5 | 8 | | |
| 24 | 3 | 37 | 7 | 3 | 8 | 1 | 12 | | |
| 24 | 3 | 44 | 3 | 10 | 10 | 11 | 4 | | |
| 23 | 3 | 35 | 6 | 8 | 10 | 3 | 2 | | |
| 22 | 3 | 33 | 5 | 5 | 8 | 1 | 8 | | |
| 22 | 3 | 37 | 3 | 10 | 5 | 9 | 4 | | |
| 21 | 3 | 33 | 9 | 9 | 4 | 0 | 5 | | |
| 21 | 3 | 36 | 3 | 10 | 4 | 9 | 4 | | |
| 20 | 2 | 18 | 9 | 0 | 5 | | | | |
| 20 | 3 | 34 | 5 | 5 | 9 | 1 | 8 | | |
| 20 | 3 | 42 | 3 | 10 | 10 | 9 | 4 | | |
| 20 | 4 | 43 | 1 | 9 | 5 | 1 | 8 | 2 | 9 |

‘Members’ gives the number of natural sequences with the particular CDP that were identified in the data base described by Gupta (Gupta, A., et al. (2004) Protein Sci, 13: 2045-58). ‘n’ gives the number of disulfides in the cluster. ‘Domain Length’ gives the number of amino acid residues for the CDP (first cys to last cys). The columns n1 through n7 list the number of non-cysteine residues that separate the cysteine residues of a cluster (‘loop length’).

Some of the intercysteine loops need to be fixed in size, while other loops can accommodate some length diversity. The length diversity that occurs in the families of natural sequences is one way to estimate what length variation is acceptable for specific loops. Such permitted length variation ranges from minus 10,9,8,7,6,5,4,3,2,1 amino acids to plus 1,2,3,4,5,6,7,8,9 or 10 amino acids.

Directed Evolution of DBPs and protein folds of pools of clones: The large number of disulfide bonding patterns (DBPs) is an additional degree of freedom that can be used to optimize HDD (‘high disulfide density’) proteins which is not available for non-HDD proteins, even those with many disulfides. One factor is that in larger proteins the disulfides are far apart and unlikely to react unless other fixed sequences fold the protein such that the cysteines are brought together at high local concentration and in the right orientation. Thus, the cysteines have a relatively less important role in folding of larger proteins. Larger proteins with hydrophobic cores tend to have many side-chain contacts that are involved in creating the 3D structure. In this so-called high information content solution, as defined by Hubert Yockey (1974), the DBP is statistically locked in place and evolutionary changes in the DBP are highly unlikely. Structure evolution is likely only available for proteins with a low information content, such proteins that have few residues that are required for structure and function. Information content of a protein, defined as the sensitivity to random mutagenesis, does not simply increase over time as a function of the evolutionary age of the protein. For example, when a gene is duplicated, one of the two copies is free to evolve and effectively has a very low information content even though its information content would be high if there were only one copy of the gene. In a low information content situation, large numbers of amino acids mutations and major changes in structure can occur, which would be lethal if they occurred in a single copy gene. The information content of a protein depends also on the specific functional aspect that is being considered, some functions (ie catalysis) having a much higher information content than others (ie vaccine based on a 9AA T-cell epitope). Redundancy is common in venomous animals, each of which typically has well over 100 different toxins derived from the same or different genes in it's venom. Redundancy likely helps the rapid evolution of HDD proteins, either as multiple copies of the same gene, and/or single copies of different genes encoding a wide diversity of toxins.

A pool of clones that has been selected for binding to a target may have only part of a domain (a sub- or micro-domain, or one or more loops) providing the binding function. The best clones in a typical 10e10 library would on average have only about 7 amino acids that are fully optimized. This is because the maximum (average) information content that can be added in one cycle of panning is the size of the library (ie 10e10). Multiple cycles of library generation and screening are generally required to accumulate information content beyond that. Three cycles of 10e10 may in theory yield up to 10e30 information content, but typically the number would be much less than due to practical limitations to the additivity. Typically, most of the amino acids in a domain are not directly contacting the target and they could be replaced by a variety of amino acids if not all. One goal of structural evolution is to evolve the DBP of the non-binding parts to result in a modified structure that yields higher affinity target binding, without creating any changes in the amino acid sequence of the parts that bind the target.

A preferred approach is to encourage the formation of multiple structures from each single sequence, either in the first cycle or after the diversity has been reduced by one or more cycles of panning so that one has a large number of (>10e4) copies of each phage clone, each copy being able to adopt a different DBP and structure. One way to increase the diversity of structures in a library before panning is to suddenly add a high concentration of oxidizing agent to the library after the library has been heated for 10-30 seconds in order to remove any partially folded structures that may have formed. The sudden formation of disulfides, before the protein has had a chance to anneal and explore its folding pathways, should lead to increased diversity, although the average quality of the resulting folds may be reduced by this approach. The opposite approach is used to obtain homogenous folding and typically involves a gradual removal of the reducing agents by dialysis leading to gradual folding and gradual sulfhydryl oxidation. This approach can also involve a gradual decline in temperature, similar to annealing of oligonucleotides. If DBP-diversification is applied to the library in the first round of panning, it is important to create a large library excess, for example 10e5 fold more particles than the number of different clones (typically 10e9-10e10)), to cover the large number of different structures that can be created from each sequence.

Diversification of DBPs:_The spectrum and distribution of DBPs can be diversified by subjecting aliquots of the same library to a diversity of different conditions. These conditions could include a range of pHs, temperature, oxidizing agents, reducing agents such as DTT (dithiothreitol), BME (betamercaptoethanol), glutathione, polyethyleneglycol (molecular crowding, so infrequent DBP can become more frequent), etc.

Multi-scaffold libraries: To identify microprotein domains that bind with high affinity to a target, multi-scaffold libraries can be employed according to the following three step process:

1. Build sub-libraries based on multiple scaffolds or Cysteine Distance Patterns (CDPs) and various randomization schemes.

2. Identify initial hits by panning a number of sub-libraries on the target of interest. This can be done by panning each library separately or by panning a mixture of sub-libraries.

3. Initial hits are optimized via affinity maturation, which is an iterative process encompassing mutagenesis and selection or screening.

The use of multi-scaffold libraries differs significantly from traditional approaches that focus on individual scaffolds. In single scaffold libraries most library members share a similar overall architecture or fold and they differ mainly in their amino acid side chains. Examples of single scaffold libraries were based on fibronectin (Koide, A., et al. (1998) J Mol Biol, 284: 1141-51), lipocalins (Beste, G., et al. (1999) Proc Natl Acad Sci US A, 96: 1898-903), or protein A-domains (Nord, K., et al. (1997) Nat Biotechnol, 15: 772-). Many additional scaffolds have been described in Binz, H. K., et al. (2005) Nat Biotechnol, 23: 1257-68. In some cases, single scaffold libraries contained members that show small differences in the length of individual loops for instance CDRs in antibody libraries. Single-scaffold libraries tend to cover a limited amount of shape space. As a result, one frequently obtains low affinity binders. These molecules don't match the shape of their target particularly well. However, the amino acids that form the contact area have been optimized to partially compensate for the lack of shape complementary. Many publications describe efforts to increase library size (ie ribosome display, combinatorial phage libraries) in order to improve the amino acid diversity in the contact area between the scaffold and the target. Initial hits resulting from single scaffold libraries can be further optimized by affinity maturation. However, this process is typically focused on small changes in external, CDR-like loops in the binding protein and does not affect the overall structure of the domain. There are no examples where affinity maturation of fixed scaffolds leads to major changes in the overall fold and structure of the binding protein; in rare cases where a major change did occur, such clones are generally eliminated because their immunogenicity and manufacturing properties are considered to be unpredictable.

Multi scaffold libraries contain clones with a diversity of (often unrelated) scaffolds, with large differences in overall architecture. In general, each CDP represents a different shape and each Sub-library contains an ensemble of mutants that sparsely samples the sequence space around a particular CDP. By testing molecules with many different shapes (from many sub-libraries, each with a different CDP), one increases the chance of identifying binding proteins whose structure closely complements the surface of the target. Because each sub-library represents a relatively small sample of the sequence space surrounding a CDP, it is unlikely that one obtains optimum binding sequences from this process. Initial hits from multi-scaffold libraries mimic the shape of their target but the fine structure of the contact surface between the hit and the target may be suboptimal. As a consequence, it is likely that further improvements in binding affinity can be accomplished during subsequent affinity maturation that is focused on optimizing a particular protein's sequence without dramatically changing its architecture. Simplistically stated, the goal is to find the best structure that fits the target, and then find the best sequences that fit this structure and provide optimal complementarity with the target.

Experimental approaches to finding novel scaffolds: Another way to approach library design is to let the proteins compute the best solutions themselves, by letting a diversity of designs compete. The fully folded and well-expressed proteins are selected and sequenced. The designs with the highest fraction of folded proteins (corrected for the input numbers) are preferred. There are several different approaches to finding the preferred CDP and sequence motif:

Approach 1: Random CDP, Random Sequence

The random spacing and sequence approach is not based on the spacings or sequences present in natural diversity and is therefore able to find novel and existing cys-spacing patterns in proportion to their ability to accept random sequence.

The approach involves making broad, open libraries, like a 10e10 display library with design CX(0-8)CX(0-8)CX0-8)CX(0-8)CX(0-8)C, followed by selection for 25-35AA total length using agarose gels, expression in E. coli, then (optionally) removing all of the unfolded proteins from the display library using a free thiol column, (or screening individual clones for expression level) and sequencing of 200-1000 clones encoding proteins that are well expressed and fully folded.

All of the distance patterns occur at similar frequencies in the library. We expect to find a strong bias in the spacing/distance patterns that occur in natural proteins but many spacing patterns will be novel. For example, if distance pattern A allows only 0.01% folded proteins and pattern B yields 10% folded proteins, clones with pattern B should occur 1000-fold more frequently than clones with pattern B. Sequencing 1000 clones should be sufficient to identify 10-30 spacings that are the most capable of folding, regardless of the loop sequences. Many spacing patterns found with this approach are likely to be novel and would then be used to make separate libraries based on these spacings. Novel spacings found by this approach would typically be combined with spacings based on natural families in the next approach.

Approach 2: Natural CDP, Random sequence

The CDPs for 10-100 specific natural families are synthesized using random AA compositions (ie NNN, NNK, NNS or similar codons), then converted into libraries as a single pool, selected or screened for folding and expression as described above, followed by sequencing of the best folded and expressed clones. This approach results in a ranking of the scaffolds of natural families for their ability to accept random sequence. This approach tends to yield a higher average level of quality because the fraction of folded clones will be much higher than the random CDP approach, but it cannot evaluate as many scaffolds.

After selecting the preferred spacing patterns, we would determine which non-cys residues are required in a specific spacing pattern to improve folding.

Approach 3: Natural CDP, Natural AA sequence mixtures

The spacing patterns for 10-100 specific natural families are synthesized using the natural mix of AA compositions that occur at each position (as determined from alignments), then converted into libraries as a single pool, selected or screened for folding and expression as described above, followed by sequencing of the best folded and expressed clones. This approach tends to yield the highest average level of quality and the fraction of folded clones will be much higher than in the previous approaches, but it is more or less limited to a high density search of the sequence space nature has already explored.

The highest quality libraries (ie immediately useful for commercial targets) would results from synthesizing the natural families (natural CDP) with all of the fixed non-cys residues, but with some variation in each position. The sequence analysis of the well-folded clones will then tell us which of the fixed residues are truly required and in which residues variation is allowed.

Structure Evolution: The folding of disulfide containing proteins into a well-defined 3-D structure largely depends on the nature of the reducing environment present, both in vivo and in vitro. For example, reduction of disulfide bonds can lead to a complete loss of protein structure, underlining the importance of disulfide bonds for the maintenance of structure. On the opposite end, during the folding of a fully reduced and unfolded protein, a multitude of theoretical disulfide isomers are possible due to the oxidation of cysteines that come in close contact during folding. There are three theoretical disulfide isomers for a protein containing four cysteines, 15 isomers with six cysteines, 105 isomers with eight cysteines etc. Such diverse and often non-productive isomers are also observed during the protein folding process, but only one combination of cysteine pairings is usually represented in the native conformation. This is why disulfide isomerization is regarded as a major problem by most researchers during in vitro refolding studies. However, disulfide isomerization can be utilized for the evolution of structural diversity of disulfide-rich microproteins. Due to their small size and high-disulfide content these proteins often rely solely on the covalent linkages of cysteines to maintain a folded conformation. Many microproteins completely lack a hydrophobic core, which is regarded as a common underlying force for the folding of large proteins. Distinct disulfide isomers have been experimentally observed in a single member of the microprotein families Somatomedin B and snake conotoxins (Y. Kamikubo, et al. (2004) Biochemistry, 43: 6519-34; J. L. Dutton, et al. (2002) J Biol Chem, 277: 48849-57). However, these publications describe the presence of multiple isomers as a problem to be fixed, not as an opportunity to exploit for protein design. Generally applicable concepts and experimental procedures can therefore be developed to use disulfide isomerization as a driving force for structural evolution of microproteins.

Structural evolution by disulfide shuffling: See FIGS. 152, 153, 154. The following section provides a specific experimental approach to utilize disulfide isomers for structural evolution. After secretion of phage particles fused to a particular microprotein, these particles are subjected to highly reducing conditions by incubating the mixture at millimolar concentrations of reduced glutathione, a redox active and disulfide-containing tripeptide. Phage particles are then purified from reducing agent in a buffer containing millimolar concentrations of EDTA to prevent air oxidation of free thiols. This library will contain a large number of reduced and structurally diverse polypeptide chains. After contacting these reduced mixtures of isomers, the library is then subjected to oxidizing conditions, e.g. millimolar concentrations of oxidized glutathione, during target binding, to lock in favorable microprotein conformations by oxidation of their thiols. This approach selects for microprotein binders that initially interact with their targets in their reduced state and are then locked in the binding conformation by rapid oxidation. The pool of selected microproteins is shape-complementary to the target protein, and this process is called disulfide-dependent target-induced folding. The best binders are selected and subjected to additional cycles of directed evolution (mutagenesis and panning) until reaching an active and fully oxidized conformation in a target-independent manner, such that the target is no longer needed to induce the desired conformation, resulting in a protein that is easier to manufacture.

Alternatively, the phage library is subjected to a buffer of intermediate redox potential to allow disulfide shuffling. This can be easily achieved by choosing a buffer composition with varying ratios of oxidized and reduced glutathione. This will allow only partial oxidation of a subset of cysteine residues and subsequent disulfide shuffling, e.g. breaking and reformation of existing bonds favoring the accumulation of the most disulfide bonds. Therefore a pool of many different structural combinations (dependent on the number of cysteine residues of a given microprotein) is present under such conditions. The most potent clones will then be selected and subjected to another round of disulfide shuffling (with or without amino acid sequence optimization).

Covalent target binding through disulfide bonds:_Contrary to a long-held view, recent work has shown that the specific reduction of disulfide bonds can occur in the extracellular environment (P. J. Hogg (2003) Trends Biochem Sci, 28: 210-4). Endothelial cells were shown to secrete a reducing activity into their supernatants, which could be identified as thrombospondin-1, a glycoprotein with a redox active thiol in its calcium-binding domain (J. E. Pimanda, et al. (2002) Blood, 100: 2832-8). Remarkably, the free thiol of thrombospondin-1 controls the length of the adhesion protein von Willebrand factor by reducing intermolecular disulfide bonds. These observations can be utilized to covalently link novel microproteins to disulfide-containing target proteins. The approach would be to select for partially reduced and redox active microproteins which bind in the vicinity of disulfide bonds in target proteins. For example, after binding to a target protein, a phage display library of microprotein variants would be selected to resist washing under oxidizing conditions but to be specifically eluted upon washing under reducing conditions. Thus, during protein evolution, some disulfide bonds will be formed that stabilize microprotein structures, while others will be selected against to select for redox active free thiols.

The evolution of structural diversity refers to changes in structure experienced by a specific clone. The structure change is typically dependent on sequence change but even two identical sequences can adopt different structures. The structure differences can be at the level of disulfide bonding pattern or fold, which is generally due to structurally significant changes in loop length. Structure evolution differs from structural diversity (such as used by many multi-scaffold libraries) where multiple scaffold structures are used but each clone always adopts the structure of it's parental sequence: In structural evolution each clone can have a different structure from it's parental sequence.

FIG. 155 shows the dominant 3SS bonding pattern (18 different natural families) and the disulfide variants that can be created from it in one step. Most of the naturally occurring families are within 1 step of the dominant pattern (14 25 36). FIG. 155 also shows the 4SS variants that can be created by adding 1 disulfide to the dominant 3SS pattern (14 25 36), without changing any of the existing disulfides. 11/15 of the naturally occurring 4SS bonding patterns can be obtained by adding 1 disulfide to the dominant 3SS pattern without breaking any of the 3SS disulfide bonds. Since there are 105 total, the data suggest a strong preference for addition of a disulfide to a pre-existing 3SS protein. I think this analysis should be able to answer if the preferred path is the reverse, which is the deletion of a disulfide from a 4SS protein to create a 3SS protein). Unless the incompleteness of the database has affected these results (possible), it appears that the 14 25 36 and its 4SS derivatives obtained by addition of 1 disulfide are preferred starting points.

Microprotein build-up approaches: The goal of the build-up approach is to obtain stepwise affinity maturation of the binding protein for the target. At each cycle a library is created which adds a pair of cysteines plus a randomized sequence (typically a new loop) to the product from the previous selection cycle, followed by library panning to select the clones with the highest affinity for or activity on the target. The starting point can be a single sequence or a pool of sequences, and the sequence of the randomized area of the starting point can be known or unknown.

Creating 1-disulfide (‘1SS’) proteins as starting points: Novel microproteins with 2 or more disulfides can be created from single disulfide-containing proteins using a build-up approach. One build-up approach begins with a protein that contains two fixed cysteine residues (for a 1-disulfide or ‘1SS’ protein). Optionally, this protein can have the same intercysteine spacing or length (called ‘span’, which excludes the cysteines) as found in one loop of a preferred (typically natural) disulfide bonding pattern. Such similarity makes it easy to graft the 1SS peptide into a pre-existing 2SS, 3SS, 4SS or higher order scaffold. The spans for 1SS libraries are typically from 0 to 20 amino acids in length, preferably 5,6,7,8,9,10,11,12,13,14,15 and more preferably 7,8,9,10,11,12 and ideally 9,10,11 amino acids long. There can be additional randomization of residues outside of the pair of cysteines (ie outside of the loop or ‘span’). The initial 1SS protein is typically fully or partially randomized between the cysteines but sometimes it contains fixed amino acids (other than the cysteines) that provide folding or affinity to target molecule(s).

Build-up from 1SS to 2SS or higher scaffolds: One way to mature a previously selected 1SS protein is to provide two new cys residues in fixed positions, or in a variety of preferred positions as a library. Typically the residues flanking these two new cysteines as well as the new loop would be randomized.

Proteins with an uneven number of cysteines tend to be toxic and/or poorly expressed and are efficiently removed by the expression host. Thus, even if one encodes a random number of cysteines, only DNA sequence encoding an even number of cysteines are expressed as functional phage particles. Thus, one way to expand a previously selected (pool of) 1SS peptide(s) into a (pool of) 2SS peptide(s) is to create a library with a single third fixed cysteine as well as a larger (and variable) number of randomized residues, some of which are statistically expected to encode a Cys residue. A known fraction of these randomized positions will encode for cysteine residues, and, following the removal of sequences with an uneven number of cysteines by phage growth, 2SS proteins with a second pair of cysteines will constitute >50%, preferably >60-80% or sometimes even >90-95% of the phage library. The new cysteine(s) and/or the newly randomized area can either or both be on the N-terminal side of the starting protein, or either or both on the C-terminal side of the protein, or, less typically, inside the starting protein sequence. It is possible for the disulfide bonding pattern to change during the build-up process. The original disulfide bond(s) may be replaced by disulfide bonds linking different cysteines (new DBP).

Extension approach: Proteins (of any length or disulfide number) that bind to the target can be extended by fusing them to a randomized library sequence, which typically comprises one (or more) pair(s) of cysteines separated by a number of random positions and optionally with variable spacing. Libraries of such proteins are selected for enhanced binding affinity to a target molecule. This approach is likely to result in a second binding site of different sequence that folds separately from the first binding site.

Dimerization approach: Especially for targets that are homo-multimers or located on the cell surface, it is attractive to duplicate a previously selected binding site, creating a dimer, trimer, tetramer, pentamer or hexamer of identical disulfide-containing sequences, each able to bind to the same site on the target. If the target can be bound simultaneously at multiple sites, then the avidity of the binding increases. Optimal avidity typically requires that the spacing between binding sites is optimized by testing a variation of spacers of different length and optionally different composition. An example of a homo-dimeric microprotein that binds to human VEGF is described herein. A spacer composed of Gly-Ser is used between the binding sites and the length can be adjusted to provide optimal avidity for the dimeric VEGF target.

Series of existing CDPs: It is possible to add disulfides in such a way that the spacing (‘Cysteine Distance Pattern’, CDP) of each 1SS, 2SS or 3SS construct is the same as the CDP of an existing family of proteins, such that, for example, each stage of the buildup uses a natural CDP. It is also possible to graft the selected 1SS or 2SS protein into an existing 3SS, 4SS or 5SS scaffold in a place with similar loop length. Disulfides can be added with the goal of changing the existing disulfide bonding pattern, creating a library of structural variants or DBP variants, or maintaining the existing bonding pattern. Control over the DBP depends largely on whether the new cysteine pair and the new randomized sequence are added only on one end of the starting protein (tending to conserve the existing DBP) or whether they are added on both sides of the existing protein (ie one cysteine on each side), which tends to lead to changes in DBP. If one wants to conserve existing disulfide bond(s), then it helps to leave some extra spacer residues between the old cysteine pairs and the newly added cysteine pair(s). Such as spacer can have any sequence, but a glycine rich spacer is preferred (ie multimers of GGS or GGGGS). If the target molecule is dimeric (soluble) or cell-bound, then a spacer that is long enough to allow both microprotein motifs to bind to their target result in simultaneous binding at both sites, resulting in increased avidity or apparent affinity.

Build up by Megaprimer method: The Megaprimer methods allows the creation of new libraries from old libraries, avoiding the complexities arising from the presence of a library of sequences. A PCR fragment is generated containing the pool of previously selected 1SS proteins and this fragment is overlapped with a new DNA fragment (oligo or PCR product) encoding a new library with one or two new Cys residues. A ssDNA runoff PCR product (‘Megaprimer’) created from this overlap fragment, containing ends that are homologous to the vector, is annealed to the vector and used to drive a Kunkel-like polymerase extension reaction, using a template containing a stop-codon in the area to be replaced by the Megaprimer. Alternatively, a pair of unique restriction sites can be used to create a new library within a library of previously selected vectors. The genetic fusion to phage protein pIII or pVIII allows presentation of the protein on the phage capsid. Proteins with an even number of cysteines can be selected by: i) phage growth, ii) affinity selection, iii) free thiol purification, and/or iv) screening of DNA sequences. One or multiple cycles of this approach can be used to build the disulfide content up from 1 SS, 2SS, 3SS, 4SS, 5SS, 6SS or a higher number of disulfides. Any disulfide number can be used as the starting point.

A number of specific exemplary build-up process are described below.

The 234 Design Process: See FIG. 138. One preferred approach is called ‘234’, because it involves first creating and panning a 2-disulfide library containing a mixture of all three bonding patterns, then selecting a pool of the best clones, which is used to create a new library with additional (partially) randomized amino acid positions and one additional pair of cysteines, thus forming a three-disulfide library which can adopt up to 15 different structures, some of which would have the original four cysteines forming a different bonding pattern, thus enabling structural evolution of the original 2SS sequence. Each ‘library extension segment’ typically encodes several codons encoding a mixture of amino acids (ie encoded by an NNK, NNS, or similar mixed codon) plus one or more cysteines (located on the outside) and can be added at the 5′ or N-terminal end of the previously selected pool of sequences, or on the 3′ or C-terminal side of the previously selected pool of sequences, or at both ends. In order to avoid free thiols, it is desirable that an even number of cysteines (2,4,6) is added to each clone. This can be done by adding library extension segments to both ends (1 cysteine and 4-5 randomized codons on each end), or as one segment encoding two (or 4 or 6) cysteines and 6-8 ambiguous codons (encoding a desired mixture of amino acids) that is added to only the C-terminal end or only to the N-terminal end. This process can be repeated multiple times.

The 234 directed evolution process thus comprises of the following steps: initial library construction (2SS), target panning, (optional: screening of individual clones and pooling of the best), extension library construction (3SS), target panning, (optional: screening of individual clones and pooling of the best), extension library construction (4SS), target panning, and final screening of individual clones to identify the best 4SS binder.

Many variations of this process can be devised. It is possible to use 4,5,6,7 or more disulfides, or, for example, to make two-disulfide jumps instead of 1-disulfide jumps, or to pan one library against one target and the following library against a second target, in which the targets can be related or unrelated.

A preferred approach is to make a 2SS library with a CDP that is also found in (and preferably common) in natural 3SS protein, and to make a 3SS library with a CDP that is also found in natural 4SS proteins; this way one can be reasonably certain that the 2SS proteins can be matured into 3SS and that the 3SS proteins can be matured into 4SS proteins.

The 3x0-8 and 4x0-8 Design Processes: See FIG. 139. The ‘3x0-8’ and ‘4x0-8’ preferred design processes aim to create all of the 15 3-disulfide structures or all of the 105 4-disulfide structures in order to present maximal structure diversity and sequence diversity to the panning targets. The same approach can be extended to the 5-, 6-, or even 7-disulfide microproteins (5x0-8, 6x0-8, 7x0-8).

Analysis of the loop lengths of all of the natural 3-disulfide microproteins shows that the loops tend to range in size from 0-10 amino acids. The averages for the five loops (C1-C2, C2-C3, C3-C4 and C5-C6) are very similar (ranging from 0-8 to 3-12 after some of the longest loops are eliminated because they are undesirable), although between different scaffold families there are sharp differences in the size of the loops. For example, loop C1-C2 in conotoxins is 6AA long versus 0AA in anato domains, even though both have the same disulfide bonding pattern.

The sequence motif C1 x₀₋₈ C2 x₃₋₁₀ C3 x₀₋₁₀ C4 x₀₋₈ C5 x₀₋₉ C6 is predicted to cover over 90% of the natural 3SS protein sequences and the vast majority of all unknown 3SS microproteins with useful properties. The library construction process is easier with loops with equal length, such as 0-8, resulting in a library sequence motif of C1 x₀₋₈ C2 x₀₋₈ C3 x₀₋₈ C4 x₀₋₈ C5 x₀₋₈ C6, or the 4SS version of this design which is C1 x₀₋₈ C2 x₀₋₈ C3 x₀₋₈ C4 x₀₋₈ C5 x₀₋₈ C6 x₀₋₈ C7 x₀₋₈ C8. Other loop lengths that can be used are 0-10, 0-9, 0-8, 0-7, 0-6, 0-5, 0-4, 1-5, 1-6, 1-7, 1-8, 1-9, or 1-10 although most loop lengths are expected to work.

This type of library is expected to contain a large number of sequences that fold heterogeneously, meaning they are able to adopt multiple different structures and cannot be produced in homogenous form easily. This heterogeneity is a disadvantage for protein production but the increased diversity is an advantage for panning and early ligand discovery.

In traditional display libraries of synthetic protein diversity, all of the clones share the same fixed protein scaffold. While a huge diversity of sequences is created, they all share the same structure and no significant structural diversity is present. In contrast, the 3x0-8 and 4x0-8 libraries contain an approximately equal mixture of 15 or even 105 very different structures.

A typical phage display library contains 10e9 to 10e10 different clones, typically each having a different sequence. However, what is panned is a pool of about 10e13 phage particles containing on average about 1000-10,000 copies of each sequence or clone. This number of copies is called the ‘number of library equivalents’. Each of the 1000-10,000 copies of the same sequence can adopt a different structure, due to the folding heterogeneity that is mediated by disulfide bond formation. The effective library size of 3x0-8, 4x0-8 or 5x0-8 libraries is thus 10, 100, or 1000 fold greater than single scaffold libraries. A library of this design is thus expected to contain all or most of the theoretically possible structures, disulfide bonding patterns and folds.

It is possible to narrow the range of length range of the loops in order to keep the average protein small, prevent undesired structures from forming and to increase the frequency of desired structures. Intermediate loop lengths can be used, such as 2-6, 2-7, 2-8, 2-9, or 2-10 amino acids, or 3-4, 3-5, 3-6, 3-7, 3-8, 3-9 or 3-10 amino acids, or 4-5, 4-6, 4-7, 4-8, 4-9 or 4-10 amino acids, or 5-6, 5-7, 5-8, 5-9 or 5-10 amino acids.

It is also possible to pick a single fixed loop length for the library, typically 1,2,3,4,5,6,7,8,9 or 10 amino acids long.

A complementary approach to keep the average protein size small is to use DNA fragment sizing gels to select DNA fragments encoding an upper limit of 20,21,22,23,24,25,26,27,28,29,30,31,32,33,34,35,36,37,38,39,40,41,42,43,44,45,50,55,60 amino acids and a lower limit of 13,14,15,16,17,18,19,20,21,22,23,24,25,26,27,28,29,30,31,32,33,34, or 35 amino acids.

The 4×6 Design Process: See FIG. 140. A preferred approach is the ‘3×6’ or ‘4×6’ process, which starts with a library that has 3 or 4 disulfides and a fixed loop size of 6 amino acids that can have variable sequence. The protein sequence motif for the 4×6 library is C1 x₆C2x₆C3x₆C4x₆C5x₆C6x₆C7x₆C8 (subscript means the number of amino acid positions which can contain a mixture of bases (often encoded by NNK, NNS or a similar ambiguous codon; numbers after the C refer to the order of the cysteines in the protein from N- to C-terminus). In natural families of microproteins, cysteines that are bonded together are separated on the protein chain backbone by an average of 10-14 amino acids (average 12); we call this distance the ‘disulfide span’. The span is rarely less than about 8-9 amino acids. When neighboring cysteines disulfide bond, they form a sub-domain which is undesirable for most applications because it has its own thermal and protease instability profile. These undesirable subdomains can be eliminated by choosing a loop length that is too short to allow neighboring cysteines to bond, ie less than 9 amino acids. A fixed spacing of 6 AA appears to be especially favorable, because it prevents sub-domains and creates multiple places where (non-neighboring) cysteines are spaced 12 amino acids apart, which appears to be ideal since it is the average in natural proteins. Eliminating the subdomains removes the 69 worst 4SS disulfide bonding pattern and can only give the 36 best 4SS disulfide bonding patterns. Fixed spacings of 4,5,7 or 8 amino acids or combinations thereof are also feasible.

The vast majority of the known natural 3SS toxins would be contained in a single ‘all-scaffold’ library with the following composition: C1-(x₀₋₁₀)-C2-(x₂₋₁₂)—C3-(x₀₋₁₀)-C4-(x₀₋₁₀)-C5-(x₀₋₁₂)—C6. Such a library would additionally contain the vast majority of unknown natural toxins and an even larger number of non-naturally occurring toxins. The average length of proteins encoded by such a library would be: 1+5+1+7+1+5+1+5+1+5+1=33 amino acids.

To create shorter proteins, it would be possible to use a higher molar ratio of the oligos encoding the short sequences to those encoding the long sequences, or to limit the maximum loop length to only 8 aa rather than 10-12 aa.

Similarly, an all-scaffold library with the following composition would comprise the vast majority of 4-disulfide HDD toxins, with 105 different disulfide bonding patterns and over a thousand potential folds:

C1-(x₀₋₁₀)-C2-(x₀₋₁₀)-C3-(x₀₋₁₀)-C4-(x₀₋₁₀)-C5-(x₀₋₁₀)-C6-(x₀₋₁₀)-C7-(x₀₋₁₀)-C8

And a 5-disulfide ‘all-scaffold’ library would be specified by

C1-(x₀₋₁₀)-C2-(x₀₋₁₀)-C3-(x₀₋₁₀)-C4-(x₀₋₁₀)-C5-(x₀₋₁₀)-C6-(x₀₋₁₀)-C7-(x₀₋₁₀)-C8-(x₀₋₁₀)-C9-(x₀₋₁₀)-C10.

The x typically refers to a desirable mixture of amino acids. Although one can use NNN codons to encode the mixture of amino acids, other codons have advantages. Each codon offers a different mixture of amino acids.

For example, NNK decreases the frequency of stop codons 3-fold. Different codons are useful for different applications. A mix favoring hydrophilic amino acids is desirable, avoidance of stop codons, tryptophans, other hydrophobic amino acids and avoidance of cysteines in the loops is also desirable. Molecular biologists know how to select the codons that yield the mixture that is desired. The codons that would typically be used to select contain A,C,G,T or the mixed-base letter N,M,K,S,W,Y,R,B,D,V or H as the first base in the codon, and contain A,C,G,T or the mixed-base letter N,M,K,S,W,Y,R,B,D,V or H as the second base in the codon, and contain A,C,G,T or the mixed-base letter N,M,K,S,W,Y,R,B,D,V or H as the third base in the codon, resulting in a large number of possible codons each encoding a different mixture of amino acids.

The loop sequences of natural HDD proteins contain a small number of fixed residues that are likely to play a role in protein folding. The previous approach simply uses random codons and lets the diversity supply these residues if they truly are important for folding. This random codon approach will result in lower library quality compared to libraries that use the natural composition of amino acids for each position, but may be the best at exploring the potential for novel folds.

However, if, for example, a W is required for folding or function but an NNK codon is used in that position, only 1/64 clones in the library meet this requirement, so the effective size of the library is reduced 64-fold, which may be sufficient to prevent obtaining useful binders. It is therefore likely to be important that any residues that appear to be fixed in natural sequences are also fixed in the library.

An alternative approach to the use of random codons (NNK or one of the many others described above) is to synthesize oligonucleotides with the exact consensus sequence of the loop of a specific protein family. This approach requires that loop 2 designs are only incorporated in the loop 2 location of the library, and loop 3 sequences only in the loop 3 location. This can be achieved if the cysteines, where the overlap reaction occurs, each are encoded by a different one of the three cysteine codons. One to three bases before or after the cys codon can be fixed as well, in order to provide a more efficient overlap PCR reaction. The overlap reaction efficiency can limit the diversity of the library so this is an important risk which cannot be detected or controlled easily. In general, the addition of a few bases is an effective way to reduce the serious risk of low library diversity.

After mixing all of the loop sequences for the different families and incorporating them by overlap PCR, all of the synthetic loop sequences should only occur in their natural position. This library approach results in the shuffling of loops from different families relative to each other.

Increasing Library Diversity: The power of natural and directed evolution is related to the diversity that is subjected to selection pressure. Selections from a larger number of more diverse clones generally yield better outcomes. Organisms use multiple approaches to increase the diversity of protein structures beyond the number of genes. This expanded natural diversity provides more solutions for selection to act on and increases the power of natural evolution.

There are many different ways in which we can increase the diversity of structures that can be obtained from the same number of clones or number of sequences, with the goal of increasing the power of directed evolution.

This principle can be applied to the optimization of single genes, multi-gene pathways, whole genomes (prokaryotic, archaeal, eukaryotic) and even whole communities of organisms (ie microbial communities).

In general, expression of a single gene yields a variety of different mRNA sequences. This can be due to multiple promoters, due to alternative splicing, trans-splicing, or degradation. Each mRNA sequence can fold differently, adopting a variety of different structures and the outcome can also be modulated by the presence of other RNAs (micro-, tRNAs or mRNAs) as well as proteins that interact with RNA. Each of these mRNA structures can be translated somewhat differently, through the presence of multiple translation start and stop signals, variants with different pausing on the ribosome or a low but variable degree of misincorporation of amino acids, including ‘non-natural’ amino acids. In addition, each protein translation product can fold differently, some aggregating, some misfolding, some being degraded by proteases, some ubiquitinated and some folding into multiple stable structures. An important and practical differentiation mechanism is the derivatization of proteins, the chemical alteration of amino acid side chains and the chemical linking of small molecules such as sugars and polymers like PEG to the protein chain. These chemical approaches can be applied to the entire library (most) or to purified single proteins.

When applied to a library they can increase diversity dramatically, especially if applied sparingly, so that a heterogenous population results. For example, the non-exhaustive conjugation of a PEG or carbohydrate molecule to a Lysine residue on a protein library containing 5 lysines results in 5-factorial+1 types of molecules (122 variants). The best variants are selected by panning and now variants of the labeling recipe are applied to library equivalents, pools of clones or to single clones in order to discover which recipe gives the best results. In addition, the sequence of the proteins is evolved and selected for retention and improvement of the desired activity. The best mutant, for example, would have lost the four lysines that do not contribute to the activity and have kept the lysine that, when derivatized, results in an increased level of activity. All of the reagents that are used for derivatization of proteins (ie Pierce Chemical on-line catalog) can in principle be used for this approach. There is a fine balance between unique, stable structures for cellular function and diversity and some instability which can accelerate cellular evolution.

Each of these mechanisms is a potential point for experimental intervention: each of these controls was set at it's current level of variation by natural evolution but it's diversity could be increased or decreased depending on the goals of directed evolution.

An area of specific commercial interest is the directed evolution of binding proteins using display libraries (phage, yeast, bacterial surface, polysome, ribosome, pro-fusion, or gene-fusion libraries). It has been well-established that the frequency and quality of the best selected clones correlates directly with the size of the library. The larger the library, the higher the number of binders and the better the best will be. Because of this, a variety of approaches have been developed to create larger and larger libraries, such as the recombination method used to combine two immunoglobulin libraries of 10e6 clones into a single library of 10e12 clones. However, in this example all of the library proteins have the same immunoglobulin fold, which focuses the diversity into a single structure that is beneficial for some applications ie whole antibody products) but not suitable for creating a diversity of different structures. Rather than increasing the number of clones in the library, it is also possible to increase the effective library size by increasing the number of structures that can be created from a single sequence.

Rather than increasing library diversity by increasing the number of clones, an alternative approach to increasing library diversity is to increase the diversity of structures adopted by each clone. This can be obtained using destabilized proteins, which are more similar to a molten globule in that they exist as a large diversity of structures, each at a fraction of time. This approach allows searching of a much larger space including novel backbone structures that would not be accessed in a library of highly structured proteins. This more global search allows the identification of more globally optimal folds and further directed evolution can be used to create stably folded and homogeneously manufacturable variants of this novel fold.

The target is typically a protein, but could also be nucleic acid (DNA, RNA, PNA), carbohydrate, lipid, metabolite, or any biological or non-biological material). Because the library protein is (partially) unstructured, it adopts many different structures, each for a small fraction of time. This increases the molecular diversity of the library and favors the use of a large number of library equivalents. For panning a standard phage library one typically uses 100 library equivalents, or 10e12 phage if the library is 10e10 diversity. It has been found experimentally that this 100-fold excess is necessary to allow reliable recovery of a specific (structured) clone from a library. For high affinity clones one can use a lower excess, and for low affinity clones one should use a higher excess.

In contrast to other approaches for creating diversity, we will call this ‘temporal diversity’, because the diversity is obtained by multiple structures each occupying a fraction of time. The creation of diverse structures from the same single gene is an important principle for biological evolution and exists at many levels of biological organization.

Expanding the Diversity of Display Libraries:_Phage libraries typically contain about 10e14 phage with a diversity of 10e10 different sequences. It is well-established that affinity chromatography can select a single sequence expressing a binding protein out of such a library (10e10 enrichment). Since virtually 100% of the phage that can bind at high affinity will be bound by the affinity column, one can also predict that a single copy of a phage can also reliably be selected by this approach (10e14 enrichment).

A phage displayed peptide would typically exist in 10e3-10e6 different unstable conformations, only one of which binds to the column. Because column binding stabilizes the active conformation of the peptide, such peptides can be enriched efficiently, yielding an enrichment 10e17-10e20). Flexibility in the backbone conformation thus increases the effective library size to 10e20. After the first panning round, the diversity is typically already 1 000-fold reduced, so that in subsequent libraries each clone is represented by 1000 or more copies, which means that all of the different temporary structures that the proteins can adopt are statistically well represented. Over the course of further directed evolution the goal is to select for clones that spend an increasing fraction of their time in the structures with high affinity for the target. The goal is to gradually improve the affinity as well as the stability of the protein using various mutation approaches combined with selection.

Target-Induced Folding: The structure of the microprotein can be induced by target binding (by forming the disulfides after target binding), or the structure of the microprotein can be optimized while bound to it's target.

Binding to a target invariably involves some degree of induced fit and thus is expected to stabilize some of the disulfides (those in the part that is bound) and destabilize other disulfides, resulting in differential sensitivity to reducing agents. Titrating in reducing and oxidizing agents (at various concentrations and time intervals) allows rapid reducing and reoxidizing of the least stable disulfides, which, if there is a change in bonding pattern, results in structural adaptation and a better fit to the bound target, This approach increases the survival of clones with the best binding affinity.

For production, it may be desirable that the folding of the protein is evolved to be target-independent.

Optimizing the amino acid composition of microproteins:_Most proteins or protein domains comprise a hydrophobic core that is critical for protein stability and conformation. The hydrophobic core of these proteins contains a high fraction of hydrophobic amino acids. Amino acids can be characterized based on their hydrophobicity. A number of scales have been developed. A commonly used scale was developed by (Levitt, M (1976) J Mol Biol 104, 59, #3233), which is listed in (Hopp, T P, et al. (1981) Proc Natl Acad Sci USA 78, 3824, #3232). Hydrophobic residues can be further divided into the aliphatic residues leucine, isoleucine, valine, and methionine, and the aromatic residues tryptophan, phenylalanine, and tyrosine. FIG. 1 compares the abundance of amino acids in all proteins as published in Brooks, D J, et al. (2002) Mol Biol Evol 19, 1645, #3234 with the average amino acid abundance that was calculated for 8550 microprotein domains that are contained in the data base published in Gupta, A., et al. (2004) Protein Sci, 13: 2045-58.

See FIG. 13: Prevalence of amino acids in proteins. This figure reveals that microproteins tend to have a significantly lower abundance of aliphatic hydrophobic amino acids relative to other proteins, which has not been appreciated in the art. In contrast, the abundance of aromatic hydrophobic amino acids (W, F, Y) is similar to average proteins. This low abundance of aliphatic amino acids reflects the fact that microprotein structures are stabilized by several disulfide bonds, which obviates the need for a hydrophobic core. It reveals that several other amino acid residues that contain aliphatic carbon atoms (glutamate, lysine, alanine) also occur with reduced abundance in microproteins relative to other proteins.

Utility of scaffolds with low hydrophobicity: Reducing the abundance of aliphatic amino acids in proteins can significantly increase their utility in pharmaceutical and other applications. Many proteins have a tendency to form aggregates during folding. This can be aggravated when the protein is produced at high concentrations in a heterologous host and when the protein is renatured in vitro. Aggregation and misfolding can significantly reduce the yield of protein during commercial production. By reducing the fraction of aliphatic amino acids in a protein sequence, one can reduce the propensity to form aggregates and thus one can increase the yield of correctly folded protein.

Proteins with a low abundance of aliphatic amino acids have a lower immunogenicity relative to other proteins. Aliphatic amino acids tend to increase the binding of peptides to MHC, which is a critical step in the formation of an immune reaction. As a consequence, proteins containing a low fraction of aliphatic amino acids tend to contain fewer T cell epitopes relative to most other proteins.

Aliphatic residues have a propensity to form hydrophobic interactions. As a consequence, proteins with a large fraction of aliphatic amino acids are more likely to bind to other proteins, membranes, and other surfaces in a non-specific manner. Aliphatic residues that are exposed on the surface of a protein have a particularly high tendency to make non-specific binding interactions with other proteins. Most of the amino acids in a microprotein have some surface exposure due to the small size of microproteins.

Accordingly, the present invention provides a non-natural protein containing a single domain of 20-60 amino acids which has 3 or more disulfides, and wherein the protein binds to a human serum-exposed protein and has less than 5% aliphatic amino acids. Where desired, the a non-natural protein contains less than 4%, 3%, 2% or even 1% aliphatic amino acids. In addition, the present invention provides libraries of non-natural protein having such properties.

Identification of scaffolds with low hydrophobicity: Although most microproteins contain fewer aliphatic amino acids compared to most normal proteins, there is significant variation in the content of aliphatic amino acids between different microprotein families. Table 4 lists some families of microproteins that particularly useful as starting points for the engineering of pharmaceutical proteins with a low abundance of aliphatic residues.

Design of Proteins of Low Immunogenicity: Proteins of low immunogenicity are more desirable as therapeutics because they are less likely to elicit undesired immune response when administered into humans. In some aspects, the subject microproteins with desired target binding specificities are generally less immunogenic than proteins capable of binding to the same target but without the desired cysteine bonding pattern or fold. In one embodiment, the subject microproteins are 1-fold less, preferably 2-fold less, preferably 3-fold less, preferably 5-fold less, preferably 10-fold less, preferably 100-fold less, preferably 500-fold less, and even more preferably 1000-fold less immunogenic. In some embodiments, the microproteins of low immunogenicity are HDD proteins described herein.

The immunogenicity of proteins can be predicted using programs such as TEPITOPE, which, based on a large set of affinity measurements, calculate the binding affinity of all overlapping nine amino acid peptides derived from an immunogen to all major human HMC class II alleles (Sturniolo et al. 1999; xvww.biovatioii.com; www.epivax.com; www.algonomics.com). Such programs are widely used for the prediction and removal of human T-cell epitopes and their use is encouraged by the FDA.

Using these algorithms, we found that microproteins having 25-90 residues and more than 10% cysteine, typically have 316-fold lower predicted affinity for binding to MHCII than average proteins. The red curve in FIG. 166 shows the predicted immunogenicity of all 26,000 human proteins, with a median length of 372 amino acids. The blue curve shows the predicted immunogenicity of all 10,500 microproteins, with a median length of 38 amino acids. The green curve shows the predicted immunogenicity for a non-natural group of protein fragments with the same length distribution as the microproteins, but composed of randomly chosen human sequences. Comparison of the mean score for each group shows that the one-log reduced size of the microproteins alone leads to a 67-fold reduction in immunogenicity, and the amino acid composition of the microproteins yields an additional 4.7-fold reduction. FIG. 167 top panel shows that aliphatic hydrophobic amino acids (I,V,M,L) are ranked as the strongest contacts in the TEPITOPE algorithm (Sturniolo et al 1999), contributing most to the predicted immunogenicity. FIG. 167 bottom panel shows that these aliphatic residues are also the most underrepresented in microproteins compared to human proteins, accounting for most of the composition-derived one-log reduction in predicted immunogenicity.

The low level of aliphatic hydrophobic residues in microproteins is made possible by their lack of a hydrophobic core that is typical for other proteins. Instead, microproteins contain a small number of cysteines, which crosslink to form intrachain disulfides. This replacement of a large number of hydrophobic amino acids with a few disulfides reduces the minimum size at which the proteins are stable, allowing microproteins to be smaller and reducing the frequency of aliphatic amino acids, resulting in the three logs in reduction in predicted immunogenicity.

The reduced immunogenicity can be measured by a variety of indications, including e.g., 1) the capacity of the antigen presenting cell (APC) such as a dendritic cell (DC) to release peptides from the immune protein (antigen processing); 2) the presence of T-cell epitopes in these peptides which determines binding to HLA II molecules; 3) the number of naïve T cells in blood that recognize the peptide-HLAII complex on the APC surface; and 4) the level of antibodies in serum.

There exists numerous ways for lowing protein immunogenicity, all of which are applicable for HDD and non-HDD proteins. One approach is to add disulfides via computer modeling and rational design. Another approach is to improve existing disulfides by fine-tuning the protein using directed evolution or rational design. It may be possible to protect the disulfides from chemical attack by putting them in the interior of the protein or flanking the cysteines with amino acid side chains that have a protective effect. The immunogenicity of proteins can also be predicted using programs such as TEPITOPE or Propred, which, based on a large set of affinity measurements, calculate the binding affinity of all overlapping nine amino acid peptides derived from an immunogen to all major human HMC class II alleles (other programs are used for MHC class I). See Sturniolo, T., et al. (1999) Generation of tissue-specific and promiscuous HLA ligand databases using DNA microarrays and virtual HLA class II matrices. Nature Biotechnol, 17: 555. See also www.algonomics.com, www.biovation.com, www.epivax.com and www.genencor.com. Such programs are widely used for the prediction and removal of human T-cell epitopes and their use is encouraged by the FDA.

Yet another approach for generating less immunogenic microproteins is via intra-protein crosslinking using chemical crosslinking agents. A wide variety of crosslinkers are available from commercial vendors such as Pierce. Applicable crosslinkers include arginine-reactive cross-linkers, homobifunctional crosslinking agents such as amine-reactive homobifunctional crosslinking agents, sulfhydryl-reactive homobifunctional crosslinking agents, hetero-bifunctional crosslinking agent such as amine-carboxyl reactive heterobifunctional crosslinking agents and amino-group reactive heterobifunctional crosslinking agents.

Yet still another approach is to make a small protein with multiple binding sites and separate each domain into two or three binding sites. For instance, one face of the domain binds one target and the other half binds another target. The two faces can be designed in parallel (ie in separate libraries simultaneously) and then merged into one domain. The alternative is to design the two faces successively, creating one library in the residues on face 1 and panning this library for binding to target 1, selecting one or more of the best clones and creating a new library 2 in the remaining amino acids, those that were not used for library 1, followed by panning against target 2 and screening for binders to target 2 and retention of binding against target 1. Because the amino acids for face 1 tend to be interdigitated with the amino acids for face 2, the construction of these libraries into a pool of clones with different sequences can be readily performed if one keeps certain amino acids fixed, so that these fixed bases can provide the required contacts for overlap extension by PCR. Since the cysteines tend to be fixed, these are the logical choice as the overlap points for the different oligonucleotides. However, an overlap works better if it has 4 or more bases, so it is useful to fix one additional amino acid on either side of the cysteine. The scaffold for a two-face library thus has three sets of amino acids and bases: ones for face 1/library 1, ones for face 2/library2, and fixed ones for combining the two libraries by overlap extension. It is in principle possible to use restriction sites, but the overlap approach will generally work better.

Still another approach is to decrease protein size by minimizing the length of the intercysteine loops. A typical approach is to use a range of loop lengths in the library, some of which occur naturally and some that are shorter than what is found naturally.

Still another approach is to increasing hydrophilicity. Most of the HDD proteins are highly hydrophilic and this may be important for function (specificity, non-immunogenicity) as well as for folding of the protein. The hydrophilicity can be controlled by choosing the mix of amino acids used in each position in the protein library, picking (a mix of) the desired codons for the synthesis of the oligonucleotides. A good general approach is to mimic the natural composition of each amino acid position, but one can skew this to favor certain desired residues. Clones can be screened for size and for hydrophilicity by DNA sequencing. The various approaches described above can be employed alone or in combination.

Any of the subject microproteins can be employed for further modification. Non-limiting examples are HDD proteins such as modified A-domains, LNR/DSL/PD, TNFR, Anato, Beta Integrin, Kunitz, and the animal toxin families Toxin 1, 2, 3, 4, 5, 6, 7, 8, 9, 10, 11, 12, Myotoxins, Conotoxins, Delta- and Omega-Atracotoxins. The deimmunization approaches described here can be applied to a wide variety of human or primate proteins, such as cytokines, growth factors, receptor extracellular domains, chemokines, etc. It can also be applied to other non-HDD scaffold proteins, such as immunoglobulins including Fibronectin III, and to Ankyrin, Protein A, Ubiquitin, Crystallin, Lipocalin. Provided that immunogenicity can be minimized, non-human scaffolds are preferred over (near-) native human proteins and human-derived scaffolds because of the reduced potential for cross-reaction of the immune response with the native human protein.

A number of methods are available for assaying for a reduce immunogenicity of HDD proteins. For example, one can assay for protein degration by human or animal APCs. This assay involves addition of the protein of interest to human or animal antigen presenting cells, APC-derived lysosomes or APC proteases and looking for degradation of the protein, for example by SDS-PAGE. The APCs can be dendritic cells derived from blood monocytes, or obtained via other standard methods. One can use animal rather than human APC, or use cell lysates rather than whole cells, or use one or more purified enzymes or cell-fractions such as lysosomes. Degradation of the protein is most easily determined by denaturing SDS-PAGE gel analysis. Degraded proteins will run faster, at lower apparent molecular weight on the gel. The protein of interest needs to be detected in the large amount of cellular proteins. One way is to fluorescently or radioactively label each clone (radioactive: 3H, 14C, 35S; dyes and fluorescent labels like FITC, Rhodamine, Cy5, Cy3, etc.) or any other suitable chemical labels, so that only the protein of interest and its degradation products are visible on the gel upon UV exposure or autoradiography. It is also possible to use peptide-tagged proteins which can be detected using an antibody in Western blots.

Another approach to determine immunogenicity is to assay for the propensity of protein aggregation. Protein aggregation is easily determined by light scattering and can be performed with a dynamic light scattering instrument (DLS) or a spectrophotometer (ie OD 300-600 versus OD 280).

One can also assay for the level of T-cell stimulation and cytokine activation. Cytokine activation is measured on human PBMC's by FACS for the presence of activation antigens for dendritic cells (CD83 etc), T cell activation (CD69, IL-2r, etc.) as well as the presence of many co-stimulatory factors (CD28, CD80, CD86), all of which indicate that the immune system has been stimulated. Further the cells can be examined for production of cytokines such as IL-2,4,5,6,8,10, TNF alpha, beta, IFN gamma, Il-1 beta etc. using standard ELISA assays. The regular mitogens, and LPS etc. can serve as good controls.

Furthermore, one can assay for binding to Toll-receptors. Binding of the therapeutic protein to Toll-like receptors 1-9 (TLR1-TLR9) is a useful indicator of innate immunity. A number of commercial vendors such as Invivogen provide all of the transgenic Toll-receptors hooked up reporter genes in cellular constructs.

In addition, one can perform animal studies to assess protein immunogenicity by directly injecting the proteins into a host animal, such as rabbit and mouse.

The following provides an example of eEngineering of microproteins with low binding affinity for HLA II. See FIG. 161. Helper T cell activation is a key step and essential for the initiation of an immune reaction against a foreign protein. T cell activation involves the uptake of an antigen by an antigen presenting cell (APC), the degradation of the antigen into peptides, and the display of the resulting peptides on the surface of APCs as complex with proteins of the human leukocyte antigen DR group (HLA-DR). HLA-DR molecules contain multiple binding pockets that interact with presented peptides. The specificity of these HLA-DR pockets can be measured in vitro and the resulting specificity profiles can be used to predict the binding affinity of peptides to various HLA-DR types (Hammer, J. (1995) Curr Opin Immunol, 7: 263-9). Computer programs have been described that allow one to identify HLA-DR binding sequences (Sturniolo, T., et al. (1999) Nat Biotechnol, 17: 555-61). The current invention exploits these algorithms with the goal of modifying the sequences of microproteins in a way that reduces binding to HLA-DR while maintaining the desired pharmacological and other properties of the parent microprotein. As a first step the sequence of the parent microprotein is analyzed using a HLA-DR prediction algorithm. All possible single amino acid mutations of non-cysteine residues in the parent sequence are being compared with the parent sequence, and binding to HLA-DR types is predicted. Goal is to identify a set of mutations, that are predicted to reduce binding to HLA-DR types that occur at high frequency in the patient population that will be treated with the parent microprotein or with its derivatives. Subsequently, one constructs a combinatorial library where variants in the library contain one or more mutations that are predicted to reduce HLA-DR binding. It may be advantageous to construct several sub-libraries that contain subsets of the planned mutations. The resulting library or the sub-libraries can then be screened to identify variants that bind to the appropriate target. In addition, one can screen library members for stability, solubility, expression level, and other properties that are important for the final properties. Prior to screening, one can also subject the combinatorial library to phage panning or similar enrichment method to isolate combinatorial variants that retain the desired target-binding affinity and specificity. This process will identify variants of the parent microprotein that retain all desired properties of the parent protein but that are predicted to have reduced binding to HLA-DR and consequently reduced immunogenicity. Optionally, one can subject the resulting improved variants to a subsequent round of removal of HLA-DR binding sequences. This subsequent round can be a simply a repeat of the procedure described above. As an alternative, one can limit the second combinatorial library to mutations that were identified during round one of the process as compatible with the desired microprotein function and that were predicted to further reduce HLA-DR binding. By limiting the second round of the process to these pre-selected mutations one can construct smaller libraries and increase the frequency of isolating improved variants.

TABLE 4
Microprotein families with low abundance of aliphatic amino acids

| | | | Aliphatic amino | | |
|---|---|---|---|---|---|
| | | | acids | | |
| PFAM | Family size | Length | (%) | Description | Source |
| PF02977 | 3 | 27.0 | 0.00 | Carboxypeptidase A inh. | plants |
| PF05374 | 4 | 19.0 | 2.63 | Mu-Conotoxin | cone snails |
| | | | | fungal cellulose binding | |
| PF00734 | 42 | 18.1 | 4.07 | domain | fungal |
| PF00187 | 228 | 36.2 | 4.93 | chitin recognition protein | plants |
| PF06357 | 7 | 33.0 | 6.06 | omega-atratoxin | spiders |
| PF05294 | 11 | 32.6 | 7.24 | Scorption short toxin | scorpions |
| PF05453 | 6 | 24.0 | 7.64 | BmTXKS1 toxin family | scorpions |
| PF05353 | 5 | 42.2 | 8.06 | Delta atratoxin | |
| PF05375 | 24 | 29.5 | 8.63 | Pacifastin inhibitor | locust |
| PF00200 | 285 | 64.1 | 8.68 | Disintegrin | snakes |
| PF01033 | 68 | 35.6 | 9.00 | Somatomedin | mammalian |
| PF00304 | 105 | 44.8 | 9.08 | Gamma-thionin | plants |

Average proteins contain 26.1% aliphatic amino acids.

Methods to Reduce the Fraction of Hydrophobic Amino Acids in Therapeutic Proteins

As described above, one way to create microproteins with a low abundance of aliphatic amino acids is by starting with scaffolds and libraries that contain few aliphatic amino acids. In addition, one can reduce the abundance of aliphatic amino acids in a protein using a variety of protein engineering techniques. For instance, one can construct protein libraries such that one or several aliphatic amino acids have been replaced with random codons that allow for many hydrophilic amino acids to occur. Of particular interest are ambiguous codons which allow a large fraction of hydrophilic amino acids but a low fraction of aliphatic or hydrophobic amino acids. For example, the codon VVK allows the occurrence of 12 amino acids (alanine, aspartate, glutamate, glycine, histidine, lysine, asparagine, proline, glutamine, arginine, serine, threonine) and it avoids all aliphatic and aromatic amino acids. One can isolate proteins with desirable properties from such libraries and thus reduce the abundance of aromatic hydrophobic and aliphatic hydrophobic amino acids. One can also construct combinatorial protein libraries that randomize multiple amino acid positions that contain aliphatic amino acids. By determining the sequence and performance of multiple variants from such libraries, one can identify positions in said protein that allow replacement with hydrophilic amino acids.

Methods to Evaluate Scaffold Utility

Create design based on a specific family of natural sequences. In each amino acid position a mixture of amino acids is used that reflects the natural diversity of amino acids at that position. This is done by choosing the single most suitable codon. An HA tag is added to the N-terminal end of the protein and a His6 tag is added to the C-terminal end.

Oligonucleotides encoding these protein designs are synthesized. 1-30 different designs are constructed simultaneously, singly or as a mixture of different designs.

Expression of the Subject Composition

Intracellular Versus Extracellular Environment

Disulfide bonds are mainly found in secreted (extracytosolic) proteins. Their formation is catalyzed by a number of enzymes present in the endoplasmic reticulum (ER) of multicellular organisms. On the other hand, disulfide bonds are generally not found in cytosolic proteins under non-stress conditions. This is due to the presence of reductive systems such as glutathione reductase and thioredoxin reductase, which protect free cysteines from oxidation. For example, ribonucleotide reductase forms a disulfide bond during its reaction cycle and reduction of this disulfide bond is essential for the reaction to proceed (Prinz, J Biol. Chem. 272(25):15661).

Natural microproteins are expressed by bacteria, animals (sanemones, snails, insects, scorpions, snakes) and plants. However, heterologous expression of recombinant microproteins has generally been performed in E. coli, although Bacillus subtilis, yeast (Saccharomyces, Kluyveromyces, Picchia), and filamentous fungi such as Aspergillus and Fusarium, as well as mammalian cell lines such as CHO, COS or PerC6 could also be used for expression of microproteins. In the literature examples heterologously expressed microproteins are typically produced in the cytoplasm of E. coli.

An alternative to recombinant expression is chemical synthesis. Microproteins are small enough to allow chemical synthesis and could be manufactured by synthesis at an economically viable cost.

Unrelated products that contain disulfides (most Ig-domain-containing products, including Ab fragments and whole Abs) are generally produced in mammalian tissue culture or in E. coli by secretion into the periplasm or into the medium. Secreted products have a signal peptide which is proteolytically removed, leaving the N-terminal residue unformylated. In contrast. Proteins produced in the cytoplasm of E. coli frequently retain the N-terminal formyl-Methionine, depending on the amino acid(s) following the fMet. The literature describes which amino acids following the fMet result in fMet removal.

While Microproteins are almost completely absent from bacteria and archaea (some exceptions), all of the hydrophilic microproteins can readily be made in E. coli.

There are a few bacterial microproteins, such as the heat-stable enterotoxin from E. coli (called ST-Ia and ST-Ib) and related enterobacteria. Heat stable enterotoxins such as STa (PFAM 02048) and STb are unrelated on the sequence level. Sequence alignments of St-!a show a 72aa precursor. The protein is processed by two independent proteolytic cleavage events to yield the mature toxin, which contains three disulfide bonds with a topology of 14 25 36. The motif for ST-Ia is CxxxxxxxxxxxxxxxxxxxxCCxxCCxxxCxxC.

A promising way to express microproteins and to secrete microproteins into the media may be to use the ST-Ia promoter and leader peptide and precursor, but hooked up to a different microprotein, replacing the current 3SS 14 25 36 module with a different microprotein. ST-Ia is secreted into the medium (not periplasm), which is very rare for E. coli and explains how the disulfides are formed. It is likely to have a specialized leader peptide that allows it to be secreted from E. coli via one the 3 or 4 different specialized secretion systems. Hooked up to there microproteins, this leader peptide may allow efficient secretion and disulfide bond formation of other microproteins as well and may be useful for rapid screening of culture supernatants.

Microproteins can be produced in a variety of expression systems including prokaryotic and eukaryotic systems. Suitable expression hosts are for instance yeast, fungi, mammalian cell culture, insect cells. Of particular interest are bacterial expression systems using E. coli, Bacillus or other host organisms. Heterologous expression of microproteins is typically performed in the cytoplasm of E. coli. The disulfide bonds generally do not form inside the cytoplasm, since it is a reductive environment, but they are formed after the cells are lysed. The characterization and purification of microproteins can be facilitated by heating the cells after protein expression. This process leads to cell lysis and to the precipitation of most E. coli proteins. (Silverman, J., et al. (2005) Nat Biotechnol) . The expression level of different microproteins in E. coli can be compared using colony screens, if the microprotein is fused to a reporter like GFP or an enzyme like HRP, beta-lactamase, or Alkaline Phosphatase. Of particular interest are heat and protease stable enzymes as they allow to assay the stability of microproteins under conditions of heat or protease stress. Examples are calf intestinal alkaline phosphatase or a thermostable variant of beta-lactamase (Amin, N., et al. (2004) Protein Eng Des Sel, 17: 787-93). The fusion of microproteins to enzymes or reporters also facilitates the analysis of their binding properties as one can detect target-bound microproteins by the presence of the reporter enzyme. Microproteins can be expressed as a fusion with one or more epitope tags. Examples are HA-tag, His-tag, myc-tag, strep-tag, E-tag, T7-tag. Such tags facilitate the purification of samples and they can be used to measure binding properties using sandwich ELISAs or other methods. Many other assays have been described to detect binding properties of protein or peptide ligand and these methods can be applied to microproteins. Examples are surface plasmon resonance, scintillation proximity assays, ELISAs, AlphaScreen (Perkin Elmer), Betagalactosidase enzyme fragment complementation assay (CEDIA).

Heterologous expression of microproteins is typically performed in the cytoplasm of E. coli. The disulfide bonds generally do not form inside the cytoplasm, since it is a reductive environment, but they are formed after the cells are lysed. The expression level of different microproteins in E. coli can be compared using colony screens, if the microprotein is fused to a reporter like GFP or an enzyme like HRP or Alkaline Phosphatase (preferably a heat stable version such as calf intestinal alkaline phosphatase).

The invention also encompasses fusion proteins comprising cysteine-containing scaffolds disclosed herein and fragments thereof. Such fusion may be between two or more scaffolds of the invention and a related or unrelated scaffolds. Useful fusion partners include sequences that facilitate the intracellular localization of the polypeptide, or prolong serum half life reactivity or the coupling of the polypeptide to an immunoassay support or a vaccine carrier.

Variation in Stability of Disulfide Bonds

In general, there is certain variation in the stability of disulfide bonds in proteins. For example, disulfide bonds in secreted proteins tend to be more stable than “unwanted” disulfide bonds in cytosolic proteins. In general, disulfide bonds are resistant to reduction if they are buried and according to Wedemeyer et al. disulfide bonds are generally buried. Thus, disulfide bonds in secretory proteins are rather resistant to reduction if fully folded, and low concentrations of denaturant have to be added to induce local unfolding which will make disulfide bonds accessible.

When a protein with multiple disulfide bonds is targeted to the cytosol in its folded state and the protein remains folded during uptake, its disulfide bonds may be resistant to reduction. A prerequisite for this is that none of the disulfide bonds are accessible to reducing agent. In the cytosol, thioredoxin and glutathione serve as direct oxidants for disulfide bonds. Due to their larger molecular weight compared to DTT, access to buried disulfide bonds in folded proteins should be limited.

The accessibility of disulfide bonds in proteins can be determined in silico using crystal structures or experimentally by NMR and can be compared with a titration of the denaturation sensitivity (ie D50 is the concentration of reducing agent at which 50% of the wildtype disulfides are present and 50% are not present.

Covalent Binding to Targets

Some proteins are able to covalently bind to other proteins by the exchange of disulfide bonds, resulting in exceptional binding affinity. One useful example is minicollagen, in which a c-terminal tail sequence binds covalently to an N-terminal head sequence, leading to the formation of 6 disulfides between the two proteins. See FIG. 113.

Screening and Characterization Tools

The protein libraries and the individual protein clones that come out of the early cycles of the 234, 3x0-8, 4x0-8 and 4×6 approaches described above tend to fold heterogeneously.

To some extent, one can ignore the heterogeneity and continue to evolve the proteins by directed evolution until proteins with the desired properties are obtained, notably high affinity (typically picomolar) and high specificity, but also homogenous folding and high expression level, so that the protein can be manufactured.

Methods to Construct and Pan Phage Libraries

Types of Display

A large variety of methods has been described that allow one to identify binding molecules in a large library of variants. One method is chemical synthesis. Library members can be synthesized on beads such that each bead carries a different peptide sequence. Beads that carry ligands with a desirable specificity can be identified using labeled binding partners. Another approach is the generation of sub-libraries of peptides which allows one to identify specific binding sequences in an iterative procedure (Pinilla, C., et al. (1992) BioTechniques, 13: 901-905). More commonly used are display methods where a library of variants is expressed on the surface of a phage, protein, or cell. These methods have in common, that that DNA or RNA coding for each variant in the library is physically linked to the ligand. This enables one to detect or retrieve the ligand of interest and then determine its peptide sequence by sequencing the attached DNA or RNA. Display methods allow one skilled in the art to enrich library members with desirable binding properties from large libraries of random variants. Frequently, variants with desirable binding properties can be identified from enriched libraries by screening individual isolates from an enriched library for desirable properties. Examples of display methods are fusion to lac repressor (Cull, M., et al. (1992) Proc. Natl. Acad. Sci. USA, 89: 1865-1869), cell surface display (Wittrup, K. D. (2001) Curr Opin Biotechnol, 12: 395-9). Of particular interest are methods were random peptides or proteins are linked to phage particles. Commonly used are M13 phage (Smith, G. P., et al. (1997) Chem Rev, 97: 391-410) and T7 phage (Danner, S., et al. (2001) Proc Natl Acad Sci USA, 98: 12954-9). There are multiple methods available to display peptides or proteins on M13 phage. In many cases, the library sequence is fused to the N-terminus of peptide pIII of the M13 phage. Phage typically carry 3-5 copies of this protein and thus phage in such a library will in most cases carry between 3-5 copies of a library member. This approach is referred to as multivalent display. An alternative is phagemid display where the library is encoded on a phagemid. Phage particles can be formed by infection of cells carrying a phagemid with a helper phage. (Lowman, H. B., et al. (1991) Biochemistry, 30: 10832-10838). This process typically leads to monovalent display. In some cases, monovalent display is preferred to obtain high affinity binders. In other cases multivalent display is preferred (O'Connell, D., et al. (2002) J Mol Biol, 321: 49-56).

A variety of methods have been described to enrich sequences with desirable characteristics by phage display. One can immobilize a target of interest by binding to immunotubes, microtiter plates, magnetic beads, or other surfaces. Subsequently, a phage library is contacted with the immobilized target, phage that lack a binding ligand are washed away, and phage carrying a target specific ligand can be eluted by a variety of conditions. Elution can be performed by low pH, high pH, urea or other conditions that tend to break protein-protein contacts. Bound phage can also be eluted by adding E. coli cells such that eluting phage can directly infect the added E. coli host. An interesting protocol is the elution with protease which can degrade the phage-bound ligand or the immobilized target. Proteases can also be utilized as tools to enrich protease resistant phage-bound ligands. For instance, one can incubate a library of phage-bound ligands with one or more (human or mouse) proteases prior to panning on the target of interest. This process degrades and removes protease-labile ligands from the library (Kristensen, P., et al. (1998) Fold Des, 3: 321-8). Phage display libraries of ligands can also be enriched for binding to complex biological samples. Examples are the panning on immobilized cell membrane fractions (Tur, M. K., et al. (2003) Int J Mol Med, 11: 523-7), or entire cells (Rasmussen, U. B., et al. (2002) Cancer Gene Ther, 9: 606-12; Kelly, K. A., et al. (2003) Neoplasia, 5: 437-44). In some cases one has to optimize the panning conditions to improve the enrichment of cell specific binders from phage libraries (Watters, J. M., et al. (1997) Immunotechnology, 3: 21-9). Phage panning can also be performed in live patients or animals. This approach is of particular interest for the identification of ligands that bind to vascular targets (Arap, W., et al. (2002) Nat Med, 8: 121-7).

Cloning Methods to Construct Libraries

The literature describes a large variety of methods that allow one skilled in the art to generate libraries of DNA sequences that encode libraries of peptide ligands. Random mixtures of nucleotides can be utilized to synthesize oligonucleotides that contain one or multiple random positions. This process allows one to control the number of random positions as well as the degree of randomization. In addition, one can obtain random or semi-random DNA sequences by partial digestion of DNA from biological samples. Random oligonucleotides can be used to construct libraries of plasmids or phage that are randomized in pre-defined locations. This can be done by PCR fusion as described in (de Kruif, J., et al. (1995) J Mol Biol, 248: 97-105). Other protocols are based on DNA ligation (Felici, F., et al. (1991) J Mol Biol, 222: 301-10; Kay, B. K., et al. (1993) Gene, 128: 59-65). Another commonly used approach is Kunkel mutagenesis where a mutagenized strand of a plasmid or phagemid is synthesized using single stranded cyclic DNA as template. See, Sidhu, S. S., et al. (2000) Methods Enzymol, 328: 333-63; Kunkel, T. A., et al. (1987) Methods Enzymol, 154: 367-82.

Kunkel mutagenesis uses templates containing randomly incorporated uracil bases which can be obtained from E. coli strains like CJ236. The uracil-containing template strand is preferentially degraded upon transformation into E. coli while the in vitro synthesized mutagenized strand is retained. As a result most transformed cells carry the mutagenized version of the phagemid or phage. A valuable approach to increase diversity in a library is to combine multiple sub-libraries. These sub-libraries can be generated by any of the methods described above and they can be based on the same or on different scaffolds.

A useful method to generate large phage libraries of short peptides has been recently described (Scholle, M. D., et al. (2005) Comb Chem High Throughput Screen, 8: 545-51). This method is related to the Kunkel approach but it does not require the generation of single stranded template DNA that contains random uracil bases. Instead, the method starts with a template phage that carries one or more mutations close to the area to be mutagenized and said mutation renders the phage non-infective. The method uses a mutagenic oligonucleotide that carries randomized codons in some positions and that correct the phage-inactivating mutation in the template. As a result, only mutagenized phage particles are infective after transformation and very few parent phage are contained in such libraries. This method can be further modified in several ways. For instance, one can utilize multiple mutagenic oligonucleotides to simultaneously mutagenize multiple discontiguous regions of a phage. We have taken this approach one step further by applying it to whole microproteins of >25, 30, 35, 40, 45, 50, 55 and 60 amino acids, instead of short peptides of <10, 15 or 20 amino acids, which poses an additional challenge. This approach now yields libraries of more than 10e10 transformants (up to 10e11) with a single transformation, so that a single library with a diversity of 10e12 is expected from 10 transformations.

Methods for Re-Mutagenesis

A novel variation of the Scholle method is to design the mutagenic oligonucleotide such that an amber stop codon in the template is converted into an ochre stop codon, and an ochre into an amber in the next cycle of mutagenesis. In this case the template phage and the mutagenized library members must be cultured in different suppressor strains of E. coli, alternating an ochre suppressor with amber suppressor strains. This allows one to perform successive rounds of mutagenesis of a phage by alternating between these two types of stop codons and two suppressor strains.

Another novel variation of the Scholle approach involves the use of megaprimers with a single stranded phage DNA template. The megaprimer is a long ssDNA that was generated from the library inserts of the selected pool of phage from the previous round of panning. The goal is to capture the full diversity of library inserts from the previous pool, which was mutagenized in one or more areas, and transfer it to a new library in such a way that an additional area can be mutagenized. The megaprimer process can be repeated for multiple cycles using the same template which contains a stop-codon in the gene of interest. The megaprimer is a ssDNA (optionally generated by PCR) which contains 1) 5′ and 3′ overlap areas of at least 15 bases for complementarity to the ssDNA template, and 2) one or more previously selected library areas (1,2,3,4 or more) which were copied (optionally by PCR) from the pool of previously selected clones, and 3) a newly mutagenized library area that is to be selected in the next round of panning. The megaprimer is optionally prepared by 1) synthesizing one or more oligonucleotides encoding the newly synthesized library area and 2) by fusing this, optionally using overlap PCR, to a DNA fragment (optionally obtained by PCR) which contains any other library areas which were previously optimized. Run-off or single stranded PCR of the combined (overlap) PCR product is used to generate the single stranded megaprimer that contains all of the previously optimized areas as well as the new library for an additional area that is to be optimized in the next panning experiment. See FIG. 28. This approach is expected to allow affinity maturation of proteins using multiple rapid cycles of library creation generating 10e11 to 10e12 diversity per cycle, each followed by panning.

A variety of methods can be applied to introduce sequence diversity into (previously selected or naïve) libraries of microproteins or to mutate individual microprotein clones with the goal of enhancing their binding or other properties like manufacturing, stability or immunogenicity. In principle, all the methods that can be used to generate libraries can also be used to introduce diversity into enriched (previously selected) libraries of microproteins. In particular, one can synthesize variants with desirable binding or other properties and design partially randomized oligonucleotides based on these sequences. This process allows one to control the positions and degree of randomization. One can deduce the utility of individual mutations in a protein from sequence data of multiple variants using a variety of computer algorithms (Jonsson, J., et al. (1993) Nucleic Acids Res, 21: 733-9; Amin, N., et al. (2004) Protein Eng Des Sel, 17: 787-93). Of particular interest for the re-mutagenesis of enriched libraries is DNA shuffling (Stemmer, W. P. C. (1994) Nature, 370: 389-391), which generates recombinants of individual sequences in an enriched library. Shuffling can be performed using a variety modified PCR conditions and templates may be partially degraded to enhance recombination. An alternative is the recombination at pre-defined positions using restriction enzyme-based cloning. Of particular interest are methods utilizing type IIS restriction enzymes that cleave DNA outside of their sequence recognition site (Collins, J., et al. (2001) J Biotechnol, 74: 317-38. Restriction enzymes that generate non-palindromic overhangs can be utilized to cleave plasmids or other DNA encoding variant mixtures in multiple locations and complete plasmids can be re-assembled by ligation (Berger, S. L., et al. (1993) Anal Biochem, 214: 571-9). Another method to introduce diversity is PCR-mutagenesis where DNA sequences encoding library members are subjected to PCR under mutagenic conditions. PCR conditions have been described that lead to mutations at relatively high mutation frequencies (Leung, D., et al. (1989) Technique, 1: 11-15). In addition, a polymerase with reduced fidelity can be employed (Vanhercke, T., et al. (2005) Anal Biochem, 339: 9-14). A method of particular interest is based on mutator strains (Irving, R. A., et al. (1996) Immunotechnology, 2: 127-43; Coia, G., et al. (1997) Gene, 201: 203-9). These are strains that carry defects in one or more DNA repair genes. Plasmids or phage or other DNA in these strains accumulate mutations during normal replication. One can propagate individual clones or enriched populations in mutator strains to introduce genetic diversity. Many of the methods described above can be utilized in an iterative process. One can apply multiple rounds of mutagenesis and screening or panning to entire genes, or to portions of a gene, or one can mutagenize different portions of a protein during each subsequent round (Yang, W. P., et al. (1995) J Mol Biol, 254: 392-403).

Library Treatments

Known artifacts of phage panning include 1) no-specific binding based on hydrophobicity, and 2) multivalent binding to the target, either due to a) the pentavalency of the pIII phage protein, or b) due to the formation of disulfides between different microproteins, resulting in multimers, or c) due to high density coating of the target on a solid support and 3) context-dependent target binding, in which the context of the target or the context of the microproteins becomes critical to the binding or inhibition activity. Different treatment steps can be taken to minimize the magnitude of these problems. Ideally such treatments are applied to the whole library (Library Treatments), but some useful treatments that remove bad clones can only be applied to pools of soluble proteins or only to individual soluble proteins.

Libraries of microproteins are likely to contain have that contain free thiols, which can complicate directed evolution by cross-linking to other proteins. One approach is to remove the worst clones from the library by passing it over a free-thiol column, thus removing all clones that have one or more free sulfhydryls. Clones with free SH groups can also be reacted with biotin-SH reagents, enabling efficient removal of clones with reactive SH groups using Streptavidin columns. Another approach is to not remove the free thiols, but to inactivate them by capping them with sulfhydryl-reactive chemicals such as iodoacetic acid. Of particular interest are bulky or hydrophilic sulfhydryl reagents that reduce the non-specific target binding or modified variants.

Examples of context dependence are all of the constant sequences, including pIII protein, linkers, peptide tags, biotin-streptavidin, Fc and other fusion proteins that contribute to the interaction. The typical approach for avoiding context-dependence involves switching the context as frequently as practical in order to avoid buildup. This may involve alternating between different display systems (ie M13 versus T7, or M13 versus Yeast), alternating the tags and linkers that are used, alternating the (solid) support used for immobilization (ie immobilization chemistry) and alternating the target proteins itself (different vendors, different fusion versions).

Library Treatments can also be used to select for proteins with preferred qualities. One option is the treatment of libraries with proteases in order to remove unstable variants from the library. The proteases used are typically those that would be encountered in the application. For pulmonary delivery, one would use lung proteases, for example obtained by a pulmonary lavage. Similarly, one would obtain mixtures of proteases from serum, saliva, stomach, intestine, skin, nose, etc. However, it is also possible to use mixtures of single purified proteases. An extensive list of proteases is shown in Appendix E. The phage themselves are exceptionally resistant to most proteases and other harsh treatments.

For example, it is possible to select the library for the most stable structures, ie those with the strongest disulfide bonds, by exposing it to increasing concentrations of reducing agents (ie DTT or betamercaptoethanol), thus eliminating the least stable structures first. One would typically use reducing agent (ie DTT, BME, other) concentrations from 2.5 mM, to 5 mM, 10 mM, 20 mM, 30 mM, 40 mM, 50 mM, 60 mM, 70 mM, 80 mM, 90 mM or even 100 mM, depending on the desired stability.

It is also possible to select for clones that can be efficiently refolded in vitro, by reducing the entire display library with a high level of reducing agent, followed by gradually re-oxidizing the protein library to reform the disulfides, followed by the removal of clones with free SH groups, as described above. This process can be applied once or multiple times to eliminate clones that have low refolding efficiency in vitro.

One approach is to apply a genetic selection for protein expression level, folding and solubility as described by A. C. Fisher et al. (2006) Genetic selection for protein solubility enabled by the folding quality control feature of the twin-arginine translocation pathway. Protein Science (online). After panning of display libraries (optional), one would like to avoid screening thousands of clones at the protein level for target binding, expression level and folding. An alternative is to clone the whole pool of selected inserts into a betalactamase fusion vector, which, when plated on betalactam, the authors demonstrated to be selective for well-expressed, fully disulfide bonded and soluble proteins.

Following M13 Phage display of protein libraries and panning on targets for one or more cycles, there are a variety of ways to proceed:

Screening of individual phase clones by Phase ELISA. This measures the number of phage particles (using anti-M13 antibodies) that bind to an immobilized target

Transfer from M13 into T7 Phage display libraries. Any single library format tends to favor clones that can form high-avidity contacts with the target. This is the reason that screening of soluble proteins is important, although this is a tedious solution. The multivalency achieved in T7 phage display is likely very different from that achieved in M13 display, and cycling between T7 and M13 may be an excellent approach to reducing the occurrence of false positives based on valency.

Filter lift. Filter lifts can be made of bacterial colonies grown at high density on large agar plates (10e2-10e5). Small amounts of some proteins are secreted into the media and end up bound to the filter membrane (nitrocellulose or nylon). The filters are then blocked in non-fat milk, 1% Casein hydrolysate or a 1% BSA solution and incubated with the target protein that has been labeled with a fluorescent dye or an indicator enzyme (directly or indirectly via antibodies or via biotin-streptavidin). The location of the colony is determined by overlaying the filter on the back of the plate and all of the positive colonies are selected and used for additional characterization. The advantage of filter lifts is that it can be made to be affinity-selective by reading the signal after washing for different periods of time. The signal of high affinity clones ‘fades’ slowly, whereas the signal of low affinity clones fades rapidly. Such affinity characterization typically requires a 3-point assay with a well-based assay and may provide better clone-to-clone comparability than well-based assays. Gridding of colonies into an array is useful since it minimizes differences due to colony size or location.

Pharmaceutical Composition

The present invention also provides pharmaceutical compositions comprising the subject cysteine-containing proteins. They can be administered orally, intranasally, parenterally or by inhalation therapy, and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition, the pharmaceutical compositions can also contain other pharmaceutically active compounds or a plurality of compounds of the invention.

The cysteine-containing proteins of this invention also can be combined with various liquid phase carriers, such as sterile or aqueous solutions, pharmaceutically acceptable carriers, suspensions and emulsions. Examples of non-aqueous solvents include propyl ethylene glycol, polyethylene glycol and vegetable oils.

More particularly, the pharmaceutical compositions the present may be administered for therapy by any suitable route including oral, rectal, nasal, topical (including transdermal, aerosol, buccal and sublingual), vaginal, parental (including subcutaneous, intramuscular, intravenous and intradermal) and pulmonary. It will also be appreciated that the preferred route will vary with the condition and age of the recipient, and the disease being treated.

Product Formats

A wide variety of product formats (e.g., see FIG. 159) is contemplated for use in a diversity of applications including reagents, diagnostics, prophylactics, ex vivo therapeutics and specialized formats for different drug delivery approaches for in vivo therapeutics, such as intravenous, subcutaneous, intrathecal, intraocular, transcleral, intraperitoneal, transdermal, oral, buccal, intestinal, vaginal, nasal, pulmonary and other forms of drug administration.

Such product formats include domain monomers and domain multimers (products with 2,3,4,5,6,7,8,9,10,15,20,30,40,50 or even 100 domains in a single or multiple protein chains. The domains may not contain only unique sequence or structural motifs, or it may contain duplicated sequence or structure motifs, or more highly repetitive sequence or structure motifs (repeat proteins). Each domains may have a single continuous or discontinuous (spatially or sequence-defined) binding site for 1,2,3,4,5,6,7,8,9 or 10 different targets. The targets can be a therapeutic, diagnostic (in vivo, in vitro), reagent or materials target, and may be (a combination of) protein, carbohydrate, lipid, metal or any other biological or non-biological material. Domain monomers and multimers may have multiple binding sites for the same target, optionally resulting in avidity. Domain multimers may also have 1,2,3,4,5,6,7,8 or more binding sites for different targets, resulting in multispecificity. Domain multimers optionally contain peptide linkers ranging in length from 1,2,3,4,5,6,7,8,9,10,12,14,16,18,20,25,30AA. A variety of elements can be fused to these domains, such as linear or cyclic peptides containing tags (e.g. for detection or purification with antibodies or Ni-NTA).

Halflife extension formats: A preferred approach is to use fuse a peptide (linear, mono-cyclic or dicyclic, meaning it contains 0,1 or 2 disulfides) or a protein domain that provides binding to serum albumin, immunoglobulins (ie IgG), erythrocytes, or other blood molecules or serum-accessible molecules in order to extend the serum excretion halflife of the product to the desired secretion halflife duration, which may range from 1,2,4,8, or 16 hours to 1,2,3,4,5, or 6 days to 1 week, 2 weeks, 3 weeks or 1,2 3 months. An alternative approach is to design a domain such that it binds to the pharmaceutical target as well as to a halflife extension target, such as serum albumin, using different binding sites which may or may not be partially overlapping. A desirable approach is to create scaffolds that are randomized in one area and selected to bind to the halflife target (ie HSA) and these constructs are then used to randomize additional areas that are designed to bind to one or more pharmaceutical targets, resulting in a domain that bind both the halflife target as well as the pharmaceutical target. Domains that provide halflife extension by binding to serum-proteins or serum-exposed proteins can also be fused to non-microproteins, such as, for example, human cytokines, growth factors and chemokines. An optional application is to extend the halflife of such human proteins or to target the human protein to specific tissues. The affinity preferred for such an interaction may be less than (or more than) 10 uM, 1 uM, 100 nM, 10 nM, 1 nM, 0.1 nM. Another option is to fuse long, unstructured, flexible glycine-rich sequences to the domain(s) in order to extend their Stokes' hydrodynamic radius and thereby prolong their serum secretion halflife. Another option is to link domains covalently to other domains not via a peptide bond, but by disulfide bonds or other chemical linkages. Another option is to chemically conjugate small molecules (including pharmaceutically active pharmacophores), radiolabels (ie chelates) and PEG or PEG-like molecules or carbohydrates to the protein.

Alternative delivery formats: The properties of average microproteins are exceptionally well suited for most alternative (non-injectable) delivery formats (size, protease stability, solubility, hydrophilicity), and engineering would be used to further improve their potential for a specific preferred delivery format. Werle, M. et al. (2006) J. Drug Targeting 14:137-146 show that three different microproteins are highly resistant to proteases such as elastase, pepsin, chymotrypsin as well as to plasma proteases (serum) and intestinal membrane proteases (2/3). They also show that the apparent mobility coefficient (Papp) of two microproteins was 3-fold higher than expected from a standard curve created for a variety of peptides and small proteins. For transport across tissue barriers, such as nasal, transdermal, oral, buccal, intestinal or transcleral transport, the efficiency and bioavailability is primarily determined by the size of the protein. A variety of excipients have been reported to improve transport of protein pharmaceuticals up to about 10-fold, such as alkylsaccharides (Maggio, E. (2006) Drug Delivery Reports; Maggio, E. (2006) Expert Opinion in Drug Delivery 3: 1-11. Some of these transport enhancers are either GRAS or are used as food additives so their use in pharmaceuticals may not require a lengthy FDA approval process. Some of these enhancer are amphipathic/amphiphilic and able to form micelles because they have a hydrophilic part (ie carbohydrate) and a hydrophobic part (ie alkyl chain). It may be feasible to mimic this using hydrophilic and hydrophobic protein sequences that are genetically fused to microproteins and non-microprotein peptides or proteins. For example, the hydrophilic sequence could be rich in glycine (non-ionic), glutamate and aspartate (negatively charged), or lysine and arginine (positively charged), and the hydrophobic sequence could be rich in tryptophan. Proteins with a protruding hydrophobic tail (ie 5-20 tryptophan residues) may be used to obtain an extended halflife because of the insertion of the poly-tryptophan into cellular membranes, similar to hydrophobic drugs which achieve a long halflife by membrane insertion. The protein itself remains unaltered so it's binding specificity is not expected to be reduced, only it's (micro-)biodistribution is altered. An alternative approach is to conjugate to the microprotein peptides or small molecules that are known to bind and be internalized by drug transporters such as PepT1, PepT2, HPT1, ABC transporters). References are Lee, VHL (2001) Mucosal drug delivery. J Natl Cancer Inst Monogr 29:41-44; and Kunta J R and Sinko, P J (2004) Intestinal drug transporters: in vivo function and clinical importance. Current Drug Metabolism 5:109-124; Nielsen, CU and Brodin, B (2003) Di-/Tri-peptide transporters as drug delivery targets: Regulation of transport under physiological and patho-physiological conditions. Current Drug Targets 4:373-388; Blanchette, J. et al. (2004) Principles of transmucosal delivery of therapeutic agents, Biomedicine & Pharmacotherapy 58:142-152. Dietrich, C G et al. (2005); ABC of oral bioavailability: transporters as gatekeepers in the gut. Gut 52:1788-1795; Yang C Y et al. (1999) Intestinal Peptide transport systems and oral drug availability. Pharmaceutical Research 16: 1331-1343.

Microproteins are ideally suited for topical delivery because no halflife extension is required. Microproteins can be delivered via depot formulations in order to obtain continuous delivery with a single administration.

Depot formulations (such as implants, nanospheres, microspheres, and injectable solutions such as gels) can do not require that the drug (in soluble form) has an extended halflife, although some halflife extension may still be beneficial.

Polymerization of microprotein domains and polypeptide spacers of various amino acid compositions into long polymers which are viscous is expected to yield a depot from which soluble drug is slowly released. These polymers can be fused to the microprotein or they can be separate proteins. The viscous liquid would be injected subcutaneously or submuscularly. Instead of using protein polymers, one can also mix the protein with a variety of other biodegradable matrices, such as polyanhydrides or polyesters or PLG (poly(D,L-lactide-co-glycolide)) or SAIB (sucrose acetate isobutyrate) or poly-ethylene glycol (PEG) and other hydrogels, lipid foams, collagens and hyaluronic acids. The small size, high protease, mechanical and thermal resistance and high hydrophilicity make microproteins suites for challenging formulations that most other proteins cannot achieve. Because of their small size, microproteins are well suited for iontophoresis, powder gun delivery, acoustic delivery, and delivery by electroporation (Cleland, J L et al. (2001)

Emerging protein delivery methods. Current Opinion in Biotechnology 12:212-219).

Oral delivery of fusion proteins: A different approach to oral transport involves fusion of the microprotein drug to existing bacterial toxins such as Pseudomonas Exotoxin (PE38, PE40), which are capable of traversing the cell membrane and delivering the drug into the cytoplasm of the cell. This approach has been demonstrated to work for delivery of protein drugs inside cells (ie tumor cells) as well as for efficient oral delivery, meaning transfer from the intestinal lumen into the bloodstream (Mrsny, R J et al., (2002) Bacterial toxins as tools for mucosal vaccination. Drug Discovery Today 4:247-258).

Another approach to oral (and pulmonary) delivery would fuse microproteins to Fc-receptors and use the neonatal Fc receptor-mediated uptake from the intestine and transfer to the blood by transcytosis (Low, S C et al. (2005)

Oral and pulmonary delivery of FSH-Fc fusion proteins via neonatal Fc receptor-mediated transcytosis. Human Reproduction (in press).

Intracellular delivery of microproteins: Rothbard et al. have demonstrated that natural arginine-rich peptides such as HIV-tat are able to be transported across the cell membrane and that synthetic arg-rich peptides also do this. One approach to mimic this is to append an arg-rich peptide to the N- or C-terminus of the microprotein and the second approach is to increase the arginine content of the microprotein during the design of the library and to favor clones with high arg content during screening. The arginine content can be increased up to about 3%, preferably even 5%, often even 7.5%, sometimes-10% but ideally even 15, 20, 25, 30 or 35%.

Multimeric Formats: Microproteins can be multimerized for a variety of reasons including increased avidity and increased halflife. We have focused on formats where the domains are separated by a long hydrophilic spacer that is rich in glycine, but one can polymerize domains without spacers or with naturally occurring spacers.

The long glycine-rich sequence has a large hydrodynamic radius and thus mimics halflife extension by PEGylation. Each glycine-rich sequence spacer can be 20, 25, 30, 35, 40, 50, 60, 70, 80, 100, 120, 140, 160, 180, 200, 240, 280, 320 amino acids long or even longer. For homo-multimeric targets and cell-surface targets, but even for monomeric targets, it is useful to multimerize the microprotein binding site, with glycine-rich spacers located between the binding sites and (optionally) also at the N- and C-terminus. In such proteins the overall length of the glycine polymer in a protein may reach 100, 150, 200, 250, 300, 350, or even 400 amino acids. Such proteins can contain multiple different binding sites, each binding to a different site on the same target (same copy or different copies). In this way it is possible, for example, to create a protein with very long halflife which is partially due to its length and radius and partially due to the presence of (microprotein) binding sites for serum albumin or immunoglobulins or other serum-exposed proteins.

Antibodies also utilize both size and receptor binding to obtain their long halflife and both mechanisms are likely required for maximal halflife. There are a variety of methods and compositions to achieve such a polymer of binding and non-binding elements: 1) Multiple copies of the binding motif combined in a single protein chain (genetic fusion); copies can be same or different; 2) Single (or multiple) copies of a binding site are expressed as separate proteins and multimerized N-to-C-terminus by chemical coupling. Various chemical coupling methods can be used (see list of coupling agents at www.pierce.com); copies can be same or different; 3) Multiple copies of a binding site in a single protein chain, but separated by non-binding linkers; 4) The binding site and non-binding linker are each expressed as separate proteins and multimerized by chemical coupling. Various chemical coupling methods can be used (add Pierce list of coupling agents); copies can be same or different; 5) Each protein contains one binding site and one non-binding linker and these proteins are multimerized by chemical coupling. Various chemical coupling methods can be used (see wvw.pierce.com); copies can be same or different; 6) Each protein contains a binding site and, optionally, a non-binding linker' each protein has an ‘association peptide’ at both N- and C-terminus, which bind to each other to create directional linear multimers of the protein. Various peptide sequences can be used, such as SKVILF(E) or RARADADARARADADA and derivatives; copies can be same or different. SKVILF(E) homodimerizes in an antiparallel fashion (Bodenmuller et al (1986) EMBO J.), and RARARA (or [RA]n) which binds to DADADA (or [DA]n), which is derived from the RARADADARARADADA peptide reported by Narmoneve, D A et al., (2005) Self-assembling short oligopeptides and the promotion of angiogenesis. Biomaterials 26:4837-4846. Placing the [RA]ₙ polymer at one end and the [DA]ₙ polymer at the other end (C- or N-terminus) of a domain or domain multimer will create a linear, directional polymer via association of the N-terminus of one protein to the C-terminus of another copy of the same protein. If the polymers can be made so long, or crosslinked, such that they do not leave the subcutaneous injection site efficiently, then a depot or slow release formulation may be achieved. One approach is to design protease cleavage sites for serum proteases into the polymer, which will decay slowly.

Pharmaceutical Targets: The subject microproteins generally exhibit specific binding specificity towards a given target. In some embodiments, the subject microproteins are capable of binding to one target selected from the following non-limiting list: VEGF, VEGF-R1, VEGF-R2, VEGF-R3, Her-1, Her-2, Her-3, EGF-1, EGF-2, EGF-3, Alpha3, cMet, ICOS, CD40L, LFA-1, c-Met, ICOS, LFA-1, IL-6, B7.1, B7.2, OX40, IL-1b,. TACI, IgE, BAFF or BLys, TPO-R, CD19, CD20, CD22, CD33, CD28, IL-1-R1, TNFα, TRAIL-R1, Complement Receptor 1, FGFa, Osteopontin, Vitronectin, Ephrin A1-A5, Ephrin B1-B3, alpha-2-macroglobulin, CCL1, CCL2, CCL3, CCL4, CCL5, CCL6, CCL7, CXCL8, CXCL9, CXCL10, CXCL11, CXCL12, CCL13, CCL14, CCL15, CXCL16, CCL16, CCL17, CCL18, CCL19, CCL20, CCL21, CCL22, PDGF, TGFb, GMCSF, SCF, p40 (IL12/IL23), IL1b, IL1a, IL1ra, IL2, IL3, IL4, IL5, IL6, IL8, IL10, IL12, IL15, Fas, FasL, Flt3 ligand, 41BB, ACE, ACE-2, KGF, FGF-7, SCF, Netrin1,2, IFNa,b,g, Caspase-2,3,7,8,10, ADAM S1,S5,8,9,15,TS1,TS5; Adiponectin, ALCAM, ALK-1, APRIL, Annexin V, Angiogenin, Amphiregulin, Angiopoietin1,2,4, Bc1-2, BAK, BCAM, BDNF, bNGF, bECGF, BMP2,3,4,5,6,7,8; CRP, Cadherin-6,8,11; Cathepsin A,B,C,D,E,L,S,V,X; CD11a/LFA-1, LFA-3, GP2b3a, GH receptor, RSV F protein, IL-23 (p40, p19), IL-12, CD80, CD86, CD28, CTLA-4, α4β1, α4β7, TNF/Lymphotoxin, VEGF, IgE, CD3, CD20, IL-6, IL-6R, BLYS/BAFF, IL-2R, HER2, EGFR, CD33, CD52, Digoxin, Rho (D), Varicella, Hepatitis, CMV, Tetanus, Vaccinia, Antivenom, Botulinum, Trail-R1, Trail-R2, cMet, TNF-R family, such as LA NGF-R, CD27, CD30, CD40, CD95, Lymphotoxin a/b receptor, Wsl-1, TL1A/TNFSF15, BAFF-R/TNFRSF13C, TRAIL R2/TNFRSF10B, TRAIL R2/TNFRSF10B, Fas/TNFRSF6 CD27/TNFRSF7, DR3/TNFRSF25, HVEM/TNFRSF14, TROY/TNFRSF19, CD40 Ligand/TNFSF5, BCMA/TNFRSF17, CD30/TNFRSF8, LIGHT/TNFSF14, 4-1BB/TNFRSF9, CD40/TNFRSF5, GITR/TNFRSF18, Osteoprotegerin/TNFRSF11B, RANK/TNFRSF11A, TRAIL R3/TNFRSF10C, TRAIL/TNFSF10, TRANCE/RANK L/TNFSF11, 4-1BB Ligand/TNFSF9, TWEAK/TNFSF12, CD40 Ligand/TNFSF5, Fas Ligand/TNFSF6, RELT/TNFRSF19L, APRIL/TNFSF13, DcR3/TNFRSF6B, TNF RITNFRSF1A, TRAIL R1/TIFRSF10A, TRAIL R4/TNFRSF10D, CD30 Ligand/TNFSF8, GITR Ligand/TNFSF18.

GITR Ligand/TNFSF18, TACI/TNFRSF13B, NGF R/TNFRSF16, OX40 Ligand/TNFSF4, TRAIL R2/TNFRSF10B, TRAIL R3/TNFRSF10C, TWEAK R/TNFRSF12, BAFF/BLyS/TNFSF13, DR6/TIFRSF21, TNF-alpha/TNFSF1A, Pro-TNF-alpha/TNFSF1A, Lymphotoxin beta R/TNFRSF3, Lymphotoxin beta R (LTbR)/Fc Chimera, TNF RI/fRSF1A, TNF-beta TNFSF1B, PGRP-S, TNF RVTNFRSF1A, TNF RII/TNFRSF1B, EDA-A2, TNF-alpha/TNFSF1A, EDAR, XEDAR, TNF RIVTNFRSF1A.

The following Examples are intended to illustrate and not limit the invention by providing methods for making materials useful in the methods of the present invention and operative embodiments of the methods of the invention.

### EXAMPLES

### Example 1

### Randomization of CDP 6_{—}6_{—}12_{—}3_{—}2

The following example describes the design of a library based on the CDP 6_{—}6_{—}12_{—}3_{—}2. The TrEMBL data base of protein sequences was searched for partial sequences that matched the CDP 6_{—}6_{—}12_{—}3_{—}2. A total of 71 sequences matched the CDP. The amino acid prevalence was calculated for each position as shown in Table 5. For each non-cysteine position, we chose a randomization scheme based on the following criteria: a) avoid the introduction of stop codons, b) avoid the introduction of extra cysteine residues, c) allow a large number of the amino acids that were observed at >3% in the particular position, d) minimize the introduction of amino acids that have not been observed in any of the 71 natural sequences that match the CDP.

TABLE 5
Amino acid composition of CDP 6_6_12_3_2 and resulting library design.

| position | A | C | D | E | F | G | H | I | K | L | M | N | P | Q | R | S | T |
|---|---|---|---|---|---|---|---|---|---|---|---|---|---|---|---|---|---|
| 1 | 0 | 100 | 0 | 0 | 0 | 0 | 0 | 0 | 0 | 0 | 0 | 0 | 0 | 0 | 0 | 0 | 0 |
| 2 | 0 | 0 | 0 | 6 | 4 | 0 | 1 | 10 | 4 | 0 | 0 | 0 | 0 | 4 | 3 | 1 | 7 |
| 3 | 0 | 0 | 100 | 0 | 0 | 0 | 0 | 0 | 0 | 0 | 0 | 0 | 0 | 0 | 0 | 0 | 0 |
| 4 | 45 | 0 | 6 | 6 | 0 | 6 | 1 | 3 | 1 | 7 | 3 | 1 | 0 | 0 | 6 | 7 | 7 |
| 5 | 31 | 0 | 0 | 0 | 1 | 0 | 0 | 11 | 0 | 4 | 0 | 0 | 0 | 0 | 0 | 4 | 4 |
| 6 | 4 | 0 | 6 | 1 | 0 | 0 | 0 | 3 | 4 | 0 | 11 | 18 | 8 | 0 | 0 | 7 | 27 |
| 7 | 1 | 0 | 59 | 4 | 1 | 7 | 0 | 0 | 1 | 1 | 0 | 15 | 0 | 1 | 1 | 1 | 0 |
| 8 | 0 | 100 | 0 | 0 | 0 | 0 | 0 | 0 | 0 | 0 | 0 | 0 | 0 | 0 | 0 | 0 | 0 |
| 9 | 46 | 0 | 6 | 6 | 0 | 13 | 0 | 0 | 0 | 0 | 0 | 3 | 4 | 7 | 6 | 6 | 1 |
| 10 | 0 | 0 | 4 | 3 | 0 | 0 | 1 | 1 | 1 | 4 | 0 | 54 | 0 | 8 | 8 | 3 | 7 |
| 11 | 0 | 0 | 52 | 0 | 11 | 0 | 1 | 3 | 0 | 6 | 1 | 6 | 0 | 0 | 6 | 0 | 3 |
| 12 | 10 | 0 | 0 | 0 | 0 | 0 | 0 | 23 | 8 | 17 | 6 | 0 | 3 | 1 | 13 | 0 | 10 |
| 13 | 3 | 0 | 6 | 1 | 0 | 1 | 1 | 0 | 3 | 0 | 0 | 4 | 6 | 3 | 1 | 65 | 6 |
| 14 | 1 | 0 | 0 | 0 | 4 | 0 | 0 | 54 | 0 | 20 | 1 | 0 | 0 | 0 | 4 | 3 | 3 |
| 15 | 0 | 100 | 0 | 0 | 0 | 0 | 0 | 0 | 0 | 0 | 0 | 0 | 0 | 0 | 0 | 0 | 0 |
| 16 | 0 | 0 | 1 | 1 | 7 | 6 | 0 | 0 | 3 | 6 | 1 | 30 | 0 | 21 | 1 | 4 | 17 |
| 17 | 17 | 0 | 10 | 3 | 0 | 4 | 8 | 0 | 1 | 0 | 3 | 18 | 0 | 0 | 6 | 11 | 11 |
| 18 | 3 | 0 | 0 | 4 | 1 | 0 | 0 | 14 | 6 | 0 | 0 | 1 | 17 | 7 | 1 | 4 | 0 |
| 19 | 11 | 0 | 3 | 1 | 4 | 49 | 0 | 0 | 4 | 0 | 1 | 1 | 7 | 0 | 3 | 3 | 10 |
| 20 | 0 | 0 | 1 | 0 | 8 | 0 | 0 | 1 | 0 | 10 | 44 | 0 | 0 | 0 | 0 | 0 | 0 |
| 21 | 1 | 0 | 0 | 7 | 3 | 0 | 0 | 0 | 10 | 0 | 0 | 0 | 0 | 62 | 0 | 11 | 1 |
| 22 | 3 | 0 | 32 | 11 | 1 | 0 | 0 | 0 | 1 | 0 | 0 | 1 | 14 | 3 | 10 | 6 | 15 |
| 23 | 6 | 0 | 0 | 0 | 54 | 0 | 0 | 4 | 0 | 7 | 6 | 0 | 0 | 0 | 0 | 1 | 6 |
| 24 | 0 | 0 | 0 | 0 | 3 | 0 | 0 | 6 | 0 | 11 | 27 | 0 | 0 | 0 | 0 | 0 | 0 |
| 25 | 8 | 0 | 0 | 3 | 0 | 0 | 1 | 3 | 8 | 1 | 3 | 51 | 0 | 7 | 10 | 4 | 0 |
| 26 | 3 | 0 | 0 | 6 | 0 | 6 | 0 | 0 | 6 | 14 | 0 | 4 | 0 | 23 | 4 | 17 | 18 |
| 27 | 0 | 0 | 3 | 0 | 1 | 0 | 3 | 3 | 4 | 3 | 0 | 21 | 0 | 4 | 18 | 0 | 0 |
| 28 | 0 | 100 | 0 | 0 | 0 | 0 | 0 | 0 | 0 | 0 | 0 | 0 | 0 | 0 | 0 | 0 | 0 |
| 29 | 14 | 0 | 0 | 1 | 0 | 0 | 0 | 0 | 4 | 0 | 0 | 0 | 14 | 49 | 13 | 3 | 1 |
| 30 | 1 | 0 | 0 | 1 | 1 | 0 | 0 | 0 | 42 | 11 | 0 | 0 | 0 | 1 | 41 | 0 | 0 |
| 31 | 3 | 0 | 0 | 0 | 0 | 0 | 0 | 0 | 0 | 0 | 0 | 0 | 0 | 0 | 0 | 6 | 92 |
| 32 | 0 | 100 | 0 | 0 | 0 | 0 | 0 | 0 | 0 | 0 | 0 | 0 | 0 | 0 | 0 | 0 | 0 |
| 33 | 6 | 0 | 7 | 3 | 0 | 41 | 0 | 0 | 10 | 0 | 0 | 20 | 0 | 4 | 0 | 10 | 0 |
| 34 | 0 | 0 | 0 | 0 | 20 | 0 | 4 | 7 | 4 | 6 | 0 | 0 | 0 | 0 | 54 | 1 | 0 |
| 35 | 0 | 100 | 0 | 0 | 0 | 0 | 0 | 0 | 0 | 0 | 0 | 0 | 0 | 0 | 0 | 0 | 0 |

| position | V | W | library 1 | nucleotide 1 | nucleotide 2 | nucleotide 3 |
|---|---|---|---|---|---|---|
| 1 | 0 | 0 | C | T | G | T |
| 2 | 56 | 0 | VAEMTK | AG | TCA | G |
| 3 | 0 | 0 | F | T | T | C |
| 4 | 1 | 0 | VAITLPFS | TCAG | TC | T |
| 5 | 41 | 0 | VAEMTK | AG | TCA | G |
| 6 | 10 | 0 | LPHQRIMTNKSVADEG | CAG | TCAG | CG |
| 7 | 1 | 0 | DN | AG | A | C |
| 8 | 0 | 0 | C | T | G | C |
| 9 | 0 | 3 | PHQRADEG | CG | CAG | TA |
| 10 | 3 | 1 | NSKRHQ | CA | AG | CG |
| 11 | 3 | 1 | DVFY | TG | TA | T |
| 12 | 10 | 0 | LPHQRIMTNKSR | CA | TCAG | TCAG |
| 13 | 0 | 0 | SNT | A | CAG | T |
| 14 | 4 | 0 | FILV | TCAG | T | T |
| 15 | 0 | 0 | C | T | G | C |
| 16 | 0 | 0 | PHQRTNKS | CA | TAG | TCAG |
| 17 | 7 | 0 | PHQRTNKSADEG | CAG | CAG | TCAG |
| 18 | 41 | 0 | LPQVAE | CG | TCA | A |
| 19 | 1 | 0 | TSRAG | AG | CG | TCAG |
| 20 | 0 | 4 | FYLH | TC | TA | T |
| 21 | 3 | 0 | QKE | CAG | A | G |
| 22 | 1 | 0 | TNKSRADEG | AG | CAG | CG |
| 23 | 17 | 0 | FLVM | TCAG | T | G |
| 24 | 54 | 0 | VLIM | CAG | T | CG |
| 25 | 0 | 0 | NSKRHQ | CA | AG | TA |
| 26 | 0 | 0 | LPHQRIMTNKSVADEG | CAG | TCAG | CG |
| 27 | 0 | 0 | YHN | TCA | A | C |
| 28 | 0 | 0 | C | T | G | T |
| 29 | 0 | 0 | PQRAEG | CG | CAG | G |
| 30 | 0 | 0 | KR | A | AG | G |
| 31 | 0 | 0 | TS | A | A | C |
| 32 | 0 | 0 | C | T | G | T |
| 33 | 0 | 0 | NKSRDEG | AG | AG | CG |
| 34 | 0 | 0 | FYLHIN | TCA | TA | C |
| 35 | 0 | 0 | C | T | G | C |

The last three columns in the table indicate the codon mixture that results in the amino acids that are listed in column labeled “library 1”.

### Example 2

### Protein Expression and Folding in E. coli

The oligonucleotides are cloned into an expression plasmid vector which drives expression of the proteins in the cytoplasm of E. coli. The preferred promoter is T7 (Novagen pET vector series; Kan marker) in E. coli strain BL21 DE3. A preferred process for inserting these oligos is the modified Kunkel approach (Scholle, D., Kehoe, J W and Kay, B. K. (2005) Efficient construction of a large collection of phage-displayed combinatorial peptide libraries. Comb. Chem. & HTP Screening 8:545-551). A different approach is a 2-oligo PCR of the (whole or partial) vector followed by digestion of the unique restriction sites in the oligo-derived ends of the fragment, followed by ligation of the compatible, non-palindromic overhangs (efficient intra-fragment ligation). A third approach is assembly of the insert from 2 or 4 oligos by overlap PCR, digestion of the restriction enzyme sites at the ends of the assembled insert, followed by ligation into the digested vector. The ligated DNA is transformed into competent E. coli cells and after plating on LB-Kan plates and overnight growth individual colonies are picked and inoculated into 96-well plates with 2xYT media and the cultures are grown in a shaker at 37 C overnight.

The plates are heated to 80 C for 20 min and centrifuged at 6000 g to pellet the aggregated E. coli proteins.

### Example 3

### Design Steps for Antifreeze Protein

Objective: Design a library for an antifreeze repeat protein

Strategy: The starting sequence for library design is derived from an antifreeze protein from Tenebrio molitor (Genbank accession number AF160494). This protein is known to express well in Escherichia coli. Both crystal and

NMR structures are available. The protein is built from repeating units that form a cylindrical shape. The core of the structure lacks hydrophobic amino acids, but contains one disulfide bond per repeat and one invariant serine and alanine residue. The first two turns form a capping motif with three disulfide bonds. It is assumed that this capping motif forms a folding nucleus. Therefore, the first two repeats are typically kept unchanged during in vitro evolution. See FIG. 127.

In order to choose the cross-over points and to find positions for glutamine residues for Scholle mutagenesis, the structural features of antifreeze protein were analyzed.

Crossoverpoints are shown in red and were chosen to preserve the beta-sheet stack found in the structure. Thus, two loops on the opposite side of the beta stack can be mutagenized per library. Loops in the end cap can be mutagenized at a later stage using a general upstream priming site located outside the antifreeze open reading frame. In order to choose codons for mutagenesis, an alignment of 215 repeat units was downloaded from the Pfam webpage describing antifreeze protein families (PF02420 in Pfam database). The text file was analyzed using the program Profile analyzer v1.0 with settings “2,8” for cysteine positions and “12” for total length of repeat. This setting excludes the N-terminal repeat units, which contain three cysteines per 1 amino acid repeat. Consequently, the program rejects 89 sequences and analyzes the remaining 126 sequences showing the conservation and occurrence of each amino acid in the antifreeze repeat. The output was pasted into an Excel spreadsheet and used as a starting point for library design.

### Example 4

### Design Steps for Three-Finger Toxin (Erabutoxin)

Objective: Design libraries using the Three Finger Toxin scaffold

Background: Three finger toxin exhibits a unique structure with a four-disulfide core and three long loops protruding from this core. These loops are known to participate in various protein-protein interactions and can be targeted by directed evolution.

Methods: The most common cysteine spacing patterns are 10-6-16-3-10-0-4, 13-6-16-1-10-0-4 and 13-5-16-1-10-0-4. The Erabutoxin sequence TRICFNHQSSQPQTTKTCSPGESSCYNKQWSDFRGTIIERGCGCPTVKPGIKLSCCESEVCNNA is chosen as a starting sequence and falls into the 13-6-16-1-10-0-4 pattern. This sequence was chosen because it can be expressed in Escherichia coli.

Two cross-over points were chosen to allow a maximal number of mutations in the loop regions.

### Example 5

### Design Steps for Plexin

Objective: Design a library utilizing the Plexin or PSI scaffold.

Advantages of this scaffold: This scaffold offers the unique advantage to introduce length variation between individual cysteine residues. A remarkable variation in length between cysteines of the PSI fold is found in nature and therefore supports this design principle. The diversity in loop length ranks among the highest in the microprotein family. FIG. 135 shows the ‘Multi-Plexins’ that can be created by gradual length increase by the addition of AA residues.

Strategy: The Pfam database lists 468 family members. The cysteine spacing between Cys5/Cys6, Cys6/Cys7 and Cys7/8 is highly variable. It is therefore difficult to choose a starting consensus sequence. The NMR structure of the PSI domain of the Met receptor has been solved and shows a pattern of 5,2,8,2,3,5,9. This protein has been expressed in Escherichia coli, albeit at rather low levels (1 mg/9 liter of cells). The database was searched for members displaying 5,2,8,2 spacing and 99 sequences were found. However, only 11% of these have the motif 5,2,8,2,3, and only three members possess 5,2,8,2,3,5,9. Therefore, this spacing pattern was ignored and the most common spacing pattern for this family was determined. A search with 5,2,7,2,5 yields 54 sequences. These patterns are aligned in an Excel spreadsheet to derive the most common codons at each position. The last spacing is the most variable, even insertions of whole protein domains are found. The most common spacing at the last position of the 54 members with 5, 2,7,2,5 is “15”. In summary, the consensus sequence for the PSI fold was derived from family members with the pattern 5,2,7,2,5,15.

Structure “1 ss1” shows the PSI domain from the Met receptor. The cross-over points were designed to keep the most conserved family motif, CGWC, intact. This allows randomization of the first half of the scaffold. A second cross-over-point was inserted at Cys7. This allows one to maximize the randomization of cysteine spacings 5,6 and 7, which show great length variation in nature. See FIG. 119.

FIG. 120: Alignment of library consensus with consensus 5,2,8,2,3,5 (only 11 members) shows 25% identity. The greatest diversity is in the last cys spacing, which is consistent with logo and comparison with other members.

### Example 6

### Design Steps for Somatomedin

Objective: Design a library utilizing the somatomedin scaffold

Strategy: The consensus EESCKGRCGEGFNRGKECQCDELCKYYQSCCPDYESVCKPK was derived from 44 sequences with identical cysteine spacing pattern.

The cross-overpoint was chosen approximately in the middle of the protein to allow mutagenesis in the two halves of the sequence. See FIG. 121.

### Example 7

### Evaluation of Microprotein Scaffold Expression

Microprotein open reading frames for antifreeze protein (AF), three-finger toxin (TF), somatomedin (SM) and plexin (PL) were cloned into a pET30-derived vector and expressed in Escherichia coli strain BL21(DE3). Overnight cultures were diluted 1:200 into 20 ml LB, and grown for 3 hrs and then induced with 2 mM IPTG, and grown for an additional 4 hrs. Cultures were spun at 5000×g for 10 minutes and resuspended in PBS. 250 μl of the samples were heated to 80 degree C. for 30 min and spun at RT for 10 min. Supernatants from the heat step (50 μl sample) were mixed with 25 μl sample buffer with 5% BME; resuspended cells (50 A1) were directly mixed with 25 A1 sample buffer with 5% BME. The samples were boiled for 10 minutes and then loaded on 16% SDS-PAGE.

Results: See FIG. 122. From left to right (16% SDS-PAGE): Partially purified proteins: Positive control, new AF scaffold, new TF scaffold, new SM scaffold, PL(short version), control, NEB broad range, then same order for whole cell preps of the same proteins.

Conclusions: Proteins TF, SM, PL are present in the supernatant at high concentration and are highly heat-resistant.

### Example 8

### Construction of Phagemid Vector pMP0003

We constructed a vector for the efficient construction of microprotein libraries. The vector background is based on pBluescript phagemid vector. We inserted an expression cassette that is driven by a lacZ promoter. The coding sequence comprises the following elements: ompA signal peptide, short stuffer sequence that is flanked SfiI and BstXI sites, linker element, hexahistidine tag, hemagglutinin (HA) tag, amber stop codon, C-terminal fragment of pIII protein of M13 phage, stop codon. The stuffer sequence is only 40 bp long. It contains dual TAA and TGA stop codons and a unique BssHII site. The construction of large phagemid libraries is frequently limited by the availability of sufficient quantities of digested purified vector fragment. The design of pMP0003 greatly facilitates the preparation step as it avoids the need to purify vector fragment by preparative agarose gel electrophoresis. A triple digest of plasmid pMP0003 with SfiI, BstXI, and BssHII releases two very short stuffer fragments 19 and 21 bp long, which can be removed by ultrafiltration using a YM-100 column (Microcon). The presence of the BssHII site in the stuffer also leads to a significant reduction in the frequency of non-recombinant clones in libraries that are based on pMP0003.

### Example 9

### Design and Construction of Library LMB0020

Libraries of random clones can be constructed based on many microprotein sequences. The process comprises several steps: 1) identify a suitable microprotein scaffold, 2) identify residues for randomization, 3) chose a randomization scheme for each randomized position, 4) design partially random oligonucleotides that encode the microprotein scaffold and that incorporate nucleotide mixtures in particular positions according to the randomization scheme, 5) assemble the microprotein fragment, 6) restriction digest and purification, 7) ligate the fragment into digested vector fragment, 7) transformation into competent cells.

Library LMB0020 is based on the sequence of the trypsin inhibitor EETI-II, which is a member of the squash family protease inhibitors (Christmann, A., et al. (1999) Protein Eng, 12: 797-806). The crystal structure of EETI-II was inspected and 10 positions were chosen for randomization. 9 positions were randomized using the random codon NHK, which allows the introduction of 16 amino acids (A, D, E, F, H, I, K, L, M, N, P, Q, S, T, V, Y). In one position the random codon VNK was used that allows 16 amino acids (A, D, E, F, H, I, K, L, M, N, P, Q, S, T, V, Y). The resulting random sequence is: GCPXXXXXCKQDSDCXXGCVCZPXGXCGSP where X represents the codon NHK and Z represents the codon VNK. This randomization scheme allows for a theoretical diversity of over 10¹² different amino acid sequences. The gene fragment encoding the randomized trypsin inhibitor was assembled by overlap extension of two oligonucleotides with the sequence:

LMB0020F = CAGGCAGCGGGCCCGTCTGGCCCGGGTTGTCCTNHKNHK
NHKNHKNHKTGTAAACAAGACTCTGACTG,
LMB0020R = TGTAAACAAGACTCTGACTGTNHKNHKGGTTGCGTTTGC
VNKCCGNHKGGTNHKTGTGGCTCTCCGGGCCAGTCTGGTGGTTCCGGTCA
CGTGACCGGAACCACCAGACTGGCCCGGAGAGCCACAMDNACCMDNCGGM
NBGCAAACGCAACCMDNMDNACAGTCAGAGTCTTGTTTACA.

The oligonucleotides LMB0020F and LMB0020R share a complementary region of 20 nucleotides. Two steps PCR amplification was performed by annealing of two complementary primers followed by filling in reaction. The product was then amplified by using scaffold primers LIBPTF and LIBPTR, which contain the restriction sites.

The resulting product was concentrated using a YM-30 filter (Microcon) and purified by preparative agarose gel electrophoresis using 1.2% agarose.

Ten pg of product were SfiI/BstXI digested for 5 h at 50° C. and quick purified on PCR column (Qiagen) yielding ca 4 μg of purified fragment. The vector pMP0003 was prepared using QIAGEN HiSpeed Maxi Kit. 150 μg of vector DNA were SfiI/BstXI/BssHII digested for 4 h at 50° C. in 3 separate Eppendorf tubes and purified on YM-100 column (Microcon). Total yield was 112.5 μg (75%) of digested vector. Various insert to vector ratios were tested in small scale experiments to maximize the number of transformants in the library. Large scale ligations were performed in 7 ligation tubes. Each tube contains 3 μg of digested vector, 0.5 μg of digested insert (1:2.5 ratio), 40 μl of ligase buffer, 20 μl of T4 DNA ligase in 400 μl of total volume. Ligation was performed overnight at 16° C. The resulting product was purified by ethanol precipitation overnight at −20° C. in 8 tubes for each library. The ligated DNA in each tube was dissolved in 30 ml of distilled water and divided on 2×15 μl, thus yielding 16 tubes for transformation per library.

Electrocompetent E. coli ER2738 were prepared using the following process: 1) Inoculate 15 ml of prewarmed superbroth medium (SB) in a 50-ml polypropylene tube with a single E. coli colony from a glycerol stock that has been freshly streaked onto an LB agar (5 mg/l tetracycline). Add tetracycline to 30 μg/ml (90 μl of 5 mg/ml tetracycline) and grow overnight at 250 rpm on a shaker at 37° C. 2)Dilute 2.5 ml of the culture into each of four 2-liter flasks with 500 ml of SB medium, add 10 ml of 20% glucose, 5 ml of 1M MgCl₂, and 500 μl of 5 mg/ml tetracycline. Shake at 250 rpm and 37° C. until absorbance at 600 nm is about 0.9 (2 h 45 min). 3) Chill the culture as well as 4 500-ml bottles on ice for 15 min. 4) Transfer the culture into 4 500-ml bottles and spin at 4000 rpm for 20 min at 4° C. 5) Pour off the super and resuspend each pellet in 25 ml of pre-chilled 10% glycerol using 25-ml pre-chilled pipettes. Combine 2 pellets in one 250-ml bottle and add 10% glycerol to yield 250 ml. Spin as before. 6) Pour off the supernatant and repeat step 5. 7) Discard the supernatant and resuspend each pellet in the remaining volume (3.5 ml). Combine all suspensions. Use 300 μl aliquot for library electroporation. Optional: To store, aliquot 320 μl in eppendorf tubes and flash freeze them using ethanol and dry ice. Cap the tubes and store them at −80° C. 8) Plate 50 μl of cell suspension on LB argar (100 mg/l carbenicillin) to test for vector phage contamination. Plate 50 μl of cell suspension on LB argar (50 mg/l kanamycin) to test for helper phage contamination.

Electroporation of the library was performed using the following steps: 1) Place the ligated DNA (usually 16) and a corresponding number of cuvettes on ice for 10 min. 2) Add freshly prepared ER2738 cells to each ligated library sample, mix by pipeting up and down once, and transfer to a cuvette. Store on ice for 1 min. Electroporate at 2.5 kV, 25 μF, and 200 ohm. Flush the cuvette immediately with 2 ml and then with 1 ml SOC medium at room temperature. Combine 3 ml of culture in 10-ml culture tube. Shake at 300 rpm for 1 hr at 37° C. 3) Combine two 3 ml samples and transfer to 50-ml polypropylene tube. Add 9 ml of pre-warmed (37° C.) SB medium, 3 μl of 100 mg/ml carbenicillin, and μl of 5 mg/ml tetracycline. For titering of transformed bacteria, dilute 2 μl of the culture in 200 μl of SB medium, and plate 10 μl and 1 μl of this 1:100 dilution on LB agar (100 mg/l carbenicillin). Incubate the plates overnight at 37° C. Calculate the total number of transformants by counting the number of colonies, multiplying by the culture volume, and dividing by the plating volume. Shake the 15-ml culture at 300 rpm and 37° C. for 1 h, add 4.5 μl 100 mg/ml carbenicillin, and shake for an additional hour at 300 rpm and 37° C. 4) Combine two 15 ml samples and add 3 ml of VCSM13 helper phage. Transfer to a 500-ml polypropylene centrifuge bottle. Add 167 ml of pre-warmed (37° C.) SB medium, 92.5 μl of 100 mg/ml carbenicillin, and 185 μl of 5 mg/ml tetracycline. Shake the 200-ml culture at 300 rpm and 37° C. for 1.5-2 h. 5) Add 280 μl of 50 mg/ml kanamycin and continue shaking at 300 rpm and 37° C. overnight. 6)

Spin at 4000 rpm for 15 min at 4° C. Transfer the supernatant to a clean 500-ml centrifuge bottle and add 50 ml of 20% PEG-8000/NaCl 2.5M. Store on ice for 30 min. 7) Spin at 9000 rpm for 15 min at 4° C. Discard the supernatant, drain liquid by inverting centrifuge bottles on a paper towel for at least 10 min, and wipe off remaining liquid from the upper part of the centrifuge bottles with a paper towel. 8) Resuspend the phage pellet in 2 ml of 1% (w/v) bovine serum albumin (BSA) in Tris buffered saline (TBS) buffer by pipetting up and down along the side of the centrifuge bottle and transfer to a 2-ml microcentrifuge tube. Resuspend further by pipetting up and down using a 1-ml pipette tip, spin at full speed in a microcentrifuge for 5 min at 4° C., and pass the supernatant through a 0.2-μm filter into a sterile 2-ml microcentrifage tube. Store the phage preparation at 4° C. Sodium azide may be added to 0.02% (w/v) for long-term storage. The resulting library size for LMB0020 was 2.4×10⁹ transformants.

### Example 10

### Panning of Library LMB0020

1) Coat wells of a Costar 96-well ELISA plate with 0.25 μg of CD22 antigen in 25 μl of PBS. Cover the plate with plate sealer. Coating can be performed overnight at 4° C. or for 1 h at 37° C. In the first round of panning coat 2 wells per library to be screened; one well is sufficient in each of the subsequent rounds. The target concentration was lowered to 0.1 ug/well during panning rounds 3 to 6.

2) After shaking out the coating solution, block the well by adding 150 μl of TBS/BSA 3% (Tris buffered saline containing 3% bovine serum albumin). Seal and incubate for 1 h at 37° C.

3) After shaking out the blocking solution, add 50 μl of freshly prepared phage library to the well (Input sample). Seal the plate and incubate for 2 h at 37° C. In the meantime, inoculate 2 ml SB medium plus 2 μl of 5 mg/ml Tetracycline with 2 μl of an ER 2738 cell preparation and allow growth at 250 rpm and 37° C. for 2.5 h. Grow 1 culture for each library that is screened and an additional culture for input titering.

4) Shake out the phage solution, add 150 μl of TBS/Tween-20 0.05% to the well and pipette 5 times vigorously up and down. Wait 5 min, shake out, and repeat this washing step. In the first round of panning, wash in this fashion 4 times, in the second round 6 times, in the third round 8 times, and so on.

5) After shaking out the final washing solution, add 50 μl of freshly prepared 10 mg/ml trypsin in TBS, seal, and incubate for 30 min at 37° C. Pipette 10 times vigorously up and down and transfer the eluate (2×50 μl in the first round, 1×50 μl in the subsequent rounds) to the prepared 2-ml E. coli culture and incubate at room temperature for 15 min.

6) Add 6 ml of pre-warmed SB medium and 1.6 μl of 100 mg/ml carbenicillin and 6 μl of 5 mg/ml Tetracycline. Transfer the culture into a 50-ml polypropylene tube. For output titering, dilute 2 μl of the sample in 200 μl SB medium and plate 100 μl and 10 μl of this sample on LB agar (100 mg/l carbenicillin) (Output sample). In parallel, proceed with the input titering by infecting 50 μl of the prepared 2-ml E. coli culture with 1 μl of a 10⁻⁸ dilution of the phage preparation, incubate for 15 min at room temperature, and plate on LB agar (100 mg/l carbenicillin).

7) Shake the 8-ml culture at 250 rpm and 37° C. for 1 h, add 2.4 μl 100 mg/ml carbenicillin, and shake for an additional hour at 250 rpm and 37° C.

8) Add 1 ml of VCSM13 helper phage and transfer to a 500-ml polypropylene centrifuge bottle. Add 91 ml of pre-warmed (37° C.) SB medium and 46 μl of 100 mg/ml carbenicillin and 92 μl of 5 mg/ml Tetracycline. Shake the 100-culture at 300 rpm and 37° C. for 1½ to 2 h.

9) Add 140 μl of 50 mg/ml kanamycin and continue shaking at 300 rpm and 37° C. overnight.

10) Spin at 4000 rpm for 15 min at 4° C. Transfer the supernatant to a clean 500-ml centrifuge bottle and add add 25 ml of 20% PEG-8000/NaCl 2.5M. Store on ice for 30 min.

11) Spin at 9000 rpm for 15 min at 4° C. Discard the supernatant, drain inverted on a paper towel for at least 10 min, and wipe off remaining liquid from the upper part of the centrifuge bottle with a paper towel.

12) Resuspend the phage pellet in 2 ml of TBS/BSA 1% buffer by pipetting up and down along the side of the centrifuge bottle and transfer to a 2-ml microcentrifuge tube. Resuspend further by pipetting up and down using a 1-ml pipette tip, spin at full speed in a microcentrifuge for 5 min at 4° C., and pass the supernatant through a 0.2-μm filter into a sterile 2-ml microcentrifuge tube.

13) Continue from step 3) for the next round or store the phage preparation at 4° C. Sodium azide may be added to 0.02% (w/v) for long-term storage. Only freshly prepared phage should be used for each round.

Table 6 shows the phage titer of input and output solutions during 6 rounds of library panning

| | Input | Output | Recovery | |
|---|---|---|---|---|
| Round | (10¹¹) | (10⁶) | (% × 10³) | Enrichment |
| 1 | 12 | 1.9 | 0.16 | — |
| 2 | 0.45 | 0.032 | 0.007 | neg |
| 3 | 4.7 | 2.14 | 0.46 | 2.87 |
| 4 | 2.5 | 0.064 | 0.032 | neg |
| 5 | 0.52 | 1.2 | 2.3 | 14.37 |
| 6 | 0.6 | 2.0 | 3.33 | 20.8 |

### Example 11

### Screening of Individual Isolates for Target Binding

ER2738 was infected with output phage and plated on LB agar (100 mg/l carbenicillin). Plates were incubated overnight at 37 C. Subsequently, individual colonies can be screened for binding to target protein as follows:

1) Add 0.75 ml SB medium containing 50 μg/ml carbenicillin to 96 well plate with deep with deep wells. Transfer individual colonies into each well using a sterile tooth pick. 2) Shake the plate containing the bacterial cultures at 300 rpm for several hours at 37° C.

2) Spot 1 μl of each culture onto LB agar (100 mg/l carbenicillin) at 6 hours after inoculation. Incubate plates overnight at 37° C.; seal plates with parafilm and store them at 4° C. These plates were used later to retrieve and sequence isolates that showed positive ELISA signals.

3) Induce cultures by adding IPTG to 1 mM (7.5 μl of 1 M IPTG stock diluted 1:10 in water) and culture them overnight at 37 C

4) Spin down induced E. coli cultures (4000 rpm; 20 min).

5) Prepare Bugbuster solution (Novagen) (1.5 ml reagent plus 13.5 ml TBS and 15 μl of Benzonase).

6) Resuspend pellet in 150 μl bugbuster. Incubate plate at room temperature for 30 minutes and spin plate at 4000 rpm for 20 minutes.

7) Transfer 50 μl per well of supernatants to microtiter plates that have been coated overnight at 4 C with 100 ng of target protein per well in PBS and blocked with 150 ul/well of TBS containing 3% BSA for one hour.

8) Incubate plate for 2 hours at 37° C.

9) Wash 10 times with tap water.

10) Dilute biotinylated rat anti-HA antibody (3F10, Roche Biosciences) in TBS/BSA 1% (1:500 dilution). Add 50 μl of diluted antibody to wells, and incubate for 1 hour at 37° C.

11) Wash 10 times with tap water.

12) Dilute Streptavidin/HRP in TBS/BSA 1% (1:2500 dilution) and add 50 ul per well, and incubate for 30 min at 37° C.

13) Prepare ABTS solution (2.94 ml of citrate buffer+60 μl ABTS+1 μl H₂O₂).

14) Wash plate 10 times with tap water.

15) Add 50 μl substrate solution to each well.

16) Incubate at RT and read O.D. at 405 nm using an ELISA plate reader after 20 min incubation at room temperature.

Output from rounds 5 of library LMB0020 as well as from two other microprotein libraries was screened as described above. The table below shows resulting binding data for plates coated with IgG as well as BSA. Several isolates show significantly higher binding signals on plates coated with IgG relative to BSA coated wells.

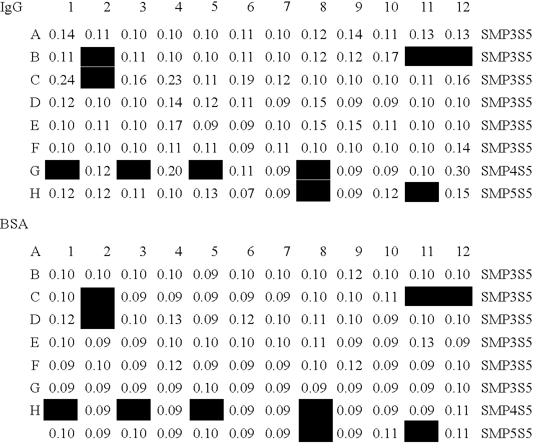

Three IgG-binding isolates were sequenced. All isolates maintained the spacing between the 6 cysteine residues of the trypsin inhibitor scaffold. All three isolates differ in their amino acid sequence, which demonstrates that the approach can yield multiple binding domains, each of which can serve as a starting point for further optimization.

LMB0020/SMP003S5.B2
GPSGPGCPILYAHCKQDSDCVTGCVCRPLGMCGSPGQSGGSGHHHHHH
LMB0020/SMP003S5.B12
GPSGPGCPSLPTPCKQDSDCDEGCVCKPNGTCGSPGQSGGSGHHHHHH
LMB0020/SMP003S5.C2
GPSGPGCPLYSPVCKQDSDCDNGCVCRPAGPCGSPGQSGGSGHHHHHH

### Example 12

### Build-Up Approach to Microprotein Design

A 1-disulfide protein (1SS) that binds to VEGF was evolved stepwise into a 2SS microprotein that is more stable to proteases and less immunogenic. FIG. 1 shows the ELISA results of two separate 2SS proteins (‘Clone 2’ and ‘Clone 7’) that were derived from a 1SS phage derived peptide (‘VEGF pept’). All three are specific for VEGF and do not show binding to other proteins such as BSA. M13 without a microprotein also does not bind to VEGF or BSA. This 2SS protein was created by moving the 1SS sequence that determined VEGF binding into a natural 2SS scaffold (alpha-conotoxin). The resulting protein is specific for VEGF and does not bind unrelated proteins, such as bovine serum albumin (BSA). Wild type phage particles (M13) do not exhibit binding to either VEGF or BSA. See FIG. 168.

### Example 13

### Library Construction by Megaprimer Mutagenesis

The Megaprimer process is a way to combine two (or more) different primers into a single large primer that is incorporated into a plasmid via homology at both of it's ends in a Kunkel-type polymerase extension reaction (except that a stopcodon-replacement can be used to make incorporation highly efficient). The Megaprimer process uses double-stranded or single stranded DNA of 60, 70, 80, 90, 100, 110 or preferably even more than 120 nucleotides or base pairs for introducing or transferring complex pools of DNA and encoded protein sequences. In our examples these pools encode microprotein libraries, but the same process can encode any DNA or protein library. The megaprimer typically comprises a pool of previously selected sequences (‘old library’) as well as a pool of newly randomized sequences (‘new library’). The Megaprimer process thus allows the blind creation of a new library from an old library—without having to sequence the old library.

Typically a PCR fragment is created from the library area (‘randomized area’) of a previously selected pool of sequences and this fragment is linked (via PCR-overlap) to a synthetic oligo encoding a newly randomized library segment (unselected), creating a dsDNA fragment containing both the new (unselected) and the old (selected) randomized areas. The same end-result can be achieved in a single PCR using primers on both sides of the ‘old library’ area, if one of the primers introduces the new library. This dsDNA PCR fragment is converted into a ssDNA Megaprimer by asymmetric or run-off PCR. The ends of this ssDNA Megaprimer are designed to have about 10-25 bases of sequence homology with the vector, ensuring insertion at the correct location.

Double stranded megaprimers are generated from two or more PCR fragments and/or synthetic oligonucleotides using overlap PCR and single-stranded DNA can be generated using denatured double-stranded PCR product and/or single-stranded DNA ‘asymmetric PCR’ (‘run-off PCR’). The asymmetric PCR amplifies the single-stranded sequence that complements the single-stranded DNA template. The megaprimer sequence can comprise a single sequence but more typically comprises a library of (for example, microprotein) sequences (as described in FIG. 143). The single-stranded template DNA (vector or phage) can be uridine-containing or it can encode for a suppressible stop codon (TAG, TAA, TGA) that is exchanged for the megaprimer sequence that does not have a stop codon. The annealed megaprimer then primes synthesis of the second strand of DNA by polymerase and ligation of the synthesized strand is used to generate covalently closed circular DNA (ccc-DNA) in the presence of a buffer, DNA polymerase, DNA ligase, and deoxynucleotide triphosphates (dNTPs). The resulting ccc-DNA is transformed into a bacterial cell line for expression of the microprotein as insoluble protein; soluble protein, or as a protein fusion.

An example of a Megaprimer result is shown in the table below. It shows amino acid sequences of a microprotein that has been mutagenized in the first 15 positions. Conserved residues that match the initial microprotein template are shaded grey. A library of microprotein sequences, including the sequences from FIG. 2 were used as the starting point for the megaprimer synthesis. Two DNA primers were used to create a PCR fragment containing the ‘old library’ area as well as a new library area: i) a primer that anneals upstream of the microprotein, and ii) a primer that contains newly randomized microprotein sequence (‘new library’) that is flanked by a microprotein-specific annealing region and a DNA template annealing region. The microprotein library input was amplified with the two primers using PCR, amplified by asymmetric PCR, and cloned into single-stranded DNA template to generate a secondary microprotein library. The resulting clones (FIG. 2 bottom) revealed microprotein sequences that were randomized in both the first and second halves of the original sequence.

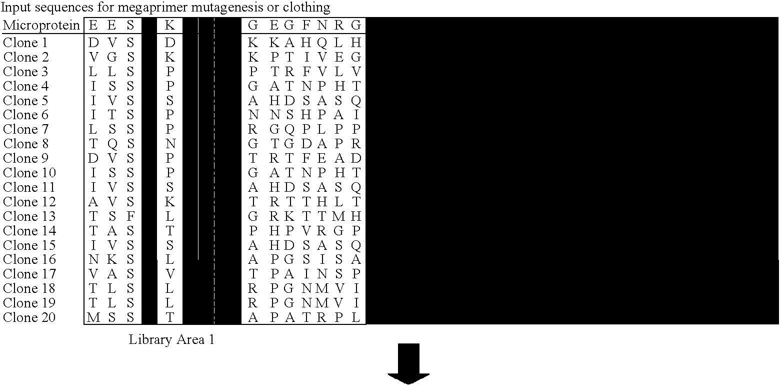

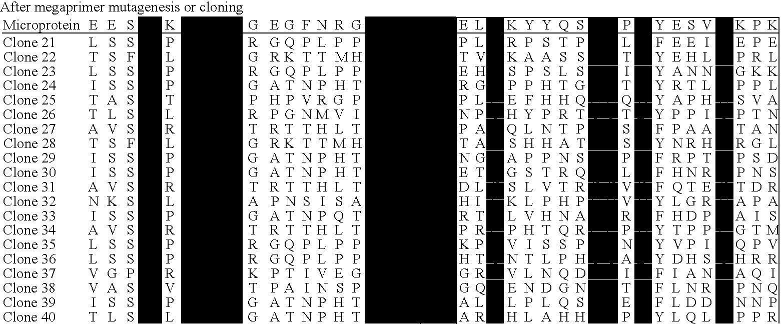

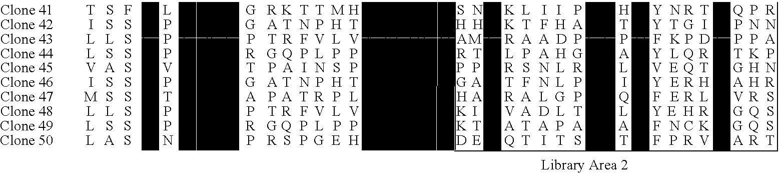

### Example 14

### Production of Microproteins

Microprotein genes were cloned into expression vector pET30 carrying the T7 promoter and transformed into E. coli strain BL21(DE3). 2 ml LB (50 mg/l kanamycin) were inoculated from frozen glycerol stocks and cultured for 4 hrs at 37 C. 200 μl of these starting cultures was added to 250 ml LB (50 mg/l kanamycin) and incubated without shaking overnight. Next morning, shaker was turned to 250 rpm and cultures were grown for an additional 1 hr. IPTG was then added to 0.5 mM final concentration and proteins were expressed for 6 hrs in a shaking incubator at 37 C. Cultures were centrifuged at 300 rpm for 15 min, resuspended in 5 ml PBS, and heated for 20 minutes at 75 C. This step leads to cell lysis and to the denaturation of most E. coli proteins. The suspension was centrifuged in an SS34 rotor at 10,000 rpm for 30 minutes. Resulting supernatants were loaded onto HiTrap columns (Pharmacia GE) charged with nickel sulfate. Proteins were eluted with imidazole as suggested by the column manufacturer. The resulting protein is >90% pure as judged by SDS PAGE under reducing conditions.

### Example 15

### Determination of Complexity of DBPs

Complexity is the cumulative disulfide span, which equals the cumulative distance between linked cysteines, measured in amino acids on the protein chain.

Complexity is a measure of the degree of crosslinking and thus of rigidity of the scaffold, a higher complexity offering higher rigidity. Because rigidity is a predictor of protease resistance, it also is a useful predictor of immunogenicity. A higher complexity predicts reduced protease degradation and lower immunogenicity.
Complexity=(Ca−Cb)+(Cc−Cd)+(Ce−Cf)

| Ca-Cb | Cc-Cd | Ce-Cf | Cg-Ch | Complexity |
|---|---|---|---|---|
| 1 2 | 3 4 | | | 2 |
| 1 3 | 2 4 | | | 4 |
| 1 4 | 2 3 | | | 4 |
| 1 6 | 2 5 | 3 4 | | 9 |
| 1 4 | 2 5 | 3 6 | | 9 |
| 1 6 | 2 4 | 3 5 | | 9 |
| 1 5 | 2 6 | 3 4 | | 9 |
| 1 5 | 2 4 | 3 6 | | 9 |
| 1 4 | 2 6 | 3 5 | | 9 |
| 1 2 | 3 4 | 5 6 | | 3 |
| 1 2 | 3 5 | 4 6 | | 5 |
| 1 2 | 3 6 | 4 5 | | 5 |
| 1 6 | 2 3 | 4 5 | | 7 |
| 1 4 | 2 3 | 5 6 | | 5 |
| 1 5 | 2 3 | 4 6 | | 7 |
| 1 3 | 2 6 | 4 5 | | 7 |
| 1 3 | 2 4 | 5 6 | | 5 |
| 1 3 | 2 5 | 4 6 | | 7 |
| 1 2 | 3 4 | 5 6 | 7 8 | 4 |

### Example 16

### Scaffolds without Repeated Motifs

Superfamilies of Toxin Families

1) uPAR/Ly6/CD59/snake toxin-receptor superfamily. Includes the families: Activin_recp; BAMBI; PLA2_inh; Toxin_{—}1; UPAR_LY6;

2) Scorpion toxin-like knottin superfamily includes the families Toxin_{—}2; Toxin_{—}17; Gamma-thionin; Defensin-2; Toxin_{—}3; Toxin_{—}5;

3)Defensin/myotoxin-like superfamily includes the families BDS_I_II; Defensin_{—}1; Defensin_beta; Toxin_{—}4;

4) Omega toxin-like superfamily includes families Toxin_{—7}; Toxin_{—}30; Toxin_{—}27; Toxin_{—}24; Toxin_{—}21; Toxin_{—}16; Toxin 12; Toxin_{—}11; Omega-toxin; Albumin_I; Toxin_{—}9;

5) Conotoxin O-superfamily consists of 3 groups of Conus peptides that belong to the same structural group. These 3 groups differ in their pharmacological properties: the w-conotoxins which inhibit calcium channels, the delta-conotoxins which slow down the inactivation rate of voltage-sensitive sodium channels and the muO-conotoxins block the voltage sensitive sodium currents.

6) Conotoxin I-superfamily includes only the Toxin 19 family.

7) Conotoxin T-superfamily includes only the Toxin 26 family.

Individual Toxin Families:

PF00087: Toxin 1

Snake Toxin. A family of venomous neurotoxins and cytotoxins. Structure is small, disulfide-rich, nearly all beta sheet. See FIG. 61.

Cxxxxx(xxxx)xxxCxxxxxxCxxxx(xxx)C(xx)xxxxxxxxCxxxC

Cxxxxx(xxxx)xxxCxxxxxxCYxkx(wf)(xx)C(xx)xxxxxxxGCxxxC

PF00451: Toxin 2

‘Scorpion toxin short’. Scorpion venoms contain a variety of peptides toxic to mammals, insects and crustaceans. Among these peptides, there is a family of short toxins (30 to 40 residues) inhibiting calcium-activated potassium channels. See FIG. 55. Topology is 1-4 2-6 3-5.

1) CxxxxxCxxxCxxxxxxxxxxCxxxxCxC

2) CxxxxxCxxxCkxxxxxxxgKCxxxKCxC

PF00537: Toxin 3

This family contains both neurotoxins and plant defensins (F. M. Assadi-Porter, et al. (2000) Arch Biochem Biophys, 376: 259-65). The mustard trypsin inhibitor, MTI-2, is plant defensin. It is a potent inhibitor of trypsin. MTI-2 is toxic for Lepidopteran insects. The scorpion toxin (a neurotoxin) binds to sodium channels and inhibits the activation mechanisms of the channels, thereby blocking neuronal transmission. See FIG. 22. Topology is 1-8 2-5 3-6 4-7.

1) C(xxx)x(xx)xxxxCxxxCxx(xx)xxCxxxCxx(x)xxxxCxxxxx(xx)xxCxC

2) C(xxx) Y(xx)xxxxCxxxCxx(xx)xxCxxxCxx(x)xxGxCxxxxx(xx)xxC(W,Y)C

PF00706: Toxin 4

Anemone neurotokins. Sea anemones produce many different neurotoxins with related structure and function. Proteins belonging to this family include the neurotoxins, of which there are several, including calitoxin and anthopleurin. The neurotoxins bind specifically to the sodium channel, thereby delaying its inactivation during signal transduction, resulting in strong stimulation of mammalian cardiac muscle contraction. Calitoxin 1 has been found in neuromuscular preparations of crustaceans, where it increases transmitter release, causing firing of the axons. Three disulphide bonds are present in this protein. This family is a member of the Defensin/myotoxin-like superfamily clan. This clan includes the following Pfam members: BDS_I_II; Defensin_{—}1; Defensin_beta; Toxin_{—}4. Sea anemones produce many different neurotoxins with related structure and function. Proteins belonging to this family include the neurotoxins, of which there are several, including calitoxin and anthopleurin. The neurotoxins bind specifically to the sodium channel, thereby delaying its inactivation during signal transduction, resulting in strong stimulation of mammalian cardiac muscle contraction. Calitoxin 1 has been found in neuromuscular preparations of crustaceans, where it increases transmitter release, causing firing of the axons. Three disulphide bonds are present in this protein. There are 25 known family members. Topology is 1-5 2-4 3-6. FIG. 87.

1) CxCxxxxxxxxxxxxxxxx(xx)xxxxC(xxx)xxxxxxCxxxxxxxxxCC

2) CxCxxxxPxxrxxxxxGxx(xx)xxxxC(xxx)xxxWxxCxxxxxxxxxCC

PF05294: Toxin 5

Scorpion short toxins. FIG. 46.

PF05453: Toxin 6

FIG. 90. This family consists of toxin-like peptides that are isolated from the venom of Buthus martensii Karsch scorpion. The precursor consists of 60 amino acid residues, with a putative signal peptide of 28 residues and an extra residue, and a mature peptide of 31 residues with an amidated C-terminal. The peptides share close homology with other scorpion K+ channel toxins and should present a common three-dimensional fold, the Cysteine-Stabilised alphabeta (CSalphabeta) motif. This family acts by blocking small conductance calcium activated potassium ion channels in their victim. Topology is 1-4 2-5 3-6. Motif is CxxCxxxCxxxxxxx(xx)C(xx)xxxxxCxC

PF05980: Toxin 7

This family consists of several short spider neurotoxin proteins including many from the Funnel-web spider (W. S. Skinner, et al. (1989) J Biol Chem, 264: 2150-55). See FIG. 64.

Topology is 1-4 2-5 3-8 6-7.

1) CxxxxxxCxxxxxxxCCxxxxxCxCxxxxxCxC

2) CxxxxxxCxxWxxxxCCxgxxYCxCxxxpxCxC

PF07365: Toxin 8

Alpha-conotoxin and precursors. This family consists of several alpha conotoxin precursor proteins from a number of Conus species. The alpha-conotoxins are small peptide neurotoxins from the venom of fish-hunting cone snails which block nicotinic acetylcholine receptors (nAChRs). FIG. 72.

PF00095: Toxin 9

This family of spider neurotoxins are thought to be calcium ion channel inhibitors.

See FIG. 63. Topology is 1-4 2-5 3-8 6-7.

1) Cxx(x)xxxxCxxxxxCCxxx(x)xCxCxxxxxCxC

2) Cxx(x) yxxxCxxgxxCCxrx(x)xCxCxxxxnCxC

PF07473: Toxin 11

This family consists of several spasmodic peptide gm9a sequences (M. B. Lirazan, et al. (2000) Biochemistry, 39: 1583-8). See FIG. 27, DBP: 1-5 2-4 3-6

Motif: CxxxCxxxxxCxxxCxC

PF07740: Toxin 12

HaTx1 is a 35 amino acid peptide toxin that was isolated from Chilean tarantula venom. It inhibits the drk1 voltage-gated K(+) channel not by blocking the pore, but by altering the energetics of gating (H. Takahashi, et al. (2000) J Mol Biol, 297: 771-80). See FIG. 50.

Topology is 1-4 2-5 3-6. Motif is CxxxxxxCxxxxx(x)CCxxxxCxxx(xxx)x(xx)xxC

PF07822: Toxin 13

The members of this family resemble neurotoxin B-IV, which is a crustacean-selective neurotoxin produced by the marine worm Cerebratulus lacteus. This highly cationic peptide is approximately 55 residues and is arranged to form two antiparallel helices connected by a well-defined loop in a hairpin structure. The branches of the hairpin are linked by four disulphide bonds. Three residues identified as being important for activity, namely Arg-17, -25 and -34, are found on the same face of the molecule, while another residue important for activity, Trp30, is on the opposite side. The protein's mode of action is not entirely understood, but it may act on voltage-gated sodium channels, possibly by binding to an as yet uncharacterized site on these proteins. Its site of interaction may also be less specific, for example it may interact with negatively charged membrane lipids. See FIG. 65.

PF07829: Toxin 14

Alpha-A conotoxin PIVA is the major paralytic toxin found in the venom produced by the piscivorous snail Conus purpurascens. This peptide acts by blocking the acetylcholine binding site of the nicotinic acetylcholine receptor (K. J. Nielsen, et al. (2002) J Biol Chem, 277: 27247-55). See FIG. 66.

Motif 1: CCxxxxxxxCxxCxCx(x)xxxxxC, Motif 2: CCgxxpxxxChpCxCx(x)xxpxxC

PF07945: Toxin 16

Janus Atracotoxin family. This family includes three peptides secreted by the spider Hadronyche versuta. These are insect-selective, excitatory neurotoxins that may function by antagonizing muscle acetylcholine receptors, or acetylcholine receptor subtypes present in other invertebrate neurons. Janus atracotoxin-Hv1c is organised into a disulphide-rich globular core (residues 3-19) and a beta-hairpin (residues 20-34). There are 4 disulphide bridges, one of which is a vicinal disulphide bridge; this is known to be unimportant in the maintenance of structure but important for insecticidal activity. There are 3 known family members. Topology is 1-6 2-7 3-4 5-8. FIG. 91.

1) CxxxxxxCxxCCxCCxxxxCxxxxxxxxxxC

2) CxgxxxpCxxCCpCCpgxxCxxxxxxgxxyC

PF08086: Toxin 17

This family consists of ergtoxin peptides which are toxins secreted by the scorpions. The ergtoxins are capable of blocking the function of K+ channels. More than 100 ergtoxins have been found from scorpion venoms and they have been classified into three subfamilies according to their primary structures (K. Frenal, et al. (2004) Proteins, 56: 367-75). There are 25 known family members. Topology is 1-4 2-6 3-7 5-8. See FIG. 60.

1) CxxxxxCxxxxxxxxCxxCCxxxxxxxxxCxxxxCxC

2) drdxCxDxxxCxxygxyxxCxxCCxxxgxxxgxCxxxxCxC

PF08087: Toxin 18

Conotoxin O-superfamily. This family consists of members of the conotoxin O-superfamily. The O-superfamily of conotoxins consists of 3 groups of Conus peptides that belong to the same structural group. These 3 groups differ in their pharmacological properties: the w-conotoxins which inhibit calcium channels, the delta-conotoxins which slow down the inactivation rate of voltage-sensitive sodium channels and the muO-conotoxins block the voltage sensitive sodium currents. See FIG. 31.

Motif 1: CxxxxxxCxxxxxCCx(xx)xxCxxxxxxC,

Motif 2: CxxxgxxCxxxxxCCx(xx)gxCxxxfxxC

PF08088: Toxin 19

Conotoxin I-superfamily. See FIG. 6. This family consists of the I-superfamily of conotoxins. This is a new class of peptides in the venom of some Conus species. These toxins are characterised by four disulfide bridges and inhibit of modify ion channels of nerve cells. The I-superfamily conotoxins is found in five or six major clades of cone snails and could possible be found in many more species.

PF08089: Toxin 20

Huwentoxin family. This family consists of the huwentoxin-II (HWTX-II) family of toxins secreted by spiders. These toxins are found in venom that secreted from the bird spider Selenocosmia huwena Wang. The HWTX-II adopts a novel scaffold different from the ICK motif that is found in other huwentoxins. HWTX-II consists of 37 amino acids residues including six cysteines involved in three disulfide bridges. See FIG. 5.

PF08091: Toxin 21

This family is a member of the Omega toxin-like clan. This family consists of insecticidal peptides isolated from spider venom. See FIG. 58. There are 4 known family members. Topology is unknown. No structures are available.

1) CxxxxxxCxxxxxCCxxxCxxxxxxCxxxxxxCxxxC

2) CxxxxxPCxnxxxCCxgxCxxxxWxCxxxxxxCskxC

PF08092: Toxin 22

See FIG. 4. This family consists of Magi peptide toxins (Magi 1, 2 and 5) isolated from the venom of Hexathelidae spider. These insecticidal peptide toxins bind to sodium channels and induce flaccid paralysis when injected into lepidopteran larvae. However, these peptides are not toxic to mice when injected intracranially at 20 pmol/g.

PF08093: Toxin 23

See FIG. 3. This family consists of toxic peptides (Magi 5) found in the venom of the Hexathelidae spider. Magi 5 is the first spider toxin with binding affinity to site 4 of a mammalian sodium channel and the toxin has an insecticidal effect on larvae, causing paralysis when injected into the larvae.

PF08094: Toxin 24

Conotoxin TVIIA/GS family. This family consists of conotoxins isolated from the venom of cone snail Conus tulipa and Conus geographus. Conotoxin TVIIA, isolated from Conus tulipa displays little sequence homology with other well-characterised pharmacological classes of peptides, but displays similarity with conotoxin GS, a peptide from Conus geographus. Both these peptides block skeletal muscle sodium channels and also share several biochemical features and represent a distinct subgroup of the four-loop conotoxins (J. M. Hill, et al. (2000) Eur J Biochem, 267: 4642-8). See FIG. 28.

1) CxxxxxxCxxxCCxxxxCxxxxxxxC

2) CxGxxxxCPPxCCxGxxCxxGxxxxC

PF08095: Toxin 25

Hefutoxin family. This family consists of the hefutoxins that are found in the venom of the scorpion Heterometrus fulvipes. These toxins, kappa-hefutoxin1 and kappa-hefutoxin2, exhibit no homology to any known toxins. The hefutoxins are potassium channel toxins and exhibit a 1-4 2-3 topology. FIG. 173.

PF08097: Toxin 26

Conotoxin T superfamily. See FIG. 2. This family consists of the T-superfamily of conotoxins. Eight different T-superfamily peptides from five Conus species were identified. These peptides share a consensus signal sequence, and a conserved arrangement of cysteine residues. T-superfamily peptides were found expressed in venom ducts of all major feeding types of Conus, suggesting that the T-superfamily is a large and diverse group of peptides, widely distributed in the 500 different Conus species.

PF08099: Toxin 27

Scorpion Calcine family. See FIG. 1. This family consists of the calcine family of scorpion toxins. The calcine family consists of Maurocalcine and Imperatoxin. These toxins have been shown to be potent effector of ryanodyne-sensitive calcium channel from skeletal muscles. These toxins are thus useful for dihydropyridine receptor/ryanodyne receptor interaction studies.

PF08116: Toxin 29

This family consists of PhTx insecticidal neurotoxins that are found in the venom of Brazilian, Phoneutria nigriventer. The venom of the Phoneutria nigrivente contains numerous neurotoxic polypeptides of 30-140 amino acids which exert a range of biological effects. While some of these neurotoxins are lethal to mice after intracerebroventricular injections, others are extremely toxic to insects of the orders Diptera and Dictyoptera but had much weaker toxic effects on mice. See FIG. 7.

PF08117: Toxin 30

Also called Ptu family. This family consists of toxic peptides that are isolated from the saliva of assassin bugs. The saliva contains a complex mixture of proteins that are used by the bug either to immobilise the prey or to digest it. One of the proteins (Ptu1) has been purified and shown to block reversibly the N-type calcium channels and to be less specific for the L- and P/Q-type calcium channels expressed in BHK cells

Topology 1-4 2-5 3-6; 3 members. See FIG. 79.

1) CxxxxxxCxxxxxxCCxxxxxCxxxxxxC

2) CxxxgxxxCxgxxkxCCxxxxxCxxyanxC

PF08119: Toxin 31

This family consists of acidic alpha-KTx short chain scorpion toxins. These toxins named parabutoxins, block voltage-gated K channels and have extremely low pI values. Furthermore, they lack the crucial pore-plugging lysine. In addition, the second important residue of the dyad, the hydrophobic residue (Phe or Tyr) is also missing. See FIG. 8.

PF08120: Toxin 32

See FIG. 9. This family consists of the tamulustoxins, which are found in the venom of the Indian red scorpion (Mesobuthus tamulus). Tamulustoxin shares no similarity with other scorpion venom toxins, although the positions of its six cysteine residues suggest that it shares the same structural scaffold. Tamulustoxin acts as a potassium channel blocker.

http:www.ncbi.nlm.nih.gov/entrez/guery.fcgi?cmd=Retrieve&db=PubMed&dopt-Abstract&list_uids=11361010

PF08396: Toxin 34

Spider toxin omega agotoxin/Tx1 family. The Tx1 family lethal spider neurotoxin induces excitatory symptoms in mice. See FIG. 10.

PF01033: Somatomedin

See FIG. 14. Somatomedin B, a serum factor of unknown function, is a small cysteine-rich peptide, derived proteolytically from the N-terminus of the cell-substrate adhesion protein vitronectin. The SMB domain contains eight Cys residues, arranged into four disulfide bonds (Y. Kamikubo, et al. (2004) Biochemistry, 43: 6519-34). It has been suggested that the active SMB domain may be permitted considerable disulfide bond heterogeneity or variability, provided that the Cys25-Cys31 disulfide bond is preserved. The three dimensional structure of the SMB domain is extremely compact and the disulfide bonds are packed in the center of the domain forming a covalently bonded core. The protein can be expressed as a soluble fusion protein with the C-terminal domain of thioredoxin.

1) Cxx(x)xCxxxxxxxxxxCxCxxxCxxxxxCCxxxxxxC

2) Cxx(x) rCxxxxxxxxCxCxxxCxxxxxCCxDxxxxC

3) Cxx(x) RCxexxxxxxxxCxCxxxCxxxxxCCxd[yf]xxxC

A 1-2 3-4 5-6 7-8 topology has been described, but other isomers are also possible and consistent with NMR structure calculations.

PF00087, PF00021: Three Finger Toxin family

See FIG. 14-18. A family of venomous neurotoxins and cytotoxins. Structure is small, disulfide-rich, nearly all beta sheet. This family is a member of the uPAR/Ly6/CD59/snake toxin-receptor superfamily clan. This clan includes the following Pfam members: Activin_recp; BAMBI; PLA2_inh; Toxin_{—}1; UPAR_LY6.

A preferred library strategy is to randomize the three longest loops, which are between Cys1-Cys2, Cys3-Cys4 and Cys5-Cys6. Two different design strategies are used: 1) the disulfide core remains intact while mutagenizing only the three loops, 2) mutagenesis in the disulfide core is allowed and may yield a higher diversity of loop arrangements. The most conserved cysteine spacing is at position n6=0 and n7=4 (‘n6’ is defined as between C6 and C7; ‘n7’ is between C7 and C8). This information is used to evaluate the remaining CDP. The most common CDP is 10,6,16 3,10,0,4 with 69 members.

1) Cxxxxxxxxxx(xxx)Cxxxx(xx)Cxxxxxxxxxxxx(x)xxxxCx(xx)CxxxxxxxxxxCCxxxxC

2) Cyxxxxxxxxx(xxx)Cpxgx(xx)Cyxkx(wf)xxxxxx(x)xxxxGCx(xt)CPxxxxxxxxxCCx(ts)DxC

PF01607, PF00187: Chitin binding proteins

There are two different cysteine-rich chitin binding families (Z. Shen, et al. (1998) J Biol Chem, 273: 17665-70); T. Suetake, et al. (2000) J Biol Chem, 275: 17929-32; T. Suetake, et al. (2002) Protein Eng, 15: 763-9). PF00187 is found in fungi and plants and includes wheat germ agglutinin. Hevein is a prototypical member containing four disulfide bonds. The family includes 382 known family members with highly conserved cysteine positions and the topology 1-4 2-5 3-6 7-8. Advantages of this family for use as a scaffold in library design include the small number (<3) of amino acids at the N-terminal position of the first cysteine and the C-terminal position of the last cysteine. The distance between individual cysteines is lower than 10 and the domain is rich in disulfide bonds (approximately 50 amino acids with four disulfide bonds). The DBP is the most common 1-4 2-5 3-6 topology. The domain is found in repeats in nature.

PF01607 is also called Peritrophin domain and is found in animals and insects as part of extracellular matrix proteins. This domain also occurs in the small peptide tachycitin. Structural comparison of tachycitin and hevein (PF00187) reveals structural similarities (see alignment). Tachycitin contains five disulfide bonds, but members of this family typically contain 3SS (see logo). Tachycitin's 3 signature SS exhibit 1-3 2-6 4-5 topology. There are 1075 known family members. The cysteine positions are highly conserved. Not many (<3) amino acids N-terminal of the first cysteine and C-terminal of last cysteine.

See FIGS. 19-21.

PF00187 Chitin binding proteins:

CxxxxxxxxCxxxxCCxxxxxCxxxxxxCxxxCxxxC

CgxqxxxxxCxxxxCCsxxGxCGxxxxyCxxxCxxxC

PF01607 Chitin binding domain:

1) Cxxx(x)xxxxxxx(x)xxxC(x)xxxxxCxxxxxxxxxCxxxxxxxxxxxxCxxxxxxxx

2) Cxxx(x)xxgxxxx(x)xxxC(x)xx[yf]xxCxxxxxxxxxCxxgxxfxxxxxxCxxxxxxxxC

PF01826: Trypsin inhibitor

This family contains trypsin inhibitors as well as a domain found in many extracellular proteins [N. D. Rawlings, et al. (2004) Biochem J, 378: 705-16]. The domain typically contains ten cysteine residues that form five disulphide bonds. The DBP is 1-7 2-6 3-5 4-10 8-9. 414 Family members are known. The cysteine positions are highly conserved. See FIG. 23.

CxxxxxxxxCxxxCxxxCxxxx(xxxxx)xxxCx(xxxxxxx)xxCxxx(x)CxCxxxxxxxxx(xx)xCxxxxxC

PF02428: Potato protein inhibitors

This family is found in repeats on the genetic level. The protein is synthesized as a large precursor protein. Proteolytic cleavage occurs within repeats, rather than between repeats, to yield the mature microprotein [E. Barta, et al. (2002) Trends Genet, 18: 600-3][N. Antcheva, et al. (2001) Protein Sci, 10: 2280-90].

A large precursor protein is synthesized, but disulfide topology for precursor is unknown.

The repeat unit was expressed and its NMR structure was solved. The fold is similar to the mature microprotein suggesting that circular permutation has occurred and that this unit was the ancestor. This is supported by the discovery of a circular permuted protein that corresponds to the repeat unit. The linker or protease site (EEKKN) is present as a disordered loop in the structure of the ancestor. See FIG. 24.

1) CxxxCxxxxxxxxCxxxxxx(x)xxxxxCxxCCxxxxxCxxxxxxxxxxC

2) CxxxCxxxxxxxxCPxxxxx(x)xxxxxCxxCCxxxxGCxxxxxxGxxxC

Due to the proteolytic processing, the sequence of the mature microprotein is different form the logo shown above:

2C2CC5C10C11C3C8C2 (mature logo-protein level)

3C3C8C12C2CC5C10C2 (repeat logo-genetic level)

PF00304: Gamma Thionin

In their mature form, these small plant proteins generally consist of about 45 to 50 amino-acid residues. The folded structure of Gamma-purothionin is characterised by a well-defined 3-stranded anti-parallel-sheet and a short helix. Three disulphide bridges are located in the hydrophobic core between the helix and sheet, forming a cysteine-stabilized-helical motif (P. B. Pelegrini, et al. (2005) Int J Biochem Cell Biol, 37: 2239-53). This structure is analogous to scorpion toxins and insect defensins (C. Bloch, Jr., et al. (1998) Proteins, 32: 334-49).

The domain shows high disulfide density with 4 disulfide bonds per approximately 50 amino acids and a topology of 1-8 2-5 3-6 4-7. The cysteine spacing between individual cysteines is smaller than 10 and therefore preferred for library design. The cysteine positions are highly conserved among different members of this family. See FIG. 25.

PF00304—Gamma-Thionin:

Motif 1: CxxxxxxxxxCxxxxxCxxxCxxxxxx(x)xxxCxx(x)xxxxCxCxxxC

Motif 2: CxxxSxxFxGxCxxxxxCxxxCxxxxxx(x)xGxCxx(x)xxxxCxCxxxC

PF02950: Omega-Conotoxin

Conotoxins are small snail neurotoxins that block ion channels. Omega-conotoxins act at presynaptic membranes and bind and block the calcium channels (W. R. Gray, et al. (1988) Annu Rev Biochem, 57: 665-700). The domain shows high disulfide density with three disulfide bonds per approximately 24 amino acids. There are more than 380 known family members. The cysteine spacing between individual cysteines is smaller than 10 and therefore preferred for library design. The cysteine positions are highly conserved among different members of this family which has a DBP of 1-4 2-5 3-6.

See FIG. 26. Motif: C(xx)xxxxxCCxx(xx)xCx(xxx)xxCC

Ziconotide is a 25AA conotoxin that has been FDA approved ‘Prialt’). Ziconotide has been in >7000 patients and is non-immunogenic (<1% incidence), which makes this a promising scaffold for new binding proteins for use in humans. The sequence and 1-4 2-5 3-6 DBP is shown in FIG. 12.

PF05374: Mu-conotoxin

Mu-conotoxins are peptide inhibitors of voltage-sensitive sodium channels (K. J. Nielsen, et al. (2002) J Biol Chem, 277: 27247-55). See FIG. 29. DBP: 1-4 2-5 3-6

Motif 1: CCxxxxxCxxxxCxxxxCC Motif 2: CCxxpxxCxxxxCxPxxCC

PF02822: Antistasin

Peptide proteinase inhibitors can be found as single domain proteins or as single or multiple domains within proteins; these are referred to as either simple or compound inhibitors, respectively (R. Lapatto, et al. (1997) Embo J, 16: 5151-61). In many cases they are synthesised as part of a larger precursor protein, either as a prepropeptide or as an N-terminal domain associated with an inactive peptidase or zymogen. The Pfam definition includes only six cysteines with a DBP of 1-4 2-5 3-6. However, most members of the family (1bx7, 1hia) contain two more N-terminal disulfides. This family can therefore be extended on the N-terminus.

The domain shows high disulfide density with 3-5 disulfide bonds per 39-54 amino acids and a topology of 1-3 2-4 5-8 6-9 7-10. The cysteine spacing between individual cysteines is smaller than 10 and therefore preferred for library design. The cysteine positions are highly conserved among different members of this family. See FIG. 32.

Members of this family are very hydrophilic which is preferred for library design (low non-specific binding, low number of T-cell epitopes). For example, hirustasin contains a total of only 6 hydrophobic residues. The crystal structure displays a near absence of secondary structure elements. This, in combination with the high number of possible disulfide isomers of 5SS, makes this a very useful scaffold for library design.

Cysteine positions are highly conserved, for 5 disulfides: C4C5C6C1C4C4C10C5C1C

PF02822—Antistasin:

1) CxxxxCxxxxxCxxxxxxCx CxxxxC(x)xxxCxxxxxxxxxCx(xxx)xCxC

2) CxxxxCxxxxxCxxxxxxCxCxxxxC(x)xxxCxxGxxxdxxgCx(xxx)xCxC

3) CxxxxCxxxxxCxxxxxxCxCxxxxC(x)xxxCpyGxxxdxxgCx(xxx)xCxC

Short version lacking the N-terminal four cysteine residues:

1) CxxxxC(x)xxxCxxxxxxxxxCx(xxx)xCxC

2) CxxxxC(x)xxxCxxGxxxdxxgCx(xxx)xCxC

3) CxxxxC(x)xxxCpyGxxxdxxgCx(xxx)xCxC

PF05039: Agouti-related

See FIG. 33. The agouti protein regulates pigmentation in the mouse hair follicle producing a black hair with a subapical yellow band. A highly homologous protein agouti signal protein (ASIP) is present in humans and is expressed at highest levels in adipose tissue where it may play a role in energy homeostasis and possibly human pigmentation (J. C. McNulty, et al. (2001) Biochemistry, 40: 15520-7; J. Voisey, et al. (2002) Pigment Cell Res, 15: 10-8).

The disulfide bond between Cys5 and Cys10 is not necessary for structure and function. Upon removal, the DBP becomes 1-4 2-5 3-8 6-7. The first three disulfide bonds form the signature cystine knot motif. The receptor binding site includes the RFF motif between Cys7 and Cys8 and a loop formed by the first 16 amino acids. The C terminus is disordered and can be removed (Note that Cys1 and Cys10 are not present in the Pfam logo).

The following logo is preferred for library design: PF05039—Agouti:

1) CxxxxxCxxxxxxCCxxCxxCxCxxxxxxCxCxxxxxxxxxC

2) CxxxxSCxxxxxxCCDPCxxCxCRFFxxxCxCRxxxxxxxxC

3) CxxxxSCxGxxxPCCDPCAxCxCRFFxxxCxCRxLxxxxxxC

An engineered protein with a shorter C-terminus and lacking cysteine 5 and cysteine 10 folds into a similar structure as the native protein. This engineered version is used as a scaffold for library design and has the following logos: CxxxxxCxxxxxxCCxxxxxCxCxxxxxxCxCx, CxxxxxCxxxxxxCCDPxxxCxCRFFxxxCxCRxx, CxGxxxCxxxxxxCCDPAxxCYCRFFxxxCxCRxx

Full-length agouti protein can be expressed as a soluble protein in Escherichia coli (R. D. Rosenfeld, et al. (1998) Biochemistry, 37: 16041-52).

PF05375: PMP inhibitors/Pacifastin

Structures of members of this family show that they are comprised of a triple-stranded antiparallel beta-sheet connected by three disulfide bridges, which defines this family as a novel family of serine protease inhibitors (G. Simonet, et al. (2002) Comp Biochem Physiol B Biochem Mol Biol, 132: 247-55; A. Roussel, et al. (2001) J Biol Chem, 276: 38893-8). See FIG. 34.

There are 39 family members. The cysteine positions are highly conserved with a disulfide topology of 1-4 2-6 3-5. The distances between individual cysteines are <10. The C-terminus is not visible in structures suggesting that it can be omitted from library design. Two strongly conserved amino acids are N15 and T29, which are involved in forming and stabilizing a protease binding loop. They can be omitted from library design to increase binding diversity.

1) CxxxxxxxxxCxxCxCxxxx(x)xxxCxxxxC

2) CxpGxxxKxxCNxCxCxxxx(x)xxxCTxxxC

PF01549: ShTK family and Stecrisp

Stecrisp exhibits a highly similar 3D structure to ShTK family, but is not part of the ShTK family (PF01549) (M. Guo, et al. (2005) J Biol Chem, 280: 12405-12). Blast search with the Stecrisp protein sequence yields 48 matches with 30-100% identity, but does not yield any ShTK family members. See FIG. 35-36.

Pfam01549 is a domain of unknown function and is found in several C. elegans proteins. The domain is 30 amino acids long and has 6 conserved cysteine positions that form three disulphide bridges. The domain is named (by SMART) after ShK toxin. (M. Dauplais, et al. (1997) J Biol Chem, 272: 4302-9).

The domain shows high disulfide density with 3 disulfide bonds per 39 amino acids and a topology of 1-6 2-4 3-5. The cysteine spacing between individual cysteines is smaller than 10 and therefore useful for library design. The cysteine positions are highly conserved among different members of this family.

PF01549—ShTK. See FIG. 35:

1) Cx(xxx)xxx(x)xxCxxxxxx(xx)Cxxxx(x)xxxxxxxxCxxxCxxC

2) Cx(dxx)dxx(x)xxCxxxxxx(xx)Cxxxx(x)xxxxxxxCxxtCxxC

C-terminal domain of STECRISP and related sequences: see FIG. 36.

PF07974: EGF2 domain

Members of this family all belong to the EGF superfamily, which is characterised as having 6-8 cysteines forming 3-4 disulfide bonds, in the order 1-3, 2-4, 5-6, which are essential for the stability of the EGF fold. These disulphide bonds are stacked in a ladder-like arrangement. The Laminin EGF family is distinguished by having an additional disulphide bond. The function of the domains within this family remains unclear, but they are thought to largely perform a structural role. More often than not, the domains are arranged in tandem repeats in extracellular proteins.

PF07974—EGF2: See FIG. 37.

1) Cx(xxxxxx)Cxx(x)xxxCxxxx(xxxxxxxx)CxCxxx(xxxx)xxxxxC

2) Cx(xxxxxx)Cxx(x)xGxCxxxx(xxxxxxxx)CxCxxx(xxxx)xxGxxC

Other EGF-like domains:

PF00008—EGF: See FIG. 38.

1) CxxxxxCxxxxxCxxxxx(xx)xxxCxCxxx(xxxx)xxxxxC

2) CxxxxxCxxxgxCxxxxx(xx)xxxCxCxxg(xxxx)xxgxxC

PF00053—Lam-EGF: See FIG. 39. DBP: 1-3 2-4 5-6 7-8

1) CxCxxxxxxxx(xx)Cxxxxxxxxx(xxxx)CxxCxxxxxxxxCxxCxxxxxxxxxx(xxxxx)C

2) CxCxxxxxxxx(xx)Cxxxxxxxxx(xxGx)CxxCxxxxxGxxC(DE)xCxxxxxxxxxx(xxxxx)C

PF07645: Ca-EGF: See FIG. 40.

1) CxxxxxxxCxxxxxx(xx)CxxxxxxxCx(xxxx)Cxxxxxxxxxx(xxxxxxx)C

2) CxxxxxxxCxxxxxx(xx)CxNxxGx(F,Y)xCx(xxxx)Cxx(G,Y)xxxxxxx(xxxxxxx)C

PF04863: Allinase EGF-like: See FIG. 41.

1) Cxxxxxxxxxxxxxxxx(xxxx)CxCxxCxxxxxCxxxxxxC

2) Cxxxxxxxxxxxxxxxx(xxxx)CxCxxCxxxxxCxxxxxxC

PF00323: Mammalian Defensin; Defensin 1

See FIG. 45. DBP: 1-6 2-4 3-5

1) CxCxxxxCxxxxxxxxxCxxxxxxxxxCC

2) CxCRxxxCxxxErxxGxCxxxgxxxxxCC

PF01097: Arthropod Defensin; Defensin 2

See FIG. 44. DBP: 1-4 2-5 3-6

1) CxxxCxxxxxxxxxCx(xxx)xxxCxC

2) CxxHCxxxgxxGGxCxx(xx)xxxCxC

PF00711: Defensin B, Beta-Defensin

See FIG. 43. DBP: 1-4 2-5 3-6 or 1-5_{—}2-4_{—}3-6

1) CxxxxxxCxxxxCxxxxxxxxxCxxxxxxCC

2) CxxxxgxCxxxxCxxxxxxxgxCxxxxxxCC

PF08131: Defensin-like; Defensin 3 FIG. 42.

1) Cxxxx(GxCrxkxxxnCxxxxxxxCxnxxqkCC

2) CxsxxGxCrxkxxxnCxxxxxxxCxnxxqkCC

The Defensin-(like-)3 family consists of the defensin-like peptides (DLPs) isolated from platypus venom (A. M. Torres, et al. (1999) Biochem J, 341 (Pt 3): 785-94). These DLPs show similar three-dimensional fold to that of beta-defensin-12 and sodiuim-channel neurotoxin Sh1. However the side chains known to be functionally important to beta-defensin-12 and Sh1 are not conserved in DLPs. This suggests a different biological function. Consistent with this contention, DLPs have been shown to possess no anti-microbial properties and have no observable activity on rat dorsal-root-ganglion sodium-channel currents. Only three members are known, but the similarity to beta defensins makes this an attractive scaffold.

The domain shows high disulfide density with 3 disulfide bonds per approximately 36 amino acids with a topology of 1-5_{—}2-4_{—}3-6. The cysteine spacing between individual cysteines is smaller than 10 and therefore useful for library design. The cysteine positions are highly conserved among different members of this family.

PF00321: Crambins

Crambins are small, basic plant proteins, 45 to 50 amino acids in length, which include three or four conserved disulphide linkages. The proteins are toxic to animal cells, presumably attacking the cell membrane and rendering it permeable: this results in the inhibition of sugar uptake and allows potassium and phosphate ions, proteins, and nucleotides to leak from cells This family is different from gamma-thionin PF00304 (P. B. Pelegrini, et al. (2005) Int J Biochem Cell Biol, 37: 2239-53).

The domain shows high disulfide density with 4 disulfide bonds per approximately 46 amino acids. The cysteine spacing between individual cysteines is smaller than 10 and therefore useful for library design. The cysteine positions are highly conserved among different members of this family. See FIG. 46.

Cysteine positions are highly conserved, Distance between individual cysteines are around 10 and lower, topology 1-6 2-5 3-4; Domain is small with 6 cysteines

Motifs for members containing three disulfide bonds are

PF00321—Crambins:

1)xxCCxxxxxxxxxxCxxxxxxxxxCxxxxxCxxxxxxxCxxxxxx

2)xxCCxxxxxRxxYxxCxxxGxxxxxCxxxxxCxIxxxxxCxxxxxx

3)xxCCxxxxxRxxYxxCRxxGxxxxxCAxxxxCxIISGxxCPxx(Y,F)xx

Motifs for members with four disulfide bonds and the topology 1-8 2-7 3-6 4-5 are characterized by the following logos: xxCCxxxxxxxCxxxCxxxxxxxxCxxxCxCxxxxxxxC

PF06360: Raikovi

Diffusible peptide pheromones with only 6 family members, but high diversity in inter-cysteine amino acids (M. S. Weiss, et al. (1995) Proc Natl Acad Sci USA, 92: 10172-6). The cysteine positions are highly conserved with a topology of 1-4 2-6 3-5. The distance between individual cysteines is <10. See FIG. 47.

1) CxxxxxxCxxxxCxxxCxxxxxxxxCxxxxxxxxxC

2) CxxaxxxCxxxxCxxxCxxxxxxxxCxxxxxxxxxC

PF00683: TB domain

Transforming growth factor (TGF-)-binding protein-like (TB) domain comes from human fibrillin. This domain is found in fibrillins and latent TGF-binding proteins (LTBPs) which are localized to fibrillar structures in the extracellular matrix. (X. Yuan, et al. (1997) Embo J, 16: 6659-66). Repeat means that this domain is found in multiple copies in fibrillins and LTBP, but NOT in tandem. See FIG. 49.

Logo shows only 6 conserved cysteines. Three structures were analyzed (1uzq, 1apj, 1ksq): one missing cysteine is inserted between Cys1 and the Cys triplett (positions 8/12, 4/12, 9/12), and the last cysteine missing in logo. The topology is 1-3 2-6 4-7 5-8.

1) CxxxxxxxxxxxxxCCCxxxx(xx)xxxxxCxxCPxxxxxxxC

2) Cxxxxxxx(x)xxkxxCCCxxxx(xx)xxgxxCexCPxxxxxxxC

PF00093: von Willebrand factor type C domain

The vWF domain is found in various plasma proteins, complement factors, the integrins, collagen types VI, VII, XII and XIV; and other extracellular proteins (P. Bork (1993) FEBS Lett, 327: 125-30). There are 488 known family members with highly conserved cysteine residues. Structure and sequence comparisons have revealed an evolutionary relationship between the N-terminal sub-domain of the CR module and the fibronectin type 1 domain, suggesting that these domains share a common ancestry (J. M. O'Leary, et al. (2004) J Biol Chem, 279: 53857-66). See FIG. 50.

Mini-Collagen Cysteine-Rich Domain

Mini collagens are found in the cell wall of Hydra. Mini collagens contain a C-terminal cysteine-rich domain that is synthesized as intra molecular disulfide bonded precursor. The C-terminal domain is a microprotein with a unique fold (S. Meier, et al. (2004) FEBS Lett, 569: 112-6; E. Pokidysheva, et al. (2004) J Biol Chem, 279: 30395-401). Only cysteine residues are highly conserved among 16 family members. Disulfide bonds are thought to be shuffled to intermolecular disulfide bonds to form a cell wall stabilizing matrix. The disulfide topology is 1-5 2-4 3-6. The observation that C-terminal domains form intermolecular disulfide bonds with each other can be exploited to create combinatorial libraries of dimeric molecules linked by intermolecular disulfide bonds. See FIG. 136.

Motif: C3C3C3C3CC in minicollagen and C5C3C3C3C3CC in Hydra HOWA protein, where this domain occurs as a repeat.

PF03784: Cyclotide

This family contains a set of cyclic peptides with a variety of activities. The structure consists of a distorted triple-stranded beta-sheet and a cysteine-knot arrangement of the disulfide bonds (D. J. Craik, et al. (1999) J Mol Biol, 294: 1327-36). See FIG. 51.

Topology is 1-4_{—}2-5_{—}3-6

1) CxxxCxxxxCxxxxxxxCxCxxxxC

2) CxExCxxxxCxxxxxxGCxCxxxxC

PF06446: Hepcidin

Hepcidin is an antibacterial and antifungal protein expressed in the liver and is also a signaling molecule in iron metabolism. The hepcidin protein is cysteine-rich and forms a distorted beta-sheet with an unusual disulphide bond found at the turn of the hairpin.

See FIG. 52. Topology is 1-8 2-7 3-6 4-5

Motif 1: xxxCxxCCxCCxxxxCxxCC

Motif 2: FPxCxFCCxCCxxxxCGxCC

PF05353: Delta-Atracotoxin

The structure of atracotoxin comprises a core beta region containing a triple-stranded a thumb-like extension protruding from the beta region and a C-terminal helix. The beta region contains a cystine knot motif, a feature seen in other neurotoxic polypeptides. See FIG. 53.

Topology is 1-4 2-6 3-7 5-8

Motif 1: CxxxxxxCxxxxxCCCxxxCxxxxxxxxCxxxxxxxxxC

Motif 2: CxxxxxWCxxxxxCCCPxxCxxWxxxxxCxxxxxxxxxC

PF00299: Serine Protease Inhibitor

The squash inhibitors form one of a number of serine proteinase inhibitor families. They are approximately 30 residues in length and contain 6 Cys residues, which form 3 disulphide bonds. Topology is 1-4 2-5 3-6. See FIG. 56.

1) CxxxxxxCxxxxxCxxxCxCxxxx(x)xC

2) CPxxxxxCxxpxpCxxxCxCxxxx(x)xCG

PF01821: Anaphylotoxin-like domain

C3a, C4a and C5a anaphylatoxins are protein fragments generated enzymatically in serum during activation of complement molecules C3, C4, and C5. They induce smooth muscle contraction. These fragments are homologous to a three-fold repeat in fibulins. Topology is 1-4 2-5 3-6. There are 123 know members of this family. See FIG. 57.

1) CCxxxxxx(xxxx)xxCxxxxxxxx(xx)xxCxxxxxxCC

2) CCxxGxxx(xxxx)xxCxxxxxxxx(xx)xxCxxxFxxCC

PF05196: Midkine/PTN

Several extracellular heparin-binding proteins involved in regulation of growth and differentiation belong to a new family of growth factors (W. Iwasaki, et al. (1997) Embo J, 16: 6936-46). There are 33 family members. The cysteine positions are highly conserved forming a disulfide topology of 1-4 2-5 3-6. The distances between individual cysteines are <10. The NMR structure of midkine shows highly disordered N- and C-termini suggesting that these can be omitted form library design. Positively charged residues are involved in heparin binding and can be omitted from library design. See FIG. 59.

1) CxxxxxxxCxxxxxxCxxxxxxxCxxxxxxxxCxxxC

2) CxxWxxxxCxxxxxDCGxGRExxCxxxxxxxxCxxPCxW

PF02819: WAP “four-disulfide core”

While the pattern of conserved cysteines suggests that the sequences may adopt a similar fold, the overall degree of sequence similarity is low (L. G. Hennighausen, et al. (1982) Nucleic Acids Res, 10: 2677-84). There are 25 known family members. See FIG. 62.

Topology is 1-6 2-7 3-5 4-8.

1)xxxx(xx)xxxxCxxx(xxx)CxxxxxCxxxxxCCxxxC

2) CPxxx(xx)xxxxCxxx(xxx)CxxDxxCxxxxKCCxxxC

PF02048, PF07822: Toxic hairpins

Toxin 13 (PF07822) folds into a 4SS disulfide-linked alpha-helical hairpin. The SCOP database also lists heat stable enterotoxin (PF02048) as toxic hairpin with a DBP of 1-4 2-5 3-6.

The members of this family resemble neurotoxin B-IV, which is a crustacean-selective neurotoxin produced by the marine worm Cerebratulus lacteus. This highly cationic peptide is approximately 55 residues and is arranged to form two antiparallel helices connected by a well-defined loop in a hairpin structure. The branches of the hairpin are linked by four disulphide bonds. Three residues identified as being important for activity are found on the same face of the molecule, while another residue important for activity, Trp30, is on the opposite side. The protein's mode of action is not entirely understood, but it may act on voltage-gated sodium channels, possibly by binding to an as yet uncharacterized site on these proteins. See FIG. 65. Toxin 13 topology is 1-8 2-5 3-6 4-5

1) CxxxCxxxxxxCxxCxxxxxxxxxxCxxxCxxxxxxCxxxC

2) CxxxCxxxyxxCxxCxgxWxgxxgxCxxhCxxxxxxCxxxC

PF06357: Omega-atracotoxin

Omega-Atracotoxin-Hv1a is an insect-specific neurotoxin whose phylogenetic specificity derives from its ability to antagonise insect, but not vertebrate, voltage-gated calcium channels (X. Wang, et al. (1999) Eur J Biochem, 264: 488-94). Topology is 1-6_{—}2-7_{—}3-4_{—}5-8

See FIG. 66. Topology is 1-4_{—}2-5_{—}3-6. CxPxxxPCPYxxxxCCxxxCxxxxxxGxxxxxxC

PF06954: Resistin

This family consists of several mammalian resistin proteins. It has been demonstrated that increases in circulating resistin levels markedly stimulate glucose production in the presence of fixed physiological insulin levels, whereas insulin suppressed resistin expression.

Resistin contains a N-terminal alpha helix that participates in the multimerization of the C-terminal disulfide-rich part. See FIG. 67. Topology is 1-10 2-9 3-6 4-7 5-8

Only the disulfide-rich microprotein is shown. The N-terminal alpha-helix motif can be used for multimerization of microproteins.

1) CxxxxxxxxxxxCxxxxxxxxCxCxxxCxxxxxxxxCxCxCxxxxxxxxCC

2) CxxxxxxxxxxxCPxGxxxxxCxCGxxCGxWxxxxxCxCxCxxxDWxxRCC

PF00066: Notch/DSL

Extracellular domain of transmembrane protein involved in developmental processes of animals (J. C. Aster, et al. (1999) Biochemistry, 38: 4736-42; D. Vardar, et al. (2003) Biochemistry, 42: 7061-7). DSL repeat occurs in tandem (3×). Three conserved Asp or Asn residues. In the NMR structure, D12, N15, D30, D33, form a Ca2+ binding site. Only one isomer is formed in the presence of milimolar Ca2+, but multiple isomers are observed in the presence of Mg2+ or EDTA. This can be exploited for structural evolution of microproteins. There are 175 family members. The cysteine positions are highly conserved with a 1-5 2-4 3-6 topology. Not many (<3) amino acids N-terminal of first cysteine and C-terminal of last cysteine. The distance between individual cysteines are <10. See FIG. 68.

1) Cx(xx)xxxCxxxxxxxxCxxxCxxxxCxxxxxxC

2) Cx(xx)xxxCxxxxxxgxCxxxCnxxxCxxDGxDC

PF00020: TNFR

A number of proteins, some of which are known to be receptors for growth factors have been found to contain a cysteine-rich domain at the N-terminal region that can be subdivided into four (or in some cases, three) repeats containing six conserved cysteines all of which are involved in intrachain disulphide bond (M. D. Jones, et al. (1997) Biochemistry, 36: 14914-23). The domain contains six highly conserved cysteine residues with a topology of 1-2 3-5 4-6.

See FIG. 69.

1) Cxxx(x)xxxxxxx(x)xxCx(x)CxxCxx(xx)xxxxxxxCxxxxxxxC

2) Cxxx(x)x[yf]xxxxx(x)xxCx(x)CxxCxx(xx)gxxxxxxCxxxxxtxC

PF00039: Fibronectin type H domain

Fibronectin is a multi-domain glycoprotein, found in a soluble form in plasma, that binds cell surfaces and various compounds including collagen, fibrin, heparin, DNA, and actin.

See FIG. 70. 1-3 2-4 topology. Motif: CxfpfxxxxxxxxxCxxxxxxxxxxwCxxxxxxxxDxxxxxC

PF02013: Cellulose or Protein Binding Domain

Those found in aerobic bacteria bind cellulose (or other carbohydrates); but in anaerobic fungi they are protein binding domains, referred to as dockerin domains or docking domains.

1-2 3-4 topology. See FIG. 71.

Motif: Cxx(xxx)xxxyxCCxxxxxxxxxxwcxxxxxxxxDxxxxxCxxxx(xxxx)xxxxxxxxwxxxxxxxC

PF00734: Fungal cellulose binding domain

Structurally, cellulases and xylanases generally consist of a catalytic domain joined to a cellulose-binding domain (CBD) by a short linker sequence rich in proline and/or hydroxy-amino acids [N. R. Gilkes, et al. (1991) Microbiol Rev, 55: 303-15]. The CBD of a number of fungal cellulases has been shown to consist of 36 amino acid residues, and it is found either at the N-terminal or at the C-terminal extremity of the enzymes. Members of this family possess two disulfide bonds with topology 1-3 2-4. See FIG. 73.

Motif: qCGGxxxxGxxxCxxgxxCxxxxxxy

PF00219: Insulin-like growth factor binding protein

The insulin-like growth factors (IGF-I and IGF-II) bind to specific binding proteins in extracellular fluids with high affinity. Members of this family possess two disulfide bonds with topology 1-3 2-4. See FIG. 74, 75.

PF00322: Endothelin family

Endothelins (ET's) are the most potent vasoconstrictors known. These peptides which are 21 residues long contain two intramolecular disulphide bonds with a 1-4 2-3 topology. See FIG. 76.

PF02058: Guanylin precursor

Guanylin, a 15-amino-acid peptide, is an endogenous ligand of the intestinal receptor guanylate cyclase-C, known as StaR. These peptides contain two intramolecular disulphide bonds with a 1-3 2-4 topology. See FIG. 77.

PF02977: Carboxypeptidase inhibitor

Peptide proteinase inhibitors can be found as single domain proteins or as single or multiple domains within proteins; these are referred to as either simple or compound inhibitors, respectively. In many cases they are synthesised as part of a larger precursor protein, either as a prepropeptide or as an N-terminal domain associated with an inactive peptidase or zymogen. Removal of the N-terminal inhibitor domain either by interaction with a second peptidase or by autocatalytic cleavage activates the zymogen.

There are 35 known family members. Topology is 1-4 2-5 3-6. See FIG. 80.

1) CxxxxxxCxxxxxCxxxCxCxxxxxxC

2) CPxixxxCxxdxdCxxxCxCxxxxxxCg

PF06373: CART

CART consists mainly of turns and loops (ca. 40 amino acids) spanned by a compact framework composed by a few small stretches of antiparallel beta-sheet common to cystine knots. There are 13 known family members.

Topology is 1-3 2-5 4-6. See FIG. 81.

In contrast to all other families, the non-cys residues are rather conserved and this family does not appear to be a preferred choice for randomization.

Follistatin

Human Follistatin is an FDA approved product and non-immunogenic and therefore the 70-72AA Follistatin domains are attractive scaffolds. It contains a total of 36 cysteine residues, believed to be arranged into nonoverlapping sets of disulfide bridges corresponding to four autonomous folding units (FIG. 218). The first of these units, which we call Fs0, comprises the 63 N-terminal residues of the mature polypeptide and bears no sequence similarity with any other protein of known structure. In contrast, the rest of the follistatin chain appears to fold into a series of three consecutive 70-74-residue-long Follistatin domains which are structural repeats that are referred to as Fs1, Fs2, and Fs3, which display homology to the follistatin-like domain of the extracellular matrix protein BM-40 and are also found in several other extracellular matrix proteins, such as agrin, tomoregulin, and complement proteins C6 and C7. See FIG. 151. Each 69-72AA Follistatin domain has a DBP of 1-3 2-4 5-9 6-8 7-10.

PF00713: Hirudin

The hirudin family is a group of proteinase inhibitors belonging to MEROPS inhibitor family 114, clan IM; they inhibit serine peptidases of the S1 family.

Hirudin is a potent thrombin inhibitor secreted by the salivary glands of the Hirudinaria manillensis (buffalo leech) and Hirudo medicinalis (medicinal leech). It forms a stable non-covalent complex with alpha-thrombin, thereby abolishing its ability to cleave fibrinogen. The structure of hirudin has been solved by NMR, and the structure of a recombinant hirudin-thrombin complex has been determined by X-ray crystallography to 2.3A. Hirudin consists of an N-terminal globular domain and an extended C-terminal domain. Residues 1-3 form a parallel beta-strand with residues 214-217 of thrombin, the nitrogen atom of residue 1 making a hydrogen bond with the Ser 195 O gamma atom of the catalytic site. The C-terminal domain makes numerous electrostatic interactions with an anion-binding exosite of thrombin, while the last five residues are in a helical loop that forms many hydrophobic contacts. See FIG. 123.

PF06410: Gurmarin

Gurmarin is a 35-residue polypeptide from the Asclepiad vine Gymnema sylvestre. It has been utilised as a pharmacological tool in the study of sweet-taste transduction because of its ability to selectively inhibit the neural response to sweet tastants in rats

There are 2 known family members. Topology is 1-4 2-5 3-6. See FIG. 82.

1) CxxxxxxCxxxxxxCCxxxxCxxxxxxxxxC

2) CxxxxxxCxxxxxxCCxxxxCxxxxwwxxxC

PF08027: Albumin-1

The albumin I protein, a hormone-like peptide, stimulates kinase activity upon binding a membrane bound 43 kDa receptor. The structure of this domain reveals a knottin like fold, comprise of three beta strands. There are 34 known family members. Topology is 1-4 2-5 3-6. See FIGS. 83-84.

PF08098: Neurotoxin (ATX III)

This family consists of the Anemonia sulcata toxin III (ATX III) neurotoxin family. ATX III is a neurotoxin that is produced by sea anemone; it adopts a compact structure containing four reverse turns and two other chain reversals, but no regular alpha-helix or beta-sheet. A hydrophobic patch found on the surface of the peptide may constitute part of the sodium channel binding surface. There are 2 known family members. Topology is 1-4 2-5 3-6.

FIG. 85. Motif: CCxCxxxxxxxxCxxxxxxxxxxC

PF01147: CHH/MIH/GIH neurohormone

Arthropods express a family of neuropeptides which include, hyperglycemichormone (CHH), molt-inhibiting hormone (MIH), gonad-inhibiting hormone (GIH) and mandibular organ-inhibiting hormone (MOIH) from crustaceans and ion transport peptide (ITP) from locust.

There are 131 known family members. Topology is 1-5 2-4 3-6. See FIG. 86.

PF04736: Eclosion

Eclosion hormone is an insect neuropeptide that triggers the performance of ecdysis behaviour, which causes shedding of the old cuticle at the end of a molt. There are 5 known family members. Topology is 1-5 2-4 3-6. No structures are available. See FIG. 88.

1) CxxxCxxCxxxxxxxxxxxxCxxxCxxxxxxxxxxC

2) CxxnCxqCkxmxgxxfxgxxCxxxCxxxxgxxxpxC

PF01160: Endogenous opioid neuropeptide

Vertebrate endogenous opioid neuropeptides are released by post-translational proteolytic cleavage of precursor proteins. The precursors consist of the following components: a signal sequence that precedes a conserved region of about 50 residues; a variable-length region; and the sequence of the neuropeptide itself. Sequence analysis reveals that the conserved N-terminal region of the precursors contains 6 cysteines, which are probably involved in disulphide bond formation. It is speculated that this region might be important for neuropeptide processing. There are 50 known family members. Topology is 1-4 2-5 3-6. No structures are available. See FIG. 89.

1) CxxxCxxCxxxxxxxxxxxxxxxCxxxCxxxxxxxxxxxxC

2) CxxxCxxCxxxxxxxxxxxxxxxCxlxCxxxxxxxxxWxxC

PF08037: Mollusk pheromone

This family consists of the attractin family of water-borne pheromone. Mate attraction in Aplysia involves a long-distance water-borne signal in the form of the attractin peptide, that is released during egg laying. These peptides contain 6 conserved cysteines and are folded into 2 antiparallel helices. The second helix contains the IEECKTS sequence conserved in Aplysia attractins. There are 5 known family members. Topology is 1-6 2-5 3-4. FIG. 90.

1) CxxxxxxxxCxxxxxxCxxxxxCxxxxxxCxxxxxxxC

2) CdxxxxxsxCqrnxxxxCxxaxxCxxxieeCktsxxexC

PF03913: AMBV Protein

Amb V is an Ambrosia sp (ragweed) protein. AmbV has been shown to contain a C-terminal helix as the major T cell epitope. Free sulfhydryl groups also play a major role in the T cell recognition of cross-reactivity T cell epitopes within these related allergens

There are 3 known family members. Topology is 1-7 2-5 3-6 4-8. FIG. 92.

1) CxxxxxxCCxxxxxxC(x)xxxxCxxxxxxCxxxC

2) CgxxxxyCCxxxgxyC(x)xxxxCyxxxxxCxxxC

Appendix B: HDD domains containing duplicated motifs

PF01437: Plexin PSI

A cysteine rich repeat found in several different extracellular receptors (J. Stamos, et al. (2004) Embo J, 23: 2325-35; J. P. Xiong, et al. (2004) J Biol Chem, 279: 40252-4). The function of the repeat is unknown. Three copies of the repeat are found in Plexin. Two copies of the repeat are found in mahogany protein. A related C. elegans protein contains four copies of the repeat. The Met receptor contains a single copy of the repeat. The Pfam alignment shows 6 highly conserved cysteine residues that may form three conserved disulphide bridges, whereas an additional two cysteines are observed at positions 5 and 7 and may be involved in forming a disulfide bond. Topology is 1-4_{—}2-8_{—}3-6_{—}5-7 (structure 1shy). Semaphorin (structure 1olz) contains only three disulfide bonds with topology 1-4_{—}2-6_{—}3-5. See FIG. 93.

1) CxxxxxCxxCxxxxxx(x)xCxxCxxxxxCxxxx(xxxxxx)xCxxxx(xxxxxxxxxx)xxxxxxC

2) CxxxxxCxxCxxxxxx(x)xCxWCxxxxxCxxxx(xxxxxx)xCxxxx(xxxxxxxxxx)xxxxxxC

The loop between Cys7 and Cys8 is very tolerant to insertions. For example, a hybrid domain is inserted between these cysteines in the integrin beta subunit structure (J. P. Xiong, et al. (2004) J Biol Chem, 279: 40252-4) and Cys8 still forms a disulfide bond with Cys2. This can be exploited to insert any sequence after Cys7.

Design: CxxxxxCxxCxxxxxx(x)xCxxCxxxxxCxxxx(xxxxxx)xCxxxxxxxx(xxxxx)(“anysequence”)C

This can be used to create multi-plexins:

First insertion: CxxxxxCxxCxxxxxx(x)xCxxCxxxxxCxxxx(xxxxxx)xCxxxxxxxx(xxxxx)(“PLEX”)C, where PLEX corresponds to CxxxxxCxxCxxxxxx(x)xCxxCxxxxxCxxxx(xxxxxx)xCxxxx(xxxxxxxxxx)xxxxxxC.

Second insertion: CxxxxxCxxCxxxxxx(x)xCxxCxxxxxCxxxx(xxxxxx)xCxxxxxxxx(xxxxx)(“PLEXIN” (“PLEXIN”))C, where (“PLEXIN” (“PLEXIN”)) corresponds to CxxxxxCxxCxxxxxx(x)xCxxCxxxxxCxxxx(xxxxxx)xCxxxx(xxxxxxxxxx)xxxxxxC inserted into CxxxxxCxxCxxxxxx(x)xCxxCxxxxxCxxxx(xxxxxx)xCxxxxxxxx(xxxxx)(“PLEX”)C after Cys7 of “PLEX”, and multiple following insertions into the inserted plexin sequence, after Cys7.

PF00088: Trefoil and Large Trefoil

A cysteine-rich module of approximately 45 amino-acid residues has been found in some extracellular eukaryotic proteins (M. D. Carr, et al. (1994) Proc Natl Acad Sci USA, 91: 2206-10; T. Yamazaki, et al. (2003) Eur J Biochem, 270: 1269-76). Human TFF3 can be expressed at high levels in the E. coli periplasm (15 mg/l culture). The module shows high disulfide density with 3 disulfide bonds per 45 amino acids and a topology of 1-5 2-4 3-6. Large trefoil consists of two adjacent modules linked by an additional disulfide bond with connectivity 1-14 2-6 3-5 4-7 8-12 9-11 10-13. The cysteine spacing between individual cysteines is smaller than 10 and therefore useful for library design. The cysteine positions are highly conserved among different members of this family. See FIGS. 94-95.

1) C(x)xxxxxxxxxCxx(x)xxxxxxxCxxxxCCxxxxx(x)xxxxxCx

2) C(x)xxxxxxRxxCxx(x)xxxxxxxCxxxxCCfxxxx(x)xxxxwCf

3) C(x)xxxxxxRxxCgx(x)xxitxxxCxxxgCC[fwy]dxxx(x)xxxxwC[fy]

Logo for large trefoil variant with two adjacent modules and an extra 1-14 disulfide linkage:

CxC(x)xxxxxxxxxCxx(x)xxxxxxxCxxxxCCxxxxx(x)xxxxxCxxxxxxxxxxxC(x)xxxxxxxxxCxx(x)xxxxxxxCxxx xCCxxxxx(x)xxxxxCxxxxxxxxC and derivatives.

FIG. 134 shows the repeated ‘Poly-Trefoil’ structures that can be created from Trefoil motifs.

PF00090: Thrombospondin 1

The module is present in the thrombospondin protein where it is repeated 3 times, in a number of proteins involved in the complement pathway as well as extracellular matrix protein. It has been shown to be involved in cell-cell interaction, inhibition of angiogenesis and apoptosis (P. Bork (1993) FEBS Lett, 327: 125-30). See FIG. 96.

The domain shows high disulfide density with 3 disulfide bonds per approximately 50 amino acids and a topology of 1-5_{—}2-6_{—}3-4 (T. M. Misenheimer, et al. (2005) J Biol Chem), The cysteine spacing between individual cysteines is smaller than 10 and therefore useful for library design. The cysteine positions are conserved among different members of this family.

CxxxCxxxxxxxxxxcxxxx(xxx)xxxxxCxxxxxx(xxx)xxxC(x)xxxxC

CxxxCxxGxxxRxxxcxxxx(Pxxx)xxxxxCxxxxxx(xxx)xxxC(x)xxxxC

CsvtCgxGxxxRxrxcxxxx(Pxxx)xxxxxCxxxxxx(xxx)xxxC(x)xxxxC

PF00228: Bowman Birk inhibitor

The Bowman-Birk inhibitor family is one of the numerous families of serine proteinase inhibitors. They have a duplicated structure and generally possess two distinct inhibitory sites. These inhibitors are primarily found in plants and in particular in the seeds of legumes as well as in cereal grains (R. F. Qi, et al. (2005) Acta Biochim Biophys Sin (Shanghai), 37: 283-92).

There are two different classes: 1) domains with 14 cysteines and the topology 1-14 2-6 3-13, 4-5 7-9 8-12 10-11 or domains with 10 cysteines and the topology 1-10 2-5 3-4 6-8 7-9. Due to these subfamilies, Cys positions in logo do not seem to be well conserved although they are for each subfamily.

The domain shows high disulfide density with 5 or 7 disulfide bonds per approximately 50 amino acids. The cysteine spacing between individual cysteines is smaller than 10 and therefore useful for library design. The cysteine positions are highly conserved among different members of this family. See FIGS. 97-98.

PF00184: Neurohypophysial hormones, C-terminal Domain

The nonapeptide hormones vasopressin and oxytocin are found in high concentrations in neurosecretory granules complexed in a 1:1 ratio with a class of disulfide-rich proteins known as neurophysins. Two closely related classes of NPs have been identified, one complexed with vasopressin and the other with oxytocin [L. Q. Chen, et al. (1991) Proc Natl Acad Sci USA, 88: 4240-4]. There are 75 members of this family and the cysteine positions are highly conserved. The cysteine-rich module is duplicated in the logo. See FIG. 99.

Both modules have homologous disulfide topology. One disulfide connects the two modules through Cys1 and Cys8. If this disulfide bond is ignored, disulfide topology for each module is 1-3, 2-6, 4-5. See FIG. 100.

The crystal structure of neurophysin revealed that one monomer consists of two homologous layers, each with four antiparallel beta-strands. The two regions are connected by a helix followed by a long loop. Monomer-monomer contacts involve antiparallel beta-sheet interactions, which form a dimer with two layers of eight beta-strands.

PF00200: Extendable and dimeric disintegrins

Disintegrins are peptides of about 50-80 amino acid residues that contain many cysteines all involved in disulphide bonds. Disintegrins contain an Arg-Gly-Asp (RGD) sequence, a recognition site of many adhesion proteins. The RGD sequence of disintegrins is postulated to interact with the glycoprotein IIb-IIIa complex.

Disintegrins are grouped according to length and cysteine content (J. J. Calvete, et al. (2005) Toxicon, 45: 1063-74).

Small: CxxxxCCxxCxxxxxxxxCxxxxxxxxx(xx)CxxxxCxC with 4SS and disulfide topology 1-4 2-6 3-7 5-8.

Medium: xCxxxxxxCCxxxxCxxxx(x)xxxCx(xxx)xxxCCxxCxxxxxxxxCxxxxxxxxxxxCxxxxxxxC

with 6SS and disulfide topology 1-5, 2-4, 3-8, 6-8, 7-11, 10-12.

Long:

xxxxxxxxxxCxCxxxxCxxxCCxxxxCxxxx(x)xxxCx(xxx)xxxCCxxCxxxxxxxxCxxxxxxxxxxxCxxxxxxxC with 7SS and disulfide topology 1-4, 2-7, 3-6, 5-11, 8-10, 9-13, 12-14

Dimeric: CCxxxxCxxxx(x)xxxCx(xxx)xxxCCxxCxxxxxxxxCxxxxxxxxxxxCxxxxxxxC with 4SS and disulfide topology 1-7,4-6, 5-10, 8-10 and two intermolecular SS involving Cys2 and Cys3 to yield dimeric integrins. See FIGS. 101 and 157. Evolutionary relationship between these different groups has been found, which is characterized by the loss/addition of disulfide bonds. Thus, this motif can be extended during in vitro evolution.

Appendix C: Scaffolds with highly repeated motifs

Cysteine-Rich Repeat Proteins (CRRPs)

PF00396: Granulin

Granulins are a family of cysteine-rich peptides of about 6 Kd which may have multiple biological activities (A. Bateman, et al. (1998) J Endocrinol, 158: 145-51). A precursor protein (known as acrogranin, for sequence see below) potentially encodes seven different forms of granulin (grnA to grnG) which are probably released by post-translational proteolytic processing. Granulins are evolutionary related to a PMP-D1, a peptide extracted from the pars intercerebralis of migratory locusts. See FIG. 103. Granulin spacing: CxxxxxxCxxxxxCCxxxxxxxxCCxxxxxxCCxxxxxCCxxxxxCxxxxxxCxx DBP: 1-3_{—}2-5_{—}4-7_{—}6-9_{—}8-11_{—}10-12

Design to expand the size (capping motif underlined; 1 repeat in italic, 1 repeat bold):

3C6C5CC8CC6CC5CC5CC8CC6CC5CC5C6C2

Design to introduce kinks: 3C6C5CCₐ4G3CC_{b}P5CC_{c}2G2CC_{d}P4C6C2

The natural 8-6-5-5 pattern or the more regular 5-5-5-5 pattern can be used. Since the structure has beta-sheets, one approach is to favor amino acids that are good beta-sheet formers and to avoid amino acids that are not beta-sheet formers. The following amino acids are preferred and can be obtained with mixed codons: valine, isoleucine, phenylalanine, tyrosine, tryptophan and threonine. FIG. 125 shows the Granulin structure.

Design assuming 5AA random loops:

3C6C5CC5CC5CC5CC5CC5CC5CC5CC5C6C2

Minimum starter protein has only two endcaps:

C6C5C6C (17 random AA)

Add minimum unit increase:

C6C5CC5C6C

Process steps: make library, pan, add randomized 5CC5 unit, pan, add 5CC5 unit, etc.

PF02420: Antifreeze Protein

Antifreeze protein is an 8 kDa protein forming a beta-helical structure (M. E. Daley, et al. (2002) Biochemistry, 41: 5515-25). An N-terminal capping motif is formed by a microprotein domain and 1-3 2-5 4-6 topology. Repeating units of 2C5C3 with disulfide connectivity 1-2 are added to this motif. Threonine is conserved because it is involved in ice binding, but can be omitted for design. Serine and Alanine are conserved because only small side chains fit inside the helix. The complete absence of a hydrophobic core is remarkable. FIG. 104 shows some Antifreeze-derived repeat proteins. FIG. 104 shows some motifs. See FIG. 127.

Natural sequence:

QCTGGADCTSCTGACTGCGNCPNA(VTCTNSQHCVKA)(NTCTGSTDCNT
A)(QTCTNSKDCFEA)(NTCTDSTNCYKA)(TACTNSSGCPGH)

The repeats are more clear when shown like this:

QCTGGADCTSCTGACTGCGNCPNA
(VTCTNSQHCVKA)
(NTCTGSTDCNTA)
(QTCTNSKDCFEA)
(NTCTDSTNCYKA)
(TACTNSSGCPGH)

Different designs (capping domain underlined; repeat italic):

1) 1C5C2C3C2C2C3(2C5C3)ₙ
2) 1C5C2C3C2C2C3(xtCxxxxCxxa)ₙ
3) QCTGGA(DCTSCTGACTGCG)(DCTSCTGACTGCG)ₙ
4) QCTGGA(DCTSCTGACTGCGA)(DCTSCTGACTGCGA)ₙ

PF00757: Furin-like domain

The furin-like cysteine rich region has been found in a variety of proteins from eukaryotes that are involved in the mechanism of signal transduction by receptor tyrosine kinases, which involves receptor aggregation. See FIG. 105.

A subset of the logo folds into a spiral-shaped repeat and is used as a scaffold for library design:

CxxxCxxxCxxxxxxCCxxxCxxxCxxxxxxxC. The topology of this motif is 1-3_{—}2-4_{—}5-7_{—}6-8. Members of this family show high conservation in their cysteine positions and spacing. This repeat can be extended by adding (CxxxCxxxCxxxxxxxC)ₙ to the C-terminus of the above motif.

PF03128: CxCxCx

This repeat contains the conserved pattern CXCXC where X can be any amino acid. The repeat is found in up to five copies in Vascular endothelial growth factor C. In the salivary glands of the dipteran Chironomus tentans, a specific messenger ribonucleoprotein (mRNP) particle, the Balbiani ring (BR) granule, can be visualized during its assembly on the gene and during its nucleocytoplasmic transport. This repeat is found over 70 copies in the balbiani ring protein 3 (see below). It is also found in some silk proteins.

The CXCXC repeat does not form disulfide bonds internally, as such a loop would only span three amino acids and no microprotein in the database has a cysteine span of 3. As shown in FIG. 109, cysteines in the CxCxCx motif are involved in the formation of a true repeat with disulfides linking different copies of the repeat. A single cysteine is typically found between CxCxCx repeats (conserved in logo, but position may vary). FIG. 106, 107, 108.

Actual: C10C1C1C8C10C1C1C8C10C1C1C3C10C1C1C6C11C

Abstracted, with beginning and end: C1C8C10C1C1C8C10C1C1C8C10C1

A model of disulfide bonded structure is show in FIG. 109.

PF05444: DUF753

Sequences which are repeated in several domains of unknown function in Drosophila.

FIG. 110.

PF01508: Paramecium

Surface antigen containing 37 copies of the above repeat. Structural role suggested. Secondary structure prediction suggests absence of alpha helices and presence of beta sheet structures. (don't know how this was done, presence of disulfides may interfere with prediction). FIGS. 111-112.

PF00526: Dicty

Several Dictyostelium species have proteins that contain conserved repeats. These proteins have been variously described as extracellular matrix protein B′, cyclic nucleotide phosphodiesterase inhibitor precursor′, prestalk protein precursor′, ‘putative calmodulin-binding protein CamBP64’, and cysteine-rich, acidic integral membrane protein precursor′ as well as ‘hypothetical protein’. See FIG. 113.

PF03860: DUF326

This family is a small cysteine-rich repeat. The cysteines mostly follow a CxxCxxxCxxCxxxCxxC pattern, though they often appear at other positions in the repeat as well. See FIG. 114.

PF02363: Cysteine-rich repeat

This Cysteine repeat CxxxCxxxCxxxC is repeated in sequences of this family, 34 times in O17970_CAEEL. The function of these repeats is unknown as is the function of the proteins in which they occur. Most of the sequences in this family are from C. elegans.

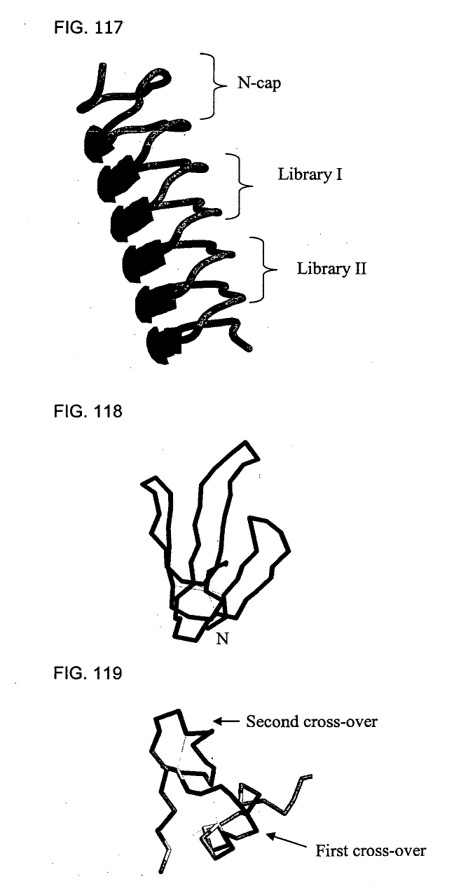

See FIG. 115-116.

| | Scaf- | | Randomiza- | Diver- | | |
|---|---|---|---|---|---|---|
| Name | fold | Cys | tion | sity | Size | Quality, % |
| LMP0020 | CB | 8 | 29 AA | 1027 | 2.6 × 107 | 78 |
| LMP0021 | CB | 8 | 29 AA | 1027 | 6.3 × 109 | 65 |
| LMS0040 | CB | 8 | 16 AA | 1019 | 2.9 × 108 | 77 |
| LMS0041 | CB | 8 | 16 AA | 1014 | na | Designed |
| LMP0040 | TF | 8 | 4 × 7 AA | 109 | na | Designed |
| LMB0030 | PL | 8 | 13 AA | 1012 | na | Designed |
| LMP0030 | PL | 8 | 8 AA | 109 | na | Designed |
| LMP0010 | TB | 6 | 23 AA | 1027 | 7.6 × 108 | 87 |
| LMS0043 | TB | 6 | 14 AA | 1018 | 5.1 × 109 | 92 |
| LMS0044 | TB | 6 | 14 AA | 1013 | 1.0 × 109 | 96 |
| LMB0020 | TI | 6 | 10 AA | 1012 | 2.4 × 109 | 92 |
| LMB0010 | BC | 4 | 12 AA | 1014 | na | Designed |
| LMP0050 | BC | 4 | 8 AA | 109 | 7.9 × 108 | 100 |

### REFERENCES

Artavanis-Tsokanas, S et al. (1995) Science 268:225-232.
Aster, J C et al. (1999) Biochemistry 38:4736.
Bensch K W et al. (1995) FEBS Lett 368:331-335.
Bork, P (1993) FEBS Lett 327:125-30
Carr, M D et al. (1994) PNAS 91:2206-2210.
Chirino A J, Ary M L, Marshall S A. (2004) Minimizing the immunogenicity of protein therapeutics. Drug Discovery Today 9:82-90
Chong J M et al. (2001) J. Biol. Chem. 277:5134-5144.
Chong, J M and Speicher, D W (2001) J. Biol. Chem. 276:5804-5813.
Conticello S G, Gilad Y, Avidan N, Ben-Asher E, Levy Z, Fainzilber M. (2001) Mechanisms for evolving hypervariability: the case of conopeptides. Mol Biol Evol. 18:120-31.
Cornet B et al. (1995) Structure 3:435-448.
DeA, et al. (1994) PNAS 91:1084-1088
Dufton M J (1984) J. Mol. Evol. 20:128-134.
Fajloun, Z et al. (2000) J. Biol. Chem. 275:39394-402.
Fitzgerald, K et al. (1995) Development 121:4275-82.
Gray W R et al. (1988) Annu Rev Biochem 57:665-700.
Guncar G et al. (1999) EMBO J. 18:793-803.
Hermeling S, Crommelin D J, Schellekens H, Jiskoot W. (2004) Structure-immunogenicity relationships of therapeutic proteins. Pharm Res. 21, 897-903
Higgins, J M et al. (1995) J. Immunol. 155:5777-85
Hoffman, W et al. (1993) Trends Biochem Sci 18:239-243.
Hugli, T E (1990) Curr Topics Microbiol Immunol. 153:181-208.
Jonassen I et al. (1995) Protein Sci 4:1587-1595.
Karrikubo, Y et al. (2004)
Kim, J I et al. (1995) J. Mol. Biol. 250:659-671.
Kimble, J et al. (1997) Annu Rev Cell Dev Biol 13:333-361.
Koduri, V & Blacklow, S C (2001) 40:12801
Lauber, T. et al. (2003) J. Mol. Biol. 328:205-219.
Léonetti et al. (1998) J Immunol, 160; 3820-3827 (1998)
Léonetti M, That R, Cotton J, Leroy S, Drevet P, Ducancel F, Boulain J C, Ménez A. (1998) Increasing immunogenicity of antigens fused to Ig-binding proteins by cell surface targeting. J. Immunol., 160; 3820-3827.
Leung-Hagesteijn, C et al. (1992) Cell 71:289-99
Liu L et al. (1997) Genomics 43:316-320.
Maillére B, Mourier G, Hervé M, Cotton J, Leroy S, Ménez A. (1995) Immunogenicity of a disulphide-containing neurotoxin: presentation to T-cells requires a reduction step. Toxicon, 4, 475-482; Maillére B. et al., unpublished data.
Maillére, B., Cotton, J., Mourier, G., Léonetti, M., Leroy, S. and Ménez, A. (1993). Role of thiols in the presentation of a snake toxin to murine T cells. J. Immunol. 150:5270-5280.
Martin L, Stricher F, Misse D, Sironi F, Pugniere M, Barthe P, Prado-Gotor R, Freulon I, Magne X, Roumestand C, Ménez A, Lusso P, Veas F, Vita C (2003) Rational design of a CD4 mimic that inhibits HIV-1 entry and exposes cryptic neutralization epitopes. Nat. Biotechnol. 21:71-6.
Ménez, A. (1991) Immunology of snake toxins, p. 35-90. In: Snake Toxins. AL Harvey (Ed), Pergamon Press, Inc., New York.
Miljanich, G, P. (2004), Ziconotide: neuronal calcium channel blocker for treating severe chronic pain. Curr. Med. Chem. 23, 3029.
Misenheimer, T M et al. (2001) J. Biol. Chem. 276:45882
Molina F et al (1996) Eur. J. Biochem. 240:125-133.
Mourier et al., (1995) Toxicon 4:475-482.
Nielsen, K J et al. (2002) J. Biol. Chem.277:27247-27255.
Pallaghy P K et al. (1993) J Mol Biol 234:405-420.
Pallaghy, P et al. Protein Sci 3:1833 (1994)
Pan, T C et al. (1993) J. Cell. Biol. 123: 1269-1277
Patten, P. A. and Schellekens, H. (2003) The immunogenicity of Biopharmaceuticals. In: Immunogenicity of Therapeutic Biological Products. Brown, F. and Mire-Sluis, A. R. (eds). Dev. Biol. Basel, Karger, 112:81-97.
Pereira, C. M., Guth, B. E. C, Sbrogio-Almeida, M. E. and Castilho, B. A. (2001) Microbiology 147:861-867.
Petersen, S V et al. (2003) Proc. Natl. Acad. Sci. USA 100:13875-80.
Rebayl, et al. (1991) Cell 67:687-699
Roszmusz, E. et al. (2002) BBRC296:156
Sands, B E & Podolsky, D K (1996) Annu. Rev. Physiol. 58:253-273.
Schultz-Chemy, S et al. (1995) J. Biol. Chem. 270:7304-7310
Schultz-Chemy, S et al. J. (1994) J. Biol. Chem. 269:26783-8
Schulz A. et al. (2005) Biopolymers 80:34-49.
Singh H, Raghava G P (2001) ProPred: prediction of HLA-DR binding sites. Bioinformatics 17: 1236-7.
Skinner W S et al., J. Biol. Chem. (1989) 264:2150-2155.
So, T., Ito, H., Hirata, M., Ueda, T. and Imoto, T. (2001) Contribution of conformational stability of hen lysozyme to induction of type 2 T-helper immune responses. Immunology 104:259-268.
Sturniolo, T., et al. (1999) Generation of tissue-specific and promiscuous HLA ligand databases using DNA microarrays and virtual HLA class II matrices. Nature Biotechnol, 17: 555
Tam, J P and Lu, Y A. Protein Sci. 7:1583 (1998)
Tax, F E et al. (1994) Nature 368:150-154.
Thai R, Moine G, Desmadril M, Servent D, Tarride J L, Ménez A, Léonetti M. (2004) Antigen stability controls antigen presentation. J. Biol. Chem. 279, 50257-50266.
Van den Hooven, H W et al. (2001) Biochemistry 40:3458-3466.
van Vlijmen H W, Gupta A, Narasimhan S, Singh J (2004). A novel database of disulfide patterns and its application to the discovery of distantly related homologs. J Mol Biol 335: 1083-92.
Vardar, D et al. (2003) Biochemistry 42:7061
White, C E et al. (1996) PNAS 93:10177.
Xu Y et al. (2000) Biochemistry 39:13669-13675.
Zaffarella G C et al. (1988) Biochemistry 27:7102-7105.
Zhu S et al. (1999) FEBS Lett 457:509-514.
Zuiderweg, E R et al. (1989) Biochemistry 28:172-85.

What is claimed is:

1. A non-naturally occurring cysteine (C)-containing protein comprising a polypeptide having no more than 35 amino acids, in which
at least 10% of the amino acids in the polypeptide are cysteines,
at least two disulfide bonds are formed by pairing intra-scaffold cysteines, and wherein said pairing yields a complexity index greater than 3.

2. A non-naturally occurring cysteine (C)-containing protein comprising a polypeptide having no more than about 60 amino acids, in which
at least 10% of the amino acids in the polypeptide are cysteines,
at least four disulfide bonds are formed by pairing cysteines contained in the polypeptide, and wherein said pairing yields a complexity index greater than 4.

3. The non-naturally occurring cysteine (C)-containing protein of claim 1 or 2, wherein the complexity index greater than 6.

4. The non-naturally occurring cysteine (C)-containing protein of claim 1 or 2, wherein the complexity index greater than 10.

5. The non-naturally occurring cysteine (C)-containing protein of claim 1 or 2 that binds specifically to a target molecule.

6. The non-naturally occurring cysteine (C)-containing protein of claim 1 or 2 that remains the target binding capability after being heated to a temperature higher than about 50° C.

7. The non-naturally occurring cysteine (C)-containing protein of claim 1 or 2 that remains the target binding capability after being heated to a temperature higher than about 80° C.

8. The non-naturally occurring cysteine (C)-containing protein of claim 1 or 2 that remains the target binding capability after being heated to a temperature higher than about 100° C. and for more than 0.1 second.

9. The non-naturally occurring cysteine (C)-containing protein of claim 1 or 2 that is conjugated to a moiety selected from the group consisting of labels, effectors, antibodies, and half-life extending moieties.

10. The non-naturally occurring cysteine (C)-containing protein of claim 1 or 2 being a monomer.

11. The non-naturally occurring cysteine (C)-containing protein of claim 1 or 2 being a multimer.

12. The non-naturally occurring cysteine (C)-containing protein of claim 1 or 2, wherein the protein comprises one type of scaffold.

13. The non-naturally occurring cysteine (C)-containing protein of claim 1 or 2, wherein the protein comprises more than one type of scaffold.

14. The non-naturally occurring cysteine (C)-containing protein of claim 1 or 2, wherein the protein comprises a target binding site and half-life extension moiety.

15. The non-naturally occurring cysteine (C)-containing protein of claim 1 or 2, wherein the protein comprises repeating units that bind to the target.

16. The non-naturally occurring cysteine (C)-containing protein of claim 1 or 2, wherein the protein comprises a half-life extension moiety selected from the group consisting of serum albumin, IgG, erythrocytes, and proteins accessible to the serum.

17. The non-naturally occurring cysteine (C)-containing protein exhibiting binding specificity towards a target distinct from the native target of the corresponding naturally-occurring cysteine (C)-containing protein or scaffold.

18. A non-natural protein containing a single domain of 20-60 amino acids which has 3 or more disulfides and binds to a human serum-exposed protein, and wherein said protein has less than 5% aliphatic amino acids.

19. A non-naturally occurring protein containing a single domain of 20-60 amino acids which has 3 or more disulfides and binds to a human serum-exposed protein, wherein said protein has a score in the T-Epitope program that is less than 90% of the average for proteins in the database.

20. A library of the non-naturally occurring protein of claim 1, 2, 18 or 19.

21. A genetic package displaying the library of claim 20.

22. A method of detecting the presence of a specific interaction between a target and an exogenous polypeptide that is displayed on a genetic package, the method comprising:
(a) providing a genetic package displaying of claim 20;
(b) contacting the genetic package with the target under conditions suitable to produce a stable polypeptide-target complex; and
(c) detecting the formation of the stable polypeptide-target complex on the genetic package, thereby detecting the presence of a specific interaction.

23. The method of claim 22 further comprising the step of isolating the genetic package that displays a polypeptide having the desired property.

24. A pharmaceutical composition comprising the non-naturally occurring cysteine (C)-containing protein of claim 1 or 2 and a pharmaceutically acceptable carrier.
